(19) United States
(12) Patent Application Publication
(10) Pub. No.: US 2020/0032347 A1
(43) Pub. Date: Jan. 30, 2020

(54) RNA profiling for individualized diet and treatment advice.

(71) Applicant: Stichting Katholieke Universiteit, Nijmegen (NL)

(72) Inventor: Wilhelmus Petrus Johannes Leenders, Nijmegen (NL)

(73) Assignee: Stichting Katholieke Universiteit, Nijmegen (NL)

(21) Appl. No.: 16/491,165

(22) PCT Filed: Mar. 7, 2018
(86) PCT No.: PCT/EP2018/055548

§ 371 Date: Sep. 5, 2019

(30) Foreign Application Priority Data
Mar. 7, 2017 (EP) 17159630.7

(51) Int. Cl. C12Q 1/6886

(52) U.S. Cl. CPC C12Q 1/6886; C12Q 2600/158; C12Q 2600/156

(57) ABSTRACT

The present invention relates to the field of medicine and molecular diagnostics. In particular, it relates to a novel RNA next generation sequencing-based profiling assay allowing simultaneous detection of transcripts and alternative splice variants thereof and mutations therein, from genes involved in disease. including genes involved in metabolism, resulting in an advice for personalized treatment with drugs targeting disease-associated molecular aberrations in combination with dietary compounds, food supplements or inhibitors of metabolism

### FIELD OF THE INVENTION

The present invention relates to the field of medicine and molecular diagnostics. In particular, it relates to a novel RNA profiling assay allowing simultaneous detection of inter alia transcripts and alternative splice variants thereof and mutations therein, from genes involved in disease, including genes involved in metabolism, resulting in a guidance for personalized treatment with drugs targeting disease-associated molecular aberrations, optionally in combination with dietary compounds, food supplements or inhibitors of metabolism.

### BACKGROUND OF THE INVENTION

Malfunctioning cells are typically distinct from healthy cells in that they have altered metabolism. As an example, cells in diabetic patients have adapted to cope with lack of glycogenesis and high extracellular glucose concentrations. In another example, aberrantly growing cells such as in hyperplasia and in cancer need to process excessive amounts of nutrients to produce nucleotides, amino acids and fatty acids for DNA/RNA synthesis, protein synthesis and membrane synthesis. To accommodate this demand, growing cells have adapted by an altered metabolism. There is a number of compounds that can serve as fuel for malfunctioning cells. These include glucose, fatty acids and amino acids, such as glutamine and glutamate. A selection of genes that are involved in cell metabolism are presented in Table I here below. It should be noted that genes involved in a metabolic pathway may also be involved in another metabolic pathway; the person skilled in the art is aware of this.

TABLE I
Selection of genes that are involved in cell metabolism
Glucose processing (GLY1: glucose to pyruvate; PPP: pentose phosphate pathway: GLY2:
pyruvate to lactate; TCA: tricarboxylic acid cycle)

| | |
|---|---|
| 1. | Transmembrane glucose transporters GLUT1 and GLUT3 (SLC2A1 and SLC2A) to ensure |
| | glucose import into the cytosol |
| 2. | Hexokinase (HK1, 2, 3), to convert glucose to glucose-6-phosphate (GLY1) |
| 3. | Glucose-6-phosphate dehydrogenase (G6PD) to convert glucose-6-phosphate to 6- |
| | phosphogluconolactone (PPP) |
| 4. | Gluconolactonase to convert 6-phosphogluconolactone to 6-phosphogluconate (PPP) |
| 5. | 6-phosphogluconate dehydrogenase (PGD) to convert 6-phosphogluconate to ribulose-5- |
| | phosphate (PPP) |
| 6. | ribulose-5-phosphateisomerase (RPIA) to convert ribulose-5-phosphate to ribose-5- |
| | phosphate (PPP) |
| 7. | ribulose-5-phosphate 3-epimerase (RPE) to convert ribulose-5-phosphate to xylulose-5- |
| | phosphate (PPP) |
| 8. | Transketolase (TKT) to convert products of step 5 and step 6 to glyceraldehyde-3- |
| | phosphate and sedoheptulose 7-phosphate (PPP) |
| 9. | Transaldolase to convert the products of step 8 to fryctose-6-phosphate and erythrose 4- |
| | phosphate (PPP) |
| 10. | Phosphoglucose isomerase (PGI) to convert glucose-6-hosphate to fructose 6-phosphate |
| | (GLY1) |
| 11. | Phosphofructokinase (PFK) to convert fructose-6-phosphate to fructose 1,6-biphosphate |
| | (GLY1) |
| 12. | Fructose bisphosphate aldolase (ALDOA) to convert fructose 1,6-biphosphate to |
| | dihydroxyacetone phosphate and glyceraldehyde-3-phosphate (GLY1) |
| 13. | Glyceraldehyde 3 phosphate dehydrogenase (GAPDH) to convert glyceraldehyde-3- |
| | phosphate to 1,3-biphosphoglycerate (GLY1) |
| 14. | Phosphoglycerate kinase (PGK) to convert 1,3-biphosphoglycerate to 3-phosphoglycerate |
| | (GLY1) |
| 15. | Phosphoglycerate mutase (PGAM1/2) to convert 3-phosphoglycerate to 2- |
| | phosphoglycerate (GLY1) |
| 16. | Enolase (ENO) to convert 2-phosphoglycerate to phosphoenolpyruvate (GLY1) |
| 17. | Pyruvate kinase (PKM1/2) to convert phosphoenolpyruvate to pyruvate (GLY1) |
| 18. | Pyruvate dehydrogenase (PDH) to convert pyruvate to Acetyl-CoA (TCA) |
| 19. | Pyruvate carboxylase (PC) to convert Acetyl-CoA to oxaloacetic acid (TCA) |
| 20. | Citrate synthase (CS) to produce citrate from Acetyl-CoA and oxaloacetic acid (TCA) |
| 21. | Acotinase (ACO1) to convert citrate to cis-acotinate and isocitrate (TCA) |
| 22. | Isocitrate dehydrogenase 1/2/3 (IDH1/2/3) to convert isocitrate to αKG (TCA) |
| 23. | αKG dehydrogenase (OGDH) to convert αKG to succinyl-CoA (TCA) |
| 24. | Succinyl-CoA synthetase (SUCLA2) to convert succinyl-CoA to succinate (TCA) |
| 25. | NADH-coenzyem Q oxidoreductase (OXPHOS) |
| 26. | Succinate-Q-oxidoreductase (OXPHOS) |
| 27. | Flavoprotein_Q oxidoreductase (OXPOS |
| 28. | Cytochrome C oxidase (OXPHOS) |
| 29. | ATP synthase (OXPHOS) |
| 30. | Succinate dehydrogenase (SDHA/B/C/D) to convert succinate to fumarate (TCA) |
| 31. | Fumarate hydratase (FH) to convert fumarate to malate (TCA) |
| 32. | Malate dehydrogenase (MDH1/2) to convert malate to oxaloacetate (TCA) |
| 33. | Pyruvate dehydrogenase kinase (PDK), to phosphorylate and block PDH (step 11) (GLY2) |
| 34. | Lactate dehydrogenase (LDHA) to produce lactate from pyruvate (GLY2) |
| 35. | Lactate dehydrogenase (LDHB) to convert lactate to pyruvate. |
| 36. | Monocarboxylate transporters MCT1 (SLC16A1) and MCT4 (SLC16A3) to transport lactate |
| | and associated protons from the cell, to regulate pH homeostasis |
| 37. | Carbonic anhydrases (CA9 and CA12) to produce HCO₃— from H₂O and CO₂ at the cell |
| | surface |
| 38. | HCO₃— importer (SLC4A10) to import HCO₃— for pH homeostasis |

Glutamine processing
There is a number of cells that also depend on the amino acid glutamine for proliferation. Genes
that are involved in glutamine metabolism include

| | |
|---|---|
| 39. | SLC1A5 or ASCT2, an membrane importer protein for glutamine |
| 40. | Glutaminase (GLS) to convert glutamine to glutamate |
| 41. | Glutamate dehydrogenase (GLUD1/2) to convert glutamate to alpha-ketoglutarate (αKG) |
| 42. | Branched chain amino acid transferase 1 and 2 (BCAT1/2) to produce glutamate from αKG |
| 43. | Excitatory amino acid transporter EAAT2 (SLC1A2) to import glutamate into the cell |
| 44. | System Xc₋ (SLC7A11) to export glutamate from the cell in exchange for cystin |

Fatty acids
Fatty acids are important building blocks for cells because they are the basis for synthesis of
phospholipid bilayers that make up membranes for nuclei, mitochondria, endoplasmic reticulum, golgi
apparatus and lysosomes and peroxisomes. Enzymes that are involved in fatty acid anabolism include

| | |
|---|---|
| 45. | CIC (SLC25A1) to transport citrate from mitochondria to cytosol |
| 46. | ATP citrate lyase (ACLY) to convert citrate to oxaloacetate and acetyl-CoA |
| 47. | Acetyl CoA carboxylase (ACACA, ACACB) to convert acetyl-CoA to malonyl-CoA |
| 48. | Fatty acid synthase (FASN) to convert Acetyl-CoA, malonyl-CoA and NADPH to palmitate |
| 49. | Fatty acid transporter (CPT1) for uptake of fatty acids |
| 50. | choline transporter (SLC5A7) to import choline |
| 51. | Choline kinase (CHKA) to convert choline to phosphatidylcholine |
| 52. | Carnitine palmitoyltransferase 2 (CPT2) to convert acylcarnitine to long chain Acyl-CoA |
| 53. | Acyl CoA dehydrogenase (VLCAD) to convert long chain acyl-CoA to 2-Enoyl-CoA |
| 54. | Trifunctionalportein (HADHA/B) to convert 2-Enoyl-CoA to medium and short chain Acyl- |
| | CoA |
| 55. | Acyl CoA dehydrogenase (SCAD, MCAD, LCAD) to convert cyl-CoA to 2-Enoyl CoA |
| 56. | 2-Enoyl-VoA hydratase to convert 2-Enoyl-CoA to 3-hydroxyacyl-CoA |
| 57. | 3-hydroxyacyl-CoA dehydrogenase (SCHAD) to convert 3-hydroxyacyl-CoA to 3-ketoacyl |
| | CoA |
| 58. | 3-ketoacyl-CoA thiolase (MCKAT) to convert 3-ketoacyl-CoA to acetyl-CoA |

### Metabolic Alterations in Cancer

Altered metabolism may be a result of cancerous transformation of cells, but for a number of cancer types it is also a cause of cancer. A well-known example of metabolic alterations (alterations within one or more metabolic pathway) resulting from cancer growth is the alterations that are a consequence of hypoxia, the lack of oxygen that occurs in growing tissues that have outgrown the vascular blood supply. Under oxygenated conditions, the transcription factors Hypoxia Inducible Factors HIF1α and HIF2α are hydroxylated by the oxygen-dependent enzyme proline hydroxylase (PHD). Proline-hydroxylated HIFs have binding sites for the VHL-E3 ubiquitin complex, resulting in HIF ubiquitinylation and proteasomal breakdown. This pathway is an important regulator of HIF-levels in cells. Under normoxic conditions glucose will be converted to pyruvate that will be processed to acetyl-CoA which enters the mitochondria for processing in the tricarboxylic acid (TCA) cycle. The TCA cycle is directly coupled to oxidative phosphorylation and yields for every mole of glucose the energy equivalent of 36 moles of ATP, CO₂ and H₂O. Full processing of glucose via this pathway does not yield carbon building blocks.

Under hypoxic conditions PHDs are inactive and as a result unhydroxylated HIF1/2a will accumulate in cells, heterodimerize with HIF-13 (ARNT) and activate genes that are needed to survive hypoxia. These genes (Table I, steps 1-38) regulate different processing of glucose, using pyruvate for lactate instead of acetyl CoA production. Conversion of pyruvate to lactate yields only 2 moles of ATP for every mole of glucose. The inefficiency of this process in terms of energy production requires extra intake of glucose, which is accomplished by increased expression of glucose transporters (GLUT1, GLUT3). Genes involved in the next steps of glucose processing are also activated (especially hexokinase 2). HIF accumulation also results in activation of the gene encoding vascular endothelial growth factor (VEGF-A), resulting in an angiogenic response.

Glycolysis in cancer is not restricted to hypoxic areas but can also occur under normoxic conditions. There is a number of causes for glycolysis in normoxic cancers, for example elevated expression of the Myc oncogene [resulting in activation of PDK (Table I, step 33) and preventing influx of acetyl-CoA into the TCA cycle], decreased function of tumor suppressors (VHL, PTEN) and elevated activity of oncogenic pathways (e.g. PI3K, AKT) all leading to increased HIF activity. Aerobic glycolysis in cancers is known as the Warburg effect.

Whereas aberrations in oncogenes and tumor suppressor genes in a cancer and conditions such as hypoxia induce metabolic alterations, these alterations depend on the specific nature of the molecular aberrations. As a consequence each tumor has its own specific metabolic demands.

Adding an extra level of complexity, instead of being a consequence of carcinogenesis, altered metabolism may also drive carcinogenesis. Hotspot mutations in isocitrate dehydrogenase 1 and 2 (Table I, step 22) in substantial percentages of diffuse gliomas of the brain, acute myeloid leukemia, chondrosarcomas and hepatic cholangiocarcinomas result in consumption of alpha ketoglutarate (α-KG) and NADPH to produce the oncometabolite D-2-hydroxyglutarate (D-2HG) that can accumulate to milliMolar concentrations. The small difference between the chemical structures of α-KG and D-2HG in combination with the high concentrations of the latter, results in competitive displacement of α-KG from α-KG-dependent enzymes that subsequently cannot function properly. Important examples are the Ten Eleven Translocation (TET)-family of enzymes that are involved in demethylation of CpG islands in the DNA, and JmJ proteins that are involved in histone demethylation. Consequently IDH-mutated cancers present with hypermethylated CpG islands and histones, resulting in deranged gene transcription profiles. Of importance, the consumption of NADPH in IDH-mutated cancer cells results in low levels of reduced glutathione and decreased resistance to reactive oxygen species (ROS). The increased activity of ROS in IDH mutated cancer cells may increase the chance of second hits in oncogenes and tumor suppressor genes, and result in cancer. Except for the known hotspot mutation that leads to 2-HG production, IDH-mutants have been described that are defective in NADPH production, but do not produce D-2HG (1).

Other examples of cancers in which mutations in metabolic enzymes are cancer drivers are phaeochromocytomas and paragangliomas, that carry inactivating mutations in one of the SDH subunits A-D (Table I, step 30) or in SDH assembly factor SDHAF2 (2). These mutations can be hereditary, leading to the HPGL/PCC syndrome, or somatic. Other mutations in metabolic genes that cause cancer occur in FH (Table I, step 31). Mutations in FH are associated with leyomyomatosis and papillary renal cell cancer (3). Mutations in VHL protein occur in clear cell renal cell cancers, and directly result in glycolysis via a defect in HIF breakdown, as described above. The IDH, SDH and FH genes can therefore be considered tumor suppressor genes.

Dietary compounds, food supplements or safe to use drugs exist that can inhibit metabolic pathways, and the use of such drugs have been considered as potentially beneficial for the treatment of cancer. Examples are deoxyglucose, inhibiting glucose uptake by the cell and preventing glycolysis (Table I, step 1 and following) (4), 3-bromopyruvate, blocking the activity of hexokinases (Table I, step 2 and following) (5, 6), 6-amino-niocotinamide (6-AN, blocking G6PD in the pentose phosphate pathway, Table I, step 3 (7)), metformin, blocking OXPHOS (8), bis-2-(5-phenylacetamido-1,2,4-thiadiazol-2-yl)ethyl sulfide (BPTES, blocking glutaminase, Table I, step 28), epigallo-3-catechin gallate (EGCG, blocking glutamate dehydrogenases and other NADPH-generating enzymes (Table I, step 41) and fatty acid synthase Table I, step 36 (9)) (10), cerulenin, inhibiting fatty acid synthase (Table I, step 48) (9). These inhibitors have been shown to have anti-tumor effects in different models of cancers.

Although these compounds have also been tested in humans, anti-cancer effects may require systemic concentrations that are not tolerated by healthy tissues. At non-toxic concentrations, treatment with metabolic inhibitors below maximal tolerated doses can however augment the activity of other treatments, such as radiotherapy, chemotherapy or targeted therapy.

Current treatment protocols for patients with cancers who cannot be cured by surgery are ineffective in that cancers generally develop resistance to treatment (11-14).

There is therefore a great need for safe and adjuvant treatments that increase the efficacy of the state of the art therapies. These state of the art therapies are applied according to guidelines that are based on the outcomes of phase III clinical trials. For some cancer types, the effects of treatment can be predicted (e.g. colon cancers with KRAS mutations do not respond to EGFR inhibitors, cancers with the BRAF-V600E mutation develop resistance to the BRAF inhibitor vemurafenib by upregulating signaling from EGFR, gliomas with hypermethylation of the DNA repair gene MGMT respond better to the DNA alkylating chemotherapy Temozolomide). Although some cancer types are now routinely analyzed for so called companion biomarkers-biomarkers based on which a personalized treatment can be initiated—analyses on such markers cannot be performed if tissue cannot be made available, such is e.g. the case in patients with inoperable cancer.

Therefore there is an urgent need for a test that measures parameters in a patient (subject) that are relevant for treatment decision making, with treatment protocols consisting of the most appropriate metabolic inhibitors, combined with the most appropriate available targeted drugs or radiotherapy or chemotherapy. Currently available tests to investigate metabolism in cancer include magnetic resonance spectroscopic imaging (MRSI), but this is a technically challenging technique that can only be performed in specialized centers and requires concomitant in depth knowledge of MR principles and cell biology. Furthermore, MRSI can only measure a limited number of metabolites. An alternative method to investigate metabolism in cancer is to make extracts of metabolites of a cancer and perform mass spectroscopy. In this case, results will be influenced by the fact that these assays are performed on tissues that have seen hypoxia after surgery.

Molecular diagnosis of cancer is currently performed by analysis of tumor DNA and aims for detection of actionable mutations. DNA analyses allow the identification of mutations and variations in metabolic enzymes such as FH, SDH and IDH, and actionable mutations and amplifications in oncogenes and tumor suppressor genes. DNA analyses can be performed using whole genome analyses or whole exome analyses but can also be performed with Molecular Inversion Probes as described in the literature (15). The technique is depicted in FIG. 1. MIPs inversely hybridize to a DNA of interest via an extension probe and a ligation probe that are connected by a backbone sequence, leaving a small gap on the target sequence. This gap is enzymatically filled and ligated, leaving a circular molecule that can be purified by exonuclease-based degradation of non-circularized nucleotide strands. PCR-based amplification of the filled gap using oligonucleotide primers in the backbone generates a library of amplicons that can be analyzed using e.g. next generation sequencing methodology. To make the assay quantitative, a unique molecular identifier (UMI) of e.g. 8 random nucleotides can be incorporated in the MIP, to allow a back calculation of all PCR products with the same UMI to one MIP. The chance of 2 different smMIPs having the same UMI is (1/4)⁸=1:65,536 which makes these UMIs unique for each MIP. MIPs with a UMI are called single molecule MIPs or smMIPs (16). The technique of smMIPs for analyses of DNA sequences is described in the literature.

DNA analyses cannot measure gene activity, e.g. activity of metabolic genes, which is regulated by epigenetic processes and the presence of transcription factors and transcription repressors (17).

### DESCRIPTION OF THE FIGURES

FIG. 1 Principle of smMIP-based targeted RNA sequencing. The procedure depends on the hybridization of molecular inversion probes consisting of a ligation and an extension probe that are connected via a backbone sequence. Capture hybridization leaves for each smMIP a gap of 112 nt that is enzymatically extended and closed by ligation. After exonuclease digestion of non-ligated probes the remaining library of circularized smMIPS is PCR-amplified with primers in the smMIP backbone. The ligation probe is flanked by a random 8N unique molecular identifier (UMI) sequence that allows correction for PCR duplicates. During PCR, for each sample a unique barcode primer is used allowing identification of sample-specific reads.

FIG. 2 A,B Inegrative Genome Viewer (IGV) representation of the VHL locus of SKRC7 and SKRC7-VHL^{HA} cells. BAM files containing whole RNAseq data from these cell lines were loaded into IGV. Note the CAA-UAA mutation, resulting in the VHL^{Q132-stop} mutation at the protein level. C and D show SeqNext representations of the same VHL locus of SKRC7 (C) and SKRC7-VHL^{HA} cells. E) bar graph showing VHL-related TPM and FPM values of SKRC7 and SKRC7-VHL^{HA}. F) Western blot of SKRC7 cells and the VHL-expressing derivative, stained with an anti-HA antibody.

FIG. 3 smMIP-based targeted RNA sequencing correlates well with whole transcriptome RNAseq. Mean smMIP-based metabolic FPM levels (A,C) and tyrosine kinase transcript FPM levels (B,D) were plotted to TPM levels of the same transcripts, extracted from whole RNAseq data. Note that the transcripts with very low FPM values (10⁻²FPM) were not detected in the RNAseq dataset. We included these transcripts in these analyses although they may have lowered the Pearson coefficient.

FIG. 4 SmMIP-based targeted RNAseq reveals decreased expression levels of glycolysis related genes a.o. SLC2A1, CA9, HK2 and LDHA in two independent duplicate experiments (A,B). Relative values were comparable to those obtained from whole transcriptome RNA seq analysis (C), which is in agreement with the correlation shown in FIG. 3. Differences in expression levels were validated on the protein level for HK2 and CA9, using tubulin as housekeeping control (D).

FIG. 5 smMIP-based targeted RNA next generation sequencing can be used for adequate variant calling. Shown are the loci containing the IDH1-R132H mutation in E478 xenografts (A) and in a clinical grade III astrocytoma (C, this mutation was confirmed by genetic analysis), whereas the IDH1-R314C mutation in E98 cells could also be identified (B).

FIG. 6 Example of smMIP analysis of RNAs encoding metabolic enzymes in two different ccRCC cell lines.

FIG. 7 smMIPs allow specific detection of splice variants. The Mel57 cell line that does not express endogenous VEGF-A, was transfected with expression plasmids pIRESneo-VEGF-A121 and pIRESneo-VEGF-A165, and cultured in medium containing neomycin to generate stable transfectants. RNA from these transfectants were subjected to smMIP profiling with a panel of smMIPs, among which smMIP121 with ligation and extension probes in exons 5 and 8 of the VEGF-A transcript, respectively, hence detecting only VEGF-A121, and smMIPs with ligation and extension probes in exons 5 and 7 of the VEGF-A transcript respectively, hence detecting only VEGF-A165. Note that smMIP121 detects VEGF-A121, but not VEGF-A165, and smMIP165 detects only VEGF-A165, but not VEGF-A121.

FIG. 8 smMIP-based targeted RNA next generation sequencing can be used for adequate diagnosis. Shown is the IDH locus containing the IDH1-R132H mutation in a clinical grade III astrocytoma. Analysis of the tyrosine kinase transcriptome reveals high expression levels of the genes encoding the tyrosine kinases NTRK2 and PDGFRA in this tumor, suggestive of responsiveness to the corresponding tyrosine kinase inhibitors.

FIG. 9 smMIP based detection of EGFR splice variants in gliomas. Shown is that in the group of gliomas there is elevated expression of EGFR in 39/75 brain tumors (52%; mean FPM 738 in positives vs mean FPM 35 in negatives, using an arbitrary cut off FPM value of 100) and expression of EGFRvIII in 12/75 brain tumors (16%; mean FPM 642 in positives vs mean FPM 0.27 in negatives, using an arbitrary cut-off value of 6).

FIG. 10 smMIP based targeted RNA sequencing can be used for accurate diagnosis and prognosis.

A) Heat map of the individual gene profiles. Unsupervised agglomerative clustering of log-transformed expression levels of the targeted genes of interest was performed. Agglomerative clustering was performed according to WardD2 method by calculating Manhattan distance between individual profiles using bio-informatic R-software scripts.
B) Kaplan-Meier curve displaying the overall survival data of the computer-generated groups A and B of the heat-map in panel a). The results show that groups A and B have different survival with high significance (Fisher's exact test; p<0.0001), demonstrating that this test has high prognostic value in gliomas. Groups A and B are here annotated as IDH-MT and IDH-WT.
C) heterozygous IDH1R132H detection in one of the samples, in this case with 38% of transcripts being from the mutant allele and 62% of transcripts from the wt allele
D) Subgroup analysis of IDH-wild-type patients with very poor survival (OS<12 months) versus IDH-wild-type patients with better prognosis (OS>14 months) showed that high expression levels of carbonic anhydrase 12 are associated with poor prognosis (p<0.001; Fisher's exact test, see Kaplan-Meier curve in D).

FIG. 11 Immunhistochemistry of tumors with high and low PSMA transcript levels. Blood vessel expression of PSMA protein is observed in blood vessels from tumors with high transcript levels and not in tumors with low transcript levels (see FPM values under the different photographs.

FIG. 12 Tyrosine kinase profiles predict sensitivity and non-sensitivity to targeted therapies in vitro. A) the astrocytoma cell line E98 expresses similar levels of MET as the renal cancer cell line SKRC17 depicted in (B). C) However, in contrast to E98 cells, SKRC17 cells do not respond to compound A with decreased proliferation rates. D) Profiling of membrane tyrosine kinases reveals that within the selected group of membrane tyrosine kinases that are measured in the assay, MET is the only one expressed by E98, whereas SKRC17 cells express an additional number of other tyrosine kinase inhibitors, including AXL, EGFRs, FGFRs.

FIG. 13 HPV RNA profiling. Profile of 29 gynecological tissues, ranging from normal uterus extirpations to ovarian cancer, endometrial cancers and cervix carcinomas. HPV16 E6/E7 RNA expression was observed in 12 samples. All HPV16-positive samples were confirmed on DNA level, but five tissues that were negative in the HPV-RNA test, were positive in the HPV-DNA test arrow heads.

### DETAILED DESCRIPTION OF THE INVENTION

The present inventors have used multiplex profiling of RNA transcripts to determine which genes that are involved in metabolism are active, and which genes that are involved in pathologies are active. The inventors have found that from the combined information in the RNA profiles, the metabolic pathways that are most prominent in the pathological tissue can be deduced, and the genes that are actively involved in pathologies can be identified. This information can result in a personalized advice to treat an individual suffering from a disease with e.g. drugs that target the product of the gene that is aberrantly expressed and is involved in disease progression. These drugs (pharmaceutical compounds) include but are not limited to drugs that are approved by the United States Food and Drug Administration (FDA) and/or the European Medicines Agency (EMA) and are known as targeted drugs to the person skilled in the art. Such treatment advice can be combined with an advice to treat the disease further with a compound that inhibits the most essential metabolic pathways in the pathological tissue. This concept is known as synthetic lethality to the person skilled in the art. Added value of the test is generated by concomitant information on mutation status of metabolic genes.

The test requires a small aliquot of an RNA of interest that may be derived from solid tissue, isolated cells or bodily fluids, including, but not limited to, saliva, urine, sperm, blood, blood platelets and cerebrospinal fluid. The sample RNA can be converted to copy-DNA (cDNA) using a method known in the art, such as using oligo-dT primers or a mixture of random hexamer oligonucleotide primers. These techniques are standard techniques and are known to the person skilled in the art ((see e.g. Green and Sambrook (2012) Molecular Cloning: A Laboratory Manual, Fourth Edition, Cold Spring Harbor Laboratory Press, NY).

The RNA of interest may be from human genes but may also be from genes of pathogens such as DNA viruses and RNA viruses, including but not limited to human immune deficiency virus (HIV); human papilloma viruses, including but not limited to the subtypes HPV16 and HPV18; hepatitis A virus; hepatitis B virus; hepatitis C virus; hepatitis E virus; Ebola virus; Epstein Bar Virus (EBV); influenza viruses; West-Nile virus, chikungunya virus, polyoma virus; cytomegalovirus; rhinovirus, but also genes from the category of oncolytic viruses that are known to persons skilled in the art to treat cancers. The RNA of interest may also be from genes of parasites, including but not limited to Plasmodium falciparum and Plasmodium vivax, parasites causing malaria, and trypanosoma. In addition, the RNA of interest may be from (pathogenic) fungi, including but not limited to Aspergillus. The RNA of interest may also be from (pathogenic) bacteria, such as Listeria, Legionella, Staphylococcus, Streptococcus, Mycobacterium and/or Yersinia.

The subject (interchangeably also referred to as patient) may be a human or an animal. Accordingly, the RNA of interest may also be from genes from domesticated, wild and farm animals and/or from genes that are present in pathogens such as the pathogens listed here above or their counterparts that cause disease in animals.

The present invention further provides for a set of single molecule molecular inversion probes (smMIPs) to detect the RNAs of interest that carry the information that is needed to formulate a treatment advice. A preferred set is selected from the group listed in Table II.

A preferred method of generating RNA profiles is by using smMIPs that can be designed with the published MIPGEN protocol (18) that selects optimal ligation and extension probe sequences that are predicted to hybridize against a cDNA of interest while leaving a gap between the ligation and extension parts of the probe. The ligation and extension parts of the probes may hybridize to any part of the cDNA, including sequences that are protein encoding and untranslated regions. Extension and ligation parts of the probes can be located in the same exon.

A preferred method is to locate the ligation and extension parts of the probes in different exons of a cDNA, which allows detection of specific splice variants.

A preferred method according to the invention is to contact a library of designed smMIPs according to the invention, that may consist of any number of smMIPs, with a population of cDNA molecules. After an initial heating and denaturation step followed by cooling, each smMIP will hybridize to its target cDNA sequence. By incubating the mixture with a DNA polymerase enzyme, all four deoxynucleotides and DNA ligase in an appropriate buffer, the extension probe part of the MIP will be extended until the 5′ end of the ligation probe is reached. The DNA ligase will then covalently link the 3′ end of the extended extension probe part to the ligation probe part, producing a circular smMIP molecule.

In the next step, a method known to the person skilled in the art, is used to remove unreacted, linear smMIPs and cDNA from the reaction mixture by exonuclease treatment, leaving a purified library of circular smMIPs.

Using a forward and a reverse oligonucleotide primer that specifically anneal to the backbone sequence that connects the ligation and extension probes parts of the MIP, a PCR amplification of the gap sequence is performed. Preferably, one of the oligonucleotide primers that are used in this PCR is equipped with a barcode, allowing easy selection of all PCR products that are obtained from a specific sample. In a next step, the library of PCR amplicons are preferably analyzed on a next generation sequencing platform that yields FASTQ files containing information on nucleotide sequences of all PCR amplicons in the sample. Using an algorithm all PCR amplicons with the same barcode are grouped, producing a list of sequences for each individual cDNA sample.

Next, using another algorithm that uses the UMI, all identical PCR products will be considered to be derived from one originating smMIP. In this manner for each original RNA sample a list can be created that contains values that represent the original number of circularized smMIPs in the original library. This number is proportional to the number of cDNAs in the original sample.

In a preferred method of interpretation, the values obtained for each individual smMIP are divided by the summated values of all smMIPs for each sample, followed by multiplying with a factor of one million, thus yielding a fragments per million value for each smMIP.

In a preferred method of interpretation, the mean FPM values of all different smMIPs that correspond to one transcript, are considered to be proportional to the number of transcripts that were present in the initial RNA sample of the analysis.

In another preferred method of interpretation, mean FPM values of individual transcripts are divided by mean FPM values of so-called house-keeping genes, to yield a relative abundance value of a transcript of interest.

In another preferred method, mean FPM values for transcripts from genes that are involved in metabolic pathways are used to deduce the predominant metabolic pathways in a tissue.

A preferred method to analyze the FASTQ files further is to detect mutations in the next generation sequencing data. Preferably, mutations are considered as relevant if they are detected in more than two reads. The sequence information as provided in the FASTQ files should not be so narrowly construed as to require inclusion of erroneously identified bases. The skilled person is capable of identifying such erroneously identified bases and knows how to correct for such errors. A list of relevant mutations in a sample can be included in a database, preferably a standard query language (SQL)-based database that allows statistical analyses, for example by multivariate analysis.

A preferred method of analysis of the database results in a list of metabolic pathways that are active in a tissue or in a person with a disease and that can be used to give a dietary advice to relieve the symptoms of the disease and to improve the efficacy of other therapies.

Another preferred method of analysis of the database results in a list of aberrancies that can be treated with available pharmacological drugs.

Yet another preferred method of the invention uses a software algorithm that translates RNA profiles of diseased tissues directly to a treatment advice that can be given via an application that can be installed on a personal computer or a mobile device.

The method according to the invention can be readily implemented in routine patient care in case RNA from diseased tissue or blood platelets is available.

Accordingly, in a first aspect, the present invention provides for a method for in vitro determination of the susceptibility and/or resistance of a subject suffering from or at risk of a disease or condition for a drug to treat the disease or condition, comprising:
providing a sample from the subject,
performing RNA profiling on the sample,
   wherein the presence of an aberrant level of a transcript, an alternative splice variant and/or a mutation is an indication for the susceptibility and/or resistance.

Said method is herein referred to as the method according to the invention. “RNA profiling” is herein also referred to as targeted RNA sequencing of transcripts. An aberrant level of a transcript is a level of transcription that can either be higher or lower than the transcript level as compared to a reference sample and/or as compared to the level of transcript in a healthy subject.

Preferably, in a method according to the invention, RNA profiling is performed by multiplex mRNA sequencing, targeting multiple regions of interest. The sample RNA of interest may first be converted to copy-DNA (cDNA) using a method known in the art, such as using oligo-dT primers or a mixture of random hexamer oligonucleotide primers. The RNA of interest may be from human genes but may also be from genes of pathogens such as DNA viruses and RNA viruses, including but not limited to human immune deficiency virus (HIV); human papilloma viruses, including but not limited to the subtypes HPV16 and HPV18; hepatitis A virus; hepatitis B virus; hepatitis C virus; hepatitis E virus; Ebola virus; Epstein Bar Virus (EBV); influenza viruses; West-Nile virus, chikungunya virus, polyoma virus; cytomegalovirus; rhinovirus, but also genes from the category of oncolytic viruses that are known to persons skilled in the art to treat cancers. The RNA of interest may also be from genes of parasites, including but not limited to Plasmodium falciparum and Plasmodium vivax, parasites causing malaria, and trypanosoma. In addition, the RNA of interest may be from (pathogenic) fungi, including but not limited to Aspergillus. The RNA of interest may also be from (pathogenic) bacteria, such as Listeria, Legionella, Staphylococcus, Streptococcus, Mycobacterium and/or Yersinia.

Preferably, in a method according to the invention, the multiplex mRNA sequencing is performed using molecular inversion probes (MIPs), preferably MIPs comprising a detectable moiety, preferably a unique identifier sequence of a string of 3 to 10 random nucleotides (depicted as “N” in a sequence listing), more preferably a string of 3, more preferably 4, more preferably 5, more preferably 6, more preferably 7, most preferably 8, or preferably more than 8 random nucleotides (N) adjacent to the ligation part of the MIP or to the extension part of the MIP sequence (smMIPs).

Preferably, in a method according to the invention, the aberrant level of a transcript, an alternative splice variant and/or a mutation is linked to a an aberrance in a metabolic pathway which is in turn linked to the susceptibility and/or resistance of a subject suffering from or at risk of a disease or condition for a drug. In all embodiments of the invention, a drug is as meant in the art, a pharmaceutical compound. Such pharmaceutical compound may be comprised in a pharmaceutical composition. In all embodiments of the invention, a subject is a human or an animal, preferably a human. An animal may be any animal, preferably a domestic, wild or farm animals.

Preferably, in a method according to the invention, the disease or condition is at least one selected from the group consisting of a cancer, a viral infection, a bacterial infection, an autoimmune disease and a genetic disease.

In a method according to the invention, the sample may be any appropriate sample known to the person skilled in the art, preferably selected from the group consisting of a tissue, a tumor tissue, urine, sperm, saliva, blood, blood plasma, cerebrospinal fluid, blood platelets, and/or exosomes, more preferably selected from tumor tissue and blood platelets.

In a method according to the invention, the metabolic pathway is preferably selected from the group consisting of a glucose processing pathway, a glutamine processing pathway and/or a fatty acid pathway.

Preferably, in a method according to the invention, the multiple regions of interest are within the mRNA of—glucose processing genes, glutamine processing genes, fatty acid anabolism genes, transporter genes, redox homeostasis genes, genes with potential involvement in cancer, such as oncogenes, genes involved in angiogenesis, genes involved in immune suppression, and viral genes.

Preferably, in a method according to the invention, the multiple regions of interest are within the mRNA of at least one, two, three, four, five or at least six genes selected from the group consisting of: ABAT, ACACA, ACACB, ACLY, ACO2, ACSS2, ADPGK, ALDOA, ARHGAP26, ATG4A. ATP5A1, CBR1, CBS, CHKA, CKB, CPT1A, CYCS, EGLN1, ENO1, G6PC, GAD1, GCLC, GCLM, GFPT1, GLDC, GSS, HK1, HK2, HK3, GLY1, G6PD, RGN, PGD, RPIA, RPE, TKT, PGI, ALDOA, GAPDH, PGAM1/2, ENO, PKM1/2, PDHA1, PDK1, PFKB1, PFKMb, PGAM1, PGD, PGK1, PKM, PRDX1, PRKAA1, RPIA, PC, CS, ACO1, IDH1, IDH2, IDH3A, IDH3B, IDH3G, OGDH, SUCLA2, SDHA/B/C/D, FH, MDH1, MDH2, PDK, LDHA, LDHB, SLC16A1, SLC16A3, CA9, CA12, SLC4A10, VHL, SDH, SDHAF2, HPGL/PCC, FH, CS, D-2HGDH, L-2HGDH, FH, IDH1-3A-G, MDH1-2, MYC, OGDH, SDHA-D, VHL, PHD, HIF1a, EPAS2 PDCD1, SLC1A5, ASCT2, GLS, GLUD1/2, GOT, GPI, GS, BCAT1, BCAT2, SLC1A2, SLC7A11, SLC25A1, ACLY, ACACA, ACACB, FASN, CPT1, SLC5A7, CHKA, CPT2, VLCAD, HADHA/B, SCAD, MCAD, LCAD, SCHA-D, 2-Enoyl-VoA hydratase, MCKAT, SLC16A1, SLC16A7, SLC2A1, SLC2A3, SLC5A1, SLC5A5, SLC7A1, SLC9A1, SLCA12, redox homeostasis genes: NAMPT, NAPRT1, NOX1, NOX3, NOX4A, NQO1, SOD, SOD2, CAT, TAL, TIGAR, TRX, PARP1, ALK, AXL, BRAF, KRAS, TP53, MAPK8, MYC, TP5313, FGFR1, FGFR2, IGF1-R, KDR, NTRK1, NTRK2, PDGFRA, PDGFRB, EGFR, EGFRvIII, ERBB2, ERBB3, ERBB4, MERTK, PLXND1, RET, Androgen receptor (AR), AR variant 7, AR variant 12, FOLH1, KLK3, MET, METdelta14, METdelta7-8, KIT, RON PTEN, VEGF-A121, VEGF-A144, VEGF-A165, VEGF-A189, CD274, CTLA4, HPV-E2, HPV-E6, and HPV-E7.

Preferably, in a method according to the invention, the multiple regions of interest are within the mRNA of:
glucose processing genes, such as, but not limited to: ABAT, ACACA, ACACB, ACLY, ACO2, ACSS2, ADPGK, ALDOA, ARHGAP26, ATG4A. ATP5A1, CBR1, CBS, CHKA, CKB, CPT1A, CYCS, EGLN1, ENO1, G6PC, GAD1, GCLC, GCLM, GFPT1, GLDC, GSS, HK1, HK2, HK3, GLY1, G6PD, Gluconolactonase, PGD, RPIA, RPE, TKT, PGI, ALDOA, GAPDH, PGAM1/2, ENO, PKM1/2, PDHA1, PDK1, PFKB1, PFKMb, PGAM1, PGD, PGK1, PKM, PRDX1, PRKAA1, RPIA, PC, CS, ACO1, IDH1, IDH2, IDH3A, IDH3B, IDH3G, OGDH, SUCLA2, SDHA/B/C/D, FH, MDH1, MDH2, PDK, LDHA, LDHB, SLC16A1, SLC16A3, CA9, CA12, SLC4A10, VHL, SDH, SDHAF2, HPGL/PCC, FH, CS, D-2HGDH, L-2HGDH, FH, IDH1-3A-G, MDH1-2, MYC, OGDH, SDHA-D, VHL, PHD, HIF1a, EPAS2 and/or PDCD1;
glutamine processing genes, such as, but not limited to: SLC1A5, ASCT2, GLS, GLUD1/2, GOT, GPI, GS, BCAT1, BCAT2, SLC1A2 and/or SLC7A11;
fatty acid anabolism genes, such as, but not limited to: SLC25A1, ACLY, ACACA, ACACB, FASN, CPT1, SLC5A7, CHKA, CPT2, VLCAD, HADHA/B, SCAD, MCAD, LCAD, SCHA-D, 2-Enoyl-VoA hydratase and/or MCKAT;
transporter genes, such as, but not limited to; SLC16A1, SLC16A7, SLC2A1, SLC2A3, SLC5A1, SLC5A5, SLC7A1, SLC9A1 and/or SLCA12;
redox homeostasis genes, such as, but not limited to: NAMPT, NAPRT1, NOX1, NOX3, NOX4A, NQO1, SOD, SOD2, CAT, TAL, TIGAR and/or TRX;
DNA repair genes, such as, but not limited to: PARP1;
genes with potential involvement in cancer, such as, but not limited to: ALK, AXL, BRAF, KRAS, HRAS, NRAS, GNAQ, GNA11, TP53, MAPK8, MYC, TP5313, FGFR1, FGFR2, IGF1-R, KDR, NTRK1, NTRK2, PDGFRA, PDGFRB, EGFR, EGFRvIII, ERBB2, ERBB3, ERBB4, MERTK, PLXND1, RET, Androgen receptor (AR), AR variant 7, AR variant 12, FOLH1, KLK3, MET, METdelta14, METdelta7-8, KIT, RON and/or PTEN;
genes involved in angiogenesis, such as, but not limited to: VEGF-A121, VEGF-A144, VEGF-A165 and/or VEGF-A189
genes involved in immune suppression, such as, but not limited to: CD274 and/or CTLA4; and/or,
viral genes, such as, but not limited to: HPV-E2, HPV-E6 and/or HPV-E7.

Preferably, in a method according to the invention, the presence of an aberrant level of a transcript, an alternative splice variant and/or a mutation also provides an indication for treatment with dietary compounds or phytochemicals, optionally in combination with a drug. The person skilled in the art knows that drug treatment can beneficially be combined with treatment with dietary compounds or phytochemicals.

The method according to the invention can conveniently be used for guiding treatment in a subject (personalized medicine). Accordingly, in a further aspect, the invention provides for a method of treatment of a subject suffering from or at risk of a disease or condition, comprising:
requesting performance or performing a method according to the invention, thus determining the susceptibility and/or resistance of the subject suffering from or at risk of a disease or condition for a drug to treat the disease or condition, and
treating the disease or condition of the subject with a drug where the disease or condition of the subject is susceptible to. In this aspect, all features are preferably those of the first aspect.

Preferably, in a method of treatment according to the invention, the disease or condition is at least one selected from the group consisting of: a cancer, including but not limited to glioma, meningioma, ependymoma, pilocytic astrocytoma, adenocarcinomas, sarcomas, hemangioma, head and neck cancer, breast cancer, lung cancer, prostate cancer, kidney cancer, ovarian cancer, endometrial cancer, cervical cancer, colon cancer, rectal cancer, pancreatic cancer, esophagus cancer, basal cell cancer, penile cancer, vulva cancer, melanoma, uveal melanoma, lymphoma, acute myeloid leukemia, acute lymphoblastic leukemia, cholangiocarcinoma, hepatocellular carcinoma, soft tissue sarcoma, and osteosarcoma; a viral infection; a bacterial infection; an autoimmune disease and a genetic disease.

Preferably, in a method of treatment according to the invention, the drug treatment is supplemented with treatment with dietary compounds or phytochemicals.

The invention further provides for a medicament (drug) for use in the treatment of a subject suffering from or at risk of a disease or condition, wherein:
a method according to the invention is performed or requested to be performed, thus determining the susceptibility and/or resistance of the subject suffering from or at risk of a disease or condition for a drug to treat the disease or condition, and
administrating to a subject suffering from or at risk of a disease or condition with a drug where the disease or condition of the subject is susceptible to.

Preferably, in the medicament (drug) for use according to the invention, the disease or condition is at least one selected from the group consisting of a cancer, a viral infection, a bacterial infection, an autoimmune disease and a genetic disease.

Preferably, in the medicament (drug) for use according to the invention, the drug treatment is supplemented with treatment with dietary compounds or phytochemicals.

The invention further provides for method for the production of a medicament (drug) for the treatment of a subject suffering from or at risk of a disease or condition, comprising:
requesting performance or performing a method according to the invention, thus determining the susceptibility and/or resistance of the subject suffering from or at risk of a disease or condition for a drug to treat the disease or condition, and
treating the disease or condition of the subject with a drug where the disease or condition of the subject is susceptible to.

Preferably, in the method for the production of a medicament (drug) for the treatment according to the invention, the disease or condition is at least one selected from the group consisting of a cancer, a viral infection, a bacterial infection, an autoimmune disease and a genetic disease.

Preferably, in the method for the production of a medicament (drug) for the treatment according to the invention, the drug treatment is supplemented with treatment with dietary compounds or phytochemicals.

The invention further provides for a molecular inversion probe selected from the group as set forward in Table II. The invention further provides for a set of molecular inversion probes of at least two, three, four, five, six or more selected from the group as set forward in Table II.

The invention further provides for a library of circularized molecular inversion probes obtainable by a method according to the first or second aspect of the invention.

### Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptides obtainable by expression of the genes present in SEQ ID NO: 1 containing the nucleic acid sequences coding for the polypeptides should prevail.

All patent and literature references cited in the present specification are hereby incorporated by reference in their entirety.

TABLE II
Description of the sequences

| Seq ID | | |
|---|---|---|
| NO: | Seq name | Sequence |
| 1 | ABAT_0817 | CGTTGAATTTGATTATGATGGGCCTCTGNNNNNNNNCTTCAGCTT |
| | | CCCGATATCCGACGGTAGTGTTGTCCCTCAAGGGGTCA |
| 2 | ABAT_0820 | CAACAGACCCGCCCTCGGAATCNNNNNNNNCTTCAGCTTCCCGAT |
| | | ATCCGACGGTAGTGTTTACCTGGTTGATGTGGACGGC |
| 3 | ABAT_0823 | CCTCTCCTTCATGGGCGCGTTCCATGGNNNNNNNNCTTCAGCTTC |
| | | CCGATATCCGACGGTAGTGTAAAACGCCTTAAAGACCA |
| 4 | ABAT_0827 | GCCTTCTTGGTGGACGAGGTCCAGACCGGNNNNNNNNCTTCAGCT |
| | | TCCCGATATCCGACGGTAGTGTGGAAAAAGAAGAAGAC |
| 5 | ABAT_0831 | GATTCCATACGGAATAAGCTCATTTTAATNNNNNNNNCTTCAGCT |
| | | TCCCGATATCCGACGGTAGTGTATAATGCAGCCCATGC |
| 6 | ACACA_3334 | GCTCATTTTGGAGGAATAATGGATGANNNNNNNNCTTCAGCTTCC |
| | | CGATATCCGACGGTAGTGTAGAGGCCCAAATTGAGG |
| 7 | ACACA_3360 | GCTGGGAAGTTAATCCAGTACATTGTAGANNNNNNNNCTTCAGCT |
| | | TCCCGATATCCGACGGTAGTGTATGATGGCAGCAGTTA |
| 8 | ACACA_3375 | CTCCTCCAACCTCAACCACNNNNNNNNCTTCAGCTTCCCGATATC |
| | | CGACGGTAGTGTTATTGCCTATGAACTTAACAGCGTAC |
| 9 | ACACA_3390 | GCAATGACATCACATACCGAATTGGGNNNNNNNNCTTCAGCTTCC |
| | | CGATATCCGACGGTAGTGTGGATGATCAAGGTCAGCTG |
| 10 | ACACA_3408 | GCGCTGGTTTGTGGAAGTGGAAGGAACAGNNNNNNNNCTTCAGCT |
| | | TCCCGATATCCGACGGTAGTGTAAAACATCCCGTACCT |
| 11 | ACACB_0664 | TCCCACCAGAAGCCCCCAANNNNNNNNCTTCAGCTTCCCGATATC |
| | | CGACGGTAGTGTTCTGATAACTCAGGGGAGACACCGCA |
| 12 | ACACB_0681 | GCAGGGACAGTGGAATACCTCTATANNNNNNNNCTTCAGCTTCCC |
| | | GATATCCGACGGTAGTGTTGCTCCATCCAGCGGCGGCA |
| 13 | ACACB_0698 | CCCAGAGCATCGTGCAGTTGGTCCAGANNNNNNNNCTTCAGCTTC |
| | | CCGATATCCGACGGTAGTGTCAGTATGCCAGCAACATC |
| 14 | ACACB_0714 | CCACTGTCATCATGGACCCCTTCAAGATCNNNNNNNNCTTCAGCT |
| | | TCCCGATATCCGACGGTAGTGTCTTCGAATACCTGCAG |
| 15 | ACACB_0730 | GCAGGCAGGACAGGTGTGGTTNNNNNNNNCTTCAGCTTCCCGATA |
| | | TCCGACGGTAGTGTAGACCGTGGTGACAGGACGA |
| 16 | ACLY_1628 | ACCACCTCAGCCATCCAGNNNNNNNNCTTCAGCTTCCCGATATCC |
| | | GACGGTAGTGTTTCGTCGGGCCTGTGGAAGAAGCGCCG |
| 17 | ACLY_1636 | GCTGACCTTGCTGAACCCCAAAGGGANNNNNNNNCTTCAGCTTCC |
| | | CGATATCCGACGGTAGTGTACTGCCGACTACATCTGCA |
| 18 | ACLY_1644 | GCTCCCGAGACGAGCCCTCAGTGGCTNNNNNNNNCTTCAGCTTCC |
| | | CGATATCCGACGGTAGTGTAAAGAAGGCCAAGCCT |
| 19 | ACLY_1652 | GCCAAGAACCAGGCTTTGAAGGAAGNNNNNNNNCTTCAGCTTCCC |
| | | GATATCCGACGGTAGTGTAAGGAGGGCCGCCTCACTAA |
| 20 | ACLY_1660 | GCTCGATTATGCACTGGAAGTAGAGAAGNNNNNNNNCTTCAGCTT |
| | | CCCGATATCCGACGGTAGTGTGATGAAGAAGGAAGGGA |
| 21 | ACO2_0767 | CCTGGATGACCCCGCCAGCCAGGAANNNNNNNNCTTCAGCTTCCC |
| | | GATATCCGACGGTAGTGTGGTGGCGATGAGCCACTTTG |
| 22 | ACO2_0773 | CGGTGAAAGGTGGCACAGGTGCAANNNNNNNNCTTCAGCTTCCCG |
| | | ATATCCGACGGTAGTGTGTGGATGTCATGGCTGGG |
| 23 | ACO2_0777 | GCTGCACCAATTCAAGCTATGAAGATATGNNNNNNNNCTTCAGCT |
| | | TCCCGATATCCGACGGTAGTGTTGAGCTGAAGCCACAC |
| 24 | ACO2_0783 | GCAAGGACCTGGAGGACCTGCAGATNNNNNNNNCTTCAGCTTCCC |
| | | GATATCCGACGGTAGTGTAAGGGGAGTTTGACCCAGGG |
| 25 | ACO2_0787 | CGAGACCAACCTGAAGAAACAGGGCNNNNNNNNCTTCAGCTTCCC |
| | | GATATCCGACGGTAGTGTTGGCATCAGGTGGGTGGTG |
| 26 | ACSS2_1192 | GCAATGAGCCAGGGGAGACCACTCAGATCNNNNNNNNCTTCAGCT |
| | | TCCCGATATCCGACGGTAGTGTGACTAAAGGGAAAATC |
| 27 | ACSS2_1196 | GCTGCATTGTGGTCAAGCACCTNNNNNNNNCTTCAGCTTCCCGAT |
| | | ATCCGACGGTAGTGTTCTTGGATTCCAGCTGCAGTCTT |
| 28 | ACSS2_1202 | AGCCTGTCACCAAGCATAGCCGNNNNNNNNCTTCAGCTTCCCGAT |
| | | ATCCGACGGTAGTGTGTGTTTTGTTTGAGGGGATTCCC |
| 29 | ACSS2_1206 | CGCTTTGAGACAACCTACNNNNNNNNCTTCAGCTTCCCGATATCC |
| | | GACGGTAGTGTTTTCCCATTCTTTGGTGTAGCTCCTGC |
| 30 | ACSS2_1210 | CTTGCCTAAAACCCGCTCAGNNNNNNNNCTTCAGCTTCCCGATAT |
| | | CCGACGGTAGTGTTGCCTCTACTGCTTTGTCACCTTGT |
| 31 | ALDOA_0076 | CACTGGGAGCATTGCCAAGCGGCTNNNNNNNNCTTCAGCTTCCCG |
| | | ATATCCGACGGTAGTGTAACTTGCTACTACCAGCACCA |
| 32 | ALDOA_0080 | GCCATCATGGAAAATGCCAATGTTCTGGCNNNNNNNNCTTCAGCT |
| | | TCCCGATATCCGACGGTAGTGTAGACTACCACCCAAGG |
| 33 | ALDOA_0082 | GCTCTGAGTGACCACCACATCTACNNNNNNNNCTTCAGCTTCCCG |
| | | ATATCCGACGGTAGTGTTTATGCCAGTATCTGCCAGCA |
| 34 | ALDOA_0084 | GAGGCGTCCATCAACCTCAATGCCATTNNNNNNNNCTTCAGCTTC |
| | | CCGATATCCGACGGTAGTGTCCATGCTTGCACTCAGAA |
| 35 | ALDOA_0086 | GCCTGTCAAGGAAAGTACACTCCGAGCGNNNNNNNNCTTCAGCTT |
| | | CCCGATATCCGACGGTAGTGTGCCCTGACCTTCTCCT |
| 36 | ARHGAP26_2921 | GTGCATAGGAGATGCAGAAACAGATGATGNNNNNNNNCTTCAGCT |
| | | TCCCGATATCCGACGGTAGTGTGACAAGACCAACAAAT |
| 37 | ARHGAP26_2925 | GTTTGTGGAGCCTCTGCTGGCCTTNNNNNNNNCTTCAGCTTCCCG |
| | | ATATCCGACGGTAGTGTCCAAAAAGAAAGAATCTCAGC |
| 38 | ARHGAP26_2931 | GTGAAGGGACTGCGCAGTTGGACAGCATTNNNNNNNNCTTCAGCT |
| | | TCCCGATATCCGACGGTAGTGTGGAAGCAGTAGACAGG |
| 39 | ARHGAP26_2939 | CAGCATCCTTAATTCCAGCAGCNNNNNNNNCTTCAGCTTCCCGAT |
| | | ATCCGACGGTAGTGTTGACTCCAAGCCCCCGTCCTGCA |
| 40 | ARHGAP26_2944 | GTTCACAGCAGGCACGGTCTTCGATAACGNNNNNNNNCTTCAGCT |
| | | TCCCGATATCCGACGGTAGTGTCATCCAAACCTGCACT |
| 41 | ATG4A_3103 | GCTGGTATGGATCTTAGGGAAGCAGNNNNNNNNCTTCAGCTTCCC |
| | | GATATCCGACGGTAGTGTCGTAGTCAAGTTGCCGGTGG |
| 42 | ATG4A_3107 | CAGTTGCACAGGTGTTAAAAAAACTTGCNNNNNNNNCTTCAGCTT |
| | | CCCGATATCCGACGGTAGTGTAAGAATACCAACGCATC |
| 43 | ATG4A_3110 | GTGTTTTAAGATGCCACAGTCTTTAGGGGNNNNNNNNCTTCAGCT |
| | | TCCCGATATCCGACGGTAGTGTGCTTCAAACCAGAGTA |
| 44 | ATG4A_3112 | TCCATTGCCTGCAGTCCCCACAGCGAATGNNNNNNNNCTTCAGCT |
| | | TCCCGATATCCGACGGTAGTGTAAAACCAAATAACGCG |
| 45 | ATG4A_3114 | GCCAAGCCAGAAGTGACAACCACTGGGNNNNNNNNCTTCAGCTTC |
| | | CCGATATCCGACGGTAGTGTCAAAGAAGAAAAAGACT |
| 46 | ATP5A1_1339 | GCCCGCGTACATGGGCTGAGGAATGTTNNNNNNNNCTTCAGCTTC |
| | | CCGATATCCGACGGTAGTGTCACTCATCTTCAAAAGAC |
| 47 | ATP5A1_1342 | GAGTTGGTCTGAAAGCCCCCGGTATCATTNNNNNNNNCTTCAGCT |
| | | TCCCGATATCCGACGGTAGTGTAGTGAAGAGGACAGGA |
| 48 | ATP5A1_1347 | ATGTCTCTGTTGCTCCGCCGACCNNNNNNNNCTTCAGCTTCCCGA |
| | | TATCCGACGGTAGTGTATGCTGCCCCACTTCAGTACCT |
| 49 | ATP5A1_1350 | GCTGCCCAAACCAGGGCTATGAAGCAGGTNNNNNNNNCTTCAGCT |
| | | TCCCGATATCCGACGGTAGTGTCTTACATTCCAACAAA |
| 50 | ATP5A1_1353 | GCCTTGTTGGGCACTATCAGGGCTGATNNNNNNNNCTTCAGCTTC |
| | | CCGATATCCGACGGTAGTGTGGCTATTGAAGAACAAGT |
| 51 | ATP5C1_0551 | GCTGAGAGAGAGCTGAAACCAGCTCNNNNNNNNCTTCAGCTTCCC |
| | | GATATCCGACGGTAGTGTCCGCAATGGATTCAAGTTCG |
| 52 | ATP5C1_0553 | GTTATGCTTGTTGGAATTGGTGACAANNNNNNNNCTTCAGCTTCC |
| | | CGATATCCGACGGTAGTGTAAGACAAGAAGAAACACC |
| 53 | ATP5C1_0555 | AAATTCAGGTCTGTCATCTCCNNNNNNNNCTTCAGCTTCCCGATA |
| | | TCCGACGGTAGTGTTTCTGACCAGTTTCTGGTGGCATT |
| 54 | ATP5C1_0557 | GCCAGGATGACAGCCATGGACAATGCCNNNNNNNNCTTCAGCTTC |
| | | CCGATATCCGACGGTAGTGTAAGTGCTGACAGCATGAG |
| 55 | ATP5C1_0559 | GTTCCATCCTCAGACAAGAGGTAAAGAAGNNNNNNNNCTTCAGCT |
| | | TCCCGATATCCGACGGTAGTGTGAATGCTTCTGAGATG |
| 56 | BCAT1_0990 | TAGTCACACCAGCTACCANNNNNNNNCTTCAGCTTCCCGATATCC |
| | | GACGGTAGTGTCCTTTTTTGTGTTTGCCTGGGTCCTGG |
| 57 | BCAT1_0993 | GCTGTGAGGGCAACTCTGCCGGTATTTNNNNNNNNCTTCAGCTTC |
| | | CCGATATCCGACGGTAGTGTCCCTGGCTCATCAGCTTT |
| 58 | BCAT1_0996 | GTGGAACTGGGGACTGCAAGATGGGAGGGNNNNNNNNCTTCAGCT |
| | | TCCCGATATCCGACGGTAGTGTCAAGAAGCCTACCAAA |
| 59 | BCAT1_0998 | GCATCATTCTTCCAGGAGTGACANNNNNNNNCTTCAGCTTCCCGA |
| | | TATCCGACGGTAGTGTTGGGTGTCAGCAGGTCCTGTG |
| 60 | BCAT1_1000 | GCGAGACAATACACATTCCAACTATGGAGNNNNNNNNCTTCAGCT |
| | | TCCCGATATCCGACGGTAGTGTGTCAGAGAGATACCTC |
| 61 | BCAT2_1494 | GCCCAGTGGGTGCCTACTTNNNNNNNNCTTCAGCTTCCCGATATC |
| | | CGACGGTAGTGTTCATCGAAGTGGACAAGGACTGGGTC |
| 62 | BCAT2_1497 | GCCTGCCGAGTTTCGACAAGCTGGAGTTNNNNNNNNCTTCAGCTT |
| | | CCCGATATCCGACGGTAGTGTGCCTCCACTACTCCCT |
| 63 | BCAT2_1499 | GTGGGAATTATGGGCCCACCGTNNNNNNNNCTTCAGCTTCCCGAT |
| | | ATCCGACGGTAGTGTCGCTCCTGTTCGTCATTCTCT |
| 64 | BCAT2_1501 | GCCTGGAGTGGTCAGACAGAGTCTANNNNNNNNCTTCAGCTTCCC |
| | | GATATCCGACGGTAGTGTGGTCCTCTGGCTGTATGGG |
| 65 | BCAT2_1503 | CCTGTACAAAGACAGGAACCTCCNNNNNNNNCTTCAGCTTCCCGA |
| | | TATCCGACGGTAGTGTTGGGGTGAGTTCCGGGTGGTGG |
| 66 | CA12_3467 | CCTGATGGGGAGAATAGCTGGTCCAAGANNNNNNNNCTTCAGCTT |
| | | CCCGATATCCGACGGTAGTGTCAGGAGCCCGCGAAGA |
| 67 | CA12_3470 | ACCCGCACGGCTCTGAGCACANNNNNNNNCTTCAGCTTCCCGATA |
| | | TCCGACGGTAGTGTAACAAGCAGTTTCTCCTGACCAAC |
| 68 | CA12_3472 | CACCTTCAACATGTAAAGTACAAAGGCNNNNNNNNCTTCAGCTTC |
| | | CCGATATCCGACGGTAGTGTCCATTATAACTCAGACCT |
| 69 | CA12_3474 | GCTGCTGGCTTTGGAGACAGNNNNNNNNCTTCAGCTTCCCGATAT |
| | | CCGACGGTAGTGTAACATTGAAGAGCTGCTTCCGGAGA |
| 70 | CA12_3476 | GTGGTGGTGTCCATTTGGCTTTTNNNNNNNNCTTCAGCTTCCCGA |
| | | TATCCGACGGTAGTGTCCCCCAGAGAAATGATCAACAA |
| 71 | CA9_1143 | GCCCAGTGAAGAGGATTCACCCAGAGAGGNNNNNNNNCTTCAGCT |
| | | TCCCGATATCCGACGGTAGTGTCTGCTGCTGTCACTGC |
| 72 | CA9_1145 | GAGGCTCCTGGAGATCCTCAAGAACCNNNNNNNNCTTCAGCTTCC |
| | | CGATATCCGACGGTAGTGTAGGATCCACCCGGAGAGGA |
| 73 | CA9_1148 | CCCTCTGACTTCAGCCGCTANNNNNNNNCTTCAGCTTCCCGATAT |
| | | CCGACGGTAGTGTTGTTGGCCGCCTTTCTGGAGGAGGG |
| 74 | CA9_1150 | GCGACGCAGCCTTTGAATGGGCGAGTGANNNNNNNNCTTCAGCTT |
| | | CCCGATATCCGACGGTAGTGTTGTGCCCAGGGTGTCA |
| 75 | CA9_1152 | GCAGATGAGAAGGCAGCACAGAAGGGGANNNNNNNNCTTCAGCTT |
| | | CCCGATATCCGACGGTAGTGTTGGAGTGGACAGCAGT |
| 76 | CBR1_1512 | GTTGCTGATCCCACACCCTTTCATATNNNNNNNNCTTCAGCTTCC |
| | | CGATATCCGACGGTAGTGTTGAGCCCGCGCTTCCACCA |
| 77 | CBR1_1515 | CCCCAAGCATCCTGCGTACTNNNNNNNNCTTCAGCTTCCCGATAT |
| | | CCGACGGTAGTGTTGGTCAACAACGCGGGCATCGCCTT |
| 78 | CBR1_1516 | ATCTGCCGCTGCTTAACTCTGGGCCNNNNNNNNCTTCAGCTTCCC |
| | | GATATCCGACGGTAGTGTTGCACAGAATTACTCCCTCT |
| 79 | CBR1_1517 | GTCTTTGGTTGTAAACTGCTGTGATAGTTNNNNNNNNCTTCAGCT |
| | | TCCCGATATCCGACGGTAGTGTAGGAGGAAAGTCCAAG |
| 80 | CBS_0094 | CCCTGTGGATCCGGCCCGATGCTNNNNNNNNCTTCAGCTTCCCGA |
| | | TATCCGACGGTAGTGTTCCCAGCATGCCTTCTGAGACC |
| 81 | CBS_0099 | GCTCTTGGCCAAGTGTGAGTTCTTNNNNNNNNCTTCAGCTTCCCG |
| | | ATATCCGACGGTAGTGTAGTCCCCACATCACCACACT |
| 82 | CBS_0102 | CGGAGTCACACGTGGGGGTNNNNNNNNCTTCAGCTTCCCGATATC |
| | | CGACGGTAGTGTTGGCTGCGGCAGTGAGGGGCTAT |
| 83 | CBS_0107 | GCAAGAGGGGCTGCTGTGCGGTGGCAGTGNNNNNNNNCTTCAGCT |
| | | TCCCGATATCCGACGGTAGTGTACCTACGAGGTGGAAG |
| 84 | CBS_0112 | GCTCTCGCACATCCTGGAGATGGANNNNNNNNCTTCAGCTTCCCG |
| | | ATATCCGACGGTAGTGTTGGGAATGGTGACGCTTGGG |
| 85 | CHKA_3492 | ACACCACAGCCACCCTTGGTGATGANNNNNNNNCTTCAGCTTCCC |
| | | GATATCCGACGGTAGTGTGCAGGGCCTATCTGTGGTGC |
| 86 | CHKA_3494 | GCCGGCGATTAGATACTGAAGAATTAAGNNNNNNNNCTTCAGCTT |
| | | CCCGATATCCGACGGTAGTGTTGCAGATGGGGGCTGAG |
| 87 | CHKA_3496 | GCTCAGTTACAATCTGCCCTTGGAACTGGNNNNNNNNCTTCAGCT |
| | | TCCCGATATCCGACGGTAGTGTGGTATGAAAATGCCAT |
| 88 | CHKA_3499 | CCAGTTACTTGCCTGCATTCCAAAATGANNNNNNNNCTTCAGCTT |
| | | CCCGATATCCGACGGTAGTGTGGGGATTCGACATTGG |
| 89 | CHKA_3501 | GCAAGGTTTGATGCCTATTTCCACCAGANNNNNNNNCTTCAGCTT |
| | | CCCGATATCCGACGGTAGTGTAAAGAAGAAATGTTGC |
| 90 | CKB_1938 | CCACCTGCGGGTCATCTCCATNNNNNNNNCTTCAGCTTCCCGATA |
| | | TCCGACGGTAGTGTACCACTTCCTCTTCGACAAGCCC |
| 91 | CKB_1940 | GCGACGACCTGGACCCCAACTACGTGCTGNNNNNNNNCTTCAGCT |
| | | TCCCGATATCCGACGGTAGTGTCGACGAGGAGTCCTAC |
| 92 | CKB_1945 | GGACTATGAGTTCATGTGGAACCCTCANNNNNNNNCTTCAGCTTC |
| | | CCGATATCCGACGGTAGTGTTGGTGTGGGTCAACGAGG |
| 93 | CKB_1947 | GCACAGGCGGTGTGGACACGGCTNNNNNNNNCTTCAGCTTCCCGA |
| | | TATCCGACGGTAGTGTCTACATCCTCACCTGCCCAT |
| 94 | CKB_1948 | GTGGTGGACGGAGTGAAGCTGCTNNNNNNNNCTTCAGCTTCCCGA |
| | | TATCCGACGGTAGTGTAGGTGCTTAAGCGGCTGCGAC |
| 95 | CPT1A_0611 | GGACTTCATTCCTGGAAAAAGAAGTTCNNNNNNNNCTTCAGCTTC |
| | | CCGATATCCGACGGTAGTGTGCTGACTCGGTACTCTCT |
| 96 | CPT1A_0615 | GATCTGGATGGGTATGGTCAAGATCTTTTNNNNNNNNCTTCAGCT |
| | | TCCCGATATCCGACGGTAGTGTGTGCTGTTTGGCACCG |
| 97 | CPT1A_0621 | GCACATGAGAGACAGCAAGCACATCGNNNNNNNNCTTCAGCTTCC |
| | | CGATATCCGACGGTAGTGTAAACTGGACCGGGAGGAAA |
| 98 | CPT1A_0629 | GCTGGCGCACTACAAGGACATGGGCAAGTNNNNNNNNCTTCAGCT |
| | | TCCCGATATCCGACGGTAGTGTAACACCGCAAATCTTC |
| 99 | CPT1A_0633 | GTTTGACTTGGAGAATAACCCAGAGTACGNNNNNNNNCTTCAGCT |
| | | TCCCGATATCCGACGGTAGTGTCACCTCTTCTGCCTTT |
| 100 | CYCS_3031 | GGTCTCTTTGGGCGGAAGACAGGTCAGNNNNNNNNCTTCAGCTTC |
| | | CCGATATCCGACGGTAGTGTGCGACTAAAAAGAGAAT |
| 101 | CYCS_3032 | GGGAGAGGATACACTGATGGAGTATTTGGNNNNNNNNCTTCAGCT |
| | | TCCCGATATCCGACGGTAGTGTACCGTTGAAAAGGGAG |
| 102 | CYCS_3033 | GGAAGAAAGGGCAGACTTAATAGCTTATCNNNNNNNNCTTCAGCT |
| | | TCCCGATATCCGACGGTAGTGTTACACAGCCGCCAATA |
| 103 | CYCS_3034 | CTTTTTTATGTGTACCATCCTTTAATAGNNNNNNNNCTTCAGCTT |
| | | CCCGATATCCGACGGTAGTGTTGATCTTTGTCGGCATT |
| 104 | EGLN1_3069 | CGGGCAGCTGGTCAGCCAGAAGAGTGNNNNNNNNCTTCAGCTTCC |
| | | CGATATCCGACGGTAGTGTGAGTACATCGTGCCGTG |
| 105 | EGLN1_3075 | GGAACGGGTTATGTACGTCATGTTGNNNNNNNNCTTCAGCTTCCC |
| | | GATATCCGACGGTAGTGTGGCTGCGAAACCATTGGG |
| 106 | EGLN1_3077 | GATAGACTGCTGTTTTTCTGGTCTGNNNNNNNNCTTCAGCTTCCC |
| | | GATATCCGACGGTAGTGTGGAGATGGAAGATGTGTG |
| 107 | EGLN1_3079 | GGTCGGTAAAGACGTCTTCTAGAGCCNNNNNNNNCTTCAGCTTCC |
| | | CGATATCCGACGGTAGTGTGCATATGCTACAAGGTACG |
| 108 | EGLN1_3080 | GTGAATACGAATAAATGGGATAAAGANNNNNNNNCTTCAGCTTCC |
| | | CGATATCCGACGGTAGTGTGGTGAAAAAGGTGTGAGGG |
| 109 | ENO1_1724 | GCTGTGCCCAGTGGTGCTTCAACTGGTANNNNNNNNCTTCAGCTT |
| | | CCCGATATCCGACGGTAGTGTAGACCCAGTGGCTAGAA |
| 110 | ENO1_1728 | GCGGTTCTCATGCTGGCAANNNNNNNNCTTCAGCTTCCCGATATC |
| | | CGACGGTAGTGTTTCTGGGGGTGTCCCTTGCCGTCTG |
| 111 | ENO1_1732 | GCCTGACCAGCTGGCTGACCTGTACAANNNNNNNNCTTCAGCTTC |
| | | CCGATATCCGACGGTAGTGTTTGGGAAAGCTGGCTACA |
| 112 | ENO1_1735 | GCCAATGGTTGGGGCGTCATGGTGTCTCNNNNNNNNCTTCAGCTT |
| | | CCCGATATCCGACGGTAGTGTACAGTGACCAACCCAAA |
| 113 | ENO1_1737 | CGGCAGGAACTTCAGAAACCCCTTGNNNNNNNNCTTCAGCTTCCC |
| | | GATATCCGACGGTAGTGTTGACCTGGTTGTGGGGCTGT |
| 114 | FASN_2387 | CCCAGCCCCCACCCACAANNNNNNNNCTTCAGCTTCCCGATATCC |
| | | GACGGTAGTGTTCCACAGCCTGGCTGCCTACTACATC |
| 115 | FASN_2394 | CGTGGAGCAGCTGAGGAAGGAGGGTGTNNNNNNNNCTTCAGCTTC |
| | | CCGATATCCGACGGTAGTGTTGGCAGCCGTGGGCTT |
| 116 | FASN_2423 | GCCATCCAGATAGGCCTCATAGACNNNNNNNNCTTCAGCTTCCCG |
| | | ATATCCGACGGTAGTGTCTTCCGAGATTCCATCCTAC |
| 117 | FASN_2438 | GCGTTCTTCAACGAGAGCAGTGCTGANNNNNNNNCTTCAGCTTCC |
| | | CGATATCCGACGGTAGTGTGCGTGAGGTGCTTGGCT |
| 118 | FASN_2445 | GTGCTGGCTGAGAAGGCTGNNNNNNNNCTTCAGCTTCCCGATATC |
| | | CGACGGTAGTGTGTGGGCATTTTGGTGGAGACGAT |
| 119 | FASN_2447 | GGGCCTAGAGGAGCGTGTGGCAGCNNNNNNNNCTTCAGCTTCCCG |
| | | ATATCCGACGGTAGTGTAGAGCTACCGGGCAAAG |
| 120 | G6PC_0139 | GCTGTGGGCATTAAACTCCTTTGGGTAGCNNNNNNNNCTTCAGCT |
| | | TCCCGATATCCGACGGTAGTGTCAACACATTACCTCCA |
| 121 | G6PC_0142 | GCCGACCTACAGATTTCGGTGCTTGNNNNNNNNCTTCAGCTTCCC |
| | | GATATCCGACGGTAGTGTGATAAAGCAGTTCCCTGTA |
| 122 | G6PC_0144 | GCATCTATAATGCCAGCCNNNNNNNNCTTCAGCTTCCCGATATCC |
| | | GACGGTAGTGTTGGTTGGGATTCTGGGCTGTGCAGCTG |
| 123 | G6PC_0146 | CACCCTTTGCCAGCCTCCTNNNNNNNNCTTCAGCTTCCCGATATC |
| | | CGACGGTAGTGTTTACCTTCTTCCTGTTCAGCTTCGCC |
| 124 | G6PC_0148 | CGTCTTGTCCTTCTGCAAGAGTGCGGTNNNNNNNNCTTCAGCTTC |
| | | CCGATATCCGACGGTAGTGTAGAGCTGCAAGGGGAAA |
| 125 | G6PD_0394 | CAGAGTGAGCCCTTCTTCAAGGCCACCCCNNNNNNNNCTTCAGCT |
| | | TCCCGATATCCGACGGTAGTGTACCTGGCCAAGAAGAA |
| 126 | G6PD_0397 | ACCTGCAGAGCTCTGACCGGCTGTCNNNNNNNNCTTCAGCTTCCC |
| | | GATATCCGACGGTAGTGTCCAACCGCCTCTTCTACCT |
| 127 | G6PD_0401 | GTACGTGGGGAACCCCGATGGAGANNNNNNNNCTTCAGCTTCCCG |
| | | ATATCCGACGGTAGTGTTGCAGATGCTGTGTCTGGTGG |
| 128 | G6PD_0405 | GACGTCTTCTGCGGGAGCCAGATGCANNNNNNNNCTTCAGCTTCC |
| | | CGATATCCGACGGTAGTGTACACCAAGATGATGACCAA |
| 129 | G6PD_0407 | CCAGTATGAGGGCACCTACAAGTGNNNNNNNNCTTCAGCTTCCCG |
| | | ATATCCGACGGTAGTGTGTGAGGCCTGGCGTATTTTC |
| 130 | GAD1_0451 | GAAGAGTCGCCTTGTGAGTGCCTTCAANNNNNNNNCTTCAGCTTC |
| | | CCGATATCCGACGGTAGTGTACCCCAATACCACTAACC |
| 131 | GAD1_0455 | GCACAGGTCATCCTCGATTTTTCAACCANNNNNNNNCTTCAGCTT |
| | | CCCGATATCCGACGGTAGTGTACTTTCATCACCCACAC |
| 132 | GAD1_0459 | GATAAAGTGCAATGAAAGGGGGAAAATANNNNNNNNCTTCAGCTT |
| | | CCCGATATCCGACGGTAGTGTGGAAGTTAAGACAAAGG |
| 133 | GAD1_0463 | GATGTCTCCTACGACACCGGGGACAAGGNNNNNNNNCTTCAGCTT |
| | | CCCGATATCCGACGGTAGTGTAACCCTCACAAGATGA |
| 134 | GAD1_0467 | TTCCGGATGGTCATCTCCAACCCAGCCNNNNNNNNCTTCAGCTTC |
| | | CCGATATCCGACGGTAGTGTTGTGCCAGACAGCCCTCA |
| 135 | GAPDH_1973 | CCCCTTCATTGACCTCAACTACATGGTTTNNNNNNNNCTTCAGCT |
| | | TCCCGATATCCGACGGTAGTGTCCACATCGCTCAGACA |
| 136 | GAPDH_1975 | GCTGGCGCTGAGTACGTCGTGGAGTNNNNNNNNCTTCAGCTTCCC |
| | | GATATCCGACGGTAGTGTCAATATGATTCCACCCATGG |
| 137 | GAPDH_1978 | CCATCACTGCCACCCAGAAGACNNNNNNNNCTTCAGCTTCCCGAT |
| | | ATCCGACGGTAGTGTATGAGAAGTATGACAACAGCCTC |
| 138 | GAPDH_1980 | GCCAACGTGTCAGTGGTGGACCTNNNNNNNNCTTCAGCTTCCCGA |
| | | TATCCGACGGTAGTGTTGTGGCGTGATGGCCGCGGGG |
| 139 | GAPDH_1982 | GCATTGCCCTCAACGACCACTTTGTCAANNNNNNNNCTTCAGCTT |
| | | CCCGATATCCGACGGTAGTGTAGAAGGTGGTGAAGCAG |
| 140 | GCLC_1788 | GAAAATAAAAAAGTCCGGTTGGTCCTGTCNNNNNNNNCTTCAGCT |
| | | TCCCGATATCCGACGGTAGTGTACCACGTGCGGCGGCA |
| 141 | GCLC_1792 | CCTCGCTTCAGTACCTTAACNNNNNNNNCTTCAGCTTCCCGATAT |
| | | CCGACGGTAGTGTCTCTTTGCACAATAACTTCATTTCC |
| 142 | GCLC_1796 | GATCAGTAAATCCCGATATGACTCAATAGNNNNNNNNCTTCAGCT |
| | | TCCCGATATCCGACGGTAGTGTTCTCCCTTTTACCGAG |
| 143 | GCLC_1800 | CCTACAAATTGGATTTTCTCATTCCACTGNNNNNNNNCTTCAGCT |
| | | TCCCGATATCCGACGGTAGTGTCCCTCCTCCAAACTCA |
| 144 | GCLC_1804 | GAACTAATGACAGTTGCCAGATGGATGNNNNNNNNCTTCAGCTTC |
| | | CCGATATCCGACGGTAGTGTGGAAGGTGTGTTTCCTGG |
| 145 | GCLM_1678 | GAAATGAAAGTTTCTGCAAAACTGTTCATNNNNNNNNCTTCAGCT |
| | | TCCCGATATCCGACGGTAGTGTTGCTGAACTGGGGCCG |
| 146 | GCLM_1680 | GCAAAAAGATTGTTGCCATAGGTACCNNNNNNNNCTTCAGCTTCC |
| | | CGATATCCGACGGTAGTGTTTCAGTCCTTGGAGTTGCA |
| 147 | GCLM_1682 | GCTTTCTGAAGCAAGTTTCCAAGAAGCNNNNNNNNCTTCAGCTTC |
| | | CCGATATCCGACGGTAGTGTCACAGGTAAAACCAAATA |
| 148 | GCLM_1683 | GCTACTGCGGTATTCGGTCATTGTGAAANNNNNNNNCTTCAGCTT |
| | | CCCGATATCCGACGGTAGTGTACAATTTGACATACAGC |
| 149 | GCLM_1684 | CTTACCTGTAATTTCCTTCAATATGAGAGNNNNNNNNCTTCAGCT |
| | | TCCCGATATCCGACGGTAGTGTGAGTGGGTGCCGCTGT |
| 150 | GFPT1_1220 | GCACTGGATGAAGAAGTTCACAAGCNNNNNNNNCTTCAGCTTCCC |
| | | GATATCCGACGGTAGTGTGGCCTTCAGAGACTGGAGTA |
| 151 | GFPT1_1224 | GCCCTCTGTTGATTGGTGTACGGAGTGNNNNNNNNCTTCAGCTTC |
| | | CCGATATCCGACGGTAGTGTGAAAGTCAAGATACCA |
| 152 | GFPT1_1228 | CACTCCAGATGGAACTCCAGCAGATCANNNNNNNNCTTCAGCTTC |
| | | CCGATATCCGACGGTAGTGTATAGAACACACCAATCGC |
| 153 | GFPT1_1234 | GTTTGCCCTTATGATGTGTGATGATCGGNNNNNNNNCTTCAGCTT |
| | | CCCGATATCCGACGGTAGTGTCACAAACACAGTTGGCA |
| 154 | GFPT1_1238 | GCTCTTCAGCAAGTGGTTGCTCGGNNNNNNNNCTTCAGCTTCCCG |
| | | ATATCCGACGGTAGTGTTACTTATATGCACTCTGAAGG |
| 155 | GLDC_0162 | GACGGTCCCTGCCAACATCCGTTTGAAAANNNNNNNNCTTCAGCT |
| | | TCCCGATATCCGACGGTAGTGTCTGGAGCGCCTTCTGC |
| 156 | GLDC_0168 | CAGACACGGAGGGGAAGGTGGAAGACTTNNNNNNNNCTTCAGCTT |
| | | CCCGATATCCGACGGTAGTGTACCCACAGACAATAGC |
| 157 | GLDC_0177 | GCATGATTCCACTGGGATCCTGCACCANNNNNNNNCTTCAGCTTC |
| | | CCGATATCCGACGGTAGTGTGGGTCTGTGTTCAAGAGG |
| 158 | GLDC_0183 | GCCCTGGAGACTTCGGGTCTGATGTCTNNNNNNNNCTTCAGCTTC |
| | | CCGATATCCGACGGTAGTGTACATACCCATCCACCAA |
| 159 | GLDC_0189 | ACTGAGTCGGAGGACAAGGCAGAGCTGGNNNNNNNNCTTCAGCTT |
| | | CCCGATATCCGACGGTAGTGTACGAGACCCTTCAAAAA |
| 160 | GLS_1282 | GCTGAAGGACAAGAGAAAATACCTGNNNNNNNNCTTCAGCTTCCC |
| | | GATATCCGACGGTAGTGTAAGGACGGCCCCGGGGAGA |
| 161 | GLS_1285 | GGTTGCAGATTATATTCCTCAACTGGCCANNNNNNNNCTTCAGCT |
| | | TCCCGATATCCGACGGTAGTGTCAGAGCAACATTGTTT |
| 162 | GLS_1288 | GCTGGAGCAATTGTTGTGACTTCACNNNNNNNNCTTCAGCTTCCC |
| | | GATATCCGACGGTAGTGTGCCATTGCTGTTAATGATCT |
| 163 | GLS_1292 | GCAGTTCGAAATACATTGAGTTTGATGCANNNNNNNNCTTCAGCT |
| | | TCCCGATATCCGACGGTAGTGTTAGACTTCTACTTCCA |
| 164 | GLS_1295 | GAAGGTGGTGATCAAAGGCATTCCTTTGGNNNNNNNNCTTCAGCT |
| | | TCCCGATATCCGACGGTAGTGTCTCTGGATAAGATGGG |
| 165 | GLUD1_2495 | GCGGCATCCTGCGGATCATCAAGCCCTGNNNNNNNNCTTCAGCTT |
| | | CCCGATATCCGACGGTAGTGTACGACCCCAACTTCTT |
| 166 | GLUD1_2501 | GTGAGCGGGAGATGTCCTGGATCGCTGATNNNNNNNNCTTCAGCT |
| | | TCCCGATATCCGACGGTAGTGTCAATCCCAAGAACTAT |
| 167 | GLUD1_2504 | GCTAAATGTATTGCTGTTGGTGAGTCTNNNNNNNNCTTCAGCTTC |
| | | CCGATATCCGACGGTAGTGTCATCAATGAAGCTTCTTA |
| 168 | GLUD1_2507 | GCTGACAAGATCTTCCTGGAGAGAAACANNNNNNNNCTTCAGCTT |
| | | CCCGATATCCGACGGTAGTGTTTGGAGGCCGACTGTG |
| 169 | GLUD1_2510 | GCACTCTGGCTTGGCATACACAATGGAGCNNNNNNNNCTTCAGCT |
| | | TCCCGATATCCGACGGTAGTGTTTGCTCATGTCTGTTC |
| 170 | GLUD2_2853 | GCGAGGAGCAGAAGCGGAACCGGGTNNNNNNNNCTTCAGCTTCCC |
| | | GATATCCGACGGTAGTGTACTACAGCGAGTTGGTGG |
| 171 | GLUD2_2856 | ACCGAAAATGAATTGGAAAAGATCACAAGNNNNNNNNCTTCAGCT |
| | | TCCCGATATCCGACGGTAGTGTGCACTGATGTGAGTGT |
| 172 | GLUD2_2859 | GCATTTTAGGAATGACACCAGGGTTTAGNNNNNNNNCTTCAGCTT |
| | | CCCGATATCCGACGGTAGTGTATGCACACGCCTGTGTT |
| 173 | GLUD2_2862 | TCGACTGTGACATACTGATCCCAGCTNNNNNNNNCTTCAGCTTCC |
| | | CGATATCCGACGGTAGTGTGTCTGATGGGAGTATATGG |
| 174 | GLUD2_2867 | GCCAGGCAAATTATGCACACAGCCATGAANNNNNNNNCTTCAGCT |
| | | TCCCGATATCCGACGGTAGTGTAATTTGGAAAGCATGG |
| 175 | GOT_1990 | GACCCCCGCAAGGTCAACCTGGGAGNNNNNNNNCTTCAGCTTCCC |
| | | GATATCCGACGGTAGTGTGGGCGTGGGGTGAAAT |
| 176 | GOT_1993 | CAAACAACAAGAACACACCNNNNNNNNCTTCAGCTTCCCGATATC |
| | | CGACGGTAGTGTGTGCTTCTCGTCTTGCCCTTGGGG |
| 177 | GOT_1996 | GACTCAGCCTATCAGGGCTTCGCATCNNNNNNNNCTTCAGCTTCC |
| | | CGATATCCGACGGTAGTGTCTCCTGAGTTCTCCATTG |
| 178 | GOT_1998 | CCTGCAAGTCCTTTCCCAGNNNNNNNNCTTCAGCTTCCCGATATC |
| | | CGACGGTAGTGTTGCCTGGGCCATTCGCTATTTTGTGT |
| 179 | GOT_2000 | AGCCCTCAAAACCCCTGGGACCTGGAANNNNNNNNCTTCAGCTTC |
| | | CCGATATCCGACGGTAGTGTAGGGAGCACGAATTGTGG |
| 180 | GPI_1522 | GCTTTGACCAGTGGGGAGTGGAGCTGGGANNNNNNNNCTTCAGCT |
| | | TCCCGATATCCGACGGTAGTGTTGAAGGAAATCGCCCA |
| 181 | GPI_1523 | GCTCATCAACTTCATCAAGCAGCAGNNNNNNNNCTTCAGCTTCCC |
| | | GATATCCGACGGTAGTGTCATCATCTGGGACATCAACA |
| 182 | GPI_1524 | GTGCTCATCTGCAGCCTCCTCTGTNNNNNNNNCTTCAGCTTCCCG |
| | | ATATCCGACGGTAGTGTAAGCAGCTGGCTAAGAAAATA |
| 183 | GPT_2527 | GGAGCTGCGCCAGGGTGTGAAGAANNNNNNNNCTTCAGCTTCCCG |
| | | ATATCCGACGGTAGTGTGGAGCCAGGCGGTGAG |
| 184 | GPT_2528 | GCATGGACTGAGGGCGAAGGTGCTGACGNNNNNNNNCTTCAGCTT |
| | | CCCGATATCCGACGGTAGTGTCACTAAGCCAGACCCA |
| 185 | GPT_2536 | GGGCAGAGGCCCATCACCTTNNNNNNNNCTTCAGCTTCCCGATAT |
| | | CCGACGGTAGTGTGGAGAGTGGAGTACGCAGTGCGTGG |
| 186 | GPT_2537 | CGATGCCAAGAAAAGGGCGGAGNNNNNNNNCTTCAGCTTCCCGAT |
| | | ATCCGACGGTAGTGTTTTCACCGAGGTCATCCGTGCC |
| 187 | GPT_2540 | GCTGGGTCGCCCTGGACTGTGTNNNNNNNNCTTCAGCTTCCCGAT |
| | | ATCCGACGGTAGTGTAAGGCACCTACCACTTCC |
| 188 | GS_0645 | TGGAGAAGGACTGCGCTGCAAGACCCGNNNNNNNNCTTCAGCTTC |
| | | CCGATATCCGACGGTAGTGTAACGAACACCTTCCACCA |
| 189 | GS_0648 | ACATGGTGAGCAACCAGCACCCNNNNNNNNCTTCAGCTTCCCGAT |
| | | ATCCGACGGTAGTGTTCGTGCCTGCTGCCATGTTTCGG |
| 190 | GS_0650 | GCTTGTATGCTGGAGTCAAGATTGCGGNNNNNNNNCTTCAGCTTC |
| | | CCGATATCCGACGGTAGTGTACAGATGGGCACCCCTTT |
| 191 | GS_0653 | CGAGGAGGCCATTGAGAAACTAAGCNNNNNNNNCTTCAGCTTCCC |
| | | GATATCCGACGGTAGTGTGTGTGAAGACTTTGGAGTG |
| 192 | GS_0656 | CCTCATCCGCACGTGTCTTCTCNNNNNNNNCTTCAGCTTCCCGAT |
| | | ATCCGACGGTAGTGTCTTTTCTGCTGGTGTAGCCAATC |
| 193 | GSS_0206 | GCTGTCAGCCAGAACGCTGCCTNNNNNNNNCTTCAGCTTCCCGAT |
| | | ATCCGACGGTAGTGTACCTCACAGGAGCCCACTTCCT |
| 194 | GSS_0208 | GCAGCGCAGATGGCTCCCCAGCCCTGAAANNNNNNNNCTTCAGCT |
| | | TCCCGATATCCGACGGTAGTGTCAGCACCATCAAACAG |
| 195 | GSS_0212 | GCTGTTTGTGGATGGCCAGGAAATTGNNNNNNNNCTTCAGCTTCC |
| | | CGATATCCGACGGTAGTGTGAGAAGGAAAGAAACATAT |
| 196 | GSS_0215 | GATGTGGGTGAAGAAGGGGACCAGGCCATNNNNNNNNCTTCAGCT |
| | | TCCCGATATCCGACGGTAGTGTTGGCTGGGACTAAGAA |
| 197 | GSS_0218 | GCAGGAAAAGACACTCGTGATGAACAANNNNNNNNCTTCAGCTTC |
| | | CCGATATCCGACGGTAGTGTCCTCCTACATCCTCAT |
| 198 | HIF1A_1815 | GTTTTTTATGAGCTTGCTCATCAGTTGCCNNNNNNNNCTTCAGCT |
| | | TCCCGATATCCGACGGTAGTGTGCGGGGACCGATTCAC |
| 199 | HIF1A_1821 | GCTTGGTGCTGATTTGTGAACCCATTNNNNNNNNCTTCAGCTTCC |
| | | CGATATCCGACGGTAGTGTCCGAGGAAGAACTATGAAC |
| 200 | HIF1A_1827 | GATGCTTTAACTTTGCTGGCCCCAGCNNNNNNNNCTTCAGCTTCC |
| | | CGATATCCGACGGTAGTGTCCCTTCAACAAACAGAATG |
| 201 | HIF1A_1833 | CACCATTAGAAAGCAGTTCCGCAAGCCCTNNNNNNNNCTTCAGCT |
| | | TCCCGATATCCGACGGTAGTGTTGCTGAAGACACAGAA |
| 202 | HIF1A_1839 | GCAGCTACTACATCACTTTCTTGGAAACGNNNNNNNNCTTCAGCT |
| | | TCCCGATATCCGACGGTAGTGTAAGAACTAAATCCAAA |
| 203 | HIF2A_1750 | GCCTCCATCATGCGACTGGCAATCAGCTTNNNNNNNNCTTCAGCT |
| | | TCCCGATATCCGACGGTAGTGTAGAGGAGGAAGGAGAA |
| 204 | HIF2A_1754 | CACGGTCACCAACAGAGGCCNNNNNNNNCTTCAGCTTCCCGATAT |
| | | CCGACGGTAGTGTATCTTTGACTTCACTCATCCCTGCG |
| 205 | HIF2A_1760 | GGACCAGACTGAATCCCTGTTCNNNNNNNNCTTCAGCTTCCCGAT |
| | | ATCCGACGGTAGTGTTGGGGGCTACGTGTGGCTGG |
| 206 | HIF2A_1768 | GTCTGCAAAGGGTTTTGGGGCTCGAGGNNNNNNNNCTTCAGCTTC |
| | | CCGATATCCGACGGTAGTGTCAGATCCACCATTACA |
| 207 | HIF2A_1772 | TTCCCCCCACAGTGCTACGCCANNNNNNNNCTTCAGCTTCCCGAT |
| | | ATCCGACGGTAGTGTCTGAGCGCAAATGTACCCAATG |
| 208 | HK1_0224 | TGGCCTCTCCCGGGATTTTAATCCAACNNNNNNNNCTTCAGCTTC |
| | | CCGATATCCGACGGTAGTGTCCTATTACTTCACGGAGC |
| 209 | HK1_0230 | GCACATTGATCTGGTGGAAGGAGACGNNNNNNNNCTTCAGCTTCC |
| | | CGATATCCGACGGTAGTGTAACATCGTAGCTGTGGTGA |
| 210 | HK1_0236 | GCGCTTCCTCCTCTCGGAGAGTGGCAGNNNNNNNNCTTCAGCTTC |
| | | CCGATATCCGACGGTAGTGTATAACAAGGGCACACCCA |
| 211 | HK1_0242 | GCGGGAATCTTGATCACGTGGACAANNNNNNNNCTTCAGCTTCCC |
| | | GATATCCGACGGTAGTGTGGGGAAGAGCTGTTTGATCA |
| 212 | HK1_0248 | GCTATCCTCCAGCAGCTAGGTCTGAANNNNNNNNCTTCAGCTTCC |
| | | CGATATCCGACGGTAGTGTACTTCACCAAGAAGGGATT |
| 213 | HK2_0268 | CTACCACATGCGCCTCTCTGATNNNNNNNNCTTCAGCTTCCCGAT |
| | | ATCCGACGGTAGTGTTCTCGCGTCTCCGCCTCGGTTTC |
| 214 | HK2_0274 | GTTGGGACCATGATGACCTGTGGTTATGNNNNNNNNCTTCAGCTT |
| | | CCCGATATCCGACGGTAGTGTTCATGGACCAAGGGAT |
| 215 | HK2_0283 | GCTGGTCCGTGTTCGGAATGGGAAGTGGGNNNNNNNNCTTCAGCT |
| | | TCCCGATATCCGACGGTAGTGTAAGGAGACTCATGCCA |
| 216 | HK2_0291 | GTCTCAGATTGAGAGTGACTGCCTGGCNNNNNNNNCTTCAGCTTC |
| | | CCGATATCCGACGGTAGTGTGTGGAATGTACCTGGGTG |
| 217 | HK2_0295 | GCGGCGCTCATCACTGCTGTGGNNNNNNNNCTTCAGCTTCCCGAT |
| | | ATCCGACGGTAGTGTGTGGGTGTGGATGGGACCCTCTA |
| 218 | HK3_2013 | CGTCTGTGCGGCCGTGTGNNNNNNNNCTTCAGCTTCCCGATATCC |
| | | GACGGTAGTGTTGAGCCAAGGCAGCATCCTCCTG |
| 219 | HK3_2028 | CTCTTTCCCTTGTCACCAGACGGNNNNNNNNCTTCAGCTTCCCGA |
| | | TATCCGACGGTAGTGTTTTGTGATCCCCCAAGAGGTG |
| 220 | HK3_2032 | CGGAGGCCTGTACCTGGGTGAGCTNNNNNNNNCTTCAGCTTCCCG |
| | | ATATCCGACGGTAGTGTTCTGCGTCAGCGTCGAGT |
| 221 | HK3_2041 | GGCCTCATTGTCGGAACCGGCANNNNNNNNCTTCAGCTTCCCGAT |
| | | ATCCGACGGTAGTGTGATGTCGTGAGTCTGTTGCGGG |
| 222 | HK3_2045 | GAGATCGAAAGTGACAGCCTGGCCCTGCNNNNNNNNCTTCAGCTT |
| | | CCCGATATCCGACGGTAGTGTATGTACCTGGGGGAGA |
| 223 | Housekeeping_ | GCCCAGAGCAAGAGAGGCATCCTNNNNNNNNCTTCAGCTTCCCGA |
| | ACTB_0800 | TATCCGACGGTAGTGTGCATGTGCAAGGCCGGCTT |
| 224 | Housekeeping_ | GAAGATGACCCAGATCATGTTTGAGACCTNNNNNNNNCTTCAGCT |
| | ACTB_0802 | TCCCGATATCCGACGGTAGTGTACCAACTGGGACGACA |
| 225 | Housekeeping_ | GCTACGTCGCCCTGGACTTCGAGCAAGANNNNNNNNCTTCAGCTT |
| | ACTB_0805 | CCCGATATCCGACGGTAGTGTTGCGTCTGGACCTGGCT |
| 226 | Housekeeping_ | ACCACCATGTACCCTGGCATTGCCGACANNNNNNNNCTTCAGCTT |
| | ACTB_0808 | CCCGATATCCGACGGTAGTGTTTCCAGCCTTCCTTCCT |
| 227 | Housekeeping_ | GTGGATCAGCAAGCAGGAGTATGACGANNNNNNNNCTTCAGCTTC |
| | ACTB_0810 | CCGATATCCGACGGTAGTGTAGAAGGAGATCACTGCC |
| 228 | Housekeeping_ | GCTCAGGTCCTTTTGGCCAGATCTTNNNNNNNNCTTCAGCTTCCC |
| | TUBB_1551 | GATATCCGACGGTAGTGTGAGGCGAGCAAAAAAATTAA |
| 229 | Housekeeping_ | GCCTTCACCCAAAGTGTCTGACACNNNNNNNNCTTCAGCTTCCCG |
| | TUBB_1554 | ATATCCGACGGTAGTGTACTGCCTGCAGGGCTTCCAGC |
| 230 | Housekeeping_ | GTCCCCTTCCCACGTCTCCATTTCTTTATNNNNNNNNCTTCAGCT |
| | TUBB_1557 | TCCCGATATCCGACGGTAGTGTTGACCACACCAACCTA |
| 231 | Housekeeping_ | GTGGTCGGATGTCCATGAAGGAGGTCGATNNNNNNNNCTTCAGCT |
| | TUBB_1559 | TCCCGATATCCGACGGTAGTGTAAGCCAGCAGTATCGA |
| 232 | Housekeeping_ | GCCGAAGAGGAGGCCTAAGGCAGAGNNNNNNNNCTTCAGCTTCCC |
| | TUBB_1563 | GATATCCGACGGTAGTGTTACACAGGCGAGGGCATGGA |
| 233 | IDH3A_2545 | GCCATTCAAGGACCTGGAGGAAAGTGGANNNNNNNNCTTCAGCTT |
| | | CCCGATATCCGACGGTAGTGTGGTGGTGTTCAGACAGT |
| 234 | IDH3A_2546 | TAGCAGCCGGTCACCCATCTATGAANNNNNNNNCTTCAGCTTCCC |
| | | GATATCCGACGGTAGTGTTTCAGTGGGAGGAGCGG |
| 235 | IDH3A_2547 | CCCCTTACACCGATGTAAATATTGTGNNNNNNNNCTTCAGCTTCC |
| | | CGATATCCGACGGTAGTGTGGGCTTGAAAGGCCCTTTG |
| 236 | IDH3A_2548 | CGTGCAGAGTATCAAGCTCATCACCGAGNNNNNNNNCTTCAGCTT |
| | | CCCGATATCCGACGGTAGTGTTGTCCGACCATGTGTCT |
| 237 | IDH3A_2549 | CGGAGCAACGTCACGGCGGTGCACANNNNNNNNCTTCAGCTTCCC |
| | | GATATCCGACGGTAGTGTGAAGGAGAATACAGTGG |
| 238 | IDH3A_2550 | GAGATGTACCTTGATACAGTATGNNNNNNNNCTTCAGCTTCCCGA |
| | | TATCCGACGGTAGTGTATGCCCGGAACAACCAC |
| 239 | IDH3A_2551 | GTGACTTGTGTGCAGGATTGATCGGAGNNNNNNNNCTTCAGCTTC |
| | | CCGATATCCGACGGTAGTGTGCAGAAAGCTGTAAAGAT |
| 240 | IDH3A_2552 | GTCGGTTCATGGGACGGCTCCNNNNNNNNCTTCAGCTTCCCGATA |
| | | TCCGACGGTAGTGTCCCAATTTGATGTTCTTGTTATGC |
| 241 | IDH3A_2553 | GATGCTGCGCCACATGGGACTTTTTGACCNNNNNNNNCTTCAGCT |
| | | TCCCGATATCCGACGGTAGTGTACACCAAGTGGCAACA |
| 242 | IDH3A_2554 | GCAAAATGCTCAGACTTCACAGAGGANNNNNNNNCTTCAGCTTCC |
| | | CGATATCCGACGGTAGTGTTCCTGCTCAGTGCCGTGAT |
| 243 | IDH3A_2555 | TCTACAACTGGCATTTACATCAGTCACNNNNNNNNCTTCAGCTTC |
| | | CCGATATCCGACGGTAGTGTGAGGCTGCGTGTTTTG |
| 244 | IDH3B_2791 | GCTGAGTTCCATGAAGGAGAACAANNNNNNNNCTTCAGCTTCCCG |
| | | ATATCCGACGGTAGTGTGTGTGGGGCCTGAGCTGATG |
| 245 | IDH3B_2792 | GCGGCTGAGGCGTAAGTTGGACTTATTTNNNNNNNNCTTCAGCTT |
| | | CCCGATATCCGACGGTAGTGTGAATATGGCATCTGAGG |
| 246 | IDH3B_2793 | GTGATCATTCGAGAGCAGACAGAAGGGNNNNNNNNCTTCAGCTTC |
| | | CCGATATCCGACGGTAGTGTGGAGTATAAGGGGGAG |
| 247 | IDH3B_2794 | GCGGATTGCAAAGTTCGCCTTTGACTATGNNNNNNNNCTTCAGCT |
| | | TCCCGATATCCGACGGTAGTGTCACAACAATCTAGACC |
| 248 | IDH3B_2795 | GAAACTTGGGGATGGGTTGTTCCTNNNNNNNNCTTCAGCTTCCCG |
| | | ATATCCGACGGTAGTGTGGGGTGTGATTGAGTGTTTG |
| 249 | IDH3B_2796 | GTGCAGAATCCTTACCAGTTTGATGTGCTNNNNNNNNCTTCAGCT |
| | | TCCCGATATCCGACGGTAGTGTACAAGGCCAACATCAT |
| 250 | IDH3B_2797 | GCTGGTGTGGTCCCTGGTGAGAGCTNNNNNNNNCTTCAGCTTCCC |
| | | GATATCCGACGGTAGTGTGAGACAATGATCATAGACAA |
| 251 | IDH3B_2798 | GCCATGCTGCTGTCGGCTTCNNNNNNNNCTTCAGCTTCCCGATAT |
| | | CCGACGGTAGTGTTGGCTGCTGGCCTGGTTGGGGG |
| 252 | IDH3B_2799 | GCAAGGTGCGGACTCGAGACATGGNNNNNNNNCTTCAGCTTCCCG |
| | | ATATCCGACGGTAGTGTGCAGTGGGCAGGAATATAG |
| 253 | IDH3B_2800 | GCCCTTTATTTCTTCCAACCTTGCAAGGANNNNNNNNCTTCAGCT |
| | | TCCCGATATCCGACGGTAGTGTAGATGCGGTGAAGAAG |
| 254 | IDH3G_3240 | GCACACGGTGACCATGATCCCAGGGGATNNNNNNNNCTTCAGCTT |
| | | CCCGATATCCGACGGTAGTGTGAAGGCGGTGCTCGGG |
| 255 | IDH3G_3241 | GTACCAGTGGACTTTGAAGAGGTGCACGTNNNNNNNNCTTCAGCT |
| | | TCCCGATATCCGACGGTAGTGTCAGAACAAACAATTCC |
| 256 | IDH3G_3242 | GCCCTGAAGGGCAACATCGAAACNNNNNNNNCTTCAGCTTCCCGA |
| | | TATCCGACGGTAGTGTTGCTGCATGTCAAGTCCGTCTT |
| 257 | IDH3G_3243 | CGTCATCCACTGTAAGAGCCTTCCNNNNNNNNCTTCAGCTTCCCG |
| | | ATATCCGACGGTAGTGTATGCCATCATGGCCATCCGCC |
| 258 | IDH3G_3244 | GTACAGCAGCCTGGAGCATGAGAGTNNNNNNNNCTTCAGCTTCCC |
| | | GATATCCGACGGTAGTGTAAACAACATCCTTCGCACCA |
| 259 | IDH3G_3245 | GCATTGCCGAGTATGCCTTCAAGCTGGCGNNNNNNNNCTTCAGCT |
| | | TCCCGATATCCGACGGTAGTGTCAAGGACATAGACATC |
| 260 | IDH3G_3246 | GCTTTTCCTCCAGTGCTGCAGGGAGGTGGNNNNNNNNCTTCAGCT |
| | | TCCCGATATCCGACGGTAGTGTAAGATCATCACCAAGG |
| 261 | IDH3G_3247 | CGGCCCCAGCAGTTTGATGTCATGGTGATNNNNNNNNCTTCAGCT |
| | | TCCCGATATCCGACGGTAGTGTCCAACATCATGAAACT |
| 262 | IDH3G_3248 | GTGGCTGGGGCCAACTATGGCCATGTGTANNNNNNNNCTTCAGCT |
| | | TCCCGATATCCGACGGTAGTGTTGGATAACACCACCAT |
| 263 | IDH3G_3249 | CCAACCCCACGGCCACCCTNNNNNNNNCTTCAGCTTCCCGATATC |
| | | CGACGGTAGTGTTCGTCAACAATGTCTGCGCGGGACTG |
| 264 | IDH3G_3250 | GCTGTCCTGGCATCCATGGACAATGAGNNNNNNNNCTTCAGCTTC |
| | | CCGATATCCGACGGTAGTGTTACGAGGAACACCGGCAA |
| 265 | IDH3G_3239 | CACTGACCACAGCCCCCANNNNNNNNCTTCAGCTTCCCGATATCC |
| | | GACGGTAGTGTTGCACTCCTATGCCACCTCCATCCGT |
| 266 | L2HGDH_3084 | GTCATCGTTGGTGGCGGANNNNNNNNCTTCAGCTTCCCGATATCC |
| | | GACGGTAGTGTTTTGGTTGGTGCCTGCGGACGGG |
| 267 | L2HGDH_3085 | GTTCTGGAAAAGGAGAAAGATTTAGCTGTNNNNNNNNCTTCAGCT |
| | | TCCCGATATCCGACGGTAGTGTTGTGTGGAGGTAGCCG |
| 268 | L2HGDH_3086 | TGTGTACAAGGTGCAGCCCTCCTCTNNNNNNNNCTTCAGCTTCCC |
| | | GATATCCGACGGTAGTGTCATCCATCACTTTCTATTGG |
| 269 | L2HGDH_3087 | TTCCCAGACTTCAGGCCCNNNNNNNNCTTCAGCTTCCCGATATCC |
| | | GACGGTAGTGTTATTATAAACCTGAGTCTCTGAAAGCC |
| 270 | L2HGDH_3088 | GCCATATTGTAGGGGTCTAATGGCTATTGNNNNNNNNCTTCAGCT |
| | | TCCCGATATCCGACGGTAGTGTAAGCTTATAGTAGCTG |
| 271 | L2HGDH_3089 | GCAGGTGGCTCTGTCTTGNNNNNNNNCTTCAGCTTCCCGATATCC |
| | | GACGGTAGTGTGAGGCTGATCCAGCAGGAGGAT |
| 272 | L2HGDH_3090 | GAATACAAAGGGAGAGGAAATTCGATGTCNNNNNNNNCTTCAGCT |
| | | TCCCGATATCCGACGGTAGTGTGCCCAGGATTTCCAAG |
| 273 | L2HGDH_3091 | GGCTGCACTCCTGATCCTCGAATTGNNNNNNNNCTTCAGCTTCCC |
| | | GATATCCGACGGTAGTGTCCTTCAAGAAGTATAGATGG |
| 274 | L2HGDH_3092 | GCCGGTTTCCTTTCCTAGGAGTTCNNNNNNNNCTTCAGCTTCCCG |
| | | ATATCCGACGGTAGTGTGACCGTATTTCAGAGTTGAGT |
| 275 | L2HGDH_3093 | CCCTTTGACTTCAGTGCCACAGATGNNNNNNNNCTTCAGCTTCCC |
| | | GATATCCGACGGTAGTGTGGAAATATTTATCCGGTCCC |
| 276 | L2HGDH_3094 | GCATGTTTTCTTGGTGCAACAGTGAAGTNNNNNNNNCTTCAGCTT |
| | | CCCGATATCCGACGGTAGTGTAAACGAGAGGGTTACAG |
| 277 | L2HGDH_3095 | GCCCAGCTGGAGTAAGAGCCCAGNNNNNNNNCTTCAGCTTCCCGA |
| | | TATCCGACGGTAGTGTGATTAAACTGGCATCCCAGAAT |
| 278 | L2HGDH_3096 | GGGGATATTGGAAATCGCATTCTTCATGNNNNNNNNCTTCAGCTT |
| | | CCCGATATCCGACGGTAGTGTCTTCAAAAATTCATCCC |
| 279 | L2HGDH_3097 | GCAGATGAAGTACAACAAAGATTTGNNNNNNNNCTTCAGCTTCCC |
| | | GATATCCGACGGTAGTGTGATGGAAATCTGGTAGAAG |
| 280 | L2HGDH_3098 | GCAACAAGAATGTACTAATTGCATTCTTTNNNNNNNNCTTCAGCT |
| | | TCCCGATATCCGACGGTAGTGTGCACCTTCTCCTGCTG |
| 281 | LDHA_0840 | GCATGGCCTGTGCCATCAGTATCTNNNNNNNNCTTCAGCTTCCCG |
| | | ATATCCGACGGTAGTGTGCATTCCCGATTCCTTTTGGT |
| 282 | LDHA_0842 | GTTATTGGAAGCGGTTGCAATCTGGANNNNNNNNCTTCAGCTTCC |
| | | CGATATCCGACGGTAGTGTTGAAGGGAGAGATGATGGA |
| 283 | LDHA_0844 | GCACCCAGATTTAGGGACTGATAAAGANNNNNNNNCTTCAGCTTC |
| | | CCGATATCCGACGGTAGTGTTAATGGGGGAAAGGCTGG |
| 284 | LDHA_0846 | GGATGATGTCTTCCTTAGTGTTCCTTGNNNNNNNNCTTCAGCTTC |
| | | CCGATATCCGACGGTAGTGTAACTCAAAGGCTACACAT |
| 285 | LDHA_0848 | GCATGTTGTCCTTTTTATCTGATCTGTGNNNNNNNNCTTCAGCTT |
| | | CCCGATATCCGACGGTAGTGTGGCCCGTTTGAAGAAGA |
| 286 | LDHB_0954 | GTTGGTATGGCGTGTGCTATCAGCATTCNNNNNNNNCTTCAGCTT |
| | | CCCGATATCCGACGGTAGTGTAAAACCAGGCCCTACT |
| 287 | LDHB_0956 | GCAAGAAGGGGAGAGTCGGCTCAATCNNNNNNNNCTTCAGCTTCC |
| | | CGATATCCGACGGTAGTGTAAAGGAGAAATGATGGATC |
| 288 | LDHB_0959 | GTGTGGCTGTGTGGAGTGGTGTGNNNNNNNNCTTCAGCTTCCCGA |
| | | TATCCGACGGTAGTGTCAAACACCGCGTGATTGGAA |
| 289 | LDHB_0961 | GTGTGGCTGATCTTATTGAATCCATGTTGNNNNNNNNCTTCAGCT |
| | | TCCCGATATCCGACGGTAGTGTGAATCCAGAAATGGGA |
| 290 | LDHB_0963 | GCTCAAGAAAAGTGCAGATACCCTGTGGGNNNNNNNNCTTCAGCT |
| | | TCCCGATATCCGACGGTAGTGTACAATGGTAAAGGGGA |
| 291 | MAPK8_1429 | CCTATAGGCTCAGGAGCTCAAGGAATAGTNNNNNNNNCTTCAGCT |
| | | TCCCGATATCCGACGGTAGTGTGGATGAAGCCATTAAA |
| 292 | MAPK8_1432 | GCAAATCTTTGCCAAGTGATTCAGATGNNNNNNNNCTTCAGCTTC |
| | | CCGATATCCGACGGTAGTGTCAGAGAGCTAGTTCTTAT |
| 293 | MAPK8_1435 | CGTTGACATTTGGTCAGTTGGGTGNNNNNNNNCTTCAGCTTCCCG |
| | | ATATCCGACGGTAGTGTGCACTTTGAAGATTCTTGACT |
| 294 | MAPK8_1438 | GCTGGTAATAGATGCATCTAAAAGGATCTNNNNNNNNCTTCAGCT |
| | | TCCCGATATCCGACGGTAGTGTGAAAACAGACCTAAAT |
| 295 | MAPK8_1441 | GCTCTCAGCATCCATCATCATCGTCGTCTNNNNNNNNCTTCAGCT |
| | | TCCCGATATCCGACGGTAGTGTGGAACACACAATAGAA |
| 296 | MYC_2089 | CGACTCGGTGCAGCCGTATTTCTNNNNNNNNCTTCAGCTTCCCGA |
| | | TATCCGACGGTAGTGTCTTCTCTGAAAGGCTCTCCTTG |
| 297 | MYC_2090 | TTCGAGCTGCTGCCCACCCCNNNNNNNNCTTCAGCTTCCCGATAT |
| | | CCGACGGTAGTGTACAGGAACTATGACCTCGACT |
| 298 | MYC_2093 | TCTGTGGAAAAGAGGCAGGCTCCNNNNNNNNCTTCAGCTTCCCGA |
| | | TATCCGACGGTAGTGTTGCTCTCCTCGACGGAGTCCT |
| 299 | MYC_2094 | CTGGTCCTCAAGAGGTGCCACGTCTNNNNNNNNCTTCAGCTTCCC |
| | | GATATCCGACGGTAGTGTGAGGAGGAACAAGAAGA |
| 300 | MYC_2095 | CAGTGTCAGAGTCCTGAGACAGATCAGNNNNNNNNCTTCAGCTTC |
| | | CCGATATCCGACGGTAGTGTACAGCAAACCTCCTCACA |
| 301 | MYC_2096 | GCGCCAGAGGAGGAACGAGCTAAAACGGANNNNNNNNCTTCAGCT |
| | | TCCCGATATCCGACGGTAGTGTAAGAGGGTCAAGTTGG |
| 302 | MYC_2097 | AGCCACAGCATACATCCTGTCCGTCCNNNNNNNNCTTCAGCTTCC |
| | | CGATATCCGACGGTAGTGTCGAACACACAACGTCTTGG |
| 303 | MYC_2098 | CTTGAACAGCTACGGAACTCTTGTGCGNNNNNNNNCTTCAGCTTC |
| | | CCGATATCCGACGGTAGTGTGGCCCCCAAGGTAGTTAT |
| 304 | MYC_2099 | CCTTCTAACAGAAATGTCCTGAGCAATCANNNNNNNNCTTCAGCT |
| | | TCCCGATATCCGACGGTAGTGTAAGCAGAGGAGCAAAA |
| 305 | NAMPT_2562 | CTTTGAATGCCGTGAAAAGAAGACAGAANNNNNNNNCTTCAGCTT |
| | | CCCGATATCCGACGGTAGTGTTGTCCTCCGGCCCGAGA |
| 306 | NAMPT_2565 | GGAAATGTTCTCTTCACGGTGGAAAACACNNNNNNNNCTTCAGCT |
| | | TCCCGATATCCGACGGTAGTGTACAAAGAACATTTCCA |
| 307 | NAMPT_2568 | GTAGCAGGACTTGCTCTAATTANNNNNNNNCTTCAGCTTCCCGAT |
| | | ATCCGACGGTAGTGTGAAACTTCTGGTAACTTAGATGG |
| 308 | NAMPT_2572 | GCCACCTTATCTTAGAGTTATTCAAGGGGNNNNNNNNCTTCAGCT |
| | | TCCCGATATCCGACGGTAGTGTACACAGGCACCACTAA |
| 309 | NAMPT_2575 | CGCCAGCAGGGAATTTTGTTACACTGGNNNNNNNNCTTCAGCTTC |
| | | CCGATATCCGACGGTAGTGTCTCTTGAATTGTTCCTTC |
| 310 | NAPRT1_3185 | GCCCAGGTGGAGCCACTANNNNNNNNCTTCAGCTTCCCGATATCC |
| | | GACGGTAGTGTTTTCCGGCTCCTGGGCTCTGACGGGT |
| 311 | NAPRT1_3193 | GTTCCAGGTGCCCTGGCTGGAGTNNNNNNNNCTTCAGCTTCCCGA |
| | | TATCCGACGGTAGTGTAACTTCCTAGCAGTCGCCCT |
| 312 | NAPRT1_3194 | GTCATTGGCATTGGCACCAGTGTGGTCANNNNNNNNCTTCAGCTT |
| | | CCCGATATCCGACGGTAGTGTGTCTTCCGAGCTGCTG |
| 313 | NAPRT1_3195 | CGAGGACCCCGAGAAGCAGACGTTGCCTGNNNNNNNNCTTCAGCT |
| | | TCCCGATATCCGACGGTAGTGTGAGGGCAGTGAGGTGA |
| 314 | NOX1_2825 | GCCTTCCTGAAATATGAGAAGGCCGNNNNNNNNCTTCAGCTTCCC |
| | | GATATCCGACGGTAGTGTTCTTCCCTGTTGCCTAGAAG |
| 315 | NOX1_2829 | CCCATCCAGTCCCGAAACACNNNNNNNNCTTCAGCTTCCCGATAT |
| | | CCGACGGTAGTGTCTATTCACATCATTGCACACCTGTT |
| 316 | NOX1_2833 | GCACCGGTCATTCTTTATATCTGTGNNNNNNNNCTTCAGCTTCCC |
| | | GATATCCGACGGTAGTGTGAGAGCATGAATGAGAGTCA |
| 317 | NOX1_2837 | GCTGGTTGGAGCAGGAATTGGGGTNNNNNNNNCTTCAGCTTCCCG |
| | | ATATCCGACGGTAGTGTCAGCAGGGGACTGGACAGAAA |
| 318 | NOX1_2841 | GTCTGTAGTGGGAGTTTTCTTATGTGGCCNNNNNNNNCTTCAGCT |
| | | TCCCGATATCCGACGGTAGTGTGGTCATGCAGCATTAA |
| 319 | NOX3_2954 | CGAGTTATTTTGGGTTCAACACTGGCNNNNNNNNCTTCAGCTTCC |
| | | CGATATCCGACGGTAGTGTTGGGGTGCTGGATTTTGAA |
| 320 | NOX3_2958 | CCCCACAAACACAACCACNNNNNNNNCTTCAGCTTCCCGATATCC |
| | | GACGGTAGTGTACATCGTGGCGCATTTCTTCAACCTGG |
| 321 | NOX3_2962 | GCGATTTCAACAAGAAGTTGTCATTACCANNNNNNNNCTTCAGCT |
| | | TCCCGATATCCGACGGTAGTGTGCAGAATGGCAGACAG |
| 322 | NOX3_2966 | GCGTTGCCGCGGGGATCGGAGTCACTCNNNNNNNNCTTCAGCTTC |
| | | CCGATATCCGACGGTAGTGTCTACTGGAGGCCTTTGG |
| 323 | NOX3_2970 | CAAGCAGATTGCCTACAATCACCCCANNNNNNNNCTTCAGCTTCC |
| | | CGATATCCGACGGTAGTGTTTTCTTACCGGCTGGGATG |
| 324 | NOX4a_3007 | GTCCTGCTTTTCTGGAAAACCTTCNNNNNNNNCTTCAGCTTCCCG |
| | | ATATCCGACGGTAGTGTTCCTTCTCGGTCCGGCGGGCA |
| 325 | NOX4a_3011 | ACTTCTCTTCACAACTGTTCCTGGNNNNNNNNCTTCAGCTTCCCG |
| | | ATATCCGACGGTAGTGTCTATCTGTATTTTCTCAGGCG |
| 326 | NOX4a_3015 | GCCCAGATTCCAAGCTAATTTTCCACNNNNNNNNCTTCAGCTTCC |
| | | CGATATCCGACGGTAGTGTACCAGCTCTCAGAATATTT |
| 327 | NOX4a_3019 | GAAATTCTGCCCTTCATTCAATCNNNNNNNNCTTCAGCTTCCCGA |
| | | TATCCGACGGTAGTGTATCATCCATTTACCCTCACAAT |
| 328 | NOX4a_3023 | CGGTGGAAACTTTTGTTTGATGAAATAGCNNNNNNNNCTTCAGCT |
| | | TCCCGATATCCGACGGTAGTGTGGCAAGAGAACAGACC |
| 329 | NQO1_0486 | GCGGCTTTGAAGAAGAAAGGATGGGANNNNNNNNCTTCAGCTTCC |
| | | CGATATCCGACGGTAGTGTAACCACGAGCCCAGCCAAT |
| 330 | NQO1_0488 | GCTGGAAGCCGCAGACCTTGTGATATTCNNNNNNNNCTTCAGCTT |
| | | CCCGATATCCGACGGTAGTGTCATTTCCAGAAAGGACA |
| 331 | NQO1_0490 | CATCACCACTGGTGGCAGTGGCTCCATGNNNNNNNNCTTCAGCTT |
| | | CCCGATATCCGACGGTAGTGTGTGGTTTGGAGTCCCTG |
| 332 | NQO1_0492 | GCCCGAATTCAAATCCTGGAAGGATGGAANNNNNNNNCTTCAGCT |
| | | TCCCGATATCCGACGGTAGTGTAAGGGATCCACGGGGA |
| 333 | NQO1_0494 | CAAGTCCATCCCAACTGACAACCNNNNNNNNCTTCAGCTTCCCGA |
| | | TATCCGACGGTAGTGTACACCACTGTATTTTGCTCCAA |
| 334 | OGDH_0591 | GATTCGGTGCTATTCTGCACCTGTTGNNNNNNNNCTTCAGCTTCC |
| | | CGATATCCGACGGTAGTGTAAAAACTTCAGGACAAAAA |
| 335 | OGDH_0592 | GCTGGAAAACCCCAAAAGTGTACATAAGNNNNNNNNCTTCAGCTT |
| | | CCCGATATCCGACGGTAGTGTACAAAACAGACCAGCAG |
| 336 | OGDH_0593 | CTGCCTACCAGAGTCCCCTTCNNNNNNNNCTTCAGCTTCCCGATA |
| | | TCCGACGGTAGTGTCCCTTTCTCAGTGGGACTAGTTCG |
| 337 | OGDH_0595 | GCTGATCTGGACTCCTCCGTGCCCGCTGANNNNNNNNCTTCAGCT |
| | | TCCCGATATCCGACGGTAGTGTTAGAAGCACAGCCCAA |
| 338 | OGDH_0596 | TTCCACTTGCCCACCACCACNNNNNNNNCTTCAGCTTCCCGATAT |
| | | CCGACGGTAGTGTACCATGTAGCACAGCTGGACCCCCT |
| 339 | OGDH_0597 | GCATATTGGGGTGGAGTTCATGTTCATNNNNNNNNCTTCAGCTTC |
| | | CCGATATCCGACGGTAGTGTATGGCCTGGATGAGTCTG |
| 340 | OGDH_0598 | GCAGTTCACAAATGAGGAGAAACGGACNNNNNNNNCTTCAGCTTC |
| | | CCGATATCCGACGGTAGTGTTGCGGGAGATCATCCG |
| 341 | OGDH_0599 | GCTTTGGTCTAGAAGGCTGCGAGGTACTNNNNNNNNCTTCAGCTT |
| | | CCCGATATCCGACGGTAGTGTAGAAGTTTGAGACCCCT |
| 342 | OGDH_0600 | GAGGGCGGCTGAACGTGCTTGCAAATNNNNNNNNCTTCAGCTTCC |
| | | CGATATCCGACGGTAGTGTGAAGTGGTCCTCTGAGAAG |
| 343 | OGDH_0601 | GCTGATGAGGGCTCCGGAGATGTGAAGTNNNNNNNNCTTCAGCTT |
| | | CCCGATATCCGACGGTAGTGTGAGAATGGCGTGGACT |
| 344 | OGDH_0602 | CTTGTCCTTGGTGGCCAACCCTTCNNNNNNNNCTTCAGCTTCCCG |
| | | ATATCCGACGGTAGTGTAGCTGGAACAGATCTTCTGTC |
| 345 | OGDH_0603 | GCGACACTGAAGGGAAAAAGGTAAGGCCNNNNNNNNCTTCAGCTT |
| | | CCCGATATCCGACGGTAGTGTACCGCAGGATCAATCGT |
| 346 | OGDH_0604 | GGAGTTCCGCTCACCAACATAACCCAGANNNNNNNNCTTCAGCTT |
| | | CCCGATATCCGACGGTAGTGTTGACCCCGTGGTGATGG |
| 347 | RARP1_1853 | AGCATCCCCAAGGACTCGCNNNNNNNNCTTCAGCTTCCCGATATC |
| | | CGACGGTAGTGTTGTTTCTAGGTCGTGGCGTCGGGCTT |
| 348 | RARP1_1859 | GAGTGGATGAAGTGGCGAAGAAGAAATCTNNNNNNNNCTTCAGCT |
| | | TCCCGATATCCGACGGTAGTGTCGAGTACAGTGCGAGT |
| 349 | RARP1_1868 | GCAAGGGCCAGGTCAAGGAGGAAGGTATCNNNNNNNNCTTCAGCT |
| | | TCCCGATATCCGACGGTAGTGTCAAGAGCCTTCAGGAG |
| 350 | RARP1_1877 | GCTGGACATCGAGGTGGCCTACAGTNNNNNNNNCTTCAGCTTCCC |
| | | GATATCCGACGGTAGTGTCTCTCAGATCCTGGATCTCT |
| 351 | RARP1_1883 | CCTTCAGCTAACATTAGTCTGGATGGTGNNNNNNNNCTTCAGCTT |
| | | CCCGATATCCGACGGTAGTGTGGCTTAATCCTGTTGGG |
| 352 | PC_0499 | GTGGATGTGGCAGCTGATTCCATGTCTGNNNNNNNNCTTCAGCTT |
| | | CCCGATATCCGACGGTAGTGTACCATGCTGGTCAGCT |
| 353 | PC_0507 | GCATGAGGGTGGTGCACAGCTANNNNNNNNCTTCAGCTTCCCGAT |
| | | ATCCGACGGTAGTGTTTGCTGCGGGTGTTCCCGTTGTC |
| 354 | PC_0515 | CCAAAAGCTGTTGCACTACCTCGGCCNNNNNNNNCTTCAGCTTCC |
| | | CGATATCCGACGGTAGTGTAAGACCAACATCGCCTTCC |
| 355 | PC_0524 | GCGCGTGTTTGACTACAGTGAGTACTGNNNNNNNNCTTCAGCTTC |
| | | CCGATATCCGACGGTAGTGTAGGCTGGAGCTGATGTG |
| 356 | PC_0532 | CAAGGACACCCAGGCCATGAAGGAGATGNNNNNNNNCTTCAGCTT |
| | | CCCGATATCCGACGGTAGTGTTGAGGTGGAGCTGGAG |
| 357 | PDHA1_0305 | GAAATTAAGAAATGTGACCTTCACCGGCNNNNNNNNCTTCAGCTT |
| | | CCCGATATCCGACGGTAGTGTCACTGCCTGTGCTTCAT |
| 358 | PDHA1_0308 | GCTCACGGCTTTACTTTCACCCGGGGNNNNNNNNCTTCAGCTTCC |
| | | CGATATCCGACGGTAGTGTCAGATCAGCTGTATAAACA |
| 359 | PDHA1_0311 | GTGGAAATTACCTTGTATTTTCATCTGTGNNNNNNNNCTTCAGCT |
| | | TCCCGATATCCGACGGTAGTGTCTGGGCGCTGGGATTG |
| 360 | PDHA1_0313 | GTAGATCTGGGAAGGGGCCCATCCTGATGNNNNNNNNCTTCAGCT |
| | | TCCCGATATCCGACGGTAGTGTTGTTGAGAGAGCGGCA |
| 361 | PDHA1_0316 | GGAAGAGCTGGGCTACCACATCTACTCCANNNNNNNNCTTCAGCT |
| | | TCCCGATATCCGACGGTAGTGTAAGGACAGGATGGTGA |
| 362 | PDK1_1451 | GATAATCTTCTCAGGACACCATCCGNNNNNNNNCTTCAGCTTCCC |
| | | GATATCCGACGGTAGTGTCTCTCCATGAAGCAGTTCCT |
| 363 | PDK1_1453 | GCAAGATGATCTTTACAGATACTGTGNNNNNNNNCTTCAGCTTCC |
| | | CGATATCCGACGGTAGTGTCTGGTATATCCAGAGTCTT |
| 364 | PDK1_1456 | GCTATGAAAATGCTAGGCGTCTGTGNNNNNNNNCTTCAGCTTCCC |
| | | GATATCCGACGGTAGTGTCAATTAGAATGTTACTCAAT |
| 365 | PDK1_1459 | GCGTTCCTTTGAGGAAAATTGACAGACTTNNNNNNNNCTTCAGCT |
| | | TCCCGATATCCGACGGTAGTGTCAAGAATGCAATGAGA |
| 366 | PDK1_1462 | GCCTGGAAGCATTACAACACCAACCNNNNNNNNCTTCAGCTTCCC |
| | | GATATCCGACGGTAGTGTTACGCACAATACTTCCAAGG |
| 367 | PFKB1_1411 | TGCGCCCTGGCAGCCCTGAAGGATGTTCANNNNNNNNCTTCAGCT |
| | | TCCCGATATCCGACGGTAGTGTAAGAAAAAACCTCTAG |
| 368 | PFKB1_1414 | GAGGAACTGGACAGCCACCTGTCCTACATNNNNNNNNCTTCAGCT |
| | | TCCCGATATCCGACGGTAGTGTCAGAAAACATCAGGCA |
| 369 | PFKB1_1417 | GTCACATGAAGAGGACCATCCAGACAGCNNNNNNNNCTTCAGCTT |
| | | CCCGATATCCGACGGTAGTGTAACTCAACATCAGAGGC |
| 370 | PFKB1_1420 | GCTGTCATGCGGTGCCTCCTGGCCTATTTNNNNNNNNCTTCAGCT |
| | | TCCCGATATCCGACGGTAGTGTACCAAGATAAATATCG |
| 371 | PFKB1_1422 | ACATCACCCGGGAACCTGANNNNNNNNCTTCAGCTTCCCGATATC |
| | | CGACGGTAGTGTAGCTTCCATATCTCAAGTGCCCTCTG |
| 372 | PFKMb_0914 | GCTGGGGAAGCTTCTACTTCCAGCATGCTNNNNNNNNCTTCAGCT |
| | | TCCCGATATCCGACGGTAGTGTAACCGCCTTCACAGCA |
| 373 | PFKMb_0920 | GTGGAGTGACTTGTTGAGTGACCTCCNNNNNNNNCTTCAGCTTCC |
| | | CGATATCCGACGGTAGTGTAAGGACTTTCGGGAACGAG |
| 374 | PFKMb_0926 | GCAGGATGGGTGTGGAAGCAGTGATNNNNNNNNCTTCAGCTTCCC |
| | | GATATCCGACGGTAGTGTAACCAATCACCTCAGAAGAC |
| 375 | PFKMb_0932 | CATTGGGGGCTTTGAGGCTTACACAGGGGNNNNNNNNCTTCAGCT |
| | | TCCCGATATCCGACGGTAGTGTTATGTTGGGGGCTGGA |
| 376 | PFKMb_0940 | GCTGAAGGACCAGACAGATTTTGAGCANNNNNNNNCTTCAGCTTC |
| | | CCGATATCCGACGGTAGTGTTGAACTGGATGTCTGGG |
| 377 | PGAM1_2160 | GATGTGGCTGCCAGTGGTGAGGACTTGNNNNNNNNCTTCAGCTTC |
| | | CCGATATCCGACGGTAGTGTACGAGGAGGCGAAGCG |
| 378 | PGAM1_2162 | CGCAGGTATGCAGACCTCACAGAAGATNNNNNNNNCTTCAGCTTC |
| | | CCGATATCCGACGGTAGTGTCAATAAAGCAGAAACTGC |
| 379 | PGAM1_2163 | CAGATCAAGGAGGGGAAACGTGTACTGATNNNNNNNNCTTCAGCT |
| | | TCCCGATATCCGACGGTAGTGTACAGCAACATCAGTAA |
| 380 | PGAM1_2165 | GCGCAAAGCCATGGAAGCTGTGGCTNNNNNNNNCTTCAGCTTCCC |
| | | GATATCCGACGGTAGTGTGGAGGGTCTCTCTGAAGAG |
| 381 | PGAM1_2166 | GCCGGCGGGGAGGATACTGTNNNNNNNNCTTCAGCTTCCCGATAT |
| | | CCGACGGTAGTGTGGTATTCCCATTGTCTATGAATTGG |
| 382 | PGD_2169 | GCCAATGAGGCAAAGGGAACCAAAGTGGTNNNNNNNNCTTCAGCT |
| | | TCCCGATATCCGACGGTAGTGTAAGCTGACATCGCGCT |
| 383 | PGD_2173 | GCTGCAAAAGTGGGAACTGGAGNNNNNNNNCTTCAGCTTCCCGAT |
| | | ATCCGACGGTAGTGTGCCAAGGGAATTTTATTTGTGGG |
| 384 | PGD_2177 | CCCGTCACCCTCATTGGAGAAGCTGTCTTNNNNNNNNCTTCAGCT |
| | | TCCCGATATCCGACGGTAGTGTCAGCCAATATTCTCAA |
| 385 | PGD_2181 | GTCAGCTGTTGAAAACTGCCAGGACNNNNNNNNCTTCAGCTTCCC |
| | | GATATCCGACGGTAGTGTTTATGGTGGCATCGCCCTGA |
| 386 | PGD_2183 | CCAGGGCAGTTTATCCACACCAANNNNNNNNCTTCAGCTTCCCGA |
| | | TATCCGACGGTAGTGTTTCCCATGCCCTGTTTTACCAC |
| 387 | PGI_1528 | ACCGCTTCAACCACTTCAGCNNNNNNNNCTTCAGCTTCCCGATAT |
| | | CCGACGGTAGTGTCTCACTCAGTGTACCTTCTAGTCCC |
| 388 | PGI_1533 | GTGGTTTCTCCAGGCGGCCAAGGATNNNNNNNNCTTCAGCTTCCC |
| | | GATATCCGACGGTAGTGTCAACATTGATGGAACTCACA |
| 389 | PGI_1536 | GCTGGGTATCTGGTACATCAACTGCTTTNNNNNNNNCTTCAGCTT |
| | | CCCGATATCCGACGGTAGTGTACTCTCCATTGCCCTGC |
| 390 | PGI_1539 | GCATCACAAGATCCTCCTGGCCAANNNNNNNNCTTCAGCTTCCCG |
| | | ATATCCGACGGTAGTGTTTGTGTGGGGGGAGCCAGGG |
| 391 | PGI_1542 | GCTGGCTAAGAAAATAGAGCCTGAGCTTGNNNNNNNNCTTCAGCT |
| | | TCCCGATATCCGACGGTAGTGTACCAAGCTCACACCAT |
| 392 | PGK1_0371 | CAACCAGAGGATTAAGGCTGCTGTCCNNNNNNNNCTTCAGCTTCC |
| | | CGATATCCGACGGTAGTGTCCCAGCTGTATTTCCAAAA |
| 393 | PGK1_0374 | GCTGGAGAACCTCCGCTTTCATGTGGNNNNNNNNCTTCAGCTTCC |
| | | CGATATCCGACGGTAGTGTTCCTTAGAGCCAGTTGCTG |
| 394 | PGK1_0377 | GCAGACAAGATCCAGCTCATCANNNNNNNNCTTCAGCTTCCCGAT |
| | | ATCCGACGGTAGTGTTCCATGGTAGGAGTCAATCTGCC |
| 395 | PGK1_0380 | GCTGGCTGGATGGGCTTGGACTGTGNNNNNNNNCTTCAGCTTCCC |
| | | GATATCCGACGGTAGTGTAAAGACCTAATGTCCAAAGC |
| 396 | PGK1_0383 | GTGGTGCCAGTTTGGAGCTCCTGGAAGGTNNNNNNNNCTTCAGCT |
| | | TCCCGATATCCGACGGTAGTGTCATGGATGAGGTGGTG |
| 397 | PGK2_3123 | CCAGATTACAAACAACCAGAGGATCANNNNNNNNCTTCAGCTTCC |
| | | CGATATCCGACGGTAGTGTGGTGTCAGCCTATGTCTTT |
| 398 | PGK2_3125 | GTTCCTGAAGGACTGTGTAGGCGCAGNNNNNNNNCTTCAGCTTCC |
| | | CGATATCCGACGGTAGTGTAATGGAGCCAAGGCAGTAG |
| 399 | PGK2_3128 | GCTAAAGCCTTGGAAAACCCAGTGAGANNNNNNNNCTTCAGCTTC |
| | | CCGATATCCGACGGTAGTGTGCTAGGGGACGTCTATGT |
| 400 | PGK2_3131 | GTTTGACGAGAACGCTCAGGTTGGAAAAGNNNNNNNNCTTCAGCT |
| | | TCCCGATATCCGACGGTAGTGTATGGAGATTGGTGCTT |
| 401 | PGK2_3134 | GATAAAGTCAGCCATGTCAGCACTGGNNNNNNNNCTTCAGCTTCC |
| | | CGATATCCGACGGTAGTGTGGGATGCCTTTGCTAAGGG |
| 402 | PKM_1091 | CCCAACCCCAGAGAACCAANNNNNNNNCTTCAGCTTCCCGATATC |
| | | CGACGGTAGTGTAGAAGTCCCCAGCGCCGTTCCTTCCA |
| 403 | PKM_1095 | CACTAAAGGACCTGAGATCCGAACNNNNNNNNCTTCAGCTTCCCG |
| | | ATATCCGACGGTAGTGTTGGCTCGTCTGAACTTCTCTC |
| 404 | PKM_1099 | GCAGGATGTTGATATGGTGTTTGCGTNNNNNNNNCTTCAGCTTCC |
| | | CGATATCCGACGGTAGTGTCTGGTGACGGAGGTGGAAA |
| 405 | PKM_1103 | GCCAAAGGGGACTATCCTCTGGANNNNNNNNCTTCAGCTTCCCGA |
| | | TATCCGACGGTAGTGTCTGTCATCTGTGCTACTCAGAT |
| 406 | PKM_1107 | ACCTCCGGGTGAACTTTGCCATGAATGTTNNNNNNNNCTTCAGCT |
| | | TCCCGATATCCGACGGTAGTGTACGTGCCCCCATCATT |
| 407 | PRDX1_1078 | GTTGTGTTCTTCTTTTACCCTCTTGNNNNNNNNCTTCAGCTTCCC |
| | | GATATCCGACGGTAGTGTGCTGATAGGAAGATGTCTTC |
| 408 | PRDX1_1080 | CGAAGCGCACCATTGCTCAGGATTATGNNNNNNNNCTTCAGCTTC |
| | | CCGATATCCGACGGTAGTGTAAGAAACTCAACTGCCAA |
| 409 | PRDX1_1081 | GCAGATCACTGTAAATGACCTCCCTGNNNNNNNNCTTCAGCTTCC |
| | | CGATATCCGACGGTAGTGTCCCATGAACATTCCTTTGG |
| 410 | PRDX1_1082 | ACAAACATGGGGAAGTGTGCCCAGCNNNNNNNNCTTCAGCTTCCC |
| | | GATATCCGACGGTAGTGTTCTCGTTCAGGGGCCTTTTT |
| 411 | PRDX1_1083 | GCTGGGCTGTTTTAGTGCCAGGCTNNNNNNNNCTTCAGCTTCCCG |
| | | ATATCCGACGGTAGTGTTAGTTCAGGCCTTCCAGTTCA |
| 412 | PRKAA1_2662 | CAGAACCTCAAGCTTTTCAGGCATCCNNNNNNNNCTTCAGCTTCC |
| | | CGATATCCGACGGTAGTGTTCGGCACCTTCGGCAAAGT |
| 413 | PRKAA1_2666 | CACCCAACTATGCTGCACCAGAAGTANNNNNNNNCTTCAGCTTCC |
| | | CGATATCCGACGGTAGTGTGGTGGTCCATAGAGATTTG |
| 414 | PRKAA1_2670 | GAGTGCTCAGAAGAGGAAGTTCTCAGCTGNNNNNNNNCTTCAGCT |
| | | TCCCGATATCCGACGGTAGTGTAAGATATCAGGGAACA |
| 415 | PRKAA1_2674 | GGATTATGAATGGAAGGTTGTAAACCCATNNNNNNNNCTTCAGCT |
| | | TCCCGATATCCGACGGTAGTGTGAATTAAATCCACAGA |
| 416 | PRKAA1_2678 | GTGCAAATCTAATTAAAATTCTTGCACAANNNNNNNNCTTCAGCT |
| | | TCCCGATATCCGACGGTAGTGTTGCTGAGGCTCAAGGA |
| 417 | PRKAA2_2685 | CGAGAAATTCAAAATCTAAAACTCTTTCGNNNNNNNNCTTCAGCT |
| | | TCCCGATATCCGACGGTAGTGTTGGGCGTCGGCACCTT |
| 418 | PRKAA2_2688 | GCCAAGATAGCCGATTTCGGATTATCTAANNNNNNNNCTTCAGCT |
| | | TCCCGATATCCGACGGTAGTGTAAGAGATGGAAGCCAG |
| 419 | PRKAA2_2691 | GCTGCAGGTTGACCCACTNNNNNNNNCTTCAGCTTCCCGATATCC |
| | | GACGGTAGTGTTGTATGCTCTTCTTTGTGGCACCCTCC |
| 420 | PRKAA2_2695 | GCAGACAGCCCCAAAGCAAGATGNNNNNNNNCTTCAGCTTCCCGA |
| | | TATCCGACGGTAGTGTGAATAATGAACCAAGCCAGTGA |
| 421 | PRKAA2_2699 | CCACAACTGCAGAGAGCCNNNNNNNNCTTCAGCTTCCCGATATCC |
| | | GACGGTAGTGTGTTGATAACAGGAGCTATCTTTTGGAC |
| 422 | RPIA_3164 | CTACAATTGTCCATGCTGTGCAGCGNNNNNNNNCTTCAGCTTCCC |
| | | GATATCCGACGGTAGTGTACTCCAACAGCATCTGCCC |
| 423 | RPIA_3166 | CTCAATCTCATCAAGGGTGGCNNNNNNNNCTTCAGCTTCCCGATA |
| | | TCCGACGGTAGTGTCCTCGTCTGTATTCCCACTTCCTT |
| 424 | RPIA_3168 | GCTGTGAGCCAGAAGTTTGGGGNNNNNNNNCTTCAGCTTCCCGAT |
| | | ATCCGACGGTAGTGTGTGGCTGGCTATGCTAGTCGCTT |
| 425 | RPIA_3169 | GTTTGACCGGGTACACAAATGGAGTGAANNNNNNNNCTTCAGCTT |
| | | CCCGATATCCGACGGTAGTGTGGCCTATGTCCCAGTGA |
| 426 | RPIA_3171 | GGAGCAGAGTGTGTTCACCTTGAGTCTCCNNNNNNNNCTTCAGCT |
| | | TCCCGATATCCGACGGTAGTGTATCAAAATGATCCCAG |
| 427 | SDHA_1569 | GGGCATCTGCTAAAGTTTCAGATTCCATNNNNNNNNCTTCAGCTT |
| | | CCCGATATCCGACGGTAGTGTTGTCGGGGGTCCGGGG |
| 428 | SDHA_1570 | GCATTTGGCCTTTCTGAGGCAGGGTTTNNNNNNNNCTTCAGCTTC |
| | | CCGATATCCGACGGTAGTGTCACTGTTGATGGGAACAA |
| 429 | SDHA_1571 | GCACAGCTAGAAAATTATGGCATGCCGTNNNNNNNNCTTCAGCTT |
| | | CCCGATATCCGACGGTAGTGTTTTGATGCAGTGGTGGT |
| 430 | SDHA_1572 | GCCTCAAGTTTGGAAAGGGCGGGCAGNNNNNNNNCTTCAGCTTCC |
| | | CGATATCCGACGGTAGTGTAGCATGTGTTACCAAGCTG |
| 431 | SDHA_1573 | GCGATATGATACCAGCTATTTTGTGGAGNNNNNNNNCTTCAGCTT |
| | | CCCGATATCCGACGGTAGTGTTTATCAGCGTGCATTTG |
| 432 | SDHA_1574 | GCATAGAGGACGGGTCCATCCATNNNNNNNNCTTCAGCTTCCCGA |
| | | TATCCGACGGTAGTGTGACTGGCCACTCGCTATTGCA |
| 433 | SDHA_1575 | CTTCAGCTGCACGTCTGCCNNNNNNNNCTTCAGCTTCCCGATATC |
| | | CGACGGTAGTGTTTTTGCCTTGGATCTCCTGATGGAGA |
| 434 | SDHA_1576 | GTTCAGTTCCACCCTACAGGCATATATGGNNNNNNNNCTTCAGCT |
| | | TCCCGATATCCGACGGTAGTGTCTGTTGTTGCCACAGG |
| 435 | SDHA_1577 | GCGAAAGGTTTATGGAGCGATACGCCCNNNNNNNNCTTCAGCTTC |
| | | CCGATATCCGACGGTAGTGTAGGGCAGGCCTTCCTTG |
| 436 | SDHA_1578 | CGAGAAGGAAGAGGCTGTGGCCCTGAGANNNNNNNNCTTCAGCTT |
| | | CCCGATATCCGACGGTAGTGTTGTCGTGGAGAGGGAGG |
| 437 | SDHA_1579 | GCCTGGCATTTCAGAGACAGCNNNNNNNNCTTCAGCTTCCCGATA |
| | | TCCGACGGTAGTGTTGGCGTCTAGAGATGTGGTGTCTC |
| 438 | SDHA_1580 | GCGGCATTCCCACCAACTACAANNNNNNNNCTTCAGCTTCCCGAT |
| | | ATCCGACGGTAGTGTTGCACCACCTACCTCCAGAGCA |
| 439 | SDHA_1581 | GCCTCGGTACATGGTGCCAANNNNNNNNCTTCAGCTTCCCGATAT |
| | | CCGACGGTAGTGTTCCCTGTCCTCCCCACCGTGCATTA |
| 440 | SDHA_1582 | GCCTGGAGATAAAGTCCCTCCAATTAANNNNNNNNCTTCAGCTTC |
| | | CCGATATCCGACGGTAGTGTTGTACGCCTGTGGGGAGG |
| 441 | SDHA_1583 | GCTGATGGAAGCATAAGAACATCGGNNNNNNNNCTTCAGCTTCCC |
| | | GATATCCGACGGTAGTGTCTGGTTGTCTTTGGTCGGGC |
| 442 | SDHA_1584 | GCGTGTTGCAAGAAGGTTGTGGGANNNNNNNNCTTCAGCTTCCCG |
| | | ATATCCGACGGTAGTGTGCTGGGGAAGAATCTGTCATG |
| 443 | SDHA_1585 | GTCTGGAACACGGACCTGGTGGAGANNNNNNNNCTTCAGCTTCCC |
| | | GATATCCGACGGTAGTGTATGCAGAAGTCAATGCAAAA |
| 444 | SDHA_1586 | CAGAGGCACGGAAGGAGTCACNNNNNNNNCTTCAGCTTCCCGATA |
| | | TCCGACGGTAGTGTATCAGCAAGCTCTATGGAGACCTA |
| 445 | SDHA_1587 | GCCCATCCAGGGGCAACAGAAGAANNNNNNNNCTTCAGCTTCCCG |
| | | ATATCCGACGGTAGTGTTGGAGCTGCAGAACCTGATGC |
| 446 | SDHA_1588 | GGAAGGTCACTCTGGAATATAGACCCGTGNNNNNNNNCTTCAGCT |
| | | TCCCGATATCCGACGGTAGTGTGGAAGACTACAAGGTG |
| 447 | SDHA_1589 | GTGGTGATGACAGAATCAGCTTTTGNNNNNNNNCTTCAGCTTCCC |
| | | GATATCCGACGGTAGTGTGTCCTATGTGGACGTTGGCA |
| 448 | SDHB_2193 | CCCAGACAAGGCTGGAGACAAACCTCANNNNNNNNCTTCAGCTTC |
| | | CCGATATCCGACGGTAGTGTTCTCCTTGAGGCGCCGGT |
| 449 | SDHB_2195 | TGACTCTACTTTGACCTTCCGAAGATCNNNNNNNNCTTCAGCTTC |
| | | CCGATATCCGACGGTAGTGTTTGCCATCTATCGATGGG |
| 450 | SDHB_2196 | CACTCTAGCTTGCACCCGAAGGATTGNNNNNNNNCTTCAGCTTCC |
| | | CGATATCCGACGGTAGTGTTGTGGCCCCATGGTATTGG |
| 451 | SDHB_2197 | GATCTTGTTCCCGATTTGAGCAACTTCTANNNNNNNNCTTCAGCT |
| | | TCCCGATATCCGACGGTAGTGTTCTGTGGCTCTTGTGC |
| 452 | SDHB_2198 | GCAGCAGTATCTGCAGTCCATAGANNNNNNNNCTTCAGCTTCCCG |
| | | ATATCCGACGGTAGTGTCAAAAATCTACCCTCTTCCAC |
| 453 | SDHB_2199 | GCTACTGGTGGAACGGAGACAAATATCTGNNNNNNNNCTTCAGCT |
| | | TCCCGATATCCGACGGTAGTGTGAAGAAGAAGGATGAA |
| 454 | SDHB_2200 | AGCGCCTGGCCAAGCTGCANNNNNNNNCTTCAGCTTCCCGATATC |
| | | CGACGGTAGTGTAGTGCATTCTCTGTGCCTGCTGTAGC |
| 455 | SDHB_2201 | GCAGAGATCAAGAAAATGATGGCAACCTNNNNNNNNCTTCAGCTT |
| | | CCCGATATCCGACGGTAGTGTAGAGATGACTTCACAG |
| 456 | SDHB_2202 | CCAGCTCAGAGCTGAACANNNNNNNNCTTCAGCTTCCCGATATCC |
| | | GACGGTAGTGTATGAACTGCACAAGGACCTGTCCTAAG |
| 457 | SDHC_2206 | GCTTTGAGTGCAGGGGTCTCTCTTTTTGNNNNNNNNCTTCAGCTT |
| | | CCCGATATCCGACGGTAGTGTGAAGAGATGGAGCGGT |
| 458 | SDHC_2207 | GCACTGATCCACACAGCTAANNNNNNNNCTTCAGCTTCCCGATAT |
| | | CCGACGGTAGTGTTGTCCATCTGCCACCGTGGCACTGG |
| 459 | SDHC_2208 | GCCTGAAGATTCCCCAGCTATACNNNNNNNNCTTCAGCTTCCCGA |
| | | TATCCGACGGTAGTGTTTTGGAACTTGTGAAGTCCCTG |
| 460 | SDHC_2209 | CCCAGCATCATCTTCCTACACANNNNNNNNCTTCAGCTTCCCGAT |
| | | ATCCGACGGTAGTGTATCCGACACTTGATGTGGGACCT |
| 461 | SDHD_2214 | CACTTGTCACCGAGCCACCATTCNNNNNNNNCTTCAGCTTCCCGA |
| | | TATCCGACGGTAGTGTTGAGTGCCGTTTGCGGTGCCCT |
| 462 | SDHD_2215 | GTCTGCTTCCGGCTGCTTATTTGAATCCNNNNNNNNCTTCAGCTT |
| | | CCCGATATCCGACGGTAGTGTACCGACCTATCCCAGAA |
| 463 | SDHD_2216 | GTTGTTACTGACTATGTTCATGGGGATGNNNNNNNNCTTCAGCTT |
| | | CCCGATATCCGACGGTAGTGTGAGAGGGTTGTCAGTGT |
| 464 | SDHD_2217 | GCTATTTCAACTATCACGATGTGGGCNNNNNNNNCTTCAGCTTCC |
| | | CGATATCCGACGGTAGTGTCCTCACTCTTCATGGTCAC |
| 465 | SDHD_2218 | GTATGCCTCTTTGCCTCTGCTTTGTNNNNNNNNCTTCAGCTTCCC |
| | | GATATCCGACGGTAGTGTTGGCACTTTCAGCTTTAACC |
| 466 | SLC16A1_0891 | CGGCTTCTCTTATGCATTTCCCNNNNNNNNCTTCAGCTTCCCGAT |
| | | ATCCGACGGTAGTGTTTGGATTTGACCTGCATTTTGG |
| 467 | SLC16A1_0894 | CGTCTGTATTGGAGTCATTGGAGGTNNNNNNNNCTTCAGCTTCCC |
| | | GATATCCGACGGTAGTGTTGGAGGTCCTATCAGCAGTA |
| 468 | SLC16A1_0898 | CAGATCTTATTGGAAGACACCCTAAACNNNNNNNNCTTCAGCTTC |
| | | CCGATATCCGACGGTAGTGTGTTGCTGGAGCCCTCAT |
| 469 | SLC16A1_0902 | GTTGGATTCTGTGTCTATGCGGGATTCTTNNNNNNNNCTTCAGCT |
| | | TCCCGATATCCGACGGTAGTGTGACCATCTATGGGACT |
| 470 | SLC16A1_0906 | GGAGGGCCCAAGGAGGAGGAAAGNNNNNNNNCTTCAGCTTCCCGA |
| | | TATCCGACGGTAGTGTTGGCAAAAGAACAGAAA |
| 471 | SLC16A3_1117 | GCGGCTTTGTGCTTTACGNNNNNNNNCTTCAGCTTCCCGATATCC |
| | | GACGGTAGTGTATCCTGGGCGGCCTGCTGCTCAACTGC |
| 472 | SLC16A3_1120 | GCTCTGCAGTGTGTGCGTGAACNNNNNNNNCTTCAGCTTCCCGAT |
| | | ATCCGACGGTAGTGTCTTCTCCTACGCCTTCCCCAA |
| 473 | SLC16A3_1124 | CGACACCAAGGCCGCCTTCCTNNNNNNNNCTTCAGCTTCCCGATA |
| | | TCCGACGGTAGTGTGCCTGCTAGACCTGAGCGTCTTCC |
| 474 | SLC16A3_1128 | CGACCCACGTCTACATGTACGTGTTNNNNNNNNCTTCAGCTTCCC |
| | | GATATCCGACGGTAGTGTTGCAGTTCGAGGTGCTCATG |
| 475 | SLC16A3_1130 | GCATTTCCTGAAGGCTGAGCCTGAGAAAANNNNNNNNCTTCAGCT |
| | | TCCCGATATCCGACGGTAGTGTCTGGGCAACTTCTTCT |
| 476 | SLC16A7_1390 | CCTATGCATTCCCCAAAGCNNNNNNNNCTTCAGCTTCCCGATATC |
| | | CGACGGTAGTGTCTCTTGGTGCCAACAGAGTTACTCT |
| 477 | SLC16A7_1393 | GCAACCCGCCTTAACCATAATTGGCAAANNNNNNNNCTTCAGCTT |
| | | CCCGATATCCGACGGTAGTGTGGTGGTGATAGCAGGAG |
| 478 | SLC16A7_1397 | CCCTTTTTAAGCATAGAGGATTTCTGATANNNNNNNNCTTCAGCT |
| | | TCCCGATATCCGACGGTAGTGTACCCAATCAAACCACT |
| 479 | SLC16A7_1401 | GTGTTAGCAGTGTTCTCTTTGNNNNNNNNCTTCAGCTTCCCGATA |
| | | TCCGACGGTAGTGTCGACCTCGAATTCAGTACTTCTTC |
| 480 | SLC16A7_1404 | CCTTGAGCAAATCTAAACATTCGGANNNNNNNNCTTCAGCTTCCC |
| | | GATATCCGACGGTAGTGTTGTCCTGTGGGGCTATTGTG |
| 481 | SLC2A1_2721 | GCTCTGGTCCCTCTCAGTGGCCATCTTTTNNNNNNNNCTTCAGCT |
| | | TCCCGATATCCGACGGTAGTGTTCCCTGCAGTTTGGCT |
| 482 | SLC2A1_2724 | TCACCCACAGCCCTTCGTNNNNNNNNCTTCAGCTTCCCGATATCC |
| | | GACGGTAGTGTCTTCGTGTCCGCCGTGCTCATGGGCTT |
| 483 | SLC2A1_2727 | GCATCTTCGAGAAGGCGGGGGTGNNNNNNNNCTTCAGCTTCCCGA |
| | | TATCCGACGGTAGTGTGGAGAAGAAGGTCACCA |
| 484 | SLC2A1_2730 | GCCCCATCCCATGGTTCATCGTGGNNNNNNNNCTTCAGCTTCCCG |
| | | ATATCCGACGGTAGTGTGCTGGCATGGCGGGTTGTG |
| 485 | SLC2A1_2733 | TTCCATCCCCTGGGGGCTNNNNNNNNCTTCAGCTTCCCGATATCC |
| | | GACGGTAGTGTTGTGCTCCTGGTTCTGTTCTTCATCTT |
| 486 | SLC2A3_2804 | CACTGGGGTCATCAATGCTCCTGAGAANNNNNNNNCTTCAGCTTC |
| | | CCGATATCCGACGGTAGTGTATCACCCCTAGATCTTTC |
| 487 | SLC2A3_2808 | GCCCTGCGGGGTGCCTTTGNNNNNNNNCTTCAGCTTCCCGATATC |
| | | CGACGGTAGTGTGGCTGCTTTATGGGACTGTGT |
| 488 | SLC2A3_2812 | CCATTGTGCTCCAGCTCTCTCAGCAGNNNNNNNNCTTCAGCTTCC |
| | | CGATATCCGACGGTAGTGTCACCCAGGATGTATCCCAA |
| 489 | SLC2A3_2816 | ACTCTTCAGCCAGGGCCCNNNNNNNNCTTCAGCTTCCCGATATCC |
| | | GACGGTAGTGTCGCTCATGACTGTTTCTTTGTTATTAA |
| 490 | SLC2A3_2819 | GCCTGCTAAGGAGACCACCACCAANNNNNNNNCTTCAGCTTCCCG |
| | | ATATCCGACGGTAGTGTTTACCTTCTTCAAAGTCCCTG |
| 491 | SLC5A1_1305 | CATTTTCACCAAGATCTCGGCNNNNNNNNCTTCAGCTTCCCGATA |
| | | TCCGACGGTAGTGTTTGTGCTGGGCTGGCTGTTTGTCC |
| 492 | SLC5A1_1309 | CATCTTCCGAGATCCCCTCACNNNNNNNNCTTCAGCTTCCCGATA |
| | | TCCGACGGTAGTGTTTGCTTTTCACGAAGTGGGAGGCT |
| 493 | SLC5A1_1313 | GTCATGCTGGCCTCCCTCATGAGCTNNNNNNNNCTTCAGCTTCCC |
| | | GATATCCGACGGTAGTGTATTGCCTGTGTCGTCCCTTC |
| 494 | SLC5A1_1317 | AGCCCAGCAACTGTCCCACNNNNNNNNCTTCAGCTTCCCGATATC |
| | | CGACGGTAGTGTCTGTCTTCCTGCTTGCTATTTTCTGG |
| 495 | SLC5A1_1321 | CATCCTGGTGACCGTGGCTGTCTTTTNNNNNNNNCTTCAGCTTCC |
| | | CGATATCCGACGGTAGTGTTTTTGTGGGCTAGAGCAGC |
| 496 | SLC5A5_0875 | GCCAGCAAGCAGATCACTGCAGNNNNNNNNCTTCAGCTTCCCGAT |
| | | ATCCGACGGTAGTGTTCTTTCAACATGACAGATGCCGC |
| 497 | SLC5A5_0877 | CCCAGTTTTGGCTCTACTTTGCAGGGNNNNNNNNCTTCAGCTTCC |
| | | CGATATCCGACGGTAGTGTGGGAGTTCTGTCCTTCTGG |
| 498 | SLC5A5_0879 | GCTTTAACGTGTCTGTGCAGGGTNNNNNNNNCTTCAGCTTCCCGA |
| | | TATCCGACGGTAGTGTTCTTGATTTTGCCCGTACCCGT |
| 499 | SLC5A5_0881 | GCATGATGATGCAGTCAGGGCGCAAAGNNNNNNNNCTTCAGCTTC |
| | | CCGATATCCGACGGTAGTGTGACACTGCAAAGGGAA |
| 500 | SLC5A5_0883 | CATAAGTTATTTCCTAGGATTTTTCCCCCNNNNNNNNCTTCAGCT |
| | | TCCCGATATCCGACGGTAGTGTCTGGCGGAAGATTGCT |
| 501 | SLC7A1_2222 | CACTTTTGATCTGGTGGCCCTCNNNNNNNNCTTCAGCTTCCCGAT |
| | | ATCCGACGGTAGTGTTTCCCGTCATATTCCAGCTCTG |
| 502 | SLC7A1_2226 | CCCCGGCGTGCTGGCTGAAAACNNNNNNNNCTTCAGCTTCCCGAT |
| | | ATCCGACGGTAGTGTCGGCTGGAACTTAATCCTCTCCT |
| 503 | SLC7A1_2232 | GCTGGGAAGGTGCCAAGTACGNNNNNNNNCTTCAGCTTCCCGATA |
| | | TCCGACGGTAGTGTGTGGGGATCGTGGCGTCCCTCTTG |
| 504 | SLC7A1_2236 | GGCAAGCACCAATGATTCCCAGCTNNNNNNNNCTTCAGCTTCCCG |
| | | ATATCCGACGGTAGTGTCTCTCCTGGCTTACTCGTTG |
| 505 | SLC7A1_2240 | CGTGAACGTCTATCTCATGATGCNNNNNNNNCTTCAGCTTCCCGA |
| | | TATCCGACGGTAGTGTTTTCTGCTCGCAGGGTCTGCCC |
| 506 | SLC9A1_2249 | TCCCCTCACAGACTCTTCCACCANNNNNNNNCTTCAGCTTCCCGA |
| | | TATCCGACGGTAGTGTCAGTGACAGCCCCAGCTCCCA |
| 507 | SLC9A1_2255 | GCCTCATGAAGATAGGTTTCCATGTGNNNNNNNNCTTCAGCTTCC |
| | | CGATATCCGACGGTAGTGTCCGCCCTGTTAATCATTCC |
| 508 | SLC9A1_2261 | GCGGGGTGCTTGTGGGCGTGGTNNNNNNNNCTTCAGCTTCCCGAT |
| | | ATCCGACGGTAGTGTGGGAGTCCTTGCTCAATGA |
| 509 | SLC9A1_2267 | GCCCCTGGTAGACCTGTTGGCTGTNNNNNNNNCTTCAGCTTCCCG |
| | | ATATCCGACGGTAGTGTGAGGGGCCATCGCCTTCTCT |
| 510 | SLC9A1_2273 | GCAGCTGGAGCAGAAGATCAACAACTACCNNNNNNNNCTTCAGCT |
| | | TCCCGATATCCGACGGTAGTGTATCCTGAGGAACAACT |
| 511 | SLCA12_1015 | GCCTGTGTGACAAGCTGGGGAAGAATNNNNNNNNCTTCAGCTTCC |
| | | CGATATCCGACGGTAGTGTGGGAGGAGAGGTTAGATG |
| 512 | SLCA12_1019 | GCTGGGGCCTGGGAAGAAGAATGATNNNNNNNNCTTCAGCTTCCC |
| | | GATATCCGACGGTAGTGTTAAGGCTAGTGGCCGCTTGG |
| 513 | SLCA12_1023 | GCTGATGGTGGATTTCTTCAACATTTTGNNNNNNNNCTTCAGCTT |
| | | CCCGATATCCGACGGTAGTGTGGAGGAGACTAAGATGG |
| 514 | SLCA12_1027 | CGTCCTTCCTGTTGGAGCNNNNNNNNCTTCAGCTTCCCGATATCC |
| | | GACGGTAGTGTCCTTCTCCTTTTTTGCTGGCATTTTCC |
| 515 | SLCA12_1031 | CGAGTGCATGAAGATATTGAAATGACCAANNNNNNNNCTTCAGCT |
| | | TCCCGATATCCGACGGTAGTGTTGGCTGTGGACTGGCT |
| 516 | SOD_0414 | GGACTGACTGAAGGCCTGCATGGATNNNNNNNNCTTCAGCTTCCC |
| | | GATATCCGACGGTAGTGTGTGGCCTAGCGAGTTATG |
| 517 | SOD_0415 | GTGGGCCAAAGGATGAAGAGAGGCATNNNNNNNNCTTCAGCTTCC |
| | | CGATATCCGACGGTAGTGTGGTGTGGGGAAGCATTA |
| 518 | SOD_0416 | TCTCACTCTCAGGAGACCATTGCNNNNNNNNCTTCAGCTTCCCGA |
| | | TATCCGACGGTAGTGTTCCTCACTTTAATCCTCTATCC |
| 519 | SOD_0417 | GTACAAAGACAGGAAACGCTGGAAGTCNNNNNNNNCTTCAGCTTC |
| | | CCGATATCCGACGGTAGTGTGGTGTGGCCGATGTGTCT |
| 520 | SOD_0418 | CCCTTGGATGTAGTCTGAGGCCCCTTANNNNNNNNCTTCAGCTTC |
| | | CCGATATCCGACGGTAGTGTGCACACTGGTGGTCCATG |
| 521 | SOD2_0438 | GCCCTGGAACCTCACATCAACGCGNNNNNNNNCTTCAGCTTCCCG |
| | | ATATCCGACGGTAGTGTTAGCACCAGCACTAGCAGCA |
| 522 | SOD2_0439 | GCGTTGGCCAAGGGAGATGTTACAGCNNNNNNNNCTTCAGCTTCC |
| | | CGATATCCGACGGTAGTGTACCTGCCCTACGACTAC |
| 523 | SOD2_0441 | GCTCAGGTTGGGGTTGGCTTGGTTTNNNNNNNNCTTCAGCTTCCC |
| | | GATATCCGACGGTAGTGTGGACAAACCTCAGCCCTAA |
| 524 | SOD2_0443 | GGGAGAATGTAACTGAAAGATACATGGCTNNNNNNNNCTTCAGCT |
| | | TCCCGATATCCGACGGTAGTGTACTGCAAGGAACAACA |
| 525 | SOD2_0444 | GCTGAGTATGTTAAGCTCTTTATGACTGNNNNNNNNCTTCAGCTT |
| | | CCCGATATCCGACGGTAGTGTCGCTTACTACCTTCAGT |
| 526 | TAL_2770 | GAAGATTCCGGGCCGAGTATCCNNNNNNNNCTTCAGCTTCCCGAT |
| | | ATCCGACGGTAGTGTGATGCCCGCTTACCAGGA |
| 527 | TAL_2772 | TCGAGGAGCAGCACGGCATCCACNNNNNNNNCTTCAGCTTCCCGA |
| | | TATCCGACGGTAGTGTTGATAAAGATGCGATGGTGGCC |
| 528 | TAL_2774 | GTTTAGCTACAAAACCATTGTCATGGGCNNNNNNNNCTTCAGCTT |
| | | CCCGATATCCGACGGTAGTGTCATCTCCCCATTTGTT |
| 529 | TAL_2776 | CCACCTGGATGAGAAGTCTTTCCGNNNNNNNNCTTCAGCTTCCCG |
| | | ATATCCGACGGTAGTGTAAGCACTGGCCGGCTGTGAC |
| 530 | TAL_2778 | GAGGCTGGACTCCAGATCTGCACCGNNNNNNNNCTTCAGCTTCCC |
| | | GATATCCGACGGTAGTGTGAGGACCAGATGGCTGTGG |
| 531 | TIGAR_3037 | CATGAGGACAAAGCAGACCATGCATGGNNNNNNNNCTTCAGCTTC |
| | | CCGATATCCGACGGTAGTGTAGGAGAAAATAATCCAAG |
| 532 | TIGAR_3039 | CGGAGGAGAGACGCTGGACCAGGTGAAANNNNNNNNCTTCAGCTT |
| | | CCCGATATCCGACGGTAGTGTGACGGTAAAGTATGAC |
| 533 | TIGAR_3041 | GGATTAGCAGCCAGTGTCTTAGTTGTGNNNNNNNNCTTCAGCTTC |
| | | CCGATATCCGACGGTAGTGTAGCGGATCAAAAAGAACA |
| 534 | TIGAR_3043 | GAGGAAGGAAGAGAAGTTAAACCAACGGTNNNNNNNNCTTCAGCT |
| | | TCCCGATATCCGACGGTAGTGTTGAGAAGTCTGTTTGA |
| 535 | TP53I3_2466 | GTGAAGTCCTCCTGAAGGTGGCNNNNNNNNCTTCAGCTTCCCGAT |
| | | ATCCGACGGTAGTGTCTGCCCTGTCCTGTCCTGCCCT |
| 536 | TP53I3_2467 | GCAACATTTTGGGACTTGAGGCATCTGGNNNNNNNNCTTCAGCTT |
| | | CCCGATATCCGACGGTAGTGTTGAAGGAGGTGGCCAA |
| 537 | TP53I3_2468 | GCCATGGCTCTGCTCCCCGGTGGNNNNNNNNCTTCAGCTTCCCGA |
| | | TATCCGACGGTAGTGTACTTAATGCAGAGACAAGGCCA |
| 538 | TP53I3_2470 | GCTAATCCATGCAGGACTGAGTGGTGTNNNNNNNNCTTCAGCTTC |
| | | CCGATATCCGACGGTAGTGTAAGGGCTCCTCATGCCTA |
| 539 | TP53I3_2471 | GCTTCAAATGGCAGAAAAGCTTGGAGNNNNNNNNCTTCAGCTTCC |
| | | CGATATCCGACGGTAGTGTGGAAATGTTCAGGCTGGAG |
| 540 | TP53I3_2472 | GCTGGAGTTAATCTTATTCTAGACTGCNNNNNNNNCTTCAGCTTC |
| | | CCGATATCCGACGGTAGTGTTATTCCTCTGGTCACAGC |
| 541 | TP53I3_2473 | GTCGATGGGTTCTCTATGGTCTGATGGGNNNNNNNNCTTCAGCTT |
| | | CCCGATATCCGACGGTAGTGTACAAAAAAGAGGATTTC |
| 542 | TP53I3_2474 | GCTGAGGTCTAGGGACAATAAGTACANNNNNNNNCTTCAGCTTCC |
| | | CGATATCCGACGGTAGTGTAAGAACGTCAACTGCCTGG |
| 543 | TP53I3_2475 | AGGGCCCCCAACGTCTGCTNNNNNNNNCTTCAGCTTCCCGATATC |
| | | CGACGGTAGTGTGGCCCCTGTTTTCAAAGCTACTTTTT |
| 544 | TP53I3_2476 | TCGTCCTGGAACTGCCCCAGTGAANNNNNNNNCTTCAGCTTCCCG |
| | | ATATCCGACGGTAGTGTCAAATTCTGCCTCACTTCTCC |
| 545 | TRX_1250 | CTTCTCAGCCACGTGGTGTNNNNNNNNCTTCAGCTTCCCGATATC |
| | | CGACGGTAGTGTTTTGGATCCATTTCCATCGGTCCTTA |
| 546 | TRX_1251 | GTTTTTTAAGAAGGGACAAAAGGTGGGTGNNNNNNNNCTTCAGCT |
| | | TCCCGATATCCGACGGTAGTGTGCAGGTGATAAACTTG |
| 547 | TRX_1252 | GTTTTCTGAAAATATAACCAGCCATTGGCNNNNNNNNCTTCAGCT |
| | | TCCCGATATCCGACGGTAGTGTAGAGTGTGAAGTCAAA |
| 548 | FGFR2_4 | GCCGTGATCAGTTGGACTAAGGATGNNNNNNNNCTTCAGCTTCCC |
| | | GATATCCGACGGTAGTGTGTTTAGTTGAGGATACCACA |
| 549 | FGFR2_6 | CGATGGTGCGGAAGATTTTGTCAGTGAGANNNNNNNNCTTCAGCT |
| | | TCCCGATATCCGACGGTAGTGTCCACGCCTAGAGACTC |
| 550 | FGFR2_8 | GCCGGTGTTAACACCACGGACAAANNNNNNNNCTTCAGCTTCCCG |
| | | ATATCCGACGGTAGTGTGACGTAGAGTTTGTCTGCAAG |
| 551 | FGFR2_10 | ACTACCTGGAGATAGCCATTTACTGCATNNNNNNNNCTTCAGCTT |
| | | CCCGATATCCGACGGTAGTGTTTTTGAGGACGCTGGGG |
| 552 | FGFR2_12 | GCAGTGTTAAAACATGAATGACTGTGTCNNNNNNNNCTTCAGCTT |
| | | CCCGATATCCGACGGTAGTGTCGAACAGTATTCACCTA |
| 553 | VHL_20 | CGTGCTGCCCGTATGGCTCAACTNNNNNNNNCTTCAGCTTCCCGA |
| | | TATCCGACGGTAGTGTAAGAGTCCGGCCCGGAGGAACT |
| 554 | VHL_21 | GCTCTTCAGAGATGCAGGGACACNNNNNNNNCTTCAGCTTCCCGA |
| | | TATCCGACGGTAGTGTTCTTCTGCAATCGCAGTCCGCG |
| 555 | VHL_22 | GCGCCGAGGAGGAGATGGAGGNNNNNNNNCTTCAGCTTCCCGATA |
| | | TCCGACGGTAGTGTAGAACTGGGACGAGGCCGAGG |
| 556 | VHL_24 | AGTCGGGCGCCGAGGAGTCCGNNNNNNNNCTTCAGCTTCCCGATA |
| | | TCCGACGGTAGTGTGGCGTCCGGCCCGGGTGGTCTGG |
| 557 | VHL_25 | CTCAATGTTGACGGACAGCCTATTNNNNNNNNCTTCAGCTTCCCG |
| | | ATATCCGACGGTAGTGTTACCCAACGCTGCCGCCTGG |
| 558 | VHL_26 | GTCCGGAGCCTAGTCAAGCCTGAGAATTANNNNNNNNCTTCAGCT |
| | | TCCCGATATCCGACGGTAGTGTCGATGGGCTTCTGGTT |
| 559 | VHL_27 | GCGGCTGACACAGGAGCGCATTGCACATNNNNNNNNCTTCAGCTT |
| | | CCCGATATCCGACGGTAGTGTAAAGAGCGATGCCTCCA |
| 560 | VHL_28 | GCTTTTGATGGTACTGATGAGTCTTGNNNNNNNNCTTCAGCTTCC |
| | | CGATATCCGACGGTAGTGTTACGAAGATCTGGAAGACC |
| 561 | NTRK1 | GATGTGCACGCCCGGCTGCAANNNNNNNNCTTCAGCTTCCCGATA |
| | (TRKa)_33 | TCCGACGGTAGTGTAACACGGAGGCAATCGACTGCATC |
| 562 | NTRK1 | GATGGTGTACCTGGCGGGTCTGCATTTTNNNNNNNNCTTCAGCTT |
| | (TRKa)_36 | CCCGATATCCGACGGTAGTGTCTCAACCGCTTCCTCC |
| 563 | NTRK1 | CATCGTGAAGAGTGGTCTCCGTTTCGTGGNNNNNNNNCTTCAGCT |
| | (TRKa)_50 | TCCCGATATCCGACGGTAGTGTATAGCCTCCACCACCT |
| 564 | NTRK1 | GCAAAGGCTCTGGGCTCCAAGGCCANNNNNNNNCTTCAGCTTCCC |
| | (TRKa)_56 | GATATCCGACGGTAGTGTCAACAAATGTGGACGGAGAA |
| 565 | NTRK1 | GCAGGGATATCTACAGCACCGACTNNNNNNNNCTTCAGCTTCCCG |
| | (TRKa)_59 | ATATCCGACGGTAGTGTTGGCTAGCCAGGTCGCTGCGG |
| 566 | PDGFRB_69 | AGTCAACACCTCCTCAACCNNNNNNNNCTTCAGCTTCCCGATATC |
| | | CGACGGTAGTGTCTCTATACTGCCGTGCAGCCCAATG |
| 567 | PDGFRB_74 | CGGTGGTGTGGGAACGGATGTNNNNNNNNCTTCAGCTTCCCGATA |
| | | TCCGACGGTAGTGTTGCTGTTGCTGTCTCTCCTGT |
| 568 | PDGFRB_92 | CGTGGCTTTTCTGGTATCTTTGAGGACAGNNNNNNNNCTTCAGCT |
| | | TCCCGATATCCGACGGTAGTGTCATCTTTCTCACGGAA |
| 569 | PDGFRB_97 | GTCCGAGTGCTGGAGCTAAGTGAGAGCNNNNNNNNCTTCAGCTTC |
| | | CCGATATCCGACGGTAGTGTCGGTATGTGTCAGAGCTG |
| 570 | PDGFRB_101 | GCCAATGGCATGGAGTTTCTGGCCTNNNNNNNNCTTCAGCTTCCC |
| | | GATATCCGACGGTAGTGTCCAACTACATGGCCCCTTAC |
| 571 | ERBB2 | TTCGGCCCCAGCCCCCTTNNNNNNNNCTTCAGCTTCCCGATATCC |
| | (HER2)_118 | GACGGTAGTGTTCCCCACACATGACCCCAGCCCTCTAC |
| 572 | ERBB2 | GCCCTGGGACCAGCTCTTTCNNNNNNNNCTTCAGCTTCCCGATAT |
| | (HER2)_123 | CCGACGGTAGTGTACAATGGCGCCTACTCGCTGACCCT |
| 573 | ERBB2 | CACGATTTTGTGGAAGGACATCTTCNNNNNNNNCTTCAGCTTCCC |
| | (HER2)_131 | GATATCCGACGGTAGTGTAACAATACCACCCCTGTCAC |
| 574 | ERBB2 | GCAAGAAGATCTTTGGGAGCCTGGCATNNNNNNNNCTTCAGCTTC |
| | (HER2)_136 | CCGATATCCGACGGTAGTGTATGGAACACAGCGGTGTG |
| 575 | ERBB2 | GCTGGCTCCGATGTATTTGATGGTGNNNNNNNNCTTCAGCTTCCC |
| | (HER2)_157 | GATATCCGACGGTAGTGTCTGGGGGCATGGTCCA |
| 576 | NTRK2 | GCGAGAGCCCCACATGAGGAAGAACATCNNNNNNNNCTTCAGCTT |
| | (TRKb)_172 | CCCGATATCCGACGGTAGTGTAAACAGCCCTGGTACCA |
| 577 | NTRK2 | GCAACCTGCAGCACATCAATTTTACCCNNNNNNNNCTTCAGCTTC |
| | (TRKb)_179 | CCGATATCCGACGGTAGTGTCAAACCAGAAAAGGTTAG |
| 578 | NTRK2 | GCAGATCTCTTGTGTGGCGGAAAATCTNNNNNNNNCTTCAGCTTC |
| | (TRKb)_184 | CCGATATCCGACGGTAGTGTAGGTGATCCGGTTCCTAA |
| 579 | NTRK2 | CGGGGACACCACGAACAGAAGTAATNNNNNNNNCTTCAGCTTCCC |
| | (TRKb)_189 | GATATCCGACGGTAGTGTGAGTATGGGAAGGATGAGAA |
| 580 | NTRK2 | GCTGAATGCTATAACCTCTGTCCTGNNNNNNNNCTTCAGCTTCCC |
| | (TRKb)_194 | GATATCCGACGGTAGTGTATCCCCAGTACTTTGGCATC |
| 581 | PDGFRA_216 | GCACAACTGATCCCGAGACTCCTGTNNNNNNNNCTTCAGCTTCCC |
| | | GATATCCGACGGTAGTGTGAATGAGCTTGAAGGCAGGC |
| 582 | PDGFRA_226 | GTAATAATGAAACTTCCTGGACTATTTNNNNNNNNCTTCAGCTTC |
| | | CCGATATCCGACGGTAGTGTCATCCATTCTGGACTTG |
| 583 | PDGFRA_236 | CGACATCCAGAGATCACTCTATGATCGTCNNNNNNNNCTTCAGCT |
| | | TCCCGATATCCGACGGTAGTGTAAAGCACACGGAGCTA |
| 584 | PDGFRA_241 | GTGGGTACCGGATGGCCAAGCCTGNNNNNNNNCTTCAGCTTCCCG |
| | | ATATCCGACGGTAGTGTACAACCTCTACACCACA |
| 585 | PDGFRA_246 | CATCAAGAGAGAGGACGAGACCATNNNNNNNNCTTCAGCTTCCCG |
| | | ATATCCGACGGTAGTGTCTACATCATTCCTCTGCCTGA |
| 586 | FGFR1_258 | CGTCAATGTTTCAGATGCTCTCCCCTCCNNNNNNNNCTTCAGCTT |
| | | CCCGATATCCGACGGTAGTGTGAAAGCAACCGCACCC |
| 587 | FGFR1_259 | GCATCACAGGGGAGGAGGTGGANNNNNNNNCTTCAGCTTCCCGAT |
| | | ATCCGACGGTAGTGTTGTGGAAGTGGAGTCCTTCCTGG |
| 588 | FGFR1_261 | TGGCAAAGAATTCAAACCNNNNNNNNCTTCAGCTTCCCGATATCC |
| | | GACGGTAGTGTGCCCGTAGCTCCATATTGGACATCCCC |
| 589 | FGFR1_264 | CAGATAACACCAAACCAAACCNNNNNNNNCTTCAGCTTCCCGATA |
| | | TCCGACGGTAGTGTTCTATGCTTGCGTAACCAGCAGCC |
| 590 | FGFR1_265 | CATCCCTCTGCGCAGACAGGTAANNNNNNNNCTTCAGCTTCCCGA |
| | | TATCCGACGGTAGTGTTCATCTATTGCACAGGGGCCTT |
| 591 | VEGFA_121 | TGTGACAAGCCGAGGCGGTGAGCCNNNNNNNNCTTCAGCTTCCCG |
| | | ATATCCGACGGTAGTGTTCCAACATCACCATGCAGATT |
| 592 | VEGFA_121b | TCTCTCACCAGGAAAGACTGATACNNNNNNNNCTTCAGCTTCCCG |
| | | ATATCCGACGGTAGTGTTCCAACATCACCATGCAGATT |
| 593 | VEGFA_165 | GAGGCGGTGAGCCGGGCAGGAGGANNNNNNNNCTTCAGCTTCCCG |
| | | ATATCCGACGGTAGTGTAGAGCGGAGAAAGCAT |
| 594 | VEGFA_165_165b | CCTGTGGGCCTTGCTCAGAGCNNNNNNNNCTTCAGCTTCCCGATA |
| | | TCCGACGGTAGTGTAGTCCAACATCACCATGCAGATT |
| 595 | VEGFA_165b | CCACGCTGCCGCCACCACACCANNNNNNNNCTTCAGCTTCCCGAT |
| | | ATCCGACGGTAGTGTTCCGCAGACGTGTAAATGTTCCT |
| 596 | VEGF_189_189b | GTTCGAGGAAAGGGAAAGGGGCAAAAACNNNNNNNNCTTCAGCTT |
| | | CCCGATATCCGACGGTAGTGTCCATGCAGATTATGCG |
| 597 | VEGFA_ex1_5 | GGCGTGAGCCCTCCCCCTTGGGANNNNNNNNCTTCAGCTTCCCGA |
| | (6) | TATCCGACGGTAGTGTAGCAAGAGCTCCAGAGAG |
| 598 | VEGFA_ex1_5 | CGAAGTGGTGAAGTTCATGGATGTCTNNNNNNNNCTTCAGCTTCC |
| | (7) | CGATATCCGACGGTAGTGTCCTCCGAAACCATGAAC |
| 599 | VEGFA_ex1_5 | CGTTTTAATTTATTTTTGCTTGCCNNNNNNNNCTTCAGCTTCCCG |
| | (13) | ATATCCGACGGTAGTGTGTTAGGTGGACCGGTCAGCGG |
| 600 | VEGFA_ex1_5 | CACTGTGGATTTTGGAAACCAGCNNNNNNNNCTTCAGCTTCCCGA |
| | (14) | TATCCGACGGTAGTGTCCCTCTTCTTTTTTCTTAAACA |
| 601 | VEGFA_ex1_5 | GGCGCTCGGAAGCCGGGCTCATGGANNNNNNNNCTTCAGCTTCCC |
| | (15) | GATATCCGACGGTAGTGTGCGCGGGGGAAGCCGAG |
| 602 | VEGFA_ex1_5 | GCCTGGAGTGTGTGCCCACTGAGGAGNNNNNNNNCTTCAGCTTCC |
| | (16) | CGATATCCGACGGTAGTGTAGCTACTGCCATCCAA |
| 603 | VEGFA_ex1_5 | CCTACAGCACAACAAATGTGAATGCAGACNNNNNNNNCTTCAGCT |
| | (17) | TCCCGATATCCGACGGTAGTGTGATGCGGGGGCTGCTG |
| 604 | VEGFA_ex1_5 | CTGTGGGCCTTGCTCAGAGCGGANNNNNNNNCTTCAGCTTCCCGA |
| | (18) | TATCCGACGGTAGTGTCCAACATCACCATGCAGATTA |
| 605 | ADPGK_0002 | GCCAGAGCTGCCAGGCTCGGNNNNNNNNCTTCAGCTTCCCGATAT |
| | | CCGACGGTAGTGTCGGAAGAGGCGCGGGCTAGG |
| 606 | ADPGK_0004 | CACCAGCCGAGTGTCTCTGAGGNNNNNNNNCTTCAGCTTCCCGAT |
| | | ATCCGACGGTAGTGTATCCATTTCCACACGCTGGTCT |
| 607 | ADPGK_0011 | GTGGGGCCAGTTAAAAGCTCCNNNNNNNNCTTCAGCTTCCCGATA |
| | | TCCGACGGTAGTGTGGTTCTTCTTTGCGGTCCAGTTGG |
| 608 | ADPGK_0015 | TTCTCACCCAGTCAGCCTCNNNNNNNNCTTCAGCTTCCCGATATC |
| | | CGACGGTAGTGTTGACATCCCCACTGGTATTCCAGTTC |
| 609 | ADPGK_0017 | GCAACTGTGGATGGACACTGGGCCNNNNNNNNCTTCAGCTTCCCG |
| | | ATATCCGACGGTAGTGTGGTGTTCCTGATGTGGG |
| 610 | AR_0041 | AGTCGGCCCTGGAGTGCCACCCCGAGANNNNNNNNCTTCAGCTTC |
| | | CCGATATCCGACGGTAGTGTAGCAAGAGACTAGCCCCA |
| 611 | AR_0062 | TGGCGGCGGCGGCGGCGGCNNNNNNNNCTTCAGCTTCCCGATATC |
| | | CGACGGTAGTGTAGCCTGCATGGCGCGGGTGCAGCGGG |
| 612 | AR_0068 | GCTTGTACACGTGGTCAAGTGGGNNNNNNNNCTTCAGCTTCCCGA |
| | | TATCCGACGGTAGTGTGTCAGCCCATCTTTCTGAATGT |
| 613 | AR_0069 | GCTCATGGTGTTTGCCATGGGCTGGNNNNNNNNCTTCAGCTTCCC |
| | | GATATCCGACGGTAGTGTTCTAGCCTCAATGAACTGGG |
| 614 | AR_0074 | CATGGTGAGCGTGGACTTTCCGGAAATGNNNNNNNNCTTCAGCTT |
| | | CCCGATATCCGACGGTAGTGTAAAAGAAAAAATCCCAC |
| 615 | AR_0075 | CCACACCCAGTGAAGCATNNNNNNNNCTTCAGCTTCCCGATATCC |
| | | GACGGTAGTGTGCTGCATCAGTTCACTTTTGACCTGCT |
| 616 | ARV7_ARV_0009 | GCATCTCAAAATGACCAGACCCTGNNNNNNNNCTTCAGCTTCCCG |
| | | ATATCCGACGGTAGTGTGTGCGCCAGCAGAAATGAT |
| 617 | ARV12_ARV_0002 | GCAGAGATCATCTCTGTGCAAGTGNNNNNNNNCTTCAGCTTCCCG |
| | | ATATCCGACGGTAGTGTAGAGACAGCTTGTACACGTGG |
| 618 | BRAF_0106 | CCCCAAATTCTCACCAGTCCNNNNNNNNCTTCAGCTTCCCGATAT |
| | | CCGACGGTAGTGTCCAACTTGATTTGCTGTTTGTCTCC |
| 619 | BRAF_0112 | GACATGTGAATATCCTACTCTTCATGGGCNNNNNNNNCTTCAGCT |
| | | TCCCGATATCCGACGGTAGTGTGGAACAGTCTACAAGG |
| 620 | BRAF_0115 | GCTACAGTGAAATCTCGATGGAGTGGGTCNNNNNNNNCTTCAGCT |
| | | TCCCGATATCCGACGGTAGTGTACGACAGACTGCACAG |
| 621 | BRAF_0116 | CATACAGCTTTCAGTCAGATGTATATGCANNNNNNNNCTTCAGCT |
| | | TCCCGATATCCGACGGTAGTGTATAGGTGATTTTGGTC |
| 622 | BRAF_0117 | CAAACATCAACAACAGGGACCAGNNNNNNNNCTTCAGCTTCCCGA |
| | | TATCCGACGGTAGTGTGGATCCATTTTGTGGATGGCAC |
| 623 | CAT_0151 | CGGACATGGTCTGGGACTTCTGGAGCCTANNNNNNNNCTTCAGCT |
| | | TCCCGATATCCGACGGTAGTGTGAAGATGGTAACTGGG |
| 624 | CAT_0153 | CCAGGGCATCAAAAACCTNNNNNNNNCTTCAGCTTCCCGATATCC |
| | | GACGGTAGTGTGGTTTCTTTCTTGTTCAGTGATCGGGG |
| 625 | CAT_0155 | CACCAAGGTTTGGCCTCACAAGGACTANNNNNNNNCTTCAGCTTC |
| | | CCGATATCCGACGGTAGTGTTGACTATGGCATCCGGG |
| 626 | CAT_0159 | CCTGAAGGATGCACAAATTTTCATCCAGNNNNNNNNCTTCAGCTT |
| | | CCCGATATCCGACGGTAGTGTTCTGGAGAAGTGCGGAG |
| 627 | CAT_0161 | GCGGCAAGGGAGAAGGCAAATCTGTGAGGNNNNNNNNCTTCAGCT |
| | | TCCCGATATCCGACGGTAGTGTCAAGAACTTCACTGAG |
| 628 | CD274_0182 | GAGGGCCCGGCTGTTGAAGGACCAGCTNNNNNNNNCTTCAGCTTC |
| | | CCGATATCCGACGGTAGTGTCCCAGTAGAAAAACAA |
| 629 | CD274_0184 | TGGTTGTGGATCCAGTCACCTCTGNNNNNNNNCTTCAGCTTCCCG |
| | | ATATCCGACGGTAGTGTAGATCACAGATGTGAAATTGC |
| 630 | CD274_0186 | GCACTTTTAGGAGATTAGATCCTGAGGANNNNNNNNCTTCAGCTT |
| | | CCCGATATCCGACGGTAGTGTAAGCAGTGACCATCAAG |
| 631 | CD274_0188 | GGCATCCAAGATACAAACTCAAAGAAGCNNNNNNNNCTTCAGCTT |
| | | CCCGATATCCGACGGTAGTGTCACATCCTCCAAATGAA |
| 632 | CD274_0189 | CTTCTGATCTTCAAGCAGGGATTCTCNNNNNNNNCTTCAGCTTCC |
| | | CGATATCCGACGGTAGTGTCTGACATTCATCTTCCGTT |
| 633 | CTLA4_0199 | GCAAAGCAATGCACGTGGCCCAGCCTGNNNNNNNNCTTCAGCTTC |
| | | CCGATATCCGACGGTAGTGTAACACCGCTCCCATAAA |
| 634 | CTLA4_0203 | CTTCCTAGATGATTCCATCTGCACGGNNNNNNNNCTTCAGCTTCC |
| | | CGATATCCGACGGTAGTGTGCTTTGTGTGTGAGTATGC |
| 635 | CTLA4_0205 | TTGATCCAGAACCGTGCCCAGATTCNNNNNNNNCTTCAGCTTCCC |
| | | GATATCCGACGGTAGTGTTCAAGTGAACCTCACTATCC |
| 636 | CTLA4_0207 | CCCCCAACAGAGCCAGAANNNNNNNNCTTCAGCTTCCCGATATCC |
| | | GACGGTAGTGTGACTTCCTCCTCTGGATCCTTGCAGC |
| 637 | FBP1_0214 | CCCAGCTGCTCAACTCGCTCNNNNNNNNCTTCAGCTTCCCGATAT |
| | | CCGACGGTAGTGTTTGCACCTGCAGCCCCGCGCTCT |
| 638 | FBP1_0222 | GATCCCCTTGATGGATCTTCCNNNNNNNNCTTCAGCTTCCCGATA |
| | | TCCGACGGTAGTGTGTCCTCTCCAACGACCTGGTTATG |
| 639 | FBP1_0224 | GTCCTTGCCATGGACTGTGGGGTNNNNNNNNCTTCAGCTTCCCGA |
| | | TATCCGACGGTAGTGTCGTTGGAACCATTTTTGGCATC |
| 640 | FBP1_0226 | GCTCCTTATGGGGCCCGGTATGTGGGNNNNNNNNCTTCAGCTTCC |
| | | CGATATCCGACGGTAGTGTGGACAAGGATGTGAAGATA |
| 641 | FBP1_0228 | CCACTGGGAAGGAGGCCGTGTTAGACGTNNNNNNNNCTTCAGCTT |
| | | CCCGATATCCGACGGTAGTGTTCTGGTCTACGGAGGG |
| 642 | FOLH1_0243 | CCACCTCCTCCAGGATATGAANNNNNNNNCTTCAGCTTCCCGATA |
| | | TCCGACGGTAGTGTTTTGGCCTGGATTCTGTTGAGCT |
| 643 | FOLH1_0247 | CCTCTCACACCAGGTTACCCAGCAANNNNNNNNCTTCAGCTTCCC |
| | | GATATCCGACGGTAGTGTTCATTCTCTACTCCGACCCT |
| 644 | FOLH1_0251 | GTGGAGCAGCTGTTGTTCATGAAATTGTGNNNNNNNNCTTCAGCT |
| | | TCCCGATATCCGACGGTAGTGTAATGAAGTGACAAGAA |
| 645 | FOLH1_0255 | GGGATCTGGAAATGATTTTGAGGTGTTNNNNNNNNCTTCAGCTTC |
| | | CCGATATCCGACGGTAGTGTACACAACCTAACAAAAGA |
| 646 | FOLH1_0259 | GTTCAGTGAGAGACTCCAGGACTTTGACNNNNNNNNCTTCAGCTT |
| | | CCCGATATCCGACGGTAGTGTAGAAAGTATGCTGACAA |
| 647 | HP16-E7_0296 | GGTTCTAAAACGAAAGTATTTGGGTAGTCNNNNNNNNCTTCAGCT |
| | | TCCCGATATCCGACGGTAGTGTCAGGTGAAGATTTGGT |
| 648 | HP16-E2_0316 | TATTAACCACCAGGTGGTGCCAACACTGGNNNNNNNNCTTCAGCT |
| | | TCCCGATATCCGACGGTAGTGTACAAAATACTAACACA |
| 649 | HP16-E2_0318 | GGATATACAGTGGAAGTGCAGTTTGATGGNNNNNNNNCTTCAGCT |
| | | TCCCGATATCCGACGGTAGTGTTGAACTGCAACTAACG |
| 650 | HP16-E2_0320 | GTAAAAATAAAGTATGGGAAGTTCATGCGNNNNNNNNCTTCAGCT |
| | | TCCCGATATCCGACGGTAGTGTATTTGTGAAGAAGCAT |
| 651 | HP16-E2_0322 | AGCCAGACACCGGAAACCCCNNNNNNNNCTTCAGCTTCCCGATAT |
| | | CCGACGGTAGTGTAGCAGCAACGAAGTATCCTCTCCTG |
| 652 | HP16-E2_0324 | GCATTGTACATTGTATACTGCAGTGTCGTNNNNNNNNCTTCAGCT |
| | | TCCCGATATCCGACGGTAGTGTTCCTCACTGCATTTAA |
| 653 | HP16-E6_0369 | GTTACTGCGACGTGAGGTATATGACTNNNNNNNNCTTCAGCTTCC |
| | | CGATATCCGACGGTAGTGTACCAAAAGAGAACTGCAA |
| 654 | HP16-E6_0370 | GTTTTATTCTAAAATTAGTGAGTATAGACNNNNNNNNCTTCAGCT |
| | | TCCCGATATCCGACGGTAGTGTTAGAATGTGTGTACTG |
| 655 | HP16-E6_0371 | CGTTGTGTGATTTGTTAATTAGGTGTATTNNNNNNNNCTTCAGCT |
| | | TCCCGATATCCGACGGTAGTGTAGAGATGGGAATCCAT |
| 656 | HP16-E6_0372 | GTGGACCGGTCGATGTATGTCTNNNNNNNNCTTCAGCTTCCCGAT |
| | | ATCCGACGGTAGTGTGAACAGCAATACAACAAAC |
| 657 | HP16-E7_0375 | CGTAGACATTCGTACTTTGGAAGACCTGNNNNNNNNCTTCAGCTT |
| | | CCCGATATCCGACGGTAGTGTGAGGAGGAGGATGAAA |
| 658 | IGF1R_0464 | GCGGGGTGGGGGGGGAGAGAGAGTTTTNNNNNNNNCTTCAGCTTC |
| | | CCGATATCCGACGGTAGTGTACAGCCTTACGCCCACA |
| 659 | IGF1R_0467 | CATTACTCGGGGGGCCATCAGGATTGNNNNNNNNCTTCAGCTTCC |
| | | CGATATCCGACGGTAGTGTAGAGCCTCGGAGACCT |
| 660 | IGF1R_0489 | GCTCAGATGCTCCAAGGATGCACCANNNNNNNNCTTCAGCTTCCC |
| | | GATATCCGACGGTAGTGTAGGAGTGCCCCTCGGGCTT |
| 661 | IGF1R_0505 | TCTCTCTCTGGGAATGGGTCGTGGACNNNNNNNNCTTCAGCTTCC |
| | | CGATATCCGACGGTAGTGTAAAATACGGATCACAAG |
| 662 | IGF1R_0513 | GGTATGACGCGAGATATCTATGAGACAGNNNNNNNNCTTCAGCTT |
| | | CCCGATATCCGACGGTAGTGTAGATGGCCGGAGAGAT |
| 663 | KDR_0549 | GCCCAATAATCAGAGTGGCAGTGNNNNNNNNCTTCAGCTTCCCGA |
| | | TATCCGACGGTAGTGTCTGTGGGTTTGCCTAGTGTTTC |
| 664 | KDR_0557 | CCTGTGCAGCATCCAGTGGGCTGATGANNNNNNNNCTTCAGCTTC |
| | | CCGATATCCGACGGTAGTGTCGAAGCATCAGCATAAGA |
| 665 | KDR_0565 | GCAGGAGAGCGTGTCTTTGTGGTGNNNNNNNNCTTCAGCTTCCCG |
| | | ATATCCGACGGTAGTGTGGCAAATGTGTCAGCTTTG |
| 666 | KDR_0581 | GTGACTTTGGCTTGGCCCGGGATNNNNNNNNCTTCAGCTTCCCGA |
| | | TATCCGACGGTAGTGTGAGCATCTCATCTGTTACAGCT |
| 667 | KDR_0589 | GCAGGGAGTCTGTGGCATCTGAAGGCTCNNNNNNNNCTTCAGCTT |
| | | CCCGATATCCGACGGTAGTGTAAAAGTAATCCCAGATG |
| 668 | KLK3_0638 | CAGTGTGTGGACCTCCATGTTATTTCCNNNNNNNNCTTCAGCTTC |
| | | CCGATATCCGACGGTAGTGTGAGCTCACGGATGCTGTG |
| 669 | KLK3_0643 | CCTGAAGAATCGATTCCTCAGGCCAGGTGNNNNNNNNCTTCAGCT |
| | | TCCCGATATCCGACGGTAGTGTACTGCATCAGGAACAA |
| 670 | KLK3_0645 | GCTTCAAGGTATCACGTCATGGGGCAGNNNNNNNNCTTCAGCTTC |
| | | CCGATATCCGACGGTAGTGTTGACGTGTGTGCGCAAGT |
| 671 | KLK3_0646 | GTGGATCAAGGACACCATCGTGGCCAANNNNNNNNCTTCAGCTTC |
| | | CCGATATCCGACGGTAGTGTGGGTGATTCTGGGGGC |
| 672 | KLK3_0647 | CTCAAGCCTCCCCAGTTCTACNNNNNNNNCTTCAGCTTCCCGATA |
| | | TCCGACGGTAGTGTCCTTCCCTGTACACCAAGGTGGTG |
| 673 | KRAS_0653 | GATCCAACAATAGAGGATTCCTACAGGANNNNNNNNCTTCAGCTT |
| | | CCCGATATCCGACGGTAGTGTAGGTGCGGGAGAGAGG |
| 674 | KRAS_0654 | GCAGGTCAAGAGGAGTACAGTGCNNNNNNNNCTTCAGCTTCCCGA |
| | | TATCCGACGGTAGTGTGAGTGCCTTGACGATACAG |
| 675 | KRAS_0655 | CATTTGAAGATATTCACCATTATAGAGNNNNNNNNCTTCAGCTTC |
| | | CCGATATCCGACGGTAGTGTGAGAAACCTGTCTCTTGG |
| 676 | KRAS_0656 | GTGATTTGCCTTCTAGAACAGNNNNNNNNCTTCAGCTTCCCGATA |
| | | TCCGACGGTAGTGTGGGAGGGCTTTCTTTGTGTATTTG |
| 677 | KRAS_0657 | GTGGAGGATGCTTTTTATACATTGGTGAGNNNNNNNNCTTCAGCT |
| | | TCCCGATATCCGACGGTAGTGTGGTCCTAGTAGGAAAT |
| 678 | KRAS_0658 | GCATTATAATGTAATCTGGGTGTTGATGNNNNNNNNCTTCAGCTT |
| | | CCCGATATCCGACGGTAGTGTAGCAAAGACAAGACAGA |
| 679 | KRAS_0659 | GCAAAGATGGTAAAAAGAAGAAAAAGAAGNNNNNNNNCTTCAGCT |
| | | TCCCGATATCCGACGGTAGTGTGCAAAGAAGAAAAGAC |
| 680 | PDCD1_0668 | CAAAGAGAGCCTGCGGGCAGANNNNNNNNCTTCAGCTTCCCGATA |
| | | TCCGACGGTAGTGTGGACTGCCGCTTCCGTGTC |
| 681 | PDCD1_0670 | GGACACTGCTCTTGGCCCCTCTNNNNNNNNCTTCAGCTTCCCGAT |
| | | ATCCGACGGTAGTGTTGCCCTGTGTCCCTGAGCAGAC |
| 682 | PDCD1_0671 | TACCGCATGAGCCCCAGCNNNNNNNNCTTCAGCTTCCCGATATCC |
| | | GACGGTAGTGTTTCTTAGACTCCCCAGACAGGCCCT |
| 683 | PDCD1_0673 | GGAGGACCCCTCAGCCGTGCCTGTGTNNNNNNNNCTTCAGCTTCC |
| | | CGATATCCGACGGTAGTGTTGGTGGTTGGTGTCGTGG |
| 684 | PDCD1_0682 | CCAGCCGGCCAGTTCCAAACCNNNNNNNNCTTCAGCTTCCCGATA |
| | | TCCGACGGTAGTGTGGCACCTACCTCTGTGGGGC |
| 685 | TP53_0689 | TCTGGCCCCTCCTCAGCATCTTNNNNNNNNCTTCAGCTTCCCGAT |
| | | ATCCGACGGTAGTGTCTGTGGGTTGATTCCACACCCCC |
| 686 | TP53_0690 | CCCCTGCACCAGCCCCCTNNNNNNNNCTTCAGCTTCCCGATATCC |
| | | GACGGTAGTGTTGGATGATTTGATGCTGTCCCCGGACG |
| 687 | TP53_0697 | GCCTGAGGTTGGCTCTGACTGTNNNNNNNNCTTCAGCTTCCCGAT |
| | | ATCCGACGGTAGTGTGAGCGCTGCTCAGATAGCGATGG |
| 688 | TP53_0699 | CGGCGCACAGAGGAAGAGAATCTCNNNNNNNNCTTCAGCTTCCCG |
| | | ATATCCGACGGTAGTGTAGTTCCTGCATGGGCGGCATG |
| 689 | TP53_0703 | TCCCACCCCCATCTCTCCCNNNNNNNNCTTCAGCTTCCCGATATC |
| | | CGACGGTAGTGTAGCAGGGCTCACTCCAGCCACCT |
| 690 | CS_2331 | GCTTCCTCCACGAATTTGAAAGACNNNNNNNNCTTCAGCTTCCCG |
| | | ATATCCGACGGTAGTGTTCTCTCCCTTTCTTACCTCCC |
| 691 | CS_2332 | GCAACATGGCAAGACGGTGGTGGGCCAANNNNNNNNCTTCAGCTT |
| | | CCCGATATCCGACGGTAGTGTAACCAAGAATGCATCT |
| 692 | CS_2333 | GTTCTTGATCCTGATGAGGGCATCCGTTNNNNNNNNCTTCAGCTT |
| | | CCCGATATCCGACGGTAGTGTAAGGAGCAGGCCAGAA |
| 693 | CS_2334 | GCCTGAGGGCTTATTTTGGCTGCTGGNNNNNNNNCTTCAGCTTCC |
| | | CGATATCCGACGGTAGTGTGGCATGAAGGGATTGGT |
| 694 | CS_2335 | CCCATGTGGTCACCATGCTGGACAACNNNNNNNNCTTCAGCTTCC |
| | | CGATATCCGACGGTAGTGTGGCTAAGGGTGGGGAAG |
| 695 | CS_2336 | GCCCGAGCATATGCACAGGGTATNNNNNNNNCTTCAGCTTCCCGA |
| | | TATCCGACGGTAGTGTAAGAGTGGGCAAAGAGGG |
| 696 | CS_2337 | GCTACCTTGTGTTGCAGCAAAGNNNNNNNNCTTCAGCTTCCCGAT |
| | | ATCCGACGGTAGTGTGTCTCAGCTCAGTGCAGCTGTT |
| 697 | CS_2338 | GGACTGGTCTCACAATTTCACCAACATGNNNNNNNNCTTCAGCTT |
| | | CCCGATATCCGACGGTAGTGTGTACTGGGAGTTGATTT |
| 698 | CS_2339 | GTGACCATGAGGGTGGCAATGTAAGTGNNNNNNNNCTTCAGCTTC |
| | | CCGATATCCGACGGTAGTGTAGAGAAGGCAGCGGTA |
| 699 | CS_2340 | GCCTCTCCATGGACTGGCAAATCAGGAAGNNNNNNNNCTTCAGCT |
| | | TCCCGATATCCGACGGTAGTGTTACCTCACCATCCACA |
| 700 | CS_2341 | GTTACGAGACTACATCTGGAACACACTCANNNNNNNNCTTCAGCT |
| | | TCCCGATATCCGACGGTAGTGTCTGTCCTTTGCAGCAG |
| 701 | CS_2342 | GCGATATACCTGTCAGCGAGAGTTTGCNNNNNNNNCTTCAGCTTC |
| | | CCGATATCCGACGGTAGTGTAGCTGCAGAAGGAAGTTG |
| 702 | CS_2343 | GTGCCCAATGTCCTCTTAGAGCNNNNNNNNCTTCAGCTTCCCGAT |
| | | ATCCGACGGTAGTGTGTTGTTCCAGGCTATGGCCATGC |
| 703 | CS_2344 | GATGAATTACTACACGGTCCTGTTTGGGNNNNNNNNCTTCAGCTT |
| | | CCCGATATCCGACGGTAGTGTGTTGGTTGCTCAGCTGT |
| 704 | CS_2345 | GAAAGGCCCAAGTCCATGAGCACAGANNNNNNNNCTTCAGCTTCC |
| | | CGATATCCGACGGTAGTGTGTGGGGTGCTGCTCCAGT |
| 705 | CS_2346 | GAGACTGGGTGAAAGTGACTACCAGAAAGNNNNNNNNCTTCAGCT |
| | | TCCCGATATCCGACGGTAGTGTGCATTGGGTGTACTGG |
| 706 | D2HGDH_3222 | GTCCCCGTCTTTGACGAGATCATCCTNNNNNNNNCTTCAGCTTCC |
| | | CGATATCCGACGGTAGTGTGCTGTAGCAAGGTGCTGCT |
| 707 | D2HGDH_3223 | GCTGAGCCGGTATGTGGAGGAANNNNNNNNCTTCAGCTTCCCGAT |
| | | ATCCGACGGTAGTGTAGGCATGGTGGGTGGCAG |
| 708 | D2HGDH_3224 | GCTGGAGGCCTGCGGTTTCTTNNNNNNNNCTTCAGCTTCCCGATA |
| | | TCCGACGGTAGTGTAATTCTGGTTTGCCAGGCGGGCTG |
| 709 | D2HGDH_3225 | GGAAGGACAACACGGGCTATGACCTGAAGNNNNNNNNCTTCAGCT |
| | | TCCCGATATCCGACGGTAGTGTAAACGTGGCAACCAAC |
| 710 | D2HGDH_3226 | GCTGTGAACGTGGCTTTCCTCNNNNNNNNCTTCAGCTTCCCGATA |
| | | TCCGACGGTAGTGTTGTCCTGGACTGCCTGACCTCCCT |
| 711 | D2HGDH_3227 | GCATTCGAGTTCATGGATGCTGTGTGNNNNNNNNCTTCAGCTTCC |
| | | CGATATCCGACGGTAGTGTCGGTGTCCATCTTGTGTCC |
| 712 | D2HGDH_3228 | GCTCCAACGCAGGCCATGACNNNNNNNNCTTCAGCTTCCCGATAT |
| | | CCGACGGTAGTGTGGGGATGCTGGGTGAGATCCTGTCT |
| 713 | D2HGDH_3229 | GCCACCGACCAGAGGAAAGTCAANNNNNNNNCTTCAGCTTCCCGA |
| | | TATCCGACGGTAGTGTCGGTGCAAGAGAGTCCGTTTT |
| 714 | D2HGDH_3230 | TACAAGTACGACCTCTCCCTCCNNNNNNNNCTTCAGCTTCCCGAT |
| | | ATCCGACGGTAGTGTTTCCTGGAGCACGCGCTGGGCTC |
| 715 | D2HGDH_3231 | GCCAAGCACGTGGTGGGCTATGNNNNNNNNCTTCAGCTTCCCGAT |
| | | ATCCGACGGTAGTGTTGGGCCCTGAGGGAAAGGATCA |
| 716 | D2HGDH_3218 | GCACGGAGTGGGCTTCAGGAAGAGGGANNNNNNNNCTTCAGCTTC |
| | | CCGATATCCGACGGTAGTGTACCTTGGAGATGGTAACC |
| 717 | FH_0120 | GTATTATGGCGCCCAGACCGTGNNNNNNNNCTTCAGCTTCCCGAT |
| | | ATCCGACGGTAGTGTTTCGGCTCCCGGCTTGGGTG |
| 718 | FH_0121 | GAAGCGAGCGGCCGCTGAAGTAAACCNNNNNNNNCTTCAGCTTCC |
| | | CGATATCCGACGGTAGTGTGGTGAACTAAAGGTGCC |
| 719 | FH_0122 | CATTTTCCTCTCGTGGTATGGCAGACTGGNNNNNNNNCTTCAGCT |
| | | TCCCGATATCCGACGGTAGTGTAAGCTTTTGGCATCTT |
| 720 | FH_0123 | GTGAACTTGGCAGCAAGATACCTGTGCNNNNNNNNCTTCAGCTTC |
| | | CCGATATCCGACGGTAGTGTGAGGTAGCTGAAGGTAAA |
| 721 | FH_0124 | GCTGCAATAGAAGTTCATGAAGTACTGTNNNNNNNNCTTCAGCTT |
| | | CCCGATATCCGACGGTAGTGTAGCAATAGAGCAATTGA |
| 722 | FH_0125 | CGTACTCATACTCAGGATGCTGTTCCNNNNNNNNCTTCAGCTTCC |
| | | CGATATCCGACGGTAGTGTTTCCCACAGCAATGCACAT |
| 723 | FH_0126 | GCCAAGAATCTATGAGCTCGCAGCTGGAGNNNNNNNNCTTCAGCT |
| | | TCCCGATATCCGACGGTAGTGTGCACAGATCATCAAGA |
| 724 | FH_0127 | GTGGCTGCACTTACAGGCTTGCCTTTTGNNNNNNNNCTTCAGCTT |
| | | CCCGATATCCGACGGTAGTGTAAATATGCAATGACAAG |
| 725 | FH_0128 | GCCTGCAGTCTGATGAAGATAGCAAATGNNNNNNNNCTTCAGCTT |
| | | CCCGATATCCGACGGTAGTGTGAAAAGGTTGCTGCAAA |
| 726 | FH_0129 | ACCAGGAAGCAGTATCATGCCAGGCAAGNNNNNNNNCTTCAGCTT |
| | | CCCGATATCCGACGGTAGTGTGGTTGAGCTCAGTGGAG |
| 727 | FH_0130 | GTTGCTGTCACTGTCGGAGGCAGCAANNNNNNNNCTTCAGCTTCC |
| | | CGATATCCGACGGTAGTGTGGTCAGGTCTGGGAGA |
| 728 | FH_0131 | GCTGCTGGGGGATGCTTCAGTTTNNNNNNNNCTTCAGCTTCCCGA |
| | | TATCCGACGGTAGTGTGCAATGACCATGGTTGCAG |
| 729 | FH_0132 | GCTGATGAATGAGTCTCTAATGTTGGTGANNNNNNNNCTTCAGCT |
| | | TCCCGATATCCGACGGTAGTGTGTTTTCAAGCCAATGA |
| 730 | FH_0133 | GCACACAAAAATGGATCAACCTTAAAGGNNNNNNNNCTTCAGCTT |
| | | CCCGATATCCGACGGTAGTGTTGCGTGGTGGGAATCC |
| 731 | FH_0134 | GGACATGCTGGGTCCAAAGTGATTTACNNNNNNNNCTTCAGCTTC |
| | | CCGATATCCGACGGTAGTGTTAGGGTATGACAAGGCAG |
| 732 | IDH1_2593 | CTACGTGGAATTGGATCTACATAGCTATGNNNNNNNNCTTCAGCT |
| | | TCCCGATATCCGACGGTAGTGTCTGTCAAGGTTTATTG |
| 733 | IDH1_2594 | GCATAATGTTGGCGTCAAATGTGCCACTNNNNNNNNCTTCAGCTT |
| | | CCCGATATCCGACGGTAGTGTGATTAAAGAGAAACTCA |
| 734 | IDH1_2595 | ATTCTGGGTGGCACGGTCTTCAGAGAAGNNNNNNNNCTTCAGCTT |
| | | CCCGATATCCGACGGTAGTGTGCAGAAGCTATAAAGAA |
| 735 | IDH1_2596 | GCTTATGGGGATCAATACAGAGCAACTGNNNNNNNNCTTCAGCTT |
| | | CCCGATATCCGACGGTAGTGTAAATCACCAAATGGCAC |
| 736 | IDH1_2597 | GAACCCAAAAGGTGACATACCTGGTNNNNNNNNCTTCAGCTTCCC |
| | | GATATCCGACGGTAGTGTGGCTTGTGAGTGGATGGGTA |
| 737 | IDH1_2598 | GTTCCTTCCAAATGGCTCTGTCTAAGGNNNNNNNNCTTCAGCTTC |
| | | CCGATATCCGACGGTAGTGTATAACCTACACACCAAGT |
| 738 | IDH1_2599 | GCGTTTTAAAGACATCTTTCAGGAGNNNNNNNNCTTCAGCTTCCC |
| | | GATATCCGACGGTAGTGTCATGGGGATGTATAATCAAG |
| 739 | IDH1_2600 | CGACGACATGGTGGCCCAAGCTATGAAANNNNNNNNCTTCAGCTT |
| | | CCCGATATCCGACGGTAGTGTCACCAAAAACACTATTC |
| 740 | IDH1_2601 | GTCGGACTCTGTGGCCCAAGGGTATNNNNNNNNCTTCAGCTTCCC |
| | | GATATCCGACGGTAGTGTCAAGTCCCAGTTTGAAGCTC |
| 741 | IDH1_2602 | GCAGAGGCTGCCCACGGGACTGTAANNNNNNNNCTTCAGCTTCCC |
| | | GATATCCGACGGTAGTGTGGAGGCTTCATCTGGGCCTG |
| 742 | IDH1_2603 | GCTTCCATTTTTGCCTGGACCAGAGGGTNNNNNNNNCTTCAGCTT |
| | | CCCGATATCCGACGGTAGTGTATGATGACCAGCGTGCT |
| 743 | IDH1_2604 | GAAGTCTCTATTGAGACAATTGAGGCTGGNNNNNNNNCTTCAGCT |
| | | TCCCGATATCCGACGGTAGTGTAGAAAGGACAGGAGAC |
| 744 | IDH1_2605 | GCAACGTTCTGACTACTTGNNNNNNNNCTTCAGCTTCCCGATATC |
| | | CGACGGTAGTGTCAGAGCAAAGCTTGATAACAATAAAG |
| 745 | IDH1_2606 | GTTCATACCTGAGCTAAGAAGGATAATNNNNNNNNCTTCAGCTTC |
| | | CCGATATCCGACGGTAGTGTGGACTTGGCTGCTTGCAT |
| 746 | IDH2_2059 | ACCCCTGATGAGGCCCGTGTGGAAGAGTTNNNNNNNNCTTCAGCT |
| | | TCCCGATATCCGACGGTAGTGTACATCCAGCTAAAGTA |
| 747 | IDH2_2060 | GAGCCCATCATCTGCAAAAACATCCCNNNNNNNNCTTCAGCTTCC |
| | | CGATATCCGACGGTAGTGTGTGTGGCTGTCAAGTGTGC |
| 748 | IDH2_2061 | GCGACCAGTACAAGGCCACAGANNNNNNNNCTTCAGCTTCCCGAT |
| | | ATCCGACGGTAGTGTAAAGTCCCAATGGAACTATCCGG |
| 749 | IDH2_2062 | GAGTGGGAAGTGTACAACTTCCCCGCAGNNNNNNNNCTTCAGCTT |
| | | CCCGATATCCGACGGTAGTGTACCAAGCCCATCACCAT |
| 750 | IDH2_2063 | GTATGCCATCCAGAAGAAATGGCCGCTGTNNNNNNNNCTTCAGCT |
| | | TCCCGATATCCGACGGTAGTGTACCCCAAAAGATGGCA |
| 751 | IDH2_2064 | GCACTATAAGACCGACTTCGACAAGNNNNNNNNCTTCAGCTTCCC |
| | | GATATCCGACGGTAGTGTTTTTGCGCACAGCTGCTTCC |
| 752 | IDH2_2065 | GCTTTGTGTGGGCCTGCAAGAACTATGNNNNNNNNCTTCAGCTTC |
| | | CCGATATCCGACGGTAGTGTTTCAAGGACATCTTCCAG |
| 753 | IDH2_2066 | GCCCTGATGGGAAGACGATTGANNNNNNNNCTTCAGCTTCCCGAT |
| | | ATCCGACGGTAGTGTTGGTGGCTCAGGTCCTCAAGTCT |
| 754 | IDH2_2067 | CCACCAGCACCAACCCCANNNNNNNNCTTCAGCTTCCCGATATCC |
| | | GACGGTAGTGTATCCTGGCCCAGGGCTTTGGCTCCCTT |
| 755 | IDH2_2068 | GCTGGAGAAGGTGTGCGTGGAGACGGTNNNNNNNNCTTCAGCTTC |
| | | CCGATATCCGACGGTAGTGTGGGAGCACCAGAAGGG |
| 756 | IDH2_2069 | GCAATGTGAAGCTGAACGAGCACTTCCTGNNNNNNNNCTTCAGCT |
| | | TCCCGATATCCGACGGTAGTGTGGGGAAGCTGGATGGG |
| 757 | MDH1_1041 | GTGACTGGAGCAGCTGGTCAAATTGCATNNNNNNNNCTTCAGCTT |
| | | CCCGATATCCGACGGTAGTGTTGGGAGAGGAGCGATCT |
| 758 | MDH1_1042 | GCTGTTGGATATCACCCCCATGATGGGNNNNNNNNCTTCAGCTTC |
| | | CCGATATCCGACGGTAGTGTAGGACGATAAGTCTGAAC |
| 759 | MDH1_1043 | GCAACAGATAAAGAAGACGTTGCCTTNNNNNNNNCTTCAGCTTCC |
| | | CGATATCCGACGGTAGTGTCTGTCTTTGGTAAAGATCA |
| 760 | MDH1_1044 | GCAAATGTGAAAATCTTCAAATCCCAGGGNNNNNNNNCTTCAGCT |
| | | TCCCGATATCCGACGGTAGTGTCCCTCCTGAAAGATGT |
| 761 | MDH1_1045 | GCCAATACCAACTGCCTGACTGCTTCCAANNNNNNNNCTTCAGCT |
| | | TCCCGATATCCGACGGTAGTGTGAAGGGAAGGCATGGA |
| 762 | MDH1_1046 | GATCACAACCGAGCTAAAGCTCAAATTGNNNNNNNNCTTCAGCTT |
| | | CCCGATATCCGACGGTAGTGTAAATACGCCAAGAAGTC |
| 763 | MDH1_1047 | GAAACCATTCCTCGACTCAGTATCCAGANNNNNNNNCTTCAGCTT |
| | | CCCGATATCCGACGGTAGTGTCTCCATCCATCCCCAA |
| 764 | MDH1_1048 | GCTCTGAAAGATGACAGCTGGCTCAAGGNNNNNNNNCTTCAGCTT |
| | | CCCGATATCCGACGGTAGTGTCTTGGTGTGACTGCTAA |
| 765 | MDH1_1049 | GCCATGTCTGCTGCAAAAGCCATCTGTGANNNNNNNNCTTCAGCT |
| | | TCCCGATATCCGACGGTAGTGTTGCAAGGAAAGGAAGT |
| 766 | MDH1_1050 | GCAACTCCTATGGTGTTCCTGATGATCTGNNNNNNNNCTTCAGCT |
| | | TCCCGATATCCGACGGTAGTGTCATCAAGGCTCGAAAA |
| 767 | MDH1_1051 | GATTTCTCACGTGAGAAGATGGATCTTNNNNNNNNCTTCAGCTTC |
| | | CCGATATCCGACGGTAGTGTGTTTGTGTCCATGGGTGT |
| 768 | MDH1_1052 | GCCTGACTAGACAATGATGTTACTAAATGNNNNNNNNCTTCAGCT |
| | | TCCCGATATCCGACGGTAGTGTAGAATAAGACCTGGAA |
| 769 | MDH1_1053 | GCTATACTTAAATTACTTGTGAAAAACAANNNNNNNNCTTCAGCT |
| | | TCCCGATATCCGACGGTAGTGTGAAGAAAAAGAAAGTG |
| 770 | MDH2_1470 | GCCACTTTCACTTCTCCTGAAGAACNNNNNNNNCTTCAGCTTCCC |
| | | GATATCCGACGGTAGTGTTCCCGCTCCAGCCATGCTCT |
| 771 | MDH2_1471 | CCACATCGAGACCAAAGCCNNNNNNNNCTTCAGCTTCCCGATATC |
| | | CGACGGTAGTGTTAAAGTAGCTGTGCTAGGGGCCTCTG |
| 772 | MDH2_1472 | GTGGTAGTTATTCCGGCTGGAGTCCNNNNNNNNCTTCAGCTTCCC |
| | | GATATCCGACGGTAGTGTCTGACCCTCTATGATATCGC |
| 773 | MDH2_1473 | GCTGCCTGTGCCCAGCACTGCNNNNNNNNCTTCAGCTTCCCGATA |
| | | TCCGACGGTAGTGTACCTGAACAGCTGCCTGACTGCCT |
| 774 | MDH2_1474 | CATTGGTGGCCATGCTGGGAAGACCATCANNNNNNNNCTTCAGCT |
| | | TCCCGATATCCGACGGTAGTGTTTCAACACCAATGCCA |
| 775 | MDH2_1475 | CAGCTGACAGCACTCACTGGNNNNNNNNCTTCAGCTTCCCGATAT |
| | | CCGACGGTAGTGTTGATCTGCGTCATTGCCAATCCGGG |
| 776 | MDH2_1476 | GCTTTGTCTTCTCCCTTGTGGATGNNNNNNNNCTTCAGCTTCCCG |
| | | ATATCCGACGGTAGTGTAAGGTGGACTTTCCCCAGGAC |
| 777 | MDH2_1477 | GCTGCTGCTTGGGAAAAAGGGCATCGAGANNNNNNNNCTTCAGCT |
| | | TCCCGATATCCGACGGTAGTGTATGGCGTATGCCGGCG |
| 778 | MDH2_1478 | GCTGAAGGCCTCCATCAAGAAGGGGGANNNNNNNNCTTCAGCTTC |
| | | CCGATATCCGACGGTAGTGTAGGAAACGGAATGTACC |
| 779 | MDH2_1479 | GCATCATGTCACTGCAAAGCCGTTGCAGANNNNNNNNCTTCAGCT |
| | | TCCCGATATCCGACGGTAGTGTAGGAGAAGATGATCTC |
| 780 | VHL_3329 | AAAGACCTGGAGCGGCTGACACAGNNNNNNNNCTTCAGCTTCCCG |
| | | ATATCCGACGGTAGTGTCCGAGTGTATACTCTGAAAGA |
| 781 | VHL_3330 | GCTTTTGATGGTACTGATGAGTCTTGATNNNNNNNNCTTCAGCTT |
| | | CCCGATATCCGACGGTAGTGTACGAAGATCTGGAAGAC |
| 782 | MET_control_ | GTGACTTCTGCCACATTACCTGACNNNNNNNNCTTCAGCTTCCCG |
| | intron_1_0002 | ATATCCGACGGTAGTGTCCTGTAGCAAGTATTTTCGCC |
| 783 | MET_control_ | CACACACACACACACACACACCAGCNNNNNNNNCTTCAGCTTCCC |
| | intron_19_0025 | GATATCCGACGGTAGTGTGATAGCGCTCTCATGGCTTG |
| 784 | MET_control_ | GCATTTGAAGGATCAAACAATCAACATCNNNNNNNNCTTCAGCTT |
| | intron_2_0057 | CCCGATATCCGACGGTAGTGTGGAAAGATACCTGATAA |
| 785 | EGFR_0403 | GCCCTGGGGATCGGCCTCTTCATNNNNNNNNCTTCAGCTTCCCGA |
| | | TATCCGACGGTAGTGTACCTGTGCCATCCAAACTGCAC |
| 786 | EGFR_0405 | GCACGGTGTATAAGGGACTCTGGATNNNNNNNNCTTCAGCTTCCC |
| | | GATATCCGACGGTAGTGTTGCTGCAGGAGAGGGAGCTT |
| 787 | EGFR_0406 | GCCAACAAGGAAATCCTCGATGAAGCNNNNNNNNCTTCAGCTTCC |
| | | CGATATCCGACGGTAGTGTAAAAGATCAAAGTGCTGGG |
| 788 | EGFR_0407 | TCTGCCTCACCTCCACCGTGCANNNNNNNNCTTCAGCTTCCCGAT |
| | | ATCCGACGGTAGTGTAAGTTAAAATTCCCGTCGCTATC |
| 789 | EGFR_0408 | GCTCCCAGTACCTGCTCAACTGGTGTGTNNNNNNNNCTTCAGCTT |
| | | CCCGATATCCGACGGTAGTGTTGGACAACCCCCACGT |
| 790 | EGFR_0410 | GCAGAAGGAGGCAAAGTGCCTATCAANNNNNNNNCTTCAGCTTCC |
| | | CGATATCCGACGGTAGTGTAGGACCGTCGCTTGGTGCA |
| 791 | EGFR_0411 | GAGTTGATGACCTTTGGATCCAAGCCNNNNNNNNCTTCAGCTTCC |
| | | CGATATCCGACGGTAGTGTCGGAAGAGAAAGAATACCA |
| 792 | EGFR_0414 | GCCAAGTCCTACAGACTCCAACNNNNNNNNCTTCAGCTTCCCGAT |
| | | ATCCGACGGTAGTGTCATGGTCAAGTGCTGGATGATAG |
| 793 | EGFR_0417 | GACAGCATAGACGACACCTTCCTCCCAGNNNNNNNNCTTCAGCTT |
| | | CCCGATATCCGACGGTAGTGTAGTGCAACCAGCAACAA |
| 794 | EGFR_0420 | GCCACCAAATTAGCCTGGACAACCCTGNNNNNNNNCTTCAGCTTC |
| | | CCGATATCCGACGGTAGTGTAGAGACCCACACTACCA |
| 795 | EGFR_0383 | GGAAATTACCTATGTGCAGAGGAATTATGNNNNNNNNCTTCAGCT |
| | | TCCCGATATCCGACGGTAGTGTCTGGAGGAAAAGAAAG |
| 796 | EGFR_0385 | GCAAATAAAACCGGACTGAAGGAGCNNNNNNNNCTTCAGCTTCCC |
| | | GATATCCGACGGTAGTGTGTGGCTGGTTATGTCCTCAT |
| 797 | EGFR_0386 | GCCCTGTGCAACGTGGAGAGCATCNNNNNNNNCTTCAGCTTCCCG |
| | | ATATCCGACGGTAGTGTCTACGAAAATTCCTATGCCTT |
| 798 | EGFR_0389 | GCCTGGTCTGCCGCAAATTCCGAGACGAANNNNNNNNCTTCAGCT |
| | | TCCCGATATCCGACGGTAGTGTCCAGAAACTGACCAAA |
| 799 | ERBB4_0067 | GTCACTGGTATTCATGGGGACCCTTNNNNNNNNCTTCAGCTTCCC |
| | | GATATCCGACGGTAGTGTCACTGACATTTGCCCAAAA |
| 800 | ERBB4_0071 | ACAACACTCTTCAGCACAATCAACCAGANNNNNNNNCTTCAGCTT |
| | | CCCGATATCCGACGGTAGTGTTGCTTATCCTCAAGCAA |
| 801 | ERBB4_0077 | GTGGGCTCTTCATTCTGGTCATTGTGGGNNNNNNNNCTTCAGCTT |
| | | CCCGATATCCGACGGTAGTGTACCCATGCCATCCAAA |
| 802 | ERBB4_0085 | GCCAATTAAATGGATGGCTCTGGAGTGNNNNNNNNCTTCAGCTTC |
| | | CCGATATCCGACGGTAGTGTGCAGCCCGTAATGTCTTA |
| 803 | ERBB4_0087 | TGCCTCAGCCTCCCATCTGCACTANNNNNNNNCTTCAGCTTCCCG |
| | | ATATCCGACGGTAGTGTGAGTGACGTTTGGAGCTATGG |
| 804 | ERBB4_0088 | GCTGAGTTTTCAAGGATGGCTCGAGNNNNNNNNCTTCAGCTTCCC |
| | | GATATCCGACGGTAGTGTAGAGAAAGGAGAACGTT |
| 805 | ERBB4_0089 | GCTTCCCAGTCCAAATGACAGCAAGNNNNNNNNCTTCAGCTTCCC |
| | | GATATCCGACGGTAGTGTGTTGGATGATTGATGCTGAC |
| 806 | ERBB4_0058 | GGGTGCTACTGCTGAGATTTTTGATGACTNNNNNNNNCTTCAGCT |
| | | TCCCGATATCCGACGGTAGTGTCCATGTCAGGAAACCA |
| 807 | ERBB4_0094 | CAGTAGCACCCAGAGGTACAGTGCTGACCNNNNNNNNCTTCAGCT |
| | | TCCCGATATCCGACGGTAGTGTCCAACTAGCACAATTC |
| 808 | ERBB4_0060 | GCAAGATATTGTTCGGAACCCATGGCCTTNNNNNNNNCTTCAGCT |
| | | TCCCGATATCCGACGGTAGTGTACAGAAAAGATGGAAA |
| 809 | MERTK_0533 | GCTGAGTAATGGCTCAGTCATGATTTTTNNNNNNNNCTTCAGCTT |
| | | CCCGATATCCGACGGTAGTGTCCCACCAACTGAAGTCA |
| 810 | MERTK_0537 | GTCCACAATGCTACGTGCACAGTGAGGANNNNNNNNCTTCAGCTT |
| | | CCCGATATCCGACGGTAGTGTAAGCAGCAGGATGGAG |
| 811 | MERTK_0540 | AATCCTTCTGTCGGCGAGCCATNNNNNNNNCTTCAGCTTCCCGAT |
| | | ATCCGACGGTAGTGTGTGGATTTATTTTGATTGGGTTG |
| 812 | MERTK_0546 | GCGAGATGACATGACTGTCTGTGTTGNNNNNNNNCTTCAGCTTCC |
| | | CGATATCCGACGGTAGTGTACAGGACCAAAGCATA |
| 813 | MERTK_0547 | GCCTGTTAAATGGATCGCCATAGNNNNNNNNCTTCAGCTTCCCGA |
| | | TATCCGACGGTAGTGTCTTCATCGAGATTTAGCTGCTC |
| 814 | MERTK_0549 | GAAGACTGCCTGGATGAACTGTATGANNNNNNNNCTTCAGCTTCC |
| | | CGATATCCGACGGTAGTGTGTGATGTGTGGGCATTTGG |
| 815 | MERTK_0550 | CTCTTAGAAAGTTTGCCTGACGTTCGGNNNNNNNNCTTCAGCTTC |
| | | CCGATATCCGACGGTAGTGTTGGCCACAGGTTGAAGCA |
| 816 | MERTK_0554 | GCTGACGACTCCTCAGAAGGCTCAGNNNNNNNNCTTCAGCTTCCC |
| | | GATATCCGACGGTAGTGTTCTGCTGCAGTCACAGCTG |
| 817 | MERTK_0527 | TCGCTTCCTTCAGCATAACCAGTGTGNNNNNNNNCTTCAGCTTCC |
| | | CGATATCCGACGGTAGTGTCTCAATCAGTGTACCTAAT |
| 818 | MERTK_0530 | CGAACAGCCTGAAAAATCCCCCNNNNNNNNCTTCAGCTTCCCGAT |
| | | ATCCGACGGTAGTGTTCCTCACTTTACTAAGCAGCCTG |
| 819 | PLXND1_0604 | TTCCGCCCTTCCCCCCCAACNNNNNNNNCTTCAGCTTCCCGATAT |
| | | CCGACGGTAGTGTCAGCCTGCCCAGCCTCAGTGGCAT |
| 820 | PLXND1_0605 | TCACCATCTACGACTGCAGCCNNNNNNNNCTTCAGCTTCCCGATA |
| | | TCCGACGGTAGTGTTTTGGTCACCAGATTGCCTACTGC |
| 821 | PLXND1_0589 | CAGACCCCTGCACGGAGCTGANNNNNNNNCTTCAGCTTCCCGATA |
| | | TCCGACGGTAGTGTTTCTCCTACGTGCTGCCCCTGGTC |
| 822 | PLXND1_0612 | CCGGGGAGCCTCTCACCCTCGTTANNNNNNNNCTTCAGCTTCCCG |
| | | ATATCCGACGGTAGTGTGAGGTGGCTGTGGCTGAGG |
| 823 | PLXND1_0613 | GCTGCGACATCCAGATTGTCTCTGACAGANNNNNNNNCTTCAGCT |
| | | TCCCGATATCCGACGGTAGTGTGGCCAAGAGGGAGAAG |
| 824 | PLXND1_0614 | TTCAACCAGACCATCGCCACACNNNNNNNNCTTCAGCTTCCCGAT |
| | | ATCCGACGGTAGTGTTACCGGGTCAAGATAGGCCAAGT |
| 825 | PLXND1_0616 | GCAAAGGCTTCGCTGAGCTGCAGANNNNNNNNCTTCAGCTTCCCG |
| | | ATATCCGACGGTAGTGTGTCCTGCTGCTGCTCTCCGTG |
| 826 | PLXND1_0617 | GGAGTATAAGCACTTCGTGACCCGCACNNNNNNNNCTTCAGCTTC |
| | | CCGATATCCGACGGTAGTGTTGCTGCAGATGGAGGAG |
| 827 | PLXND1_0618 | TCCCAGACCCTCAACTCCCAGGGNNNNNNNNCTTCAGCTTCCCGA |
| | | TATCCGACGGTAGTGTACATGACAGATCTCACCAAGGA |
| 828 | PLXND1_0622 | GTCCATCTGCATGTACAGCTGTCTGCGGGNNNNNNNNCTTCAGCT |
| | | TCCCGATATCCGACGGTAGTGTTACTACACCAGCATCA |
| 829 | PLXND1_0626 | TTCGCCTCCAGCACACAGANNNNNNNNCTTCAGCTTCCCGATATC |
| | | CGACGGTAGTGTATGGACTCGCTGAGCGTGCGGGCCAT |
| 830 | PLXND1_0630 | CTTTTTCGACTTCCTGGAGGAGCAGGCNNNNNNNNCTTCAGCTTC |
| | | CCGATATCCGACGGTAGTGTTGCTCCCGGAAATCTACC |
| 831 | PLXND1_0632 | TCATCGCGCAGGCCTTCATCGACGCNNNNNNNNCTTCAGCTTCCC |
| | | GATATCCGACGGTAGTGTACCCCGACACCCTACACATC |
| 832 | PLXND1_0634 | CTGGCCGAGGAGTCGAGGAAATACCAGAANNNNNNNNCTTCAGCT |
| | | TCCCGATATCCGACGGTAGTGTATTCGCCAACCAACAA |
| 833 | PLXND1_0636 | GGTGGTGGCTTTGATGGAGGACAACATCTNNNNNNNNCTTCAGCT |
| | | TCCCGATATCCGACGGTAGTGTAACACCAATGTGGCCA |
| 834 | RET_0681 | TCTCCCAGCACCAAGACCNNNNNNNNCTTCAGCTTCCCGATATCC |
| | | GACGGTAGTGTCTGCCCCCTGTCCTGTGCAGTCAGC |
| 835 | RET_0683 | GCTTCCCTGAGGAGGAGAAGTGCTTCTGNNNNNNNNCTTCAGCTT |
| | | CCCGATATCCGACGGTAGTGTGCGATGTTGTGGAGAC |
| 836 | RET_0687 | ATTGTATGGGGCCTGCAGCCAGGNNNNNNNNCTTCAGCTTCCCGA |
| | | TATCCGACGGTAGTGTAAAGGCAGAGCAGGGTA |
| 837 | RET_0690 | CGGCTTGTCCCGAGATGTTTATGNNNNNNNNCTTCAGCTTCCCGA |
| | | TATCCGACGGTAGTGTCTCATCTCATTTGCCTGGCAG |
| 838 | RET_0691 | GATCATATCTACACCACGCAAAGTGATGNNNNNNNNCTTCAGCTT |
| | | CCCGATATCCGACGGTAGTGTGAGGGGCGGAAGATGA |
| 839 | RET_0693 | GGAGATGTACCGCCTGATGCNNNNNNNNCTTCAGCTTCCCGATAT |
| | | CCGACGGTAGTGTTGGTCTTTTGGTGTCCTGCTGTGGG |
| 840 | RET_0695 | CACCGCTGGTGGACTGTAATAATGCCNNNNNNNNCTTCAGCTTCC |
| | | CGATATCCGACGGTAGTGTGGTGTTTGCGGACATCAG |
| 841 | RET_0697 | GGATGCTTTCACCCTCAGCGGCAAAATNNNNNNNNCTTCAGCTTC |
| | | CCGATATCCGACGGTAGTGTGAAAACAAACTCTATGGC |
| 842 | RET_0698 | GTGAAAGGTAATGGACTCACAAGGGGAANNNNNNNNCTTCAGCTT |
| | | CCCGATATCCGACGGTAGTGTACGAGAGCTGATGGCA |
| 843 | RET_0673 | GCTCCTGGGAGAAGCTCAGTNNNNNNNNCTTCAGCTTCCCGATAT |
| | | CCGACGGTAGTGTTGAGGAGGTGCCCAGCTTCCG |
| 844 | AXL_0731 | TCGTCGGACCACTGAAGCTACCNNNNNNNNCTTCAGCTTCCCGAT |
| | | ATCCGACGGTAGTGTCTGTGTCCTCATCTTGGCTCTCT |
| 845 | AXL_0735 | TCCTCCTCTATTCCCGGCTNNNNNNNNCTTCAGCTTCCCGATATC |
| | | CGACGGTAGTGTTCTGCATGAAGGAATTTGACCATCCC |
| 846 | AXL_0737 | GTCCGTGTGTGTGGCGGACTTCNNNNNNNNCTTCAGCTTCCCGAT |
| | | ATCCGACGGTAGTGTAGTGTACCTGCCCACTCAGATG |
| 847 | AXL_0738 | GCCATTGAGAGTCTAGCTGACCGTGTCTNNNNNNNNCTTCAGCTT |
| | | CCCGATATCCGACGGTAGTGTAGGAACTGCATGCTGAA |
| 848 | AXL_0739 | CGGGCGTGGAGAACAGCGAGATTTATGNNNNNNNNCTTCAGCTTC |
| | | CCGATATCCGACGGTAGTGTAAGATGCCAGTCAAGTGG |
| 849 | AXL_0740 | GGACTGTATGCCTTGATGTCGCGGTGCTNNNNNNNNCTTCAGCTT |
| | | CCCGATATCCGACGGTAGTGTGGGGTGACAATGTGGG |
| 850 | AXL_0742 | AGCTGACCCCCCAACCCANNNNNNNNCTTCAGCTTCCCGATATCC |
| | | GACGGTAGTGTGTTTTACAGAGCTGCGGGAAGATTTGG |
| 851 | AXL_0744 | TTCCCACCCCACGCCTTATCNNNNNNNNCTTCAGCTTCCCGATAT |
| | | CCGACGGTAGTGTTCAGCCTGCTGATAGGGGCTCCCC |
| 852 | AXL_0727 | GCCCGAAGACAGGACTGTGGCNNNNNNNNCTTCAGCTTCCCGATA |
| | | TCCGACGGTAGTGTAGCTCAGAATCACCTCCCTGCA |
| 853 | AXL_0717 | TCCCCCTGGCCACGGCTCCANNNNNNNNCTTCAGCTTCCCGATAT |
| | | CCGACGGTAGTGTGCTGGAGGGCTTGCCTTACTTCCT |
| 854 | EGFR_delta_14_ | GCCAGGTCTTGAAGGCTGTCCAANNNNNNNNCTTCAGCTTCCCGA |
| | 15_1808_nt_0001 | TATCCGACGGTAGTGTGTCTGCCATGCCTTGTGCT |
| 855 | EGFR_delta_14_ | GCCCTGGGGATCGGCCTCTTCATGCGAANNNNNNNNCTTCAGCTT |
| | 15_1808_nt_0002 | CCCGATATCCGACGGTAGTGTGACAAGTGCAACCTTCT |
| 856 | EGFR_delta_2_7_ | GTGGTGACAGATCACGGCTCGTGNNNNNNNNCTTCAGCTTCCCGA |
| | 265_nt_0006 | TATCCGACGGTAGTGTGAGAGCCGGAGCGAGCTCTT |
| 857 | EGFR_delta_2_7_ | GCCGCAAAGTGTGTAACGGAATAGGTANNNNNNNNCTTCAGCTTC |
| | 265_nt_0007 | CCGATATCCGACGGTAGTGTCTGGAGGAAAAGAAAG |
| 858 | EGFR_delta_12_ | GCCAAGGGAGTTTGTGGAGAACTCTGAGTNNNNNNNNCTTCAGCT |
| | 12_674_nt_0011 | TCCCGATATCCGACGGTAGTGTATTCTGAAAACCGTAA |
| 859 | EGFR_delta_12_ | CGGGGACCAGACAACTGTATCCAGTGTGNNNNNNNNCTTCAGCTT |
| | 12_674_nt_0012 | CCCGATATCCGACGGTAGTGTAACCTAGAAATCATACG |
| 860 | EGFR_delta_25_ | CACAATCAGCCTCTGAACCCNNNNNNNNCTTCAGCTTCCCGATAT |
| | 27_3123_nt_0017 | CCGACGGTAGTGTCCAAAGTTCCGTGAGTTGATCATCG |
| 861 | MET_delta_14_3_ | GTTTCCTAATTCATCTCAGAACGGTTCANNNNNNNNCTTCAGCTT |
| | 128_nt_0025 | CCCGATATCCGACGGTAGTGTAATAGTTCAACCAGATC |
| 862 | MET_delta_14_3_ | CAGTCCATTACTGCAAAATACTGTCCACANNNNNNNNCTTCAGCT |
| | 128_nt_0026 | TCCCGATATCCGACGGTAGTGTAAAAAGAGAAAGCAAA |
| 863 | MET_delta_4_5_ | GCAGGTTTTCCCAAATAGTGCACCCCTTGNNNNNNNNCTTCAGCT |
| | 1579_nt_0032 | TCCCGATATCCGACGGTAGTGTGCTTTGCAGCGCGTTG |
| 864 | MET_delta_4_5_ | ACTAGAGTTCTCCTTGGAAATGAGAGCNNNNNNNNCTTCAGCTTC |
| | 1579_nt_0033 | CCGATATCCGACGGTAGTGTGACATCAGAGGGTCGCTT |
| 865 | MET_delta_7_8_ | GCACGATGAATACTGTGTCAAACAGNNNNNNNNCTTCAGCTTCCC |
| | 2049_nt_0037 | GATATCCGACGGTAGTGTTTGAAGGAGGGACAAGGCTG |
| 866 | MET_delta_7_8_ | GCCAACCGAGAGACAAGCATCTTCANNNNNNNNCTTCAGCTTCCC |
| | 2049_nt_0038 | GATATCCGACGGTAGTGTAATGAGAGCTGCACCTTGAC |
| 867 | MET_var_1_fusion_ | ATTAGTACTTGGTGGAAAGAACCTCTCAANNNNNNNNCTTCAGCT |
| | 9_10A_2638_nt_ | TCCCGATATCCGACGGTAGTGTCCCAAACCATTTCAAC |
| | 0042 | |
| 868 | MET_var_1_fusion_ | CAGTTAGTGTCCCGAGAATGGTCATAAANNNNNNNNCTTCAGCTT |
| | 9_10A_2638_nt_ | CCCGATATCCGACGGTAGTGTAAATTCATCCAACCAAA |
| | 0043 | |
| 869 | MET_var_2_fusion_ | GTGGTGGGAGCACAATAACAGGTGTTGNNNNNNNNCTTCAGCTTC |
| | 9_10B_2664_nt_ | CCGATATCCGACGGTAGTGTCAAACCATTTCAACTGAG |
| | 0047 | |
| 870 | MET_var_2_fusion_ | GCATGTCAACATCGCTCTAATTCAGNNNNNNNNCTTCAGCTTCCC |
| | 9_10B_2664_nt_ | GATATCCGACGGTAGTGTATTCATCCAACCAAATCTTT |
| | 0048 | |
| 871 | MET_var_2_fusion_ | GCATGTCAACATCGCTCTAATTCAGNNNNNNNNCTTCAGCTTCCC |
| | 9_11_2464_nt_ | GATATCCGACGGTAGTGTCCAAACCATTTCAACTGAGT |
| | 0052 | |
| 872 | MET_var_2_fusion_ | GCCTTTTTCATGTTAGATGGGATCCTTTNNNNNNNNCTTCAGCTT |
| | 9_11_2464_nt_ | CCCGATATCCGACGGTAGTGTCTATGAAATTCATCCAA |
| | 0053 | |
| 873 | KIT_0066 | GTCACAACAACCTTGGAAGTAGTAGANNNNNNNNCTTCAGCTTCC |
| | | CGATATCCGACGGTAGTGTATAATAGCTGGCATCACGG |
| 874 | KIT_0069 | CCGAAGGAGGCACTTACACATTCCTAGNNNNNNNNCTTCAGCTTC |
| | | CCGATATCCGACGGTAGTGTGAACACCAGCAGTGGATC |
| 875 | KIT_0072 | GCACAATGGCACGGTTGAATGTAAGGNNNNNNNNCTTCAGCTTCC |
| | | CGATATCCGACGGTAGTGTTGTCCAGGAACTGAGCAGA |
| 876 | KIT_0075 | CAAATGGGAGTTTCCCAGAAACAGGCTGANNNNNNNNCTTCAGCT |
| | | TCCCGATATCCGACGGTAGTGTGTGATGATTCTGACCT |
| 877 | KIT_0078 | GCTATGGTGATCTTTTGAATTTTTTGAGNNNNNNNNCTTCAGCTT |
| | | CCCGATATCCGACGGTAGTGTAACGGGAAGCCCTCATG |
| 878 | KIT_0080 | GCCGACAAAAGGAGATCTGTGAGAATAGGNNNNNNNNCTTCAGCT |
| | | TCCCGATATCCGACGGTAGTGTAAGATCATGCAGAAGC |
| 879 | KIT_0083 | GTGAAGTGGATGGCACCTGAAAGCANNNNNNNNCTTCAGCTTCCC |
| | | GATATCCGACGGTAGTGTAGAGACTTGGCAGCCAGAAA |
| 880 | KIT_0058 | GCTGTTATGCACTGATCCGGGCTTNNNNNNNNCTTCAGCTTCCCG |
| | | ATATCCGACGGTAGTGTTTCTGCTCCTACTGCTTCGCG |
| 881 | KIT_0060 | TGCCAAGCTTTTCCTTGTTGACCGCTCCNNNNNNNNCTTCAGCTT |
| | | CCCGATATCCGACGGTAGTGTAACGAATGAGAATAAGC |
| 882 | KIT_0063 | GCTGTGCCTGTTGTGTCTGTGTNNNNNNNNCTTCAGCTTCCCGAT |
| | | ATCCGACGGTAGTGTAAGGCGGGCATCATGATCAAAAG |
| 883 | PTEN_0098 | GGATTCAAAGCATAAAAACCATTACAAGANNNNNNNNCTTCAGCT |
| | | TCCCGATATCCGACGGTAGTGTCAAGAGGATGGATTCG |
| 884 | PTEN_0100 | CATGTTGCAGCAATTCACTGTAAAGCTGGNNNNNNNNCTTCAGCT |
| | | TCCCGATATCCGACGGTAGTGTACACCGCCAAATTTAA |
| 885 | PTEN_0101 | GCCCTAGATTTCTATGGGGAAGTAAGGNNNNNNNNCTTCAGCTTC |
| | | CCGATATCCGACGGTAGTGTCCAATGGCTAAGTGAAGA |
| 886 | PTEN_0102 | GGATTATAGACCAGTGGCACTGTTGTTTNNNNNNNNCTTCAGCTT |
| | | CCCGATATCCGACGGTAGTGTTTTAAAGGCACAAGAG |
| 887 | PTEN_0103 | CCTCAGTTTGTGGTCTGCCAGCTNNNNNNNNCTTCAGCTTCCCGA |
| | | TATCCGACGGTAGTGTGTCAGAGGCGCTATGTGTATT |
| 888 | PTEN_0104 | GCCGTTACCTGTGTGTGGTGATATNNNNNNNNCTTCAGCTTCCCG |
| | | ATATCCGACGGTAGTGTTTCCAATGTTCAGTGGCGG |
| 889 | PTEN_0105 | ACCAGGACCAGAGGAAACCTCANNNNNNNNCTTCAGCTTCCCGAT |
| | | ATCCGACGGTAGTGTGTTCATGTACTTTGAGTTCCCTC |
| 890 | PTEN_0107 | AGCCAACCGATACTTTTCTCCAANNNNNNNNCTTCAGCTTCCCGA |
| | | TATCCGACGGTAGTGTGTAGAAAATGGAAGTCTATGTG |
| 891 | PTEN_0109 | GATCAGCATACACAAATTACAAAAGTCTGNNNNNNNNCTTCAGCT |
| | | TCCCGATATCCGACGGTAGTGTCGTCAAATCCAGAGGC |
| 892 | PTEN_0110 | GGACCTTTTTTTTTTTAATGGCAATAGGNNNNNNNNCTTCAGCTT |
| | | CCCGATATCCGACGGTAGTGTTGACTCTGATCCAGAGA |
| 893 | ACTB_0129 | TTGCTCCTCCTGAGCGCAAGTACTCNNNNNNNNCTTCAGCTTCCC |
| | | GATATCCGACGGTAGTGTACATCCGCAAAGACCTGTAC |
| 894 | ACTB_0123 | TCTGGCACCACACCTTCTACAATGNNNNNNNNCTTCAGCTTCCCG |
| | | ATATCCGACGGTAGTGTGTGATGGTGGGCATGGGTC |
| 895 | ACTB_0124 | AACCCCAAGGCCAACCGCGANNNNNNNNCTTCAGCTTCCCGATAT |
| | | CCGACGGTAGTGTTGAAGTACCCCATCGAGCACGGCAT |
| 896 | ACTB_0126 | GGTCATCACCATTGGCAATGAGCGGTNNNNNNNNCTTCAGCTTCC |
| | | CGATATCCGACGGTAGTGTGAGCGGGAAATCGTGCGTG |
| 897 | ACTB_0127 | GGCATCCACGAAACTACCTTCAACNNNNNNNNCTTCAGCTTCCCG |
| | | ATATCCGACGGTAGTGTTGCTTCCAGCTCCTCCCTGG |
| 898 | ACTB_0128 | CCACCATGTACCCTGGCATTGCCNNNNNNNNCTTCAGCTTCCCGA |
| | | TATCCGACGGTAGTGTACTCTTCCAGCCTTCCTTCCT |
| 899 | MET_var_1_0147 | GTTCCATAAACTCTGGATTGCATTCCTACNNNNNNNNCTTCAGCT |
| | | TCCCGATATCCGACGGTAGTGTCAGAGATTCTTACCCC |
| 900 | MET_var_1_0150 | GTGAGATGTCTCCAGCATTTTTACGGNNNNNNNNCTTCAGCTTCC |
| | | CGATATCCGACGGTAGTGTTTCTTTTCGGGGTGTTCGC |
| 901 | MET_var_1_0153 | ACTCCCATCCAGTGTCTCNNNNNNNNCTTCAGCTTCCCGATATCC |
| | | GACGGTAGTGTCTCTTAACATCTATATCCACCTTCATT |
| 902 | MET_var_1_0156 | GCTGACCATATGTGGCTGGGACTTTGGATNNNNNNNNCTTCAGCT |
| | | TCCCGATATCCGACGGTAGTGTTGGTGCCACGACAAAT |
| 903 | MET_var_1_0159 | GCTGGTGGCACTTTACTTACTTTNNNNNNNNCTTCAGCTTCCCGA |
| | | TATCCGACGGTAGTGTGTCCTGCCATGAATAAGCATTT |
| 904 | MET_var_1_0162 | GCTTTGCCAGTGGTGGGAGCACAATANNNNNNNNCTTCAGCTTCC |
| | | CGATATCCGACGGTAGTGTAGCCAACCGAGAGACAA |
| 905 | MET_var_1_0165 | GCCTTTTGAAAAGCCAGTGATGATCNNNNNNNNCTTCAGCTTCCC |
| | | GATATCCGACGGTAGTGTTTGTACCACTCCTTCCCTGC |
| 906 | MET_var_1_0169 | GCAAATTAAAGATCTGGGCAGTGAATTAGNNNNNNNNCTTCAGCT |
| | | TCCCGATATCCGACGGTAGTGTGTCCTTGGAAAAGTAA |
| 907 | MET_var_1_0170 | CCCAACTACAGAAATGGTTTCAAATGNNNNNNNNCTTCAGCTTCC |
| | | CGATATCCGACGGTAGTGTCTTGGGTTTTTCCTGTGGC |
| 908 | MET_var_1_0171 | GCAGTATCCTCTGACAGACATGTCCNNNNNNNNCTTCAGCTTCCC |
| | | GATATCCGACGGTAGTGTGCTTGTAAGTGCCCGAAGTG |
| 909 | MET_var_1_0174 | CAATTTCTGACCGAGGGAATCATCATGANNNNNNNNCTTCAGCTT |
| | | CCCGATATCCGACGGTAGTGTGAAGTCATAGGAAGAGG |
| 910 | MET_var_1_0178 | GCAAACTCAAAAGTTTACCACCAAGTCNNNNNNNNCTTCAGCTTC |
| | | CCGATATCCGACGGTAGTGTAAAAATTCACAGTCAAGG |
| 911 | MET_var_1_0181 | ACTTTCATTGGGGAGCACTATGTCCATNNNNNNNNCTTCAGCTTC |
| | | CCGATATCCGACGGTAGTGTACTCCTACAACCCGAATA |
| 912 | MET_var_1_0184 | GTATTGTTATTTAAATTACTGGATTCTANNNNNNNNCTTCAGCTT |
| | | CCCGATATCCGACGGTAGTGTCATAGTGCTAGTACTAT |
| 913 | MET_var_1_0144 | CGGTTCATCAACTTCTTTGTAGGNNNNNNNNCTTCAGCTTCCCGA |
| | | TATCCGACGGTAGTGTACAATCATACTGCTGACATACA |
| 914 | TUBB_0211 | TCCGCCGGAAGGCCTTCCTCCACTNNNNNNNNCTTCAGCTTCCCG |
| | | ATATCCGACGGTAGTGTAACAATGTCAAGACAGCCGTC |
| 915 | TUBB_0202 | CAGCTGACCCACTCACTGGNNNNNNNNCTTCAGCTTCCCGATATC |
| | | CGACGGTAGTGTTTTGTATTTGGTCAGTCTGGGGCAGG |
| 916 | TUBB_0205 | CCACCTTGTCTCAGCCACCANNNNNNNNCTTCAGCTTCCCGATAT |
| | | CCGACGGTAGTGTTACAATGCCACCCTCTCCGTCCATC |
| 917 | TUBB_0208 | TACCTCACCGTGGCTGCTNNNNNNNNCTTCAGCTTCCCGATATCC |
| | | GACGGTAGTGTCTTTATGCCTGGCTTTGCCCCTCTCAC |
| 918 | ERBB3_0013 | CCACCACTCTTTGAACTGGACCAAGNNNNNNNNCTTCAGCTTCCC |
| | | GATATCCGACGGTAGTGTCGGGGCTTCTCATTGTTGAT |
| 919 | ERBB3_0015 | GCCGAGGAGGTGTCTGTGTGANNNNNNNNCTTCAGCTTCCCGATA |
| | | TCCGACGGTAGTGTAGACATCAAGCATAATCGGCCG |
| 920 | ERBB3_0019 | CCCATCTGACAATGGCTTTGACAGTGNNNNNNNNCTTCAGCTTCC |
| | | CGATATCCGACGGTAGTGTCCCAATCTACAAGTACCCA |
| 921 | ERBB3_0025 | GCCAAGGGAATGTACTACCTTGAGGANNNNNNNNCTTCAGCTTCC |
| | | CGATATCCGACGGTAGTGTGTCATCTCTGCAGCTTG |
| 922 | ERBB3_0027 | GTGGATGGCCCTTGAGAGTATCCACTTNNNNNNNNCTTCAGCTTC |
| | | CCGATATCCGACGGTAGTGTAAACGTGCTACTCAAGTC |
| 923 | ERBB3_0034 | GCAGTTTCTGGGAGCAGTGAACGGTGCNNNNNNNNCTTCAGCTTC |
| | | CCGATATCCGACGGTAGTGTAGCCTACCAGTTGGAA |
| 924 | ERBB3_0037 | TACTCCCTCCTCCCGGGAAGGCANNNNNNNNCTTCAGCTTCCCGA |
| | | TATCCGACGGTAGTGTCGGAGATAGCGCCTACCA |
| 925 | ERBB3_0043 | CTCCTGCTCCCTGTGGCACTCNNNNNNNNCTTCAGCTTCCCGATA |
| | | TCCGACGGTAGTGTCCCCCCATGTCCATTATGCCC |
| 926 | ERBB3_0002 | GCTTTGTCACATGGACACAATTGACTGNNNNNNNNCTTCAGCTTC |
| | | CCGATATCCGACGGTAGTGTGGAAGTTTGCCATCTTC |
| 927 | ERBB3_0005 | GCCTGCCGGCACTTCAATGACAGTGGAGNNNNNNNNCTTCAGCTT |
| | | CCCGATATCCGACGGTAGTGTACATTGACCAAGACCA |
| 928 | RON_0255 | TTTTGCCCCAACCCGCCTNNNNNNNNCTTCAGCTTCCCGATATCC |
| | | GACGGTAGTGTCCCCAACTCTGTCGTCTGTGCCTTCCC |
| 929 | RON_0263 | AACTGGAGCCCTTGGGCACCCAGNNNNNNNNCTTCAGCTTCCCGA |
| | | TATCCGACGGTAGTGTTTCTGGTCTGGTGCCTGAGGG |
| 930 | RON_0265 | GGCACCTGTCTCACTCTTGAAGGCNNNNNNNNCTTCAGCTTCCCG |
| | | ATATCCGACGGTAGTGTCCTCACCGTGACTAACATGCC |
| 931 | RON_0270 | GGAGCTGCTGGCTTTACACTGCCTGGNNNNNNNNCTTCAGCTTCC |
| | | CGATATCCGACGGTAGTGTGGCTTAGGGCAGTGGAAAG |
| 932 | RON_0274 | GCACTGGTCTTCAGCTACTGGTGGNNNNNNNNCTTCAGCTTCCCG |
| | | ATATCCGACGGTAGTGTGGTCTGCGTAGATGGTG |
| 933 | RON_0279 | GTGGAGGCCTTCCTGCGAGAGGGGCTNNNNNNNNCTTCAGCTTCC |
| | | CGATATCCGACGGTAGTGTACAGTGACCGAGTCATTGG |
| 934 | RON_0281 | CCTCATCAGCTTTGGCCTGCAGGTANNNNNNNNCTTCAGCTTCCC |
| | | GATATCCGACGGTAGTGTTGTGCTGGCTCTCATTGGT |
| 935 | RON_0282 | CAGTCAAGGTGGCTGACTTTGGTTNNNNNNNNCTTCAGCTTCCCG |
| | | ATATCCGACGGTAGTGTAACCCCACCGTGAAGGA |
| 936 | RON_0287 | CTCACCCATGCCAGGGAATGTACGNNNNNNNNCTTCAGCTTCCCG |
| | | ATATCCGACGGTAGTGTTGGGGGAGGTGGAGCAG |
| 937 | RON_0252 | CCACACGGGAGCCTTCGTATACTNNNNNNNNCTTCAGCTTCCCGA |
| | | TATCCGACGGTAGTGTTTCAGCCCACGCTCAGTGTCT |
| 938 | ALK_0320 | TGCCCAGAGGCTCCTTTCTCCNNNNNNNNCTTCAGCTTCCCGATA |
| | | TCCGACGGTAGTGTTGTGAGCTGGAGTATTCCCCTCC |
| 939 | ALK_0323 | GTGGAAACCGCAGCTTGTCTGCAGTGGANNNNNNNNCTTCAGCTT |
| | | CCCGATATCCGACGGTAGTGTACAACGAGGCTGCAAGA |
| 940 | ALK_0328 | CGTGTCCTTGGTGCTAGTGGNNNNNNNNCTTCAGCTTCCCGATAT |
| | | CCGACGGTAGTGTTTGCTCAGTACCACTGATGTCCC |
| 941 | ALK_0329 | GCCTGTGGCAGTGGATGGTGTTGNNNNNNNNCTTCAGCTTCCCGA |
| | | TATCCGACGGTAGTGTTGAGCTCCGAATGTCCTGG |
| 942 | ALK_0334 | GCGGGAAAGGCGGGAAGAACACCATGANNNNNNNNCTTCAGCTTC |
| | | CCGATATCCGACGGTAGTGTAACAACGCCTACCAGAA |
| 943 | ALK_0335 | GCTGTACATCCTGGTTGGGCAGCAGGGANNNNNNNNCTTCAGCTT |
| | | CCCGATATCCGACGGTAGTGTAGCCACCGACACCTACA |
| 944 | ALK_0347 | CTGCCCCGGTTCATCCTGCTGGANNNNNNNNCTTCAGCTTCCCGA |
| | | TATCCGACGGTAGTGTTGGCTGTGAAGACGCTGCCTG |
| 945 | ALK_0349 | CAGACACATGGTCCTTTGGAGTGCTGNNNNNNNNCTTCAGCTTCC |
| | | CGATATCCGACGGTAGTGTTGGAGACTTCGGGATG |
| 946 | ALK_0355 | CCCAACGTACGGCTCCTGGTTNNNNNNNNCTTCAGCTTCCCGATA |
| | | TCCGACGGTAGTGTTCTCTGTTCGAGTCCCTAGAGGGC |
| 947 | ALK_0358 | GCCCCTGGAGCTGGTCATTACGANNNNNNNNCTTCAGCTTCCCGA |
| | | TATCCGACGGTAGTGTTGCTCCTAGAGCCCTCTTCGCT |
| 948 | EGFR_0391 | GCCTGTGGGGCCGACAGCTATGAGATGGANNNNNNNNCTTCAGCT |
| | | TCCCGATATCCGACGGTAGTGTTCTACAACCCCACCAC |
| 949 | EGFR_0394 | CGTAAAGGAAATCACAGGGTTTTTGCTGANNNNNNNNCTTCAGCT |
| | | TCCCGATATCCGACGGTAGTGTAAACACTTCAAAAACT |
| 950 | forward primer | CTGGTAACGGCAATGCGGCT |
| | HMBSFw | |
| 951 | reverse primer | TTCTTCTCCAGGGCATGTTC |
| | HMBSRv | |

Sequences 1 - 949 are smMIPs

### EXAMPLES

### Example 1—Targeted smMIP-Based RNA Sequencing Yields Relevant Information on Metabolism

In the past four decades an overwhelming amount of data has become available on the molecular events that underlie carcinogenesis. Research has mainly focused on molecular alterations and their consequences for among others the PI3K/pAKT/mTOR pathway(19-22) and cell cycle control, apoptosis (23, 24) and DNA repair pathways (25, 26). Currently, numerous FDA-approved drugs are available that target cancer cells based on these genetic defects with a level of specificity that is not attainable with conventional chemotherapies (27, 28), permitting personalized medicine. Whereas targeted cancer therapies may prolong survival, it is now widely recognized that inherent genetic instability ultimately leads to therapy resistance of most cancers (11-14).

For proliferation, cancer cells need to generate ATP to maintain energy balance and ion homeostasis, import carbon and nitrogen sources for synthesis of amino acids, nucleotides and lipids (29, 30) and maintain redox potential to protect cells against oxidative stress (31). Blocking one or more of these processes may prohibit proliferation and/or sensitize cells to toxic therapy in a synthetic lethality approach. As an example, increasing oxidative stress in a cancer with metabolic inhibitors may enhance the efficacy of radiotherapy (32) or chemotherapy (33). With the increasing knowledge of deranged metabolic pathways in cancer (34-37), (adjuvant) targeting of cancer-specific metabolic pathways may be a highly interesting addition to current treatment protocols. The best-known example of cancer-specific metabolic adaptation is aerobic glycolysis, also known as the Warburg effect (38). As glycolysis is inefficient in terms of ATP production, cancer cells characteristically upregulate glucose transporters GLUT1 and/or GLUT3. Besides glucose, glutamine and fatty acids are recognized as important fuels for cancer cells (39, 40) (41, 42).

While metabolic adaptations are mostly seen as a consequence of carcinogenesis, it has been unequivocally established that metabolic alterations can also cause cancer, examples being mutations in genes encoding mitochondrial (e.g. IDH2, FH, SDH) and cytosolic (e.g. IDH1) metabolic enzymes (43, 44) (45, 46) (1, 47, 48), the latter being prominent in among others low grade gliomas and secondary glioblastomas (1, 48). Clear cell renal cell carcinoma (ccRCC) is now considered a metabolic disease with metabolic alterations resulting indirectly from inactivating mutations in or epigenetic silencing of VHL, found in ˜80% of clear cell renal cell cancers (ccRCC) (49). pVHL is a major regulator of ubiquitination and breakdown of transcription factor hypoxia inducible factors HIF-1α and HIF-2α (49). Mutations in the aforementioned metabolic enzymes and in VHL have been shown to induce epigenetic alterations that affect expression of other metabolic enzymes in an unpredictable fashion (17, 50-52).

To apply metabolic inhibitors as potential additions to the current anti-tumor armamentarium, it is of high importance to identify which metabolic pathways are active in a specific cancer in a personalized fashion. Here we applied a novel next generation-sequencing based method using single molecule molecular inversion probes (smMIPs(15)), to detect expression levels of 104 genes involved in metabolism, and concomitantly identify variants therein. As a proof of concept, we applied smMIPs to map part of the metabolic transcriptome of a VHL-defective ccRCC cell line and a corresponding VHL-rescued isogenic derivative, as well as in patient derived glioma xenograft models. We validated the technique by correlating results with whole transcriptome RNAseq data (as gold standard for transcriptome analysis) and protein expression. We further verified the ability of the assay to detect oncogenic mutations in cell lines and patient tumor tissue.

Our data show that targeted RNA sequencing of transcripts encoding metabolic enzymes using smMIPs predict the predominant metabolic pathways that are operational in cancer (53) and simultaneously allows variant detection in the targeted transcripts.

### Materials and Methods

### Cell Lines—

The cell line SKRC7 is derived from a primary human ccRCC and has been described before (54). Cells were cultured in RPMI 1640 (Lonza Group, Switzerland) supplemented with 10% fetal calf serum (FCS) (Gibco, Thermo Fisher Scientific, Waltham, Mass., USA) and 40 μg/ml gentamycin (Centrafarm, Etten-Leur, The Netherlands). An isogenic SKRC7 cell line expressing a functional haemagglutinine (HA)-tagged VHL (SKRC7-VHL^{HA}) was created by transfection with pcDNA3.1-VHL^{HA} followed by selection of stable transfectants in the same medium with 400 μg/ml geneticin (Gibco, Thermo Fisher Scientific, Waltham, Mass., USA). The patient-derived glioma xenograft models E478 and E98 have been described before (55, 56).

### Patient Material—

Use of patient material was according to the guidelines of the local ethical committee for use of patient material and was performed with informed consent. Surgically obtained tissue from a male patient with a grade III astrocytoma was snap frozen frozen in liquid nitrogen.

RNA and cDNA Preparation—

Total RNA was isolated from sections of snap-frozen E478 xenograft tissue, human tumor tissue and from 80% confluent SKRC7 and SKRC7-VHL^{HA} cells using TRIzol reagent (Life Technologies, ThermoFisher Scientific, Waltham, Mass., USA) according to the manufacturers' instructions. RNA quality was estimated based on relative levels of 28S, 18S and 5S rRNA bands on agarose gel and with Bioanalyzer assays (Agilent Technologies, Amstelveen, The Netherlands). RNA was reverse transcribed to cDNA using Superscript II reverse transcriptase (Invitrogen, ThermoFisher Scientific, Waltham, Mass., USA) and random hexamer primers (Promega, Madison, Wis., USA) according to standard protocols. Next, cDNA was purified using the NucleoSpin Gel and PCR Clean-up kit (Macherey-Nagel, DOren, Germany). For quality control, cDNA was subjected to PCR for reference gene hydroxymethylbilane Synthase (HBMS) with forward primer HMBSFw (5′-CTGGTAACGGCAATGCGGCT-3′) and reverse primer HMBSRv (5′-TTCTTCTCCAGGGCATGTTC-3′) using AmpliTaq Gold 360 master mix (Applied Biosystems, ThermoFisher Scientific, Waltham, Mass. USA).

### Whole Transcriptome RNAseq Analysis

High quality RNA with RIN scores >8 was subjected to whole transcriptome RNAseq according to standard protocols. Sequencing was performed on an Illumina Hiseq and yielded 30-50 million reads per sample (paired end sequencing protocol). The dataset was analyzed using the ‘Tuxedo’ protocol; reads were mapped against the RefSeq human genome (hg19) with TopHat and final transcript assembly was done with the Cufflinks package (57). Normalization was done with both Cuffquant and the calculation of fragments as transcript per million mapped reads (TPM) to obtain relative expression values. Occurrence of single nucleotide variants was visualized in the Integrated Genomics Viewer browser (IGV, the Broadinstitute).

smMIP Design

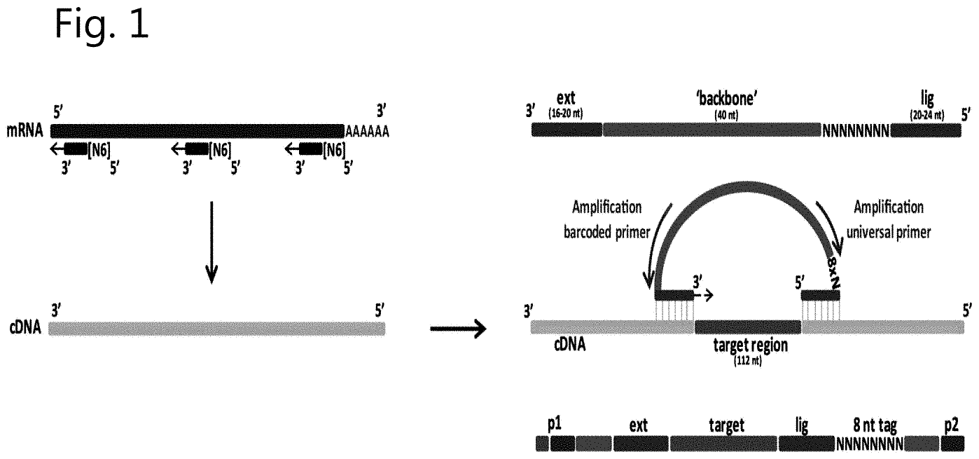

The technique of targeted RNAseq using smMIPs is depicted in FIG. 1. It is based on the hybridization of an extension and ligation probe, joined by a ‘constant’ backbone sequence in an inverted manner to a cDNA of interest, followed by gap-filling/ligation and PCR. SmMIPs against the antisense strand of 104 predicted transcripts (UCSC human genome assembly hg19) were designed based on the MIPgen algorithm as described by Boyle et al. (18). Whenever possible, smMIPs were designed with ligation and extension probes located on adjacent exons to prevent contribution of smMIP probes that hybridize to potential contaminations of genomic DNA. Transcripts of interest were encoding enzymes and transporters functioning in various metabolic pathways, including lipid metabolism, glycolysis, oxidative phosphorylation (OXPHOS), tricarboxylic acid (TCA) cycle, pentose phosphate pathway (PPP), glutaminolysis and control of reductive potential (see Table I). The smMIP set also contained probes for detection of β-actin and β-tubulin as housekeeping genes, and a number of tyrosine kinases with relevance for cancer. SmMIPs were designed with extension probes of 16 to 20 nt in length and ligation probes of 20-24 nt in length, joined by a constant backbone sequence (40 nt) with a stretch of 8 random nucleotides incorporated adjacent to the ligation probe. The random 8N sequence is incorporated to reduce all amplicons originating from one individual smMIP to one unique MIP (see below). The length of gap-fill was set at 112 nt. Whereas the design was based on full coverage, for the majority of transcripts 5-10 smMIPS per transcript were included in the panel with the target regions distributed evenly over the reading frame. For 18 transcripts (CS, D-2HGDH, L-2HGDH, FH, IDH1-3A-G, MDH1-2, MYC, OGDH, SDHA-D, VHL) smMIP sets were chosen that covered the full coding sequences.

### Capture and Library Preparation

642 smMIPs (IDT, Leuven, Belgium) were pooled at 100 μM/smMIP. The smMIP pool was phosphorylated using T4 Polynucleotide Kinase (New England Biolabs, NEB, Ipswich, Mass., USA) in T4 DNA ligase buffer (NEB) at 37° C. for 45 min, followed by inactivation for 20 min at 65° C. The capture reaction was performed with 50 ng of cDNA and an estimated 8000-fold molar excess of the phosphorylated smMIP pool (16) in a 25 μL reaction mixture containing Ampligase buffer (Epicentre, Madison, Wis., USA), dNTPs, Hemo KlenTaq enzyme (New England Biolabs, NEB, Ipswich, Mass., USA) and thermostable DNA ligase (Ampligase, Epicentre). The capture mix was incubated for 10 min at 95° C. (denaturation), followed by incubation for 18 h at 60° C., during which hybridization and concomitant primer extension and ligation occurs. Directly after this step non-circularized smMIPs, RNA and cDNA were removed by treatment with 10 U Exonuclease I and 50 U of Exonuclease III (both NEB) for 45 min at 37° C., followed by heat inactivation (95° C., 2 min). The circularized smMIP library was subjected to standard PCR with 2× iProof High-Fidelity DNA Polymerase master Mix (Bio-Rad, Hercules, Calif.) with a primer set containing a unique barcoded reverse primer for each sample. Generation of PCR products of correct size (266 bp) was validated on agarose gel electrophoresis, and PCR-libraries from different samples were pooled based on relative band intensity. The pool was then purified using AMPureXP beads (Beckman Coulter Genomics, High Wycombe, UK) according to manufacturers' instructions. The purified library was run on a TapeStation 2200 (Agilent Technologies, Santa Clara, Calif., USA) and quantified via Qubit (Life Technologies, ThermoFisher Scientific, Waltham, Mass. USA) to assess quality of the library. Reproducibility of the technique was tested by preparing biological replica libraries, using different RNA preparations from the same cell lines.

### Sequencing and Annotation

Libraries were sequenced on the Illumina NextSeq platform (Illumina, San Diego, Calif.) at the Radboudumc sequencing facility to produce 2×150 bp paired-end reads. Reads were mapped to the reference transcriptome (hg19) using the SeqNext module of JSI SequencePilot version 4.2.2 build 502 (JSI Medical Systems, Ettenheim, Germany). The random 8 nt sequence flanking the ligation probe was used to reduce PCR amplicates to one smMIP (unique reads).

### Single Nucleotide Variant (SNV) Calling and Expression Analysis—

All single nucleotide variants (SNVs) called with a minimal variant percentage of 5% detected in at least 5 unique reads (forward and reverse) were selected for further analysis. Variants were annotated and classified into synonymous or non-synonymous. Next, they were validated in whole transcriptome RNAseq data, generated from different RNA isolations from the same cell lines.

Individual read counts for each smMIP were divided by the total read count within a sample and multiplied by 10⁶ resulting in a fragment per million (FPM) value for each smMIP in a sample. We choose for this normalization procedure instead of normalization against housekeeping genes because perfect housekeeping genes do not exist. E.g. expression of metabolic genes is subject to variation, dependent on cell cycle, and the same is true for expression of genes such as actin and tubulin.

### Western Blotting—

Cell extracts were prepared from SKRC7 and SKRC7-VHL^{HA} cells by solubilizing in RIPA buffer (Cell Signaling) and protein concentrations were determined using BCA assays. 20 μg of protein was separated on 12% SDS-PAGE gels and electroblotted on nitrocellulose. After blocking in Odyssey blocking buffer (1:1 in PBS) membranes were incubated overnight in Odyssey blocking buffer containing antibodies against HK-2 (2867S, Cell signaling technology), CA9 (M75, Dr. Oosterwijk) or γ-tubulin (C20, Santa Cruz Biotechnology, Dallas, Tex.) as loading control. Antibodies were detected with secondary antibodies conjugated with Alexa680 or DyLight800, and signal was visualized with the Odyssey scanner (LI-COR).

### Statistics

FPM values for each transcript (mean FPM values from all smMIPs targeting one transcript) were correlated with TPM values (transcripts per million values for the same transcript obtained from whole RNAseq data from the same cell lines. For three samples replicate assays were performed. Correlation analyses were performed using GraphPad Prism v.5.03 (GraphPad, San Diego, Calif., USA).

### Results

SmMIP-based next generation sequencing (NGS) of genomic DNA was recently introduced in routine diagnostics in our institute to detect tumor-associated mutations in DNA (16). To investigate whether smMIPs can also be used for multiplex determination of gene expression levels, concomitant with variant detection, we designed a smMIP set for targeted detection and sequencing of transcripts encoding metabolic enzymes. To establish the strength of the technique we used the SKRC7 and SKRC7-VHL^{HA} isogenic cell line pair as prototypical cell lines in which different metabolic pathways prevail. Like 80% of ccRCCs, the SKRC7 cell line carries a defective VHL gene resulting in constitutive stabilization of HIF-1α and HIF-2α and a pseudohypoxic response (53, 54, 58). Re-introduction of VHL was expected to result in rapid HIF1/2 breakdown and repair of this metabolic aberration.

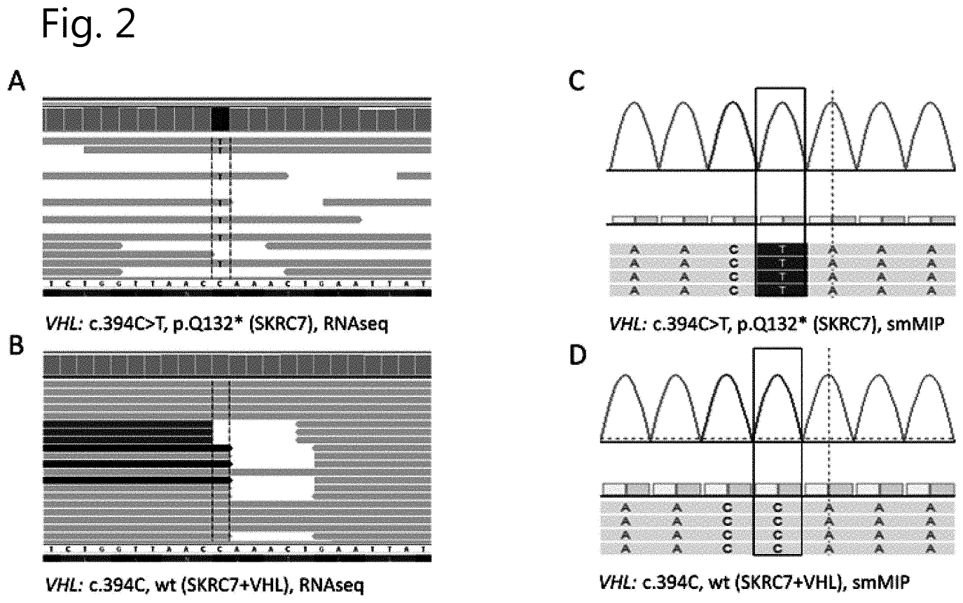

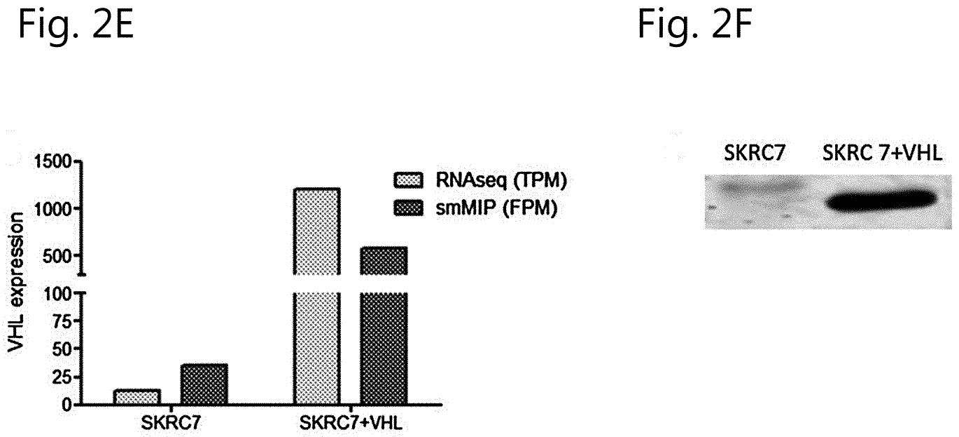

Whole RNAseq-derived gene expression data of SKRC7 cells confirmed the presence of a nonsense and functionally inactivating Q132-stop mutation in 100% of VHL transcripts (FIG. 2A) whereas only wtVHL sequence was detected in the SKRC7VHL^{HA} (FIG. 2B), and this was readily reproduced in the smMIP assay (FIG. 2C,D). Introduction of functional VHL^{HA} in SKRC7 cells resulted in 100-fold increase in VHL expression (FIG. 2E, see also western blot in FIG. 2F).

### Optimization of Library Preparation

Using an initial set of 642 smMIPs, covering 104 transcripts of interest for this study (see Table I), we tested our protocol of library preparation with 50 ng of hexamer-primed cDNA generated from 13 different RNA samples (cell line- and xenograft derived) of which also whole RNAseq datesets were available. A 25-cycle PCR with barcoded primers on the circularized smMIP library yielded PCR fragments of the expected size of 266 bp (not shown).

Based on initial experiments we also tested the procedure on 10 and 25 ng cDNA. Both conditions yielded less PCR fragments and less unique reads compared to 50 ng. We therefore continued with 50 ng cDNA input in subsequent experiments. Illumina NextSeq sequencing of the libraries generated of SKRC7 and SKRC7-VHL cells, yielded 286,000 and 69,000 annotated unique reads respectively (corrected for PCR-amplicates based on the random 8N sequence in the smMIP), which is in the range of other samples run with the same smMIP panel (not shown). For most transcripts performance of individual smMIPs was variable (see example in Table II, showing FPM values for 10 different smMIPs designed against the VHL transcript in both cell lines), a known phenomenon also in DNA smMIP NGS (16)). This was a priori reason to include at least 5 smMIPs per gene transcript in our panel, allowing transcriptome analysis using mean normalized smMIP values for each transcript. This number was a trade-off between generating expensive, large panels which would yield in part futile and irrelevant data, and too small panels resulting in under- or overestimation of transcript levels.

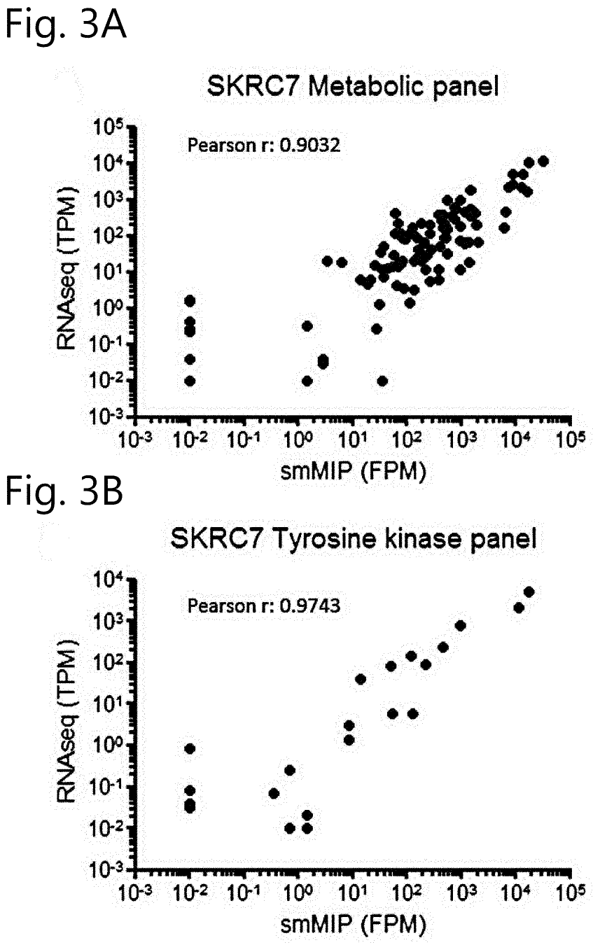

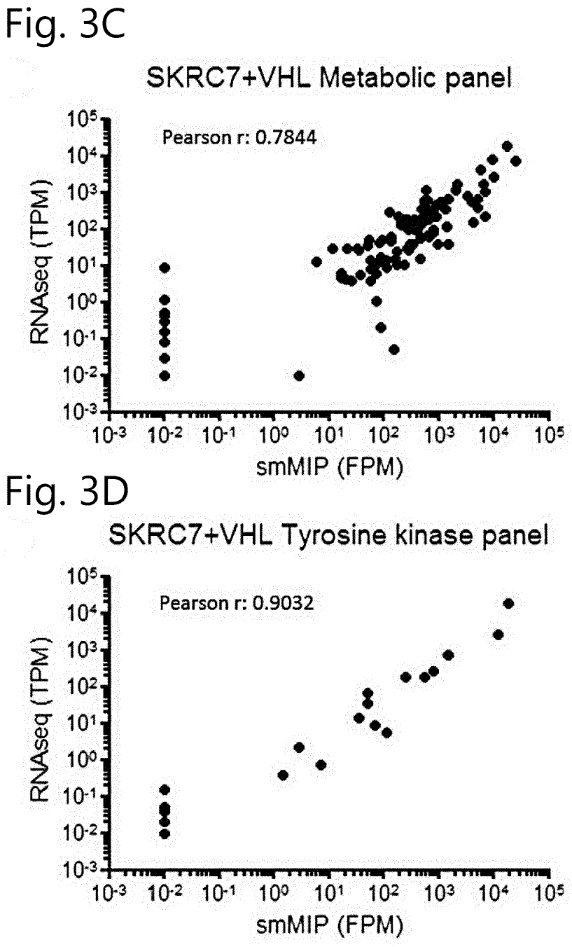

First we compared the targeted smMIP RNAseq dataset, generated with a 864 smMIP panel, to a whole transcriptome RNAseq dataset (considered as gold standard), performed on different RNA isolates from the same cell lines. The whole RNAseq dataset consisted of 3.2×10⁷ and 3.4×10⁷ reads, assigned to 44,503 different transcripts for SKRC7 and SKRC7-VHL^{HA}, respectively. For each transcript of interest, TPM (transcripts per million) values from the whole RNAseq dataset were plotted against mean FPM values from smMIP analyses. Such analysis for metabolic transcripts and tyrosine kinase transcripts separately, gave correlation coefficients of 0.903 and 0.974, respectively, for SKRC7 (FIG. 3A,B) and 0.784 and 0.903, respectively, for SKRC7-VHL^{HA} (FIG. 3C,D), suggesting that, as expected, expression of metabolic genes is subject to more variation than of tyrosine kinases. Plotting whole transcriptome RNAseq data against unique reads obtained with the best performing smMIP per transcript, or the median of unique reads for each transcript (to prevent bias by non- or poor-performing smMIPs) did not improve this correlation (not shown).

One of the appealing characteristics of targeted RNAseq using smMIPs is that panels can be expanded to detect novel transcripts of interest. To test how this affects the outcome of the assay, we added 222 smMIPs for detection and targeted sequencing of other transcripts of interest to our initial panel and re-performed the assay using newly isolated RNA from the same cell line. Relative levels of transcripts within samples correlated well between assays with the initial and the expanded smMIP set (SKRC7: r=0.903, SKRC7-VHL^{HA}: r=0.876).

Functional Validation of Targeted smMIP Data

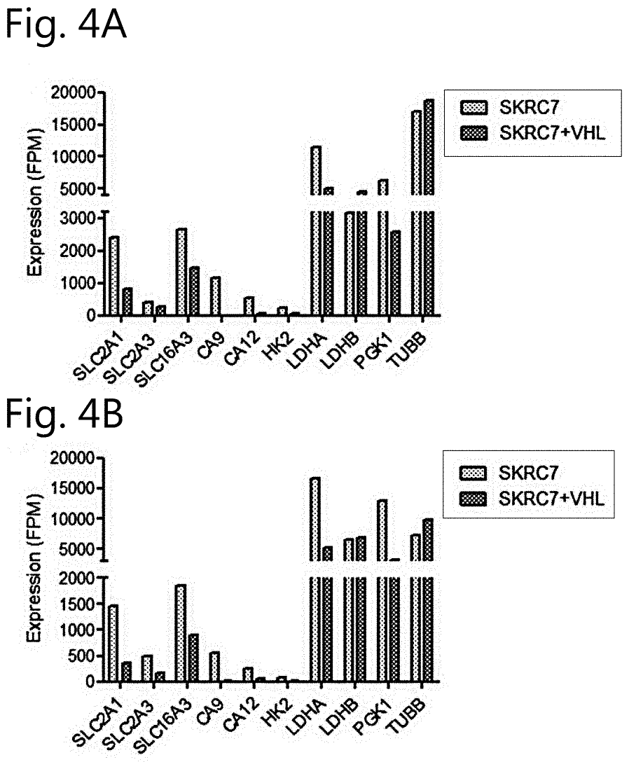

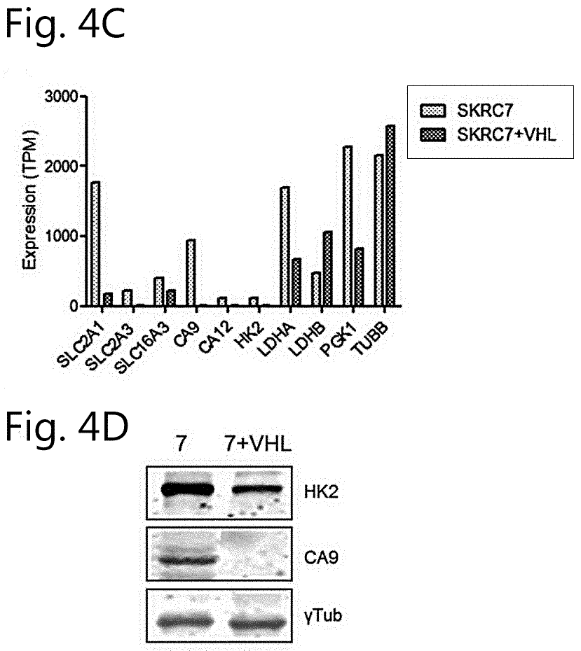

Having confirmed the validity of the smMIP dataset, we analyzed expression levels of genes involved in metabolism in SKRC7 and SKRC7-VHL^{HA} cells. FIGS. 4A and 4B show two biological duplicates of smMIP-based mean FPM values for a number of transcripts involved in glycolysis. Expression of HIF target genes glucose transporter 1 and 3 (SLC2A1 and SCL2A3), monocarboxylate transporter MCT4 (SLC16A3), carbonic anhydrases 9 and 12 (CA9, CA12), hexokinase 2 (HK2), lactate dehydrogenase A (LDH-A) and phosphoglycerate kinase (PGK1) were significantly and reproducibly reduced in SKRC7-VHL^{HA} cells relative to SKRC7 cells (FIG. 4A,B), in line with data obtained from whole transcriptome RNA seq data (FIG. 4C). Relative expression levels of CA9 and HK2 transcript levels were further confirmed on the protein level (FIG. 4D). The strong reduction of CA9, HK2 and LDHA, all target genes of HIF, was in line with expectations for a VHL-defective cell line.

### Variant Detection

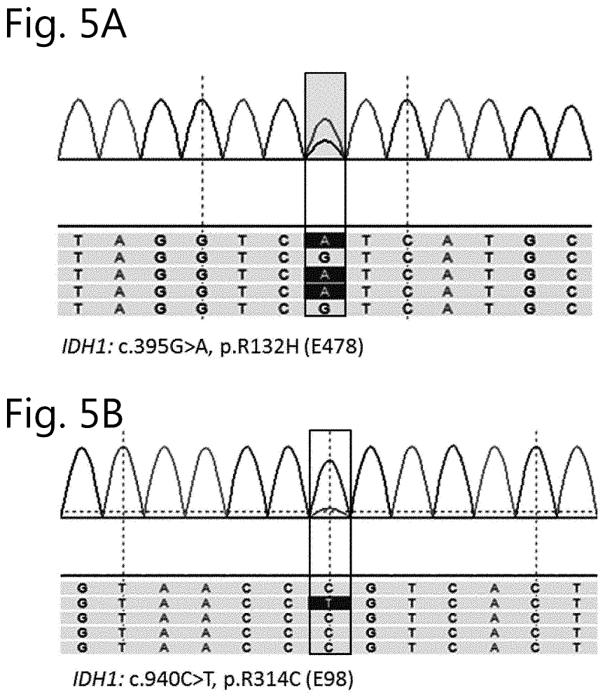

To investigate whether smMIP based RNAseq allows efficient detection of single nucleotide variants (SNVs), we performed variant calling of the smMIP library in SeqNext. Several heterozygous and homozygous variants were detected that could be validated in the whole RNAseq dataset (see VHL example in FIGS. 2C and D). We then further validated the sensitivity of the assay to detect SNVs (called a variant in relation to reference genome hg19) and performed smMIP analysis on RNA, isolated from the IDH1^{R132H} mutant oligodendroglioma line E478 (56) and the astrocytoma cell line E98, in which we previously identified a novel mutation in IDH1 (IDH1^{R314}C)(1). Both mutations were identified (FIG. 5A,B).

### Discussion

Here we present a novel approach of library generation for targeted RNA next generation sequencing using smMIPs and show that the technique yields reproducible and biologically relevant information which is qualitatively comparable to whole transcriptome RNAseq. Especially for the evaluation of relative contributions of metabolic pathways in cancer, that are amenable to epigenetic and transcription-factor based regulation, DNA sequencing may not always yield relevant information. Using an isogenic pair of cell lines differing only in VHL expression as a test case, we here show that targeted RNA seq of transcripts involved in metabolism with our smMIP panel yields relevant information on metabolic pathways with relative abundancies that are similar to that of whole transcriptome RNAseq.

Although the generation of smMIP libraries for targeted RNAseq obviously yields only a fraction of the data compared with whole transcriptome RNAseq, it has distinct advantages: 1) costs of the technique are approximately 5-10% of the cost of whole transcriptome RNAseq; 2) by designing smMIPs with ligation and extension probes localized on neighbouring exons, the library is expected not to be contaminated with heteronuclear RNA, transfer RNAs and ribosomal RNAs that may account for a large number of reads in whole transcriptome RNAseq, dependent on preprocessing; 3) the choice of extension and ligation target sequences allows the design of smMIPs that specifically detect splice variants; 4) the technique allows detection of SNVs and indels with high efficiency, once smMIPs are chosen to cover the mutated region; 5) coverage per target sequence of transcript of interest is higher than with whole transcriptome RNAseq; 6) smMIP sets may be extended with novel smMIPs of interest without affecting performance; 7) data sets are much smaller and easier to handle.

The technique was validated with an isogenic ccRCC cell line pair, differing only in expression of VHL. Our targeted smMIP analysis revealed that, as expected, expression levels of HIF target genes HK-2, CA9, CA12 and LDH-A were downregulated upon rescue of VHL, a known regulator of HIF proteolysis. This suggests that rescue of VHL function reduces flux of glucose into the glycolytic pathway.

Because altered metabolic activity in cancer can be a consequence of general oncogenic stress but also of mutations in genes encoding metabolic enzymes and conditions relating to the microenvironement (oxygen tension, access to stromal cell-derived metabolites (59)) cancer metabolism is in an extremely complex landscape (60). Nevertheless, targeting of cancer-specific metabolic pathways is gaining importance in cancer research. Since decades glycolysis has been considered the predominant metabolic pathway in cancer, but it is increasingly clear that tumors can also thrive using glutamine as carbon and nitrogen donor. Identification of the fuel-processing pathways that represent metabolic Achilles heels in cancer is important to apply metabolic inhibition in a personalized fashion. Approaches to identify metabolic pathways in clinical cancers currently include carbon tracing using ex vivo mass spectroscopy (59, 61, 62) and in vivo ¹H-, ¹³C carbon- or ³¹P-based magnetic resonance spectroscopic imaging (40, 63) but these approaches are not suitable for implementation in routine patient care. SmMIP-based transcript profiling may be a highly relevant alternative with added value in the field of cancer diagnostics as it can identify metabolic Achilles heels by simultaneously measuring smart combinations of relative gene expression levels and variants. When combined with smMIP sets that detect actionable mutations in oncogenes or tumor suppressor genes, personalized treatment protocols may be further optimized by including inhibitors of the most predominant metabolic pathways such as glycolysis (e.g. 3-bromopyruvate, dichloroacetate(64-66)), pentose phosphate pathway (e.g. 6-aminonicotinamide (33), glutaminolysis (e.g. epigallocathechin-3-gallate(67, 68)), mitochondrial oxidative phosphorylation (e.g. metformin(69-71)), fatty acid oxidation and lipid synthesis (e.g. cerulenin (72)).

### Example 2—Glutaminolysis in Cancers Predicts Enhanced Sensitivity to a Combination of Epigallocatechin-3-Galate (EGCG) and Radiotherapy as Compared to Radiotherapy Alone

Clear cell renal cell carcinoma (ccRCC) are relatively resistant to radiotherapy and chemotherapy. Due to dysfunctional von Hippel-Lindau (VHL) protein these tumors accumulate hypoxia-inducible factors HIF-1α and HIF-2α resulting in pseudohypoxic responses that accompanying aberrant metabolism (49, 73). This translates in expression of a set of transporters and enzymes that increase glucose uptake for use in aerobic glycolysis and lactate production, instead of oxidative phosphorylation (73). Increased uptake of glucose and its conversion to glucose-6-phosphate by the hexokinase family of enzymes leads to an increased flux through the pentose phosphate pathway (PPP), providing the cell with NADPH, the most important form of reducing power in cells, and ribose-5-phosphate (R5P), a precursor of nucleotide synthesis (33). Whereas in normal cells oxidative glucose metabolism yields mitochondrial citrate as a major carbon source for lipid biosynthesis, this pathway is blocked in VHL-deficient cells that process pyruvate towards lactate instead of acetyl-CoA for TCA cycle feeding (74). Cells with a VHL defect therefore use glutamine as a metabolic rescue pathway. During glutaminolysis, glutamine is converted to glutamate and α-KG via the sequential activities of glutaminase and glutamate dehydrogenase. Subsequently cells employ reductive carboxylation of α-ketoglutarate (α-KG) in the cytoplasm to produce isocitrate (reverse reaction of IDH1) that is converted to citrate (aconitase) and acetyl-CoA (ATP-citrate lyase) that, together with NADPH, is processed to fatty acids (75). In VHL-mutated cancers high expression levels of enzymes of the pentose phosphate pathway (PPP), combined with low levels of TCA enzymes correlates with poor survival (76).

In ccRCC differential expression of HIF-1α and HIF-2α is observed, with tumors expressing either both subtypes or exclusively HIF-2a. Part of the effects of HIF-1α and HIF-2α are overlapping, but they also have distinct effects on cell metabolism. HIF-1α causes glycolytic enzyme expression (77) and limits mitochondrial pyruvate consumption (78, 79), thereby blocking anabolic biosynthesis via this pathway. Furthermore HIF-1α inhibits cell cycle progression via inhibition of c-myc (80). HIF-2α however, does not regulate glycolysis and stimulates cell-cycle progression (81). The exact metabolic pathways may therefore differ between different VHL-mutated cancers. Unraveling the metabolic pathways of cancer cells that facilitate malignant behavior is of high importance, since these may be amenable for targeting with the aim to inhibit cell growth and tumor progression, or induce oxidative stress sensitizing cells to radiotherapy or chemotherapy.

Here we investigated the metabolic pathways in two VHL impaired ccRCC cell lines, SKRC-17 (expressing only HIF-2) and SKRC-7 (expressing both HIF-1 and HIF2) (54, 58). Expression of metabolic enzymes was explored with smMIP sequencing, and carbon sources that are essential for proliferation of these cells were identified. Results show that SKRC-7 cells use glucose for lactate production (high levels of enzymes for glucose-to-pyruvate-to-lactate). Gene expression profiles of SKRC-17 suggest that cells use glucose mainly for the pentose phosphate pathway. Both cell types have high levels of glutaminase and glutamate dehydrogenase, suggesting sensitivity to the glutamate dehydrogenase inhibitor EGCG. The high levels of PPP in SKRC17 suggest additional activity of 6-aminonicotinamide (6-AN).

### Materials and Methods

### Cell Culture

SKRC-7, derived from primary human RCC, and SKRC-17, derived from a soft tissue metastatic lesion of human RCC (82) both carry a non-sense mutation in VHL (Q132X in SKRC-7 and S65X in SKRC-17) and therefore lack functional pVHL. In SKRC-7 this leads to high levels of HIF-1α and HIF-2α, whereas SKRC-17 presents with high levels of HIF-2α only (54, 58). Unless stated otherwise, cells were cultured in RPMI 1640 (Lonza Group, Switzerland) supplemented with 10% fetal calf serum (FCS) (Gibco, Thermo Fisher Scientific, Waltham, Mass., USA) and 40 μg/ml gentamycin (Centrafarm, Etten-Leur, The Netherlands).

### SmMIP Sequencing

642 smMIPs (IDT, Leuven, Belgium) were pooled at 100 μM/smMIP. The smMIP pool was phosphorylated using T4 Polynucleotide Kinase (New England Biolabs, NEB, Ipswich, Mass., USA) in T4 DNA ligase buffer (NEB) at 37° C. for 45 min, followed by inactivation for 20 min at 65° C. The capture reaction was performed with 50 ng of cDNA and an estimated 8000-fold molar excess of the phosphorylated smMIP pool (16) in a 25 μL reaction mixture containing Ampligase buffer (Epicentre, Madison, Wis., USA), dNTPs, Hemo KlenTaq enzyme (New England Biolabs, NEB, Ipswich, Mass., USA) and thermostable DNA ligase (Ampligase, Epicentre). The capture mix was incubated for 10 min at 95° C. (denaturation), followed by incubation for 18 h at 60° C., during which hybridization and concomitant primer extension and ligation occurs. Directly after this step, non-circularized smMIPs, RNA and cDNA were removed by treatment with 10 U Exonuclease I and 50 U of Exonuclease III (both NEB) for 45 min at 37° C., followed by heat inactivation (95° C., 2 min). The circularized smMIP library was subjected to standard PCR with 2× iProof High-Fidelity DNA Polymerase master Mix (Bio-Rad, Hercules, Calif.) with a primer set containing a unique barcoded reverse primer for each sample. Generation of PCR products of correct size (266 bp) was validated on agarose gel electrophoresis, and PCR-libraries from different samples were pooled based on relative band intensity. The pool was then purified using AMPureXP beads (Beckman Coulter Genomics, High Wycombe, UK) according to manufacturers' instructions. The purified library was run on a TapeStation 2200 (Agilent Technologies, Santa Clara, Calif., USA) and quantified via Qubit (Life Technologies, ThermoFisher Scientific, Waltham, Mass. USA) to assess quality of the library.

Reproducibility of the technique was tested by preparing biological replica libraries, using different RNA preparations from the same cell lines.

### Western Blottinq

To verify transcript levels observed in the smMIP sequencing data on protein level, western blots for some of the interesting enzymes active in glycolysis (HK2, PKM2, GAPDH), glutaminolysis (GLUD) and reverse carboxylation (IDH1) were performed. Cells were cultured in 6 well plates till 80% confluency, then cells were harvested and processed to cell lysates by scraping in 100 μl 10 mM Tris-HCL pH 7.5 and 0.32 M sucrose and sonicating on ice (3 cycles of 30 sec max power and 30 sec off, Bioruptor, Diagenode). Lysates were centrifuged (14000 rpm, 10 min, 4° C.) and supernatants were subjected to BCA assays (Pierce, Thermo Fisher Scientific, Waltham, Mass., USA) for protein concentration measurements. 20 μg of total cytosolic protein was subjected to SDS-PAGE and electroblotted onto a nitrocellulose membrane (Whatman Optitran BA-S85, GE healthcare, Little Chalfont, UK). After blocking in Odyssey Blocking buffer (LI-COR biosciences, Licoln, Nebr., USA) in PBS (1:1) the membrane was incubated with mouse-anti-HA (1:500, Sc-7932, Santa cruz, Dallas, Tex., USA), rabbit-anti-GLUD (1:400, GTX105765, GeneTex Inc, San Antonio, Tex., USA), rabbit-anti-IDH1 (1:1000, HPA035248, Sigma Aldrich, St. Louis, Mo., USA), mouse-anti-GAPDH (1:10,000, ab8245, Abcam, Cambridge, US), rabbit-anti-HK2 (1:1000, 2867S, Cell Signaling Technology, Danvers, Mass., USA), rabbit-anti-PKM2 (1:1000, D78A4, Cell Signaling Technology, Danvers, Mass., USA) and goat-anti-γtubulin (1:5000, sc-7396, Santa Cruz, Dallas, Tex., USA) in blocking buffer, followed by incubation with goat-anti-mouseDyLight800 (1:10.000, Thermo Fisher Scientific, Waltham, Mass., USA), goat-anti-rabbitAlexa680 (1:10.000, Invitrogen, Waltham, Mass., USA), or donkey-anti-goatAlexa680 (1:10,000, Invitrogen, Waltham, Mass., USA) in blocking buffer. After washing, blots were analyzed on the Odyssey scanner (LI-COR biotechnology, Lincoln, Nebr., USA). Signals were corrected for γ-tubulin and the mean of 3 independent experiments is plotted. Statistical significance was determined with an unpaired Student's T-test.

### Cell Proliferation Assays

Cellular protein content was determined as a measure of cell proliferation, using suforhodamine B (SRB) assays as described in (83). Sensitivity to EGCG was determined by adding a concentration range of EGCG (0-50 μM) or DMSO solvent one day after seeding 1,000 cells per well in 96-wells plates (Nunc, Roskilde, Denmark). An SRB assay was performed after 3 days, and IC50 values were determined in GraphPad Prism using the sigmoidal dose response with variable slope nonlinear regression analysis.

Furthermore cell proliferation over 8 days in presence or absence of EGCG was determined. Cells were seeded at 1,000 cells per well and at day 1 and day 5 after seeding the medium was changed for medium with or without 10 μM EGCG. Controls were incubated with DMSO. Protein content was determined every 2 days. Experiments were performed in triplicate and statistical significance was determined using one-way ANOVA with bonferroni correction.

To determine the sensitivity of the cells to glutamine or glucose deprivation, the regular medium was changed for either D-glucose depleted (0 g/L D-glucose and 4 g/L L-glutamine), L-glutamine depleted (1 g/L L-glutamine and 5 g/L D-glucose) or regular RPMI medium supplemented with 10% FCS and antibiotics with or without 10 μM EGCG one day after seeding the cells. Again protein content was determined every 2 days. Experiments were performed in triplicate and statistical significance was determined using one-way ANOVA with bonferroni correction.

### Cellular and Mitochondrial Respiration

Cells were grown till 80% confluency in culture flasks, and after trypsinization 1.5*10⁶ cells were resuspended in culture medium and transferred to the thermostated (37° C.) chamber of an Oxygraph-2k equipped with Datlab recording and analysis software (Oroboros Instruments, Innsbruck, Austria). The basal respiration was measured and then the remaining mitochondrial respiration was inhibited with 2.5 μM complex V inhibitor oligomycin. Then maximal respiration was measured by sequential addition of 0.5 μM mitochondrial uncoupler FCCP. Subsequently 0.5 μM complex I inhibitor Rotenone and 2.5 μM complex III inhibitor Antimycin A were added to completely shut down the electron transport chain. The remaining oxygen consumption is due to non-mitochondrial respiration. Two separate experiments were performed and significance was determined with an unpaired Student's T test.

### Radiotherapy Experiments

Since SKRC-7 and SKRC-17 cells are unable to grow as colonies, sensitivity to radiotherapy was analyzed by monitoring cell proliferation with the xCELLigence. This method has been shown to measure effects of radiotherapy that correlate with the conventional clonogenic assays (84). Cells were plated at 1,000 cells per well and left to adhere overnight. Then cells were treated with 10 μM EGCG for 24 hrs, after which they were irradiated with 4 Gy (IR, 3.1Gy/min; XRAD 320 ix, Precision XRT; N. Brandford, Conn., USA). The cell index was measured from the moment of seeding for 200 hrs in real time with intervals of 15 min, fresh medium supplemented with 10 μM EGCG was added every 72 hours. Cell index was normalized to the moment of applying radiotherapy, and cell growth was calculated by performing linear regression in GraphPad Prism. The experiment was performed with two internal duplicates, and statistical significance was determined with an unpaired Student's T test.

### Results

### VHL Rescue Causes Differential Changes in Metabolism of SKRC-7 and SKRC-17

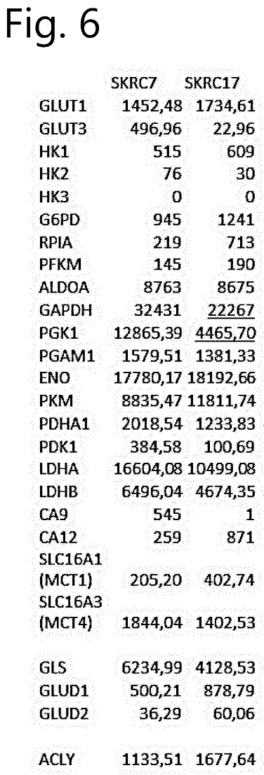

SKRC7 and SKRC17 present with different expression profiles (FIG. 6A). Levels of PGK1 and PDK1 transcripts were 3-fold lower in SKRC17 than in SKRC7, suggesting relatively inefficient processing of glucose to pyruvate in SKRC17. On the other hand, in this cell line enzymes of the PPP (G6PD and RPIA) were upregulated compared SKRC7. To test whether this difference is reflected in altered sensitivity to the PPP inhibitor 6-AN, we tested this compound in proliferation assays. Of note, whereas SKRC-7 cells surprisingly responded with an increase of cell proliferation, SKRC-17 cells responded by significantly decreased cell proliferation. The high levels of glutamine and glutamate-processing enzymes suggest sensitivity to the GLUD1 inhibitor EGCG. A combination of EGCG and 6-AN was able to completely block cell growth (FIG. 6B).

### Example 3—smMIP-Based Targeting Sequencing Allows the Distinction of Splice Variants

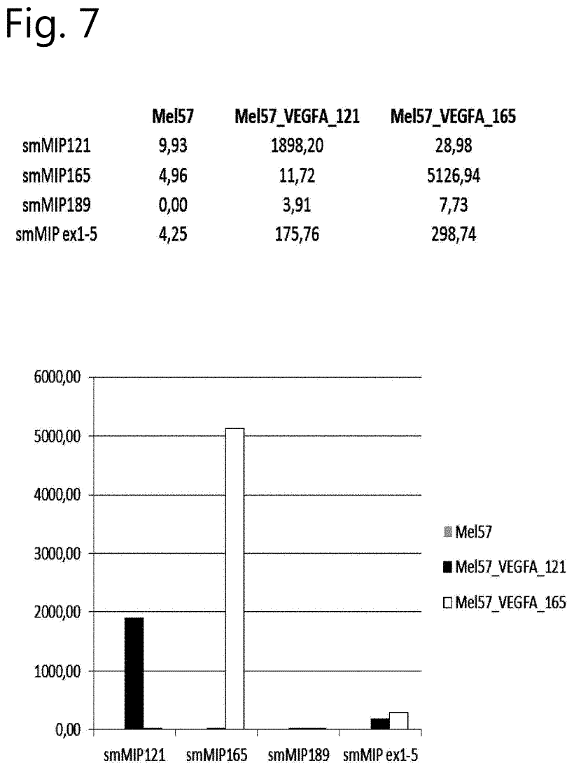

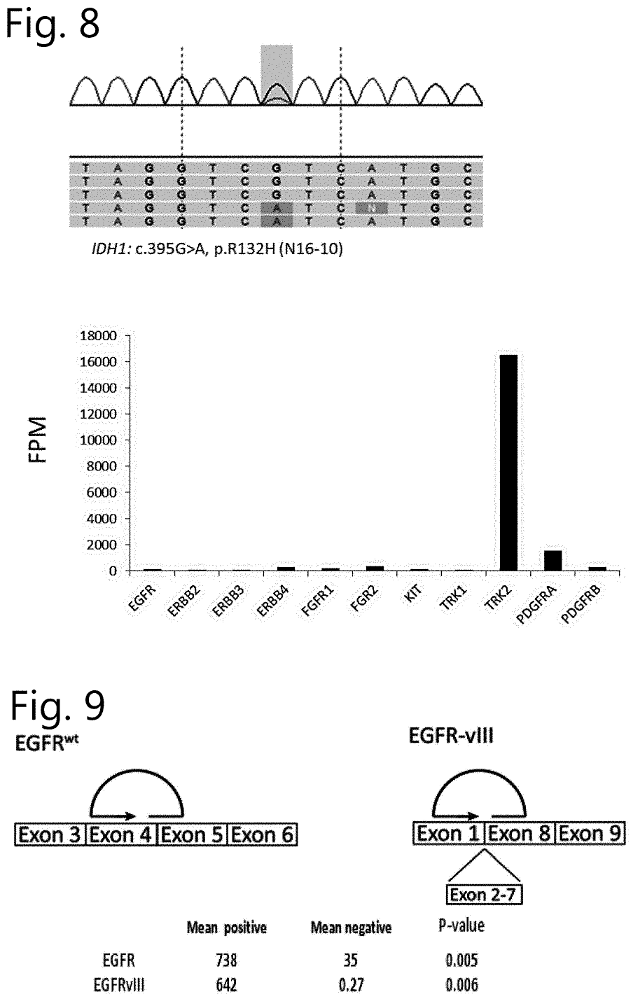

The melanoma cell line Mel57 expresses low levels of vascular endothelial growth factor (VEGF-A). VEGF-A consists of different splice variants, consisting of exons 1-5,8 (VEGF-A121), exons 1-5,7,8 (VEGF-165) and exons 1-8 (VEGF-189). These variants have differential activities, ranging from vessel dilatation to full neo-angiogenesis (85). We designed smMIP165 that has its ligation and extension probes in exon 5 and 7, smMIP121 that has its ligation and extension probes in exon 5 and 8, and smMIP189, that has its ligation and extension probes in exon 5 and 6. We performed the smMIP assay with a panel including smMIP121, smMIP165, smMIP189 and 5 smMIPs located in exons 1-5, recognizing all isoforms of VEGF on RNA isolated from the Mel57 cell line and from cell lines expressing the different VEGF isoforms, as described in (85). FIG. 7 and the accompanying table show that the different isoform-specific smMIPs specifically recognize the splice variants.

Cancer cells can induce changes in RNA splicing events if these give the cells a growth advantage. These changes may be an inherent characteristic of a cancer, but may also be selected under pressure of treatment. An example is EGFR variant III that results from an intragenic deletion in the EGFR gene that results in loss of exons 2 to 7 in the mature transcript. Whereas 50% of glioblastomas are characterized by amplification of the EGFR oncogene, in 50% of this population expression of EGFRvIII is found. By placing extension and ligation probes of an individual smMIP in exons 1 and 8, respectively, only the exon 1-8 fusion product is detected, because the backbone sequence of 40 nucleotides is physically not able to bridge exons 2-7 in the wild-type transcript (FIG. 9 shows that in the group of gliomas there is elevated expression of EGFR in 39/75 brain tumors (52%; mean FPM 738 in positives vs mean FPM 35 in negatives, using an arbitrary cut off FPM value of 100) and expression of EGFRvIII in 12/75 brain tumors (16%; mean FPM 642 in positives vs mean FPM 0.27 in negatives, using an arbitrary cut-off value of 6). Thus, smMIP based detection of EGFRvIII is highly specific and highly sensitive.

Another example is androgen receptor in prostate cancer. Patients with castration-resistant prostate cancer are treated with enzalutamide. However, a change in splicing that results in loss of exon 7 (ARv7) results in resistance to enzalutamide. By designing a smMIP with extension and ligation probe arms in exons 6 and 8, respectively, Arv7 is readily detected in cell lines derived from enzalutamide—resistant cancers, while it is not detected in any other cancer type (FPM=277 and 640 in VCAP and DuCAP prostate cancer lines, respectively vs mean FPM=0.01 in 130 other cancers).

### Example 4—smMIP Based Targeting Sequencing can be Used for Accurate Diagnosis

A sample of brain tumor tissue, obtained from patient N16-10 who signed informed consent for the study, was snap-frozen directly after surgery. RNA was isolated from the tissue via the Trizol protocol, followed by cDNA synthesis and preparation of the smMIP library. After Illumina next generation sequencing a mutation in the IDH1 gene was identified that corresponds to the hotspot IDH1R132H mutation. The same analysis revealed low levels of carbonic anhydrase 9 and hexokinase 2, indicating lack of hypoxic responses. Furthermore the sample shows low ratios of glutaminase/glutamate dehydrogenase, suggesting that the tumor was using glutamate for metabolism, and therefore suggesting sensitivity to glutamate dehydrogenase inhibitors such as EGCG and chloroquine. Furthermore, the data show high expression levels of TrkB and, to a lesser extent, PDGFRα. The absence of hypoxia suggests that the tumor was not of a World Health Organization guidelines-defined grade IV type. The presence of high levels of tyrosine kinases suggests astrocytoma, and based on the data a diagnosis of IDH1-mutated grade III astrocytoma was made, concordant with the original diagnosis that was set on histopathology.

### Example 5—smMIP Based Targeting Sequencing can be Used for Accurate Diagnosis and Prognosis

The data of example 4 were expanded with 74 additional samples of brain tumor tissues, obtained from patients who all signed informed consent for the study. The samples were snap-frozen directly after surgery and treated similarly to what has been described in example 4: RNA was isolated from the tissues via the Trizol protocol, followed by cDNA synthesis, preparation of the smMIP libraries and barcoded PCR. After Illumina next generation sequencing FASTQ files were processed by SeqNext software (JSI SequencePilot version 4.2.2 build 502 [JSI Medical Systems, Ettenheim, Germany]). All reads were mapped against the human genome (version hg19) and against manually added variant transcripts (e.g. EGFRvIII, Arv7, METd7/8, METd14). Thus, for every tumor sample a list of targeted transcript levels was generated and a list of all detected mutations/variations. From all 75 patients fully documented clinical follow-up was available.

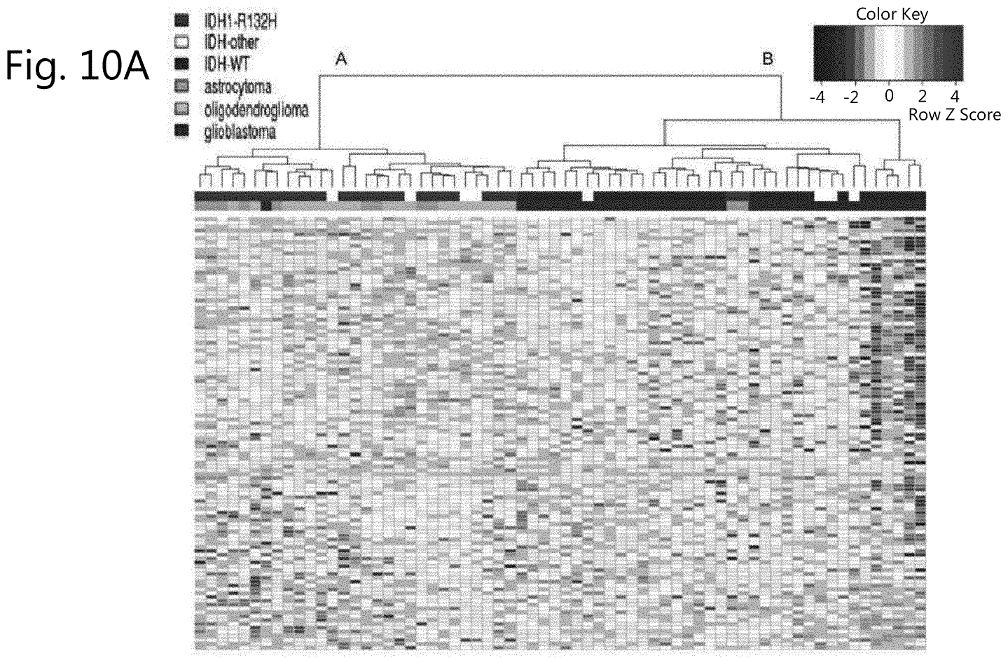

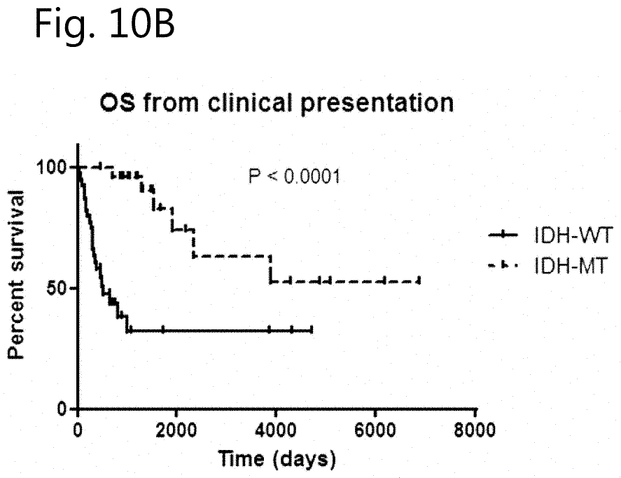

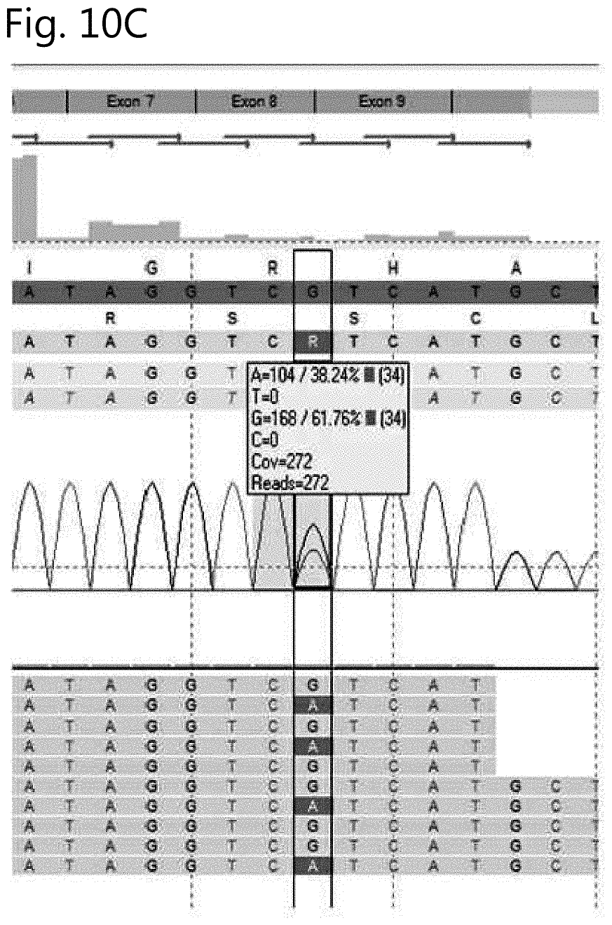

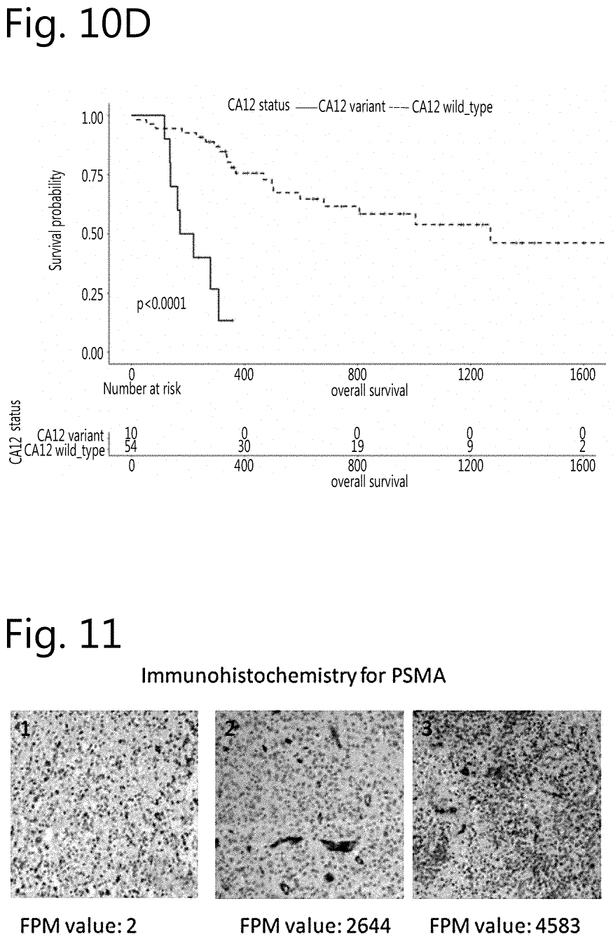

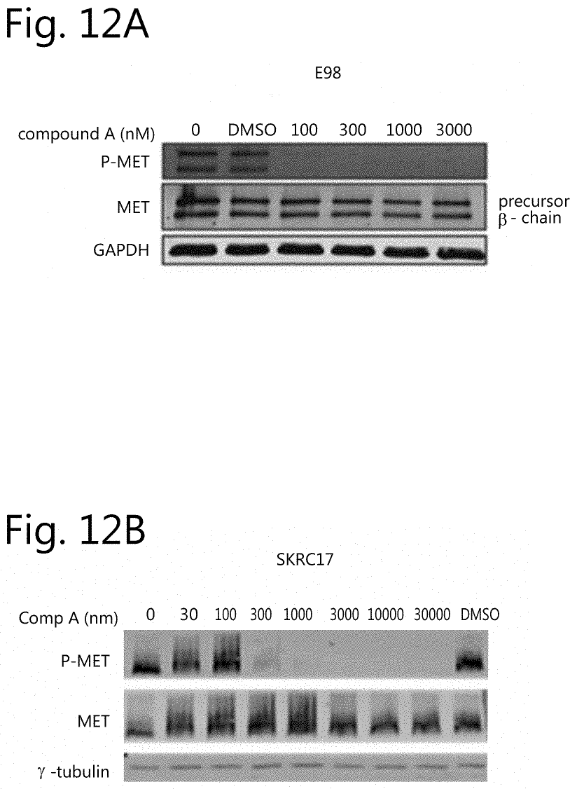

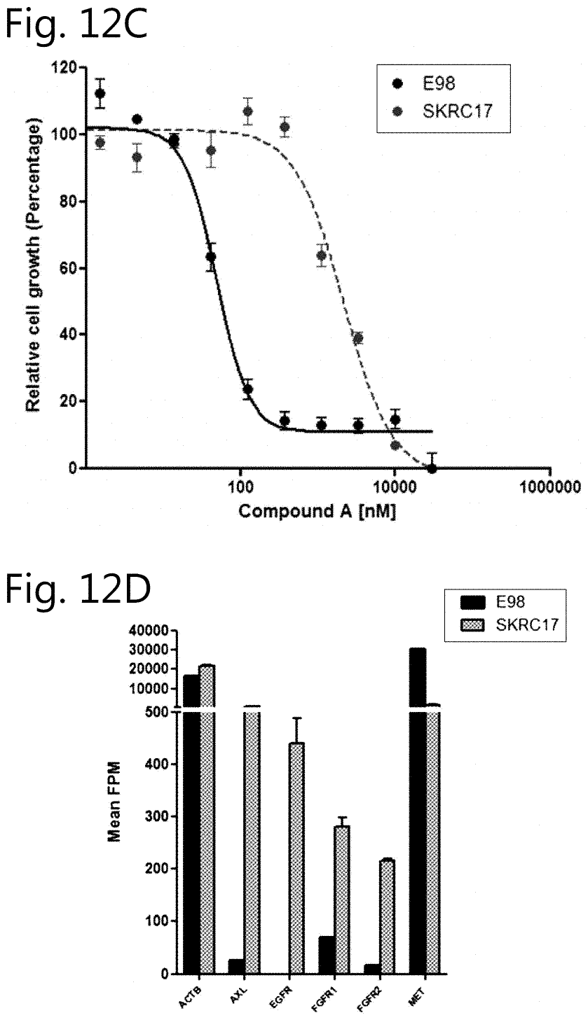

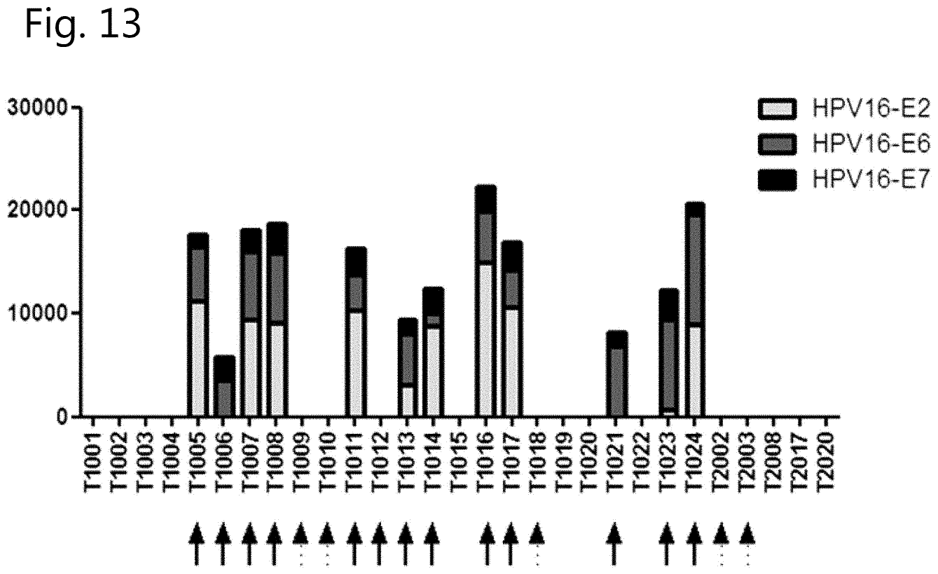

In a first step, we performed unsupervised agglomerative clustering of log-transformed expression levels of the targeted genes of interest. Agglomerative clustering was performed according to Ward's method by calculating Manhattan distance between individual profiles using bio-informatic R-software scripts. The profiles were translated in a heat map which is represented in FIG. 10a. As is shown the computer generates two main groups A and B, that are subdivided in a number of subgroups.

In a next step, potential associations of the clusters with overall survival was investigated by now including survival data for the patients (overall survival, counted from first diagnosis) and generating a Kaplan-Meyer curve. The results in FIG. 10b show that the computer-generated groups have different survival with high significance (Fisher's exact test; p<0.0001). This shows that for gliomas this test has high prognostic value.

In a third step, associations between groups and mutations were analyzed by including the list of all detected mutations in a sample. Groups A and B were distinguished by mutations in the isocitrate dehydrogenase genes (IDH1 R132 and IDH2R172) with high significance (p<10E-11). FIG. 10c shows an example of the heterozygous IDH1R132H detection in one of the samples, in this case with 38% of transcripts being from the mutant allele and 62% of transcripts from the wt allele. The difference in transcript frequency is attributable to genetically normal stromal cells with only wild type IDH transcripts.

In a fourth step we performed a subgroup analysis to further refine prognosis. Analyzing IDH-wild-type patients with very poor survival (OS<12 months) versus IDH-wild-type patients with better prognosis (group B in the Kaplan-Meyer curves) in such subgroup analysis showed that high expression levels of carbonic anhydrase 12 are associated with extremely poor prognosis (p<0.001; Fisher's exact test, FIG. 10d).

In a fifth step we retrospectively analyzed all data with respect to molecular information that was obtained during routine patient care. All mutations that we observed on the RNA level and that are routinely tested for in glioma patient care, were confirmed with DNA sequencing technology (Table III)

The profiles also reveal expression of genes in brain tumors that are associated with other cancers. An example is the androgen receptor that is often expressed at high levels in prostate carcinoma, and prostate specific membrane protein (PSMA). Other groups have described expression of this target on blood vessels in malignant tumors, including glioma (87). To investigate this further we analyzed tumors with high and low PSMA transcript levels using immunhistochemisty. Results indeed revealed blood vessel expression of PSMA protein in blood vessels from tumors with high transcript levels, and not in tumors with low transcript levels (see three examples in FIG. 11).

TABLE III
Clinical characteristics. Diagnosis, histological type, and percentage
tumor cells were confirmed by a trained pathologist (BK). Annotations
as marked in this table are used in the heatmap of FIG. 10a.

| Sample | | Age (at time | | | % tumor |
|---|---|---|---|---|---|
| name | Sex | of surgery) | Histological type | IDH mutation | cells |
| 13-02 | M | 40 | Astrocytoma | IDH1-R132H | 70 |
| 13-03 | M | 58 | Oligodendroglioma | IDH1-R132H | 70 |
| 13-04 | F | 62 | Glioblastoma | WT | 60 |
| 13-06 | M | 53 | Oligodendroglioma | IDH2-R172K | 60 |
| 13-08 | M | 67 | Glioblastoma | WT | 70 |
| 13-09 | F | 58 | Glioblastoma | WT | 70 |
| 13-10 | M | 45 | Oligodendroglioma | IDH1-R132H | 65 |
| 13-11 | F | 67 | Glioblastoma | WT | 70 |
| 13-13 | M | 52 | Glioblastoma | IDH1-V178I | 70 |
| 13-14 | F | 64 | Glioblastoma | WT | 70 |
| 13-15 | F | 44 | Oligodendroglioma | IDH1-R132H | 50 |
| 13-16 | M | 60 | Glioblastoma | WT | 70 |
| 13-17 | M | 45 | Oligodendroglioma | IDH1-R132H | 50 |
| 13-18 | F | 49 | Oligodendroglioma | IDH1-R132H | 50 |
| 14-01 | F | 52 | Glioblastoma | WT | 80 |
| 14-02 | M | 43 | Oligodendroglioma | IDH1-R132H | 50 |
| 14-03 | F | 62 | Glioblastoma | WT | 70 |
| 14-04 | M | 72 | Glioblastoma | WT | 60 |
| 14-05 | M | 21 | Oligodendroglioma | IDH1-R132H | 70 |
| 14-06 | M | 43 | Oligodendroglioma | IDH1-R132H | 50 |
| 14-07 | M | 65 | Oligodendroglioma | IDH1-R132H | 50 |
| 14-08 | M | 50 | Astrocytoma | IDH1-R132H | 50 |
| 14-09 | F | 43 | Astrocytoma | IDH1-R132H | 60 |
| 14-10 | F | 45 | Glioblastoma | IDH1-R132H | 50 |
| 14-11 | M | 50 | Glioblastoma | WT | 60 |
| 14-12 | M | 59 | Oligodendroglioma | IDH1-R132H | 50 |
| 15-01 | M | 66 | Glioblastoma | WT | 50 |
| 15-02 | F | 61 | Glioblastoma | WT | 70 |
| 15-03 | F | 76 | Glioblastoma | WT | 40 |
| 15-04 | F | 59 | Glioblastoma | WT | 40 |
| 15-05 | M | 31 | Astrocytoma | IDH1-R132H | 70 |
| 15-06 | F | 49 | Astrocytoma | IDH1-R132H/V178I | 70 |
| 15-07 | M | 63 | Glioblastoma | IDH1-V178I | 65 |
| 15-08 | M | 55 | Astrocytoma | IDH1-R132H | 60 |
| 15-09 | M | 70 | Glioblastoma | WT | 70 |
| 15-10 | F | 68 | Oligodendroglioma | IDH1-R132H | 70 |
| 15-12 | M | 46 | Glioblastoma | WT | 70 |
| 15-13 | F | 78 | Glioblastoma | WT | 80 |
| 15-14 | M | 79 | Glioblastoma | WT | 70 |
| 15-15 | F | 58 | Glioblastoma | WT | 70 |
| 15-16 | M | 25 | Astrocytoma | IDH1-R132H/V178I | 50 |
| 15-17 | M | 68 | Glioblastoma | WT | 60 |
| 15-18 | F | 64 | Glioblastoma | WT | 70 |
| 16-01 | M | 61 | Glioblastoma | WT | 70 |
| 16-02 | M | 47 | Glioblastoma | WT | 70 |
| 16-03 | F | 46 | Astrocytoma | IDH1-R132H | 25 |
| 16-04 | M | 59 | Oligodendroglioma | IDH1-R132H | 60 |
| 16-05 | M | 51 | Glioblastoma | WT | 50 |
| 16-06 | F | * | Astrocytoma | IDH1-R132H | 50 |
| 16-07 | M | 74 | Glioblastoma | IDH1-Y183C | 60 |
| 16-08 | F | 69 | Glioblastoma | WT | 50 |
| 16-09 | F | 49 | Glioblastoma | WT | 70 |
| 16-10 | M | * | Astrocytoma | IDH1-R132H | 60 |
| 16-11 | M | 67 | Glioblastoma | WT | 50 |
| 16-12 | M | 23 | Astrocytoma | IDH1-R132H | 60 |
| 16-13 | M | 60 | Glioblastoma | WT | 70 |
| 16-14 | F | 60 | Oligodendroglioma | IDH2-R172M | 70 |
| 16-15 | F | 61 | Oligodendroglioma | IDH1-R132H | 70 |
| 16-16 | M | 58 | Glioblastoma | IDH1-V178I | 40 |
| 16-17 | F | 18 | Oligodendroglioma | IDH2-R172K | 40 |
| 16-18 | * | 30 | Oligodendroglioma | IDH2-R172W | 70 |
| 16-19 | M | 48 | Glioblastoma | WT | 70 |
| 17-01 | M | 58 | Oligodendroglioma | IDH1-R132H | 50 |
| 17-02 | M | 40 | Astrocytoma | IDH1-R132H/V178I | 70 |
| 17-03 | F | 76 | Glioblastoma | WT | 65 |
| 17-04 | M | 42 | Oligodendroglioma | IDH1-R132H | 70 |
| 17-05 | M | 59 | Astrocytoma | WT | 70 |
| 17-06 | M | 65 | Glioblastoma | WT | 70 |
| 17-07 | M | 63 | Glioblastoma | WT | 70 |

Abbreviations M, male; F, female; IDH, isocitrate dehydrogenase; WT, IDH-wild type.
* data not available

### Example 6—Metabolism in IDH-Mutated Glioma

Detailed analysis of expression of metabolic genes revealed that in the group of long survivors low levels of glucose importers, carbonic anhydrase and hexokinase 2 were expressed, indicating lack of hypoxia. Furthermore in this group high levels of glutamate dehydrogenase and aminobutyric acid aminotranferase (ABAT) RNA were observed, suggesting that these tumors use neurotransmitters (glutamate and GABA) for their catabolism, inducing sensitivity to glutamate dehydrogenase- and ABAT inhibitors (epigallocatechin-3-gallate, vigabatrine).

### Example 7—Tyrosine Kinases in Glioma

In the group of IDH-mutated gliomas high expression levels of TrkB (mean FPM 15833 vs 4708 in IDHmut vs IDHwt, p=2×10E-11) were detected, suggesting sensitivity to the TrkB inhibitor entrectinib. EGFR expression was higher in IDHwt tumor (609 vs 116 in IDHwt vs IDHmut cancers, p=0.004), whereas EGFRvIII was exclusively expressed in this group (201 vs 0.16 in IDHwt vs IDHmut, p=0.0005, see also FIG. 9).

Whereas in the group of IDH wild type cancers EGFR/EGFRvIII expression was observed in 52% of samples, the tyrosine kinase MET is observed in 9/75 brain tumors (12%). Of interest, profiles showed that tumors that expressed relatively high levels of MET, were low in EGFR and vice versa (correlation coefficient r=−0.95). An interesting further finding was occurrence of a mutation in BRAF (BRAF-V600E) in one glioma. Wild-type BRAF is a crucial signaling intermediate that processes signals from activated membrane tyrosine kinases (e.g. EGFR, MET) to the nucleus, thereby inducing cell proliferation. BRAF-V600E is an auto-active molecule that signals to the nucleus without input from receptortyroinse kinases in an uncontrolled fashion. This BRAF mutation occurs in 50% of melanoma cancers and can be inhibited by the targeted drug vemurafenib. The glioma containing this mutation did not express MET nor EGFR. Although anecdotal, this case suggests susceptibility to vemurafenib and unsusceptibility to EGFR or MET inhibitors.

### Example 7—Tyrosine Kinase Profiles Predict Sensitivity and Non-Sensitivity to Targeted Therapies In Vitro

The astrocytoma cell line E98 carries an auto-activating mutation in c-MET (22) and is highly sensitive to the multispecific VEGFR2/MET kinase inhibitor cabozantinib (86) and the MET-selective inhibitor Compound A (88). This sensitivity is reflected in decreased MET phosphorylation on western blot and decreased proliferation rates in vitro and delayed tumor development in vivo. The renal cancer cell line SKRC17 expresses similar levels of MET, phosphorylation of which is effectively inhibited by Compound A (FIG. 12). Yet, SKRC17 cells do not respond to compound A with decreased proliferation rates. Profiling of membrane tyrosine kinases reveals that within the selected group of membrane tyrosine kinases that are measured in the assay, MET is the only one expressed by E98, whereas SKRC17 cells express an additional number of other tyrosine kinase inhibitors, including AXL, EGFRs, FGFRs. These results suggest that targeted inhibition of one receptor tyrosine kinase is only effective in the absence of rescue kinases, and that effective treatment of a cancer requires concomitant blockade of all membrane receptor tyrosine kinases on a cell.

### Example 8—HPV RNA Profiling Reduces False Positive Outcomes of HPV-DNA-Based Population Based Screening

The Netherlands is one of the first countries implementing detection of high risk human papilloma viruses (hrHPV) in cervical swabs in a population-wide screening program, to allow early detection and preventive treatment of cervical cancers. The life-time risk of an HPV infection is 80%, and in the group of participating women 8% will test positive in this assay. It is known on forehand that 90% of these women are overtreated because HPV infections may resolve spontaneously. Furthermore, sex with an HPV positive partner will result in positivity in the sensitive PCR-based HPV DNA detection tests, but does not mean that the virus will actually infects cervical epithelials cells.

To discriminate between contamination and actual cellular infection, we designed smMIPs for detection of hrHPV transcripts E2, E6 and E7, based on available knowledge that loss of E2 gene expression is associated with chromosomal integration in infected cells and overexpression of the HPV-E6 and E7 oncoproteins. With the entire panel of smMIP probes, supplemented with hrHPV-detecting smMIP probes, we profiled a series of 29 gynecological tissues, ranging from normal uterus extirpations to ovarian cancer, endometrial cancers and cervix carcinomas (FIG. 13). Samples were profiled blinded to pathology. HPV16 E6/E7 RNA expression was observed in 12 samples. In retrospect, these all were squamous cell carcinomas of the cervix. In a next step we analyzed all samples using the HPV-DNA PCR test. All HPV16-positive samples were confirmed on the DNA level, but 5 tissues that were negative in the HPV-RNA test, were positive in the HPV-DNA test (arrow heads). In retrospect, these samples consisted of two normal uteri and two endometrium carcinomas (that indeed are known not to be HPV-induced). These data clearly show that HPV-RNA screening is capable of reducing the number of false positive testing of HPV-DNA screening.

In a second step we investigated the sensitivity of HPV-testing by profiling 1, 10, 100, 1000, and 10,000 Hela cells, derived 490 years ago from a woman with an HPV-18 positive cervix carcinoma. Profiling of only 1 cell already detected 69 unique HPV18 reads, increasing to 168, 1419, 36767 reads in 10, 100 and 1000 cells respectively.

### REFERENCES

1. van Lith S A, Navis A C, Lenting K, Verrijp K, Schepens J T, Hendriks W J, et al. Identification of a novel inactivating mutation in Isocitrate Dehydrogenase 1 (IDH1-R314C) in a high grade astrocytoma. Sci Rep. 2016; 6:30486.
2. Bardella C, Pollard P J, Tomlinson I. SDH mutations in cancer. Biochim Biophys Acta. 2011; 1807(11):1432-43.
3. Linehan W M, Rouault T A. Molecular pathways: Fumarate hydratase-deficient kidney cancer—targeting the Warburg effect in cancer. Clin Cancer Res. 2013; 19(13):3345-52.
4. Chuang J H, Chou M H, Tai M H, Lin T K, Liou C W, Chen T, et al. 2-Deoxyglucose treatment complements the cisplatin- or BH3-only mimetic-induced suppression of neuroblastoma cell growth. Int J Biochem Cell Biol. 2013; 45(5):944-51.
5. Lis P, Dylag M, Niedzwiecka K, Ko Y H, Pedersen P L, Goffeau A, et al. The HK2 Dependent “Warburg Effect” and Mitochondrial Oxidative Phosphorylation in Cancer: Targets for Effective Therapy with 3-Bromopyruvate. Molecules. 2016; 21(12).
6. Pedersen P L. 3-Bromopyruvate (3BP) a fast acting, promising, powerful, specific, and effective “small molecule” anti-cancer agent taken from labside to bedside: introduction to a special issue. J Bioenerg Biomembr. 2012; 44(1):1-6.
7. Haghighat N, McCandless D W. Effect of 6-aminonicotinamide on metabolism of astrocytes and C6-glioma cells. Metab Brain Dis. 1997; 12(1):29-45.
8. Song I S, Han J, Lee H K. Metformin as an anticancer drug: A Commentary on the metabolic determinants of cancer cell sensitivity to glucose limitation and biguanides. J Diabetes Investig. 2015; 6(5):516-8.
9. Flavin R, Peluso S, Nguyen P L, Loda M. Fatty acid synthase as a potential therapeutic target in cancer. Future Oncol. 2010; 6(4):551-62.
10. Burchardt P, Rzezniczak J, Dudziak J, Dzumak A, Marchlewski T, Ganowicz-Kaatz T, et al. Evaluation of plasma PCSK9 concentrations, transcript of LDL receptor, as well as the total number of monocyte LDL receptors in acute coronary syndrome patients. Cardiol J. 2016; 23(6):604-9.
11. Lord C J, Ashworth A. Mechanisms of resistance to therapies targeting BRCA-mutant cancers. Nat Med. 2013; 19(11):1381-8.
12. Hata A N, Niederst M J, Archibald H L, Gomez-Caraballo M, Siddiqui F M, Mulvey H E, et al.

Tumor cells can follow distinct evolutionary paths to become resistant to epidermal growth factor receptor inhibition. Nat Med. 2016; 22(3):262-9.
13. Chong C R, Janne P A. The quest to overcome resistance to EGFR-targeted therapies in cancer. Nat Med. 2013; 19(11):1389-400.
14. Bouwman P, Jonkers J. Molecular pathways: how can BRCA-mutated tumors become resistant to PARP inhibitors? Clin Cancer Res. 2014; 20(3):540-7.
15. Hiatt J B, Pritchard C C, Salipante S J, O'Roak B J, Shendure J. Single molecule molecular inversion probes for targeted, high-accuracy detection of low-frequency variation. Genome Res. 2013; 23(5):843-54.
16. Eijkelenboom A, Kamping E J, Kastner-van Raaij A W, Hendriks-Cornelissen S J, Neveling K, Kuiper R P, et al. Reliable Next-Generation Sequencing of Formalin-Fixed, Paraffin-Embedded Tissue Using Single Molecule Tags. J Mol Diagn. 2016; 18(6):851-63.
17. Tonjes M, Barbus S, Park Y J, Wang W, Schlotter M, Lindroth A M, et al. BCAT1 promotes cell proliferation through amino acid catabolism in gliomas carrying wild-type IDH1. Nat Med. 2013; 19(7):901-8.
18. Boyle E A, O'Roak B J, Martin B K, Kumar A, Shendure J. MIPgen: optimized modeling and design of molecular inversion probes for targeted resequencing. Bioinformatics. 2014; 30(18):2670-2.
19. Luo J, Manning B D, Cantley L C. Targeting the PI3K-Akt pathway in human cancer: rationale and promise. Cancer Cell. 2003; 4(4):257-62.
20. Carracedo A, Pandolfi P P. The PTEN-PI3K pathway: of feedbacks and cross-talks.

Oncogene. 2008; 27(41):5527-41.
21. Yin Y, Shen W H. PTEN: a new guardian of the genome. Oncogene. 2008; 27(41):5443-53.
22. Navis A C, van Lith S A, van Duijnhoven S M, de Pooter M, Yetkin-Arik B, Wesseling P, et al. Identification of a novel MET mutation in high-grade glioma resulting in an auto-active intracellular protein. Acta Neuropathol. 2015; 130(1):131-44.
23. Soussi T, Wiman K G. Shaping genetic alterations in human cancer: the p53 mutation paradigm. Cancer Cell. 2007; 12(4):303-12.
24. Sherr C J, McCormick F. The RB and p53 pathways in cancer. Cancer Cell. 2002; 2(2):103-12.
25. Boulton S J. Cellular functions of the BRCA tumour-suppressor proteins. Biochem Soc Trans. 2006; 34(Pt 5):633-45.
26. Burgess D J. Chromosome instability: Tumorigenesis via satellite link. Nat Rev Cancer. 2011; 11(3):158.
27. Casaletto J B, McClatchey A I. Spatial regulation of receptor tyrosine kinases in development and cancer. Nat Rev Cancer. 2012; 12(6):387-400.
28. Rocca A, Farolfi A, Bravaccini S, Schirone A, Amadori D. Palbociclib (PD 0332991): targeting the cell cycle machinery in breast cancer. Expert Opin Pharmacother. 2014; 15(3):407-20.
29. Payen V L, Porporato P E, Baselet B, Sonveaux P. Metabolic changes associated with tumor metastasis, part 1: tumor pH, glycolysis and the pentose phosphate pathway. Cell Mol Life Sci. 2016; 73(7):1333-48.
30. Patra K C, Hay N. The pentose phosphate pathway and cancer. Trends Biochem Sci. 2014; 39(8):347-54.
31. Acharya A, Das I, Chandhok D, Saha T. Redox regulation in cancer: a double-edged sword with therapeutic potential. Oxid Med Cell Longev. 2010; 3(1):23-34.
32. Hao J, Graham P, Chang L, Ni J, Wasinger V, Beretov J, et al. Proteomic identification of the lactate dehydrogenase A in a radioresistant prostate cancer xenograft mouse model for improving radiotherapy. Oncotarget. 2016.
33. Lucarelli G, Galleggiante V, Rutigliano M, Sanguedolce F, Cagiano S, Bufo P, et al. Metabolomic profile of glycolysis and the pentose phosphate pathway identifies the central role of glucose-6-phosphate dehydrogenase in clear cell-renal cell carcinoma. Oncotarget. 2015; 6(15):13371-86.
34. Cairns R A, Harris I S, Mak T W. Regulation of cancer cell metabolism. Nat Rev Cancer. 2011; 11(2):85-95.
35. Yang C, Ko B, Hensley C T, Jiang L, Wasti A T, Kim J, et al. Glutamine oxidation maintains the TCA cycle and cell survival during impaired mitochondrial pyruvate transport. Mol Cell. 2014; 56(3):414-24.
36. Shanware N P, Mullen A R, DeBerardinis R J, Abraham R T. Glutamine: pleiotropic roles in tumor growth and stress resistance. J Mol Med (Berl). 2011; 89(3):229-36.
37. Wise D R, DeBerardinis R J, Mancuso A, Sayed N, Zhang X Y, Pfeiffer H K, et al. Myc regulates a transcriptional program that stimulates mitochondrial glutaminolysis and leads to glutamine addiction. Proc Natl Acad Sci USA. 2008; 105(48):18782-7.
38. Lunt S Y, Vander Heiden M G. Aerobic glycolysis: meeting the metabolic requirements of cell proliferation. Annu Rev Cell Dev Biol. 2011; 27:441-64.
39. Roodink I, van der Laak J, Kusters B, Wesseling P, Verrijp K, de Waal R, et al.

Development of the tumor vascular bed in response to hypoxia-induced VEGF-A differs from that in tumors with constitutive VEGF-A expression. Int J Cancer. 2006; 119(9):2054-62.
40. Hamans B, Navis A C, Wright A, Wesseling P, Heerschap A, Leenders W. Multivoxel (1)H MR spectroscopy is superior to contrast-enhanced MRI for response assessment after anti-angiogenic treatment of orthotopic human glioma xenografts and provides handles for metabolic targeting. Neuro Oncol. 2013; 15(12):1615-24.
41. Altman B J, Stine Z E, Dang C V. From Krebs to clinic: glutamine metabolism to cancer therapy. Nat Rev Cancer. 2016.
42. Bensaad K, Favaro E, Lewis C A, Peck B, Lord S, Collins J M, et al. Fatty acid uptake and lipid storage induced by HIF-1alpha contribute to cell growth and survival after hypoxia-reoxygenation. Cell Rep. 2014; 9(1):349-65.
43. Saxena N, Maio N, Crooks D R, Ricketts C J, Yang Y, Wei M H, et al. SDHB-Deficient Cancers: The Role of Mutations That Impair Iron Sulfur Cluster Delivery. J Natl Cancer Inst. 2016; 108(1).
44. Lussey-Lepoutre C, Hollinshead K E, Ludwig C, Menara M, Morin A, Castro-Vega L J, et al. Loss of succinate dehydrogenase activity results in dependency on pyruvate carboxylation for cellular anabolism. Nat Commun. 2015; 6:8784.
45. Cancer Genome Atlas Research N, Linehan W M, Spellman P T, Ricketts C J, Creighton C J, Fei S S, et al. Comprehensive Molecular Characterization of Papillary Renal-Cell Carcinoma. N Engl J Med. 2016; 374(2):135-45.
46. Kampjarvi K, Makinen N, Mehine M, Valipakka S, Uimari O, Pitkanen E, et al. MED12 mutations and F H inactivation are mutually exclusive in uterine leiomyomas. Br J Cancer. 2016; 114(12):1405-11.
47. Molenaar R J, Botman D, Smits M A, Hira V V, van Lith S A, Stap J, et al. Radioprotection of IDH1-Mutated Cancer Cells by the IDH1-Mutant Inhibitor AGI-5198. Cancer Res. 2015; 75(22):4790-802.
48. van Lith S A, Navis A C, Verrijp K, Niclou S P, Bjerkvig R, Wesseling P, et al. Glutamate as chemotactic fuel for diffuse glioma cells: are they glutamate suckers? Biochim Biophys Acta. 2014; 1846(1):66-74.
49. Kim W Y, Kaelin W G. Role of VHL gene mutation in human cancer. J Clin Oncol. 2004; 22(24):4991-5004.
50. Laukka T, Mariani C J, Ihantola T, Cao J Z, Hokkanen J, Kaelin W G, Jr., et al. Fumarate and Succinate Regulate Expression of Hypoxia-inducible Genes via TET Enzymes. J Biol Chem. 2016; 291 (8):4256-65.
51. Losman J A, Kaelin W G, Jr. What a difference a hydroxyl makes: mutant IDH, (R)-2-hydroxyglutarate, and cancer. Genes Dev. 2013; 27(8):836-52.
52. Robinson C M, Ohh M. The multifaceted von Hippel-Lindau tumour suppressor protein.

FEBS Lett. 2014; 588(16):2704-11.
53. Hakimi A A, Reznik E, Lee C H, Creighton C J, Brannon A R, Luna A, et al. An Integrated Metabolic Atlas of Clear Cell Renal Cell Carcinoma. Cancer Cell. 2016; 29(1):104-16.
54. Grabmaier K, M C AdW, Verhaegh G W, Schalken J A, Oosterwijk E. Strict regulation of CAIX(G250/MN) by HIF-1alpha in clear cell renal cell carcinoma. Oncogene. 2004; 23(33):5624-31.
55. Claes A, Schuuring J, Boots-Sprenger S, Hendriks-Cornelissen S, Dekkers M, van der Kogel A J, et al. Phenotypic and genotypic characterization of orthotopic human glioma models and its relevance for the study of anti-glioma therapy. Brain Pathol. 2008; 18(3):423-33.
56. Navis A C, Niclou S P, Fack F, Stieber D, van Lith S, Verrijp K, et al. Increased mitochondrial activity in a novel IDH1-R132H mutant human oligodendroglioma xenograft model: in situ detection of 2-H G and alpha-K G. Acta Neuropathol Commun. 2013; 1:18.
57. Trapnell C, Roberts A, Goff L, Pertea G, Kim D, Kelley D R, et al. Differential gene and transcript expression analysis of RNA-seq experiments with TopHat and Cufflinks. Nat Protoc. 2012; 7(3):562-78.
58. Sjolund J, Johansson M, Manna S, Norin C, Pietras A, Beckman S, et al. Suppression of renal cell carcinoma growth by inhibition of Notch signaling in vitro and in vivo. The Journal of clinical investigation. 2008; 118(1):217-28.
59. Tardito S, Oudin A, Ahmed S U, Fack F, Keunen O, Zheng L, et al. Glutamine synthetase activity fuels nucleotide biosynthesis and supports growth of glutamine-restricted glioblastoma.

Nat Cell Biol. 2015; 17(12):1556-68.
60. Martinez-Outschoorn U E, Peiris-Pages M, Pestell R G, Sotgia F, Lisanti M P. Cancer metabolism: a therapeutic perspective. Nat Rev Clin Oncol. 2016.
61. Chaumeil M M, Radoul M, Najac C, Eriksson P, Viswanath P, Blough M D, et al. Hyperpolarized (13)C M R imaging detects no lactate production in mutant IDH1 gliomas: Implications for diagnosis and response monitoring. Neuroimage Clin. 2016; 12:180-9.
62. Yizhak K, Chaneton B, Gottlieb E, Ruppin E. Modeling cancer metabolism on a genome scale. Mol Syst Biol. 2015; 11(6):817.
63. Esmaeili M, Hamans B C, Navis A C, van Horssen R, Bathen T F, Gribbestad I S, et al.

IDH1 R132H mutation generates a distinct phospholipid metabolite profile in glioma. Cancer Res. 2014; 74(17):4898-907.
64. Ganapathy-Kanniappan S, Vali M, Kunjithapatham R, Buijs M, Syed L H, Rao P P, et al. 3-bromopyruvate: a new targeted antiglycolytic agent and a promise for cancer therapy. Curr Pharm Biotechnol. 2010; 11(5):510-7.
65. Ganapathy-Kanniappan S, Kunjithapatham R, Geschwind J F. Anticancer efficacy of the metabolic blocker 3-bromopyruvate: specific molecular targeting. Anticancer Res. 2013; 33(1):13-20.
66. Kankotia S, Stacpoole P W. Dichloroacetate and cancer: new home for an orphan drug?Biochim Biophys Acta. 2014; 1846(2):617-29.
67. Singh B N, Shankar S, Srivastava R K. Green tea catechin, epigallocatechin-3-gallate (EGCG): mechanisms, perspectives and clinical applications. Biochem Pharmacol. 2011; 82(12):1807-21.
68. Altman B J, Stine Z E, Dang C V. From Krebs to clinic: glutamine metabolism to cancer therapy. Nat Rev Cancer. 2016; 16(10):619-34.
69. Sosnicki S, Kapral M, Weglarz L. Molecular targets of metformin antitumor action.

Pharmacol Rep. 2016; 68(5):918-25.
70. Zhang H H, Guo X L. Combinational strategies of metformin and chemotherapy in cancers. Cancer Chemother Pharmacol. 2016; 78(1):13-26.
71. Gong J, Kelekar G, Shen J, Shen J, Kaur S, Mita M. The expanding role of metformin in cancer: an update on antitumor mechanisms and clinical development. Target Oncol. 2016; 11(4):447-67.
72. Thupari J N, Pinn M L, Kuhajda F P. Fatty acid synthase inhibition in human breast cancer cells leads to malonyl-CoA-induced inhibition of fatty acid oxidation and cytotoxicity. Biochem Biophys Res Commun. 2001; 285(2):217-23.
73. Baldewijns M M, van Vlodrop I J, Vermeulen P B, Soetekouw P M, van Engeland M, de Bruine A P. VHL and HIF signalling in renal cell carcinogenesis. J Pathol. 2010; 221(2):125-38.
74. Metallo C M, Gameiro P A, Bell E L, Mattaini K R, Yang J, Hiller K, et al. Reductive glutamine metabolism by IDH1 mediates lipogenesis under hypoxia. Nature. 2012; 481(7381):380-4.
75. Gameiro P A, Yang J, Metelo A M, Perez-Carro R, Baker R, Wang Z, et al. In vivo HIF-mediated reductive carboxylation is regulated by citrate levels and sensitizes VHL-deficient cells to glutamine deprivation. Cell metabolism. 2013; 17(3):372-85.
76. Cancer Genome Atlas Research N. Comprehensive molecular characterization of clear cell renal cell carcinoma. Nature. 2013; 499(7456):43-9.
77. Hu C J, Wang L Y, Chodosh L A, Keith B, Simon M C. Differential roles of hypoxia-inducible factor 1alpha (HIF-1alpha) and HIF-2alpha in hypoxic gene regulation. Molecular and cellular biology. 2003; 23(24):9361-74.
78. Kim J W, Tchernyshyov I, Semenza G L, Dang C V. HIF-1-mediated expression of pyruvate dehydrogenase kinase: a metabolic switch required for cellular adaptation to hypoxia. Cell metabolism. 2006; 3(3):177-85.
79. Papandreou I, Cairns R A, Fontana L, Lim A L, Denko N C. HIF-1 mediates adaptation to hypoxia by actively downregulating mitochondrial oxygen consumption. Cell metabolism. 2006; 3(3):187-97.
80. Koshiji M, Kageyama Y, Pete E A, Horikawa I, Barrett J C, Huang L E. HIF-1alpha induces cell cycle arrest by functionally counteracting Myc. EMBO J. 2004; 23(9):1949-56.
81. Gordan J D, Bertout J A, Hu C J, Diehl J A, Simon M C. HIF-2alpha promotes hypoxic cell proliferation by enhancing c-myc transcriptional activity. Cancer Cell. 2007; 11(4):335-47.
82. Ebert T, Bander N H, Finstad C L, Ramsawak R D, Old L J. Establishment and characterization of human renal cancer and normal kidney cell lines. Cancer Res. 1990; 50(17):5531-6.
83. Skehan P, Storeng R, Scudiero D, Monks A, McMahon J, Vistica D, et al. New colorimetric cytotoxicity assay for anticancer-drug screening. Journal of the National Cancer Institute. 1990; 82(13):1107-12.
84. Ibahim M J, Crosbie J C, Paiva P, Yang Y, Zaitseva M, Rogers P A. An evaluation of novel real-time technology as a tool for measurement of radiobiological and radiation-induced bystander effects. Radiation and environmental biophysics. 2016; 55(2):185-94.
85. Kusters B, de Waal R, Wesseling P, Verrijp K, Maass C, Heerschap A, et al. Differential effects of vascular endothelial growth factor a isoforms in a mouse brain metastasis model of human melanoma. Cancer Research. 2003; 63(17):5408-13.
86. Navis, A. C., Bourgonje, A., Wesseling, P., Wright, A., Hendriks, W., Verrijp, K., van der Laak, J. A., Heerschap, A., and Leenders, W. P. (2013). Effects of dual targeting of tumor cells and stroma in human glioblastoma xenografts with a tyrosine kinase inhibitor against c-MET and VEGFR2. PLoS One 8, e58262.
87. Nomura, N., Pastorino, S., Jiang, P., Lambert, G., Crawford, J. R., Gymnopoulos, M., Piccioni, D., Juarez, T., Pingle, S. C., Makale, M., et al. (2014). Prostate specific membrane antigen (PSMA) expression in primary gliomas and breast cancer brain metastases. Cancer Cell Int 14, 26.
88. Van den Heuvel, C., Navis, A. C., de Bitter, T., Amiri, H., Verrijp, K., Heerschap, A., Rex, K., Dussault, I., Caenepeel, S., Coxon, A., et al. (2017). Selective MET Kinase Inhibition in MET-Dependent Glioma Models Alters Gene Expression and Induces Tumor Plasticity. Mol Cancer Res 15, 1587-1597.

1. Method for in vitro determination of the susceptibility and/or resistance of a subject suffering from or at risk of a disease or condition for a drug to treat the disease or condition, comprising:
providing a sample from the subject,
performing RNA profiling on the sample,
wherein the presence of an aberrant level of a transcript, an alternative splice variant and/or a mutation is an indication for the susceptibility and/or resistance.

2. The method according to claim 1, wherein the RNA profiling is performed by multiplex mRNA sequencing, targeting multiple regions of interest.

3. The method according to claim 1, wherein the multiplex mRNA sequencing is performed using molecular inversion probes (MIPs), preferably comprising a detectable moiety, preferably a unique identifier sequence of random nucleotides (N) adjacent to the ligation part of the MIP or to the extension part of the MIP sequence (smMIPs).

4. The method according to claim 1, wherein the aberrant level of a transcript, an alternative splice variant and/or a mutation is linked to a an aberrance in a metabolic pathway which is in turn linked to the susceptibility and/or resistance of a subject suffering from or at risk of a disease or condition for a drug.

5. The method according to claim 1, wherein the disease or condition is at least one selected from the group consisting of a cancer, a viral infection, a bacterial infection, an autoimmune disease and a genetic disease.

6. The method according to claim 1, wherein the sample is selected from the group consisting of a tissue, a tumor tissue, urine, sperm, saliva, blood, blood plasma, cerebrospinal fluid, blood platelets, and/or exosomes, preferably selected from tumor tissue and blood platelets.

7. The method according to claim 4, wherein the metabolic pathway is a glucose processing pathway, a glutamine processing pathway and/or a fatty acid pathway.

8. The method according to claim 1, wherein the multiple regions of interest are within the mRNA of:
glucose processing genes: ABAT, ACACA, ACACB, ACLY, ACO2, ACSS2, ADPGK, ALDOA, ARHGAP26, ATG4A. ATP5A1, CBR1, CBS, CHKA, CKB, CPT1A, CYCS, EGLN1, ENO1, G6PC, GAD1, GCLC, GCLM, GFPT1, GLDC, GSS, HK1, HK2, HK3, GLY1, G6PD, Gluconolactonase, PGD, RPIA, RPE, TKT, PGI, ALDOA, GAPDH, PGAM1/2, ENO, PKM1/2, PDHA1, PDK1, PFKB1, PFKMb, PGAM1, PGD, PGK1, PKM, PRDX1, PRKAA1, RPIA, PC, CS, ACO1, IDH1, IDH2, IDH3A, IDH3B, IDH3G, OGDH, SUCLA2, SDHA/B/C/D, FH, MDH1, MDH2, PDK, LDHA, LDHB, SLC16A1, SLC16A3, CA9, CA12, SLC4A10, VHL, SDH, SDHAF2, HPGL/PCC, FH, CS, D-2HGDH, L-2HGDH, FH, IDH1-3A-G, MDH1-2, MYC, OGDH, SDHA-D, VHL, PHD, HIF1a, EPAS2 and/or PDCD1;
glutamine processing genes: SLC1A5, ASCT2, GLS, GLUD1/2, GOT, GPI, GS, BCAT1, BCAT2, SLC1A2 and/or SLC7A11;
fatty acid anabolism genes: SLC25A1, ACLY, ACACA, ACACB, FASN, CPT1, SLC5A7, CHKA, CPT2, VLCAD, HADHA/B, SCAD, MCAD, LCAD, SCHA-D, 2-Enoyl-VoA hydratase and/or MCKAT;
transporter genes; SLC16A1, SLC16A7, SLC2A1, SLC2A3, SLC5A1, SLC5A5, SLC7A1, SLC9A1 and/or SLCA12;
redox homeostasis genes: NAMPT, NAPRT1, NOX1, NOX3, NOX4A, NQO1, SOD, SOD2, CAT, TAL, TIGAR and/or TRX;
DNA repair genes: PARP1; MGMT, XRCC2, XRCC3, RAD54, H2AX, MSH2, MLH1, PMS2, MSH6.
genes with potential involvement in cancer: ALK, AXL, BRAF, KRAS, TP53, MAPK8, MYC, TP5313, FGFR1, FGFR2, IGF1-R, KDR, NTRK1, NTRK2, PDGFRA, PDGFRB, EGFR, EGFRvIII, ERBB2, ERBB3, ERBB4, MERTK, PLXND1, RET, Androgen receptor (AR), AR variant 7, AR variant 12, FOLH1, KLK3, MET, METdelta14, METdelta7-8, KIT, RON and/or PTEN;
genes involved in angiogenesis: VEGF-A121, VEGF-A144, VEGF-A165 and/or VEGF-A189
genes involved in immune suppression: CD274 and/or CTLA4; and/or,
viral genes: HPV-E2, HPV/E6 and/or HPV-E7.

9. The method according to claim 1, wherein the presence of an aberrant level of a transcript, an alternative splice variant and/or a mutation also provides an indication for treatment with dietary compounds or phytochemicals, optionally in combination with a drug.

10. A method of treatment of a subject suffering from or at risk of a disease or condition, comprising:
requesting performance or performing a method according to claim 1, thus determining the susceptibility and/or resistance of the subject suffering from or at risk of a disease or condition for a drug to treat the disease or condition, and
treating the disease or condition of the subject with a drug where the disease or condition of the subject is susceptible to.

11. (canceled)

12. The method according to claim 10 wherein the disease or condition is at least one selected from the group consisting of a cancer, preferably glioma, meningioma, ependymoma, pilocytic astrocytoma, adenocarcinomas, sarcomas, hemangioma, head and neck cancer, breast cancer, lung cancer, prostate cancer, kidney cancer, ovarian cancer, endometrial cancer, cervical cancer, colon cancer, rectal cancer, pancreatic cancer, esophagus cancer, basal cell cancer, penile cancer, vulva cancer, melanoma, uveal melanoma, lymphoma, acute myeloid leukemia, acute lymphoblastic leukemia, cholangiocarcinoma, hepatocellular carcinoma, soft tissue sarcoma or osteosarcoma; a viral infection; a bacterial infection; an autoimmune disease and a genetic disease.

13. The method according to claim 10 wherein the drug treatment is supplemented with treatment with dietary compounds or phytochemicals.

14. A molecular inversion probe selected from the group as set forward in Table II.

15. (canceled)


SEQUENCE LISTING (951 sequences)

SEQ ID NO: 1 - DNA, 83 bp, Artificial Sequence
  smMIP
  misc_feature (29)..(36): n is a, c, g, or t
cgttgaatttgattatgatgggcctctgnnnnnnnncttcagcttcccgatatccgacggtagtgttgtccctcaaggggtca

SEQ ID NO: 2 - DNA, 82 bp, Artificial Sequence
  smMIP
  misc_feature (23)..(30): n is a, c, g, or t
caacagacccgccctcggaatcnnnnnnnncttcagcttcccgatatccgacggtagtgtttacctggttgatgtggacggc

SEQ ID NO: 3 - DNA, 83 bp, Artificial Sequence
  smMIP
  misc_feature (28)..(35): n is a, c, g, or t
cctctccttcatgggcgcgttccatggnnnnnnnncttcagcttcccgatatccgacggtagtgtaaaacgccttaaagacca

SEQ ID NO: 4 - DNA, 83 bp, Artificial Sequence
  smMIP
  misc_feature (30)..(37): n is a, c, g, or t
gccttcttggtggacgaggtccagaccggnnnnnnnncttcagcttcccgatatccgacggtagtgtggaaaaagaagaagac

SEQ ID NO: 5 - DNA, 83 bp, Artificial Sequence
  smMIP
  misc_feature (30)..(37): n is a, c, g, or t
gattccatacggaataagctcattttaatnnnnnnnncttcagcttcccgatatccgacggtagtgtataatgcagcccatgc

SEQ ID NO: 6 - DNA, 81 bp, Artificial Sequence
  smMIP
  misc_feature (27)..(34): n is a, c, g, or t
gctcattttggaggaataatggatgannnnnnnncttcagcttcccgatatccgacggtagtgtagaggcccaaattgagg

SEQ ID NO: 7 - DNA, 83 bp, Artificial Sequence
  smMIP
  misc_feature (30)..(37): n is a, c, g, or t
gctgggaagttaatccagtacattgtagannnnnnnncttcagcttcccgatatccgacggtagtgtatgatggcagcagtta

SEQ ID NO: 8 - DNA, 83 bp, Artificial Sequence
  smMIP
  misc_feature (20)..(27): n is a, c, g, or t
ctcctccaacctcaaccacnnnnnnnncttcagcttcccgatatccgacggtagtgttattgcctatgaacttaacagcgtac

SEQ ID NO: 9 - DNA, 83 bp, Artificial Sequence
  smMIP
  misc_feature (27)..(34): n is a, c, g, or t
gcaatgacatcacataccgaattgggnnnnnnnncttcagcttcccgatatccgacggtagtgtggatgatcaaggtcagctg

SEQ ID NO: 10 - DNA, 83 bp, Artificial Sequence
  smMIP
  misc_feature (30)..(37): n is a, c, g, or t
gcgctggtttgtggaagtggaaggaacagnnnnnnnncttcagcttcccgatatccgacggtagtgtaaaacatcccgtacct

SEQ ID NO: 11 - DNA, 83 bp, Artificial Sequence
  smMIP
  misc_feature (20)..(27): n is a, c, g, or t
tcccaccagaagcccccaannnnnnnncttcagcttcccgatatccgacggtagtgttctgataactcaggggagacaccgca

SEQ ID NO: 12 - DNA, 83 bp, Artificial Sequence
  smMIP
  misc_feature (26)..(33): n is a, c, g, or t
gcagggacagtggaatacctctatannnnnnnncttcagcttcccgatatccgacggtagtgttgctccatccagcggcggca

SEQ ID NO: 13 - DNA, 83 bp, Artificial Sequence
  smMIP
  misc_feature (28)..(35): n is a, c, g, or t
cccagagcatcgtgcagttggtccagannnnnnnncttcagcttcccgatatccgacggtagtgtcagtatgccagcaacatc

SEQ ID NO: 14 - DNA, 83 bp, Artificial Sequence
  smMIP
  misc_feature (30)..(37): n is a, c, g, or t
ccactgtcatcatggaccccttcaagatcnnnnnnnncttcagcttcccgatatccgacggtagtgtcttcgaatacctgcag

SEQ ID NO: 15 - DNA, 79 bp, Artificial Sequence
  smMIP
  misc_feature (22)..(29): n is a, c, g, or t
gcaggcaggacaggtgtggttnnnnnnnncttcagcttcccgatatccgacggtagtgtagaccgtggtgacaggacga

SEQ ID NO: 16 - DNA, 83 bp, Artificial Sequence
  smMIP
  misc_feature (19)..(26): n is a, c, g, or t
accacctcagccatccagnnnnnnnncttcagcttcccgatatccgacggtagtgtttcgtcgggcctgtggaagaagcgccg

SEQ ID NO: 17 - DNA, 83 bp, Artificial Sequence
  smMIP
  misc_feature (27)..(34): n is a, c, g, or t
gctgaccttgctgaaccccaaagggannnnnnnncttcagcttcccgatatccgacggtagtgtactgccgactacatctgca

SEQ ID NO: 18 - DNA, 80 bp, Artificial Sequence
  smMIP
  misc_feature (27)..(34): n is a, c, g, or t
gctcccgagacgagccctcagtggctnnnnnnnncttcagcttcccgatatccgacggtagtgtaaagaaggccaagcct

SEQ ID NO: 19 - DNA, 83 bp, Artificial Sequence
  smMIP
  misc_feature (26)..(33): n is a, c, g, or t
gccaagaaccaggctttgaaggaagnnnnnnnncttcagcttcccgatatccgacggtagtgtaaggagggccgcctcactaa

SEQ ID NO: 20 - DNA, 83 bp, Artificial Sequence
  smMIP
  misc_feature (29)..(36): n is a, c, g, or t
gctcgattatgcactggaagtagagaagnnnnnnnncttcagcttcccgatatccgacggtagtgtgatgaagaaggaaggga

SEQ ID NO: 21 - DNA, 83 bp, Artificial Sequence
  smMIP
  misc_feature (26)..(33): n is a, c, g, or t
cctggatgaccccgccagccaggaannnnnnnncttcagcttcccgatatccgacggtagtgtggtggcgatgagccactttg

SEQ ID NO: 22 - DNA, 80 bp, Artificial Sequence
  smMIP
  misc_feature (25)..(32): n is a, c, g, or t
cggtgaaaggtggcacaggtgcaannnnnnnncttcagcttcccgatatccgacggtagtgtgtggatgtcatggctggg

SEQ ID NO: 23 - DNA, 83 bp, Artificial Sequence
  smMIP
  misc_feature (30)..(37): n is a, c, g, or t
gctgcaccaattcaagctatgaagatatgnnnnnnnncttcagcttcccgatatccgacggtagtgttgagctgaagccacac

SEQ ID NO: 24 - DNA, 83 bp, Artificial Sequence
  smMIP
  misc_feature (26)..(33): n is a, c, g, or t
gcaaggacctggaggacctgcagatnnnnnnnncttcagcttcccgatatccgacggtagtgtaaggggagtttgacccaggg

SEQ ID NO: 25 - DNA, 82 bp, Artificial Sequence
  smMIP
  misc_feature (26)..(33): n is a, c, g, or t
cgagaccaacctgaagaaacagggcnnnnnnnncttcagcttcccgatatccgacggtagtgttggcatcaggtgggtggtg

SEQ ID NO: 26 - DNA, 83 bp, Artificial Sequence
  smMIP
  misc_feature (30)..(37): n is a, c, g, or t
gcaatgagccaggggagaccactcagatcnnnnnnnncttcagcttcccgatatccgacggtagtgtgactaaagggaaaatc

SEQ ID NO: 27 - DNA, 83 bp, Artificial Sequence
  smMIP
  misc_feature (23)..(30): n is a, c, g, or t
gctgcattgtggtcaagcacctnnnnnnnncttcagcttcccgatatccgacggtagtgttcttggattccagctgcagtctt

SEQ ID NO: 28 - DNA, 83 bp, Artificial Sequence
  smMIP
  misc_feature (23)..(30): n is a, c, g, or t
agcctgtcaccaagcatagccgnnnnnnnncttcagcttcccgatatccgacggtagtgtgtgttttgtttgaggggattccc

SEQ ID NO: 29 - DNA, 83 bp, Artificial Sequence
  smMIP
  misc_feature (19)..(26): n is a, c, g, or t
cgctttgagacaacctacnnnnnnnncttcagcttcccgatatccgacggtagtgttttcccattctttggtgtagctcctgc

SEQ ID NO: 30 - DNA, 83 bp, Artificial Sequence
  smMIP
  misc_feature (21)..(28): n is a, c, g, or t
cttgcctaaaacccgctcagnnnnnnnncttcagcttcccgatatccgacggtagtgttgcctctactgctttgtcaccttgt

SEQ ID NO: 31 - DNA, 83 bp, Artificial Sequence
  smMIP
  misc_feature (25)..(32): n is a, c, g, or t
cactgggagcattgccaagcggctnnnnnnnncttcagcttcccgatatccgacggtagtgtaacttgctactaccagcacca

SEQ ID NO: 32 - DNA, 83 bp, Artificial Sequence
  smMIP
  misc_feature (30)..(37): n is a, c, g, or t
gccatcatggaaaatgccaatgttctggcnnnnnnnncttcagcttcccgatatccgacggtagtgtagactaccacccaagg

SEQ ID NO: 33 - DNA, 83 bp, Artificial Sequence
  smMIP
  misc_feature (25)..(32): n is a, c, g, or t
gctctgagtgaccaccacatctacnnnnnnnncttcagcttcccgatatccgacggtagtgtttatgccagtatctgccagca

SEQ ID NO: 34 - DNA, 83 bp, Artificial Sequence
  smMIP
  misc_feature (28)..(35): n is a, c, g, or t
gaggcgtccatcaacctcaatgccattnnnnnnnncttcagcttcccgatatccgacggtagtgtccatgcttgcactcagaa

SEQ ID NO: 35 - DNA, 82 bp, Artificial Sequence
  smMIP
  misc_feature (29)..(36): n is a, c, g, or t
gcctgtcaaggaaagtacactccgagcgnnnnnnnncttcagcttcccgatatccgacggtagtgtgccctgaccttctcct

SEQ ID NO: 36 - DNA, 83 bp, Artificial Sequence
  smMIP
  misc_feature (30)..(37): n is a, c, g, or t
gtgcataggagatgcagaaacagatgatgnnnnnnnncttcagcttcccgatatccgacggtagtgtgacaagaccaacaaat

SEQ ID NO: 37 - DNA, 83 bp, Artificial Sequence
  smMIP
  misc_feature (25)..(32): n is a, c, g, or t
gtttgtggagcctctgctggccttnnnnnnnncttcagcttcccgatatccgacggtagtgtccaaaaagaaagaatctcagc

SEQ ID NO: 38 - DNA, 83 bp, Artificial Sequence
  smMIP
  misc_feature (30)..(37): n is a, c, g, or t
gtgaagggactgcgcagttggacagcattnnnnnnnncttcagcttcccgatatccgacggtagtgtggaagcagtagacagg

SEQ ID NO: 39 - DNA, 83 bp, Artificial Sequence
  smMIP
  misc_feature (23)..(30): n is a, c, g, or t
cagcatccttaattccagcagcnnnnnnnncttcagcttcccgatatccgacggtagtgttgactccaagcccccgtcctgca

SEQ ID NO: 40 - DNA, 83 bp, Artificial Sequence
  smMIP
  misc_feature (30)..(37): n is a, c, g, or t
gttcacagcaggcacggtcttcgataacgnnnnnnnncttcagcttcccgatatccgacggtagtgtcatccaaacctgcact

SEQ ID NO: 41 - DNA, 83 bp, Artificial Sequence
  smMIP
  misc_feature (26)..(33): n is a, c, g, or t
gctggtatggatcttagggaagcagnnnnnnnncttcagcttcccgatatccgacggtagtgtcgtagtcaagttgccggtgg

SEQ ID NO: 42 - DNA, 83 bp, Artificial Sequence
  smMIP
  misc_feature (29)..(36): n is a, c, g, or t
cagttgcacaggtgttaaaaaaacttgcnnnnnnnncttcagcttcccgatatccgacggtagtgtaagaataccaacgcatc

SEQ ID NO: 43 - DNA, 83 bp, Artificial Sequence
  smMIP
  misc_feature (30)..(37): n is a, c, g, or t
gtgttttaagatgccacagtctttaggggnnnnnnnncttcagcttcccgatatccgacggtagtgtgcttcaaaccagagta

SEQ ID NO: 44 - DNA, 83 bp, Artificial Sequence
  smMIP
  misc_feature (30)..(37): n is a, c, g, or t
tccattgcctgcagtccccacagcgaatgnnnnnnnncttcagcttcccgatatccgacggtagtgtaaaaccaaataacgcg

SEQ ID NO: 45 - DNA, 82 bp, Artificial Sequence
  smMIP
  misc_feature (28)..(35): n is a, c, g, or t
gccaagccagaagtgacaaccactgggnnnnnnnncttcagcttcccgatatccgacggtagtgtcaaagaagaaaaagact

SEQ ID NO: 46 - DNA, 83 bp, Artificial Sequence
  smMIP
  misc_feature (28)..(35): n is a, c, g, or t
gcccgcgtacatgggctgaggaatgttnnnnnnnncttcagcttcccgatatccgacggtagtgtcactcatcttcaaaagac

SEQ ID NO: 47 - DNA, 83 bp, Artificial Sequence
  smMIP
  misc_feature (30)..(37): n is a, c, g, or t
gagttggtctgaaagcccccggtatcattnnnnnnnncttcagcttcccgatatccgacggtagtgtagtgaagaggacagga

SEQ ID NO: 48 - DNA, 83 bp, Artificial Sequence
  smMIP
  misc_feature (24)..(31): n is a, c, g, or t
atgtctctgttgctccgccgaccnnnnnnnncttcagcttcccgatatccgacggtagtgtatgctgccccacttcagtacct

SEQ ID NO: 49 - DNA, 83 bp, Artificial Sequence
  smMIP
  misc_feature (30)..(37): n is a, c, g, or t
gctgcccaaaccagggctatgaagcaggtnnnnnnnncttcagcttcccgatatccgacggtagtgtcttacattccaacaaa

SEQ ID NO: 50 - DNA, 83 bp, Artificial Sequence
  smMIP
  misc_feature (28)..(35): n is a, c, g, or t
gccttgttgggcactatcagggctgatnnnnnnnncttcagcttcccgatatccgacggtagtgtggctattgaagaacaagt

SEQ ID NO: 51 - DNA, 83 bp, Artificial Sequence
  smMIP
  misc_feature (26)..(33): n is a, c, g, or t
gctgagagagagctgaaaccagctcnnnnnnnncttcagcttcccgatatccgacggtagtgtccgcaatggattcaagttcg

SEQ ID NO: 52 - DNA, 82 bp, Artificial Sequence
  smMIP
  misc_feature (27)..(34): n is a, c, g, or t
gttatgcttgttggaattggtgacaannnnnnnncttcagcttcccgatatccgacggtagtgtaagacaagaagaaacacc

SEQ ID NO: 53 - DNA, 83 bp, Artificial Sequence
  smMIP
  misc_feature (22)..(29): n is a, c, g, or t
aaattcaggtctgtcatctccnnnnnnnncttcagcttcccgatatccgacggtagtgtttctgaccagtttctggtggcatt

SEQ ID NO: 54 - DNA, 83 bp, Artificial Sequence
  smMIP
  misc_feature (28)..(35): n is a, c, g, or t
gccaggatgacagccatggacaatgccnnnnnnnncttcagcttcccgatatccgacggtagtgtaagtgctgacagcatgag

SEQ ID NO: 55 - DNA, 83 bp, Artificial Sequence
  smMIP
  misc_feature (30)..(37): n is a, c, g, or t
gttccatcctcagacaagaggtaaagaagnnnnnnnncttcagcttcccgatatccgacggtagtgtgaatgcttctgagatg

SEQ ID NO: 56 - DNA, 83 bp, Artificial Sequence
  smMIP
  misc_feature (19)..(26): n is a, c, g, or t
tagtcacaccagctaccannnnnnnncttcagcttcccgatatccgacggtagtgtccttttttgtgtttgcctgggtcctgg

SEQ ID NO: 57 - DNA, 83 bp, Artificial Sequence
  smMIP
  misc_feature (28)..(35): n is a, c, g, or t
gctgtgagggcaactctgccggtatttnnnnnnnncttcagcttcccgatatccgacggtagtgtccctggctcatcagcttt

SEQ ID NO: 58 - DNA, 83 bp, Artificial Sequence
  smMIP
  misc_feature (30)..(37): n is a, c, g, or t
gtggaactggggactgcaagatgggagggnnnnnnnncttcagcttcccgatatccgacggtagtgtcaagaagcctaccaaa

SEQ ID NO: 59 - DNA, 82 bp, Artificial Sequence
  smMIP
  misc_feature (24)..(31): n is a, c, g, or t
gcatcattcttccaggagtgacannnnnnnncttcagcttcccgatatccgacggtagtgttgggtgtcagcaggtcctgtg

SEQ ID NO: 60 - DNA, 83 bp, Artificial Sequence
  smMIP
  misc_feature (30)..(37): n is a, c, g, or t
gcgagacaatacacattccaactatggagnnnnnnnncttcagcttcccgatatccgacggtagtgtgtcagagagatacctc

SEQ ID NO: 61 - DNA, 83 bp, Artificial Sequence
  smMIP
  misc_feature (20)..(27): n is a, c, g, or t
gcccagtgggtgcctacttnnnnnnnncttcagcttcccgatatccgacggtagtgttcatcgaagtggacaaggactgggtc

SEQ ID NO: 62 - DNA, 82 bp, Artificial Sequence
  smMIP
  misc_feature (29)..(36): n is a, c, g, or t
gcctgccgagtttcgacaagctggagttnnnnnnnncttcagcttcccgatatccgacggtagtgtgcctccactactccct

SEQ ID NO: 63 - DNA, 81 bp, Artificial Sequence
  smMIP
  misc_feature (23)..(30): n is a, c, g, or t
gtgggaattatgggcccaccgtnnnnnnnncttcagcttcccgatatccgacggtagtgtcgctcctgttcgtcattctct

SEQ ID NO: 64 - DNA, 82 bp, Artificial Sequence
  smMIP
  misc_feature (26)..(33): n is a, c, g, or t
gcctggagtggtcagacagagtctannnnnnnncttcagcttcccgatatccgacggtagtgtggtcctctggctgtatggg

SEQ ID NO: 65 - DNA, 83 bp, Artificial Sequence
  smMIP
  misc_feature (24)..(31): n is a, c, g, or t
cctgtacaaagacaggaacctccnnnnnnnncttcagcttcccgatatccgacggtagtgttggggtgagttccgggtggtgg

SEQ ID NO: 66 - DNA, 82 bp, Artificial Sequence
  smMIP
  misc_feature (29)..(36): n is a, c, g, or t
cctgatggggagaatagctggtccaagannnnnnnncttcagcttcccgatatccgacggtagtgtcaggagcccgcgaaga

SEQ ID NO: 67 - DNA, 83 bp, Artificial Sequence
  smMIP
  misc_feature (22)..(29): n is a, c, g, or t
acccgcacggctctgagcacannnnnnnncttcagcttcccgatatccgacggtagtgtaacaagcagtttctcctgaccaac

SEQ ID NO: 68 - DNA, 83 bp, Artificial Sequence
  smMIP
  misc_feature (28)..(35): n is a, c, g, or t
caccttcaacatgtaaagtacaaaggcnnnnnnnncttcagcttcccgatatccgacggtagtgtccattataactcagacct

SEQ ID NO: 69 - DNA, 83 bp, Artificial Sequence
  smMIP
  misc_feature (21)..(28): n is a, c, g, or t
gctgctggctttggagacagnnnnnnnncttcagcttcccgatatccgacggtagtgtaacattgaagagctgcttccggaga

SEQ ID NO: 70 - DNA, 83 bp, Artificial Sequence
  smMIP
  misc_feature (24)..(31): n is a, c, g, or t
gtggtggtgtccatttggcttttnnnnnnnncttcagcttcccgatatccgacggtagtgtcccccagagaaatgatcaacaa

SEQ ID NO: 71 - DNA, 83 bp, Artificial Sequence
  smMIP
  misc_feature (30)..(37): n is a, c, g, or t
gcccagtgaagaggattcacccagagaggnnnnnnnncttcagcttcccgatatccgacggtagtgtctgctgctgtcactgc

SEQ ID NO: 72 - DNA, 83 bp, Artificial Sequence
  smMIP
  misc_feature (27)..(34): n is a, c, g, or t
gaggctcctggagatcctcaagaaccnnnnnnnncttcagcttcccgatatccgacggtagtgtaggatccacccggagagga

SEQ ID NO: 73 - DNA, 83 bp, Artificial Sequence
  smMIP
  misc_feature (21)..(28): n is a, c, g, or t
ccctctgacttcagccgctannnnnnnncttcagcttcccgatatccgacggtagtgttgttggccgcctttctggaggaggg

SEQ ID NO: 74 - DNA, 82 bp, Artificial Sequence
  smMIP
  misc_feature (29)..(36): n is a, c, g, or t
gcgacgcagcctttgaatgggcgagtgannnnnnnncttcagcttcccgatatccgacggtagtgttgtgcccagggtgtca

SEQ ID NO: 75 - DNA, 82 bp, Artificial Sequence
  smMIP
  misc_feature (29)..(36): n is a, c, g, or t
gcagatgagaaggcagcacagaaggggannnnnnnncttcagcttcccgatatccgacggtagtgttggagtggacagcagt

SEQ ID NO: 76 - DNA, 83 bp, Artificial Sequence
  smMIP
  misc_feature (27)..(34): n is a, c, g, or t
gttgctgatcccacaccctttcatatnnnnnnnncttcagcttcccgatatccgacggtagtgttgagcccgcgcttccacca

SEQ ID NO: 77 - DNA, 83 bp, Artificial Sequence
  smMIP
  misc_feature (21)..(28): n is a, c, g, or t
ccccaagcatcctgcgtactnnnnnnnncttcagcttcccgatatccgacggtagtgttggtcaacaacgcgggcatcgcctt

SEQ ID NO: 78 - DNA, 83 bp, Artificial Sequence
  smMIP
  misc_feature (26)..(33): n is a, c, g, or t
atctgccgctgcttaactctgggccnnnnnnnncttcagcttcccgatatccgacggtagtgttgcacagaattactccctct

SEQ ID NO: 79 - DNA, 83 bp, Artificial Sequence
  smMIP
  misc_feature (30)..(37): n is a, c, g, or t
gtctttggttgtaaactgctgtgatagttnnnnnnnncttcagcttcccgatatccgacggtagtgtaggaggaaagtccaag

SEQ ID NO: 80 - DNA, 83 bp, Artificial Sequence
  smMIP
  misc_feature (24)..(31): n is a, c, g, or t
ccctgtggatccggcccgatgctnnnnnnnncttcagcttcccgatatccgacggtagtgttcccagcatgccttctgagacc

SEQ ID NO: 81 - DNA, 82 bp, Artificial Sequence
  smMIP
  misc_feature (25)..(32): n is a, c, g, or t
gctcttggccaagtgtgagttcttnnnnnnnncttcagcttcccgatatccgacggtagtgtagtccccacatcaccacact

SEQ ID NO: 82 - DNA, 80 bp, Artificial Sequence
  smMIP
  misc_feature (20)..(27): n is a, c, g, or t
cggagtcacacgtgggggtnnnnnnnncttcagcttcccgatatccgacggtagtgttggctgcggcagtgaggggctat

SEQ ID NO: 83 - DNA, 83 bp, Artificial Sequence
  smMIP
  misc_feature (30)..(37): n is a, c, g, or t
gcaagaggggctgctgtgcggtggcagtgnnnnnnnncttcagcttcccgatatccgacggtagtgtacctacgaggtggaag

SEQ ID NO: 84 - DNA, 82 bp, Artificial Sequence
  smMIP
  misc_feature (25)..(32): n is a, c, g, or t
gctctcgcacatcctggagatggannnnnnnncttcagcttcccgatatccgacggtagtgttgggaatggtgacgcttggg

SEQ ID NO: 85 - DNA, 83 bp, Artificial Sequence
  smMIP
  misc_feature (26)..(33): n is a, c, g, or t
acaccacagccacccttggtgatgannnnnnnncttcagcttcccgatatccgacggtagtgtgcagggcctatctgtggtgc

SEQ ID NO: 86 - DNA, 83 bp, Artificial Sequence
  smMIP
  misc_feature (29)..(36): n is a, c, g, or t
gccggcgattagatactgaagaattaagnnnnnnnncttcagcttcccgatatccgacggtagtgttgcagatgggggctgag

SEQ ID NO: 87 - DNA, 83 bp, Artificial Sequence
  smMIP
  misc_feature (30)..(37): n is a, c, g, or t
gctcagttacaatctgcccttggaactggnnnnnnnncttcagcttcccgatatccgacggtagtgtggtatgaaaatgccat

SEQ ID NO: 88 - DNA, 82 bp, Artificial Sequence
  smMIP
  misc_feature (29)..(36): n is a, c, g, or t
ccagttacttgcctgcattccaaaatgannnnnnnncttcagcttcccgatatccgacggtagtgtggggattcgacattgg

SEQ ID NO: 89 - DNA, 82 bp, Artificial Sequence
  smMIP
  misc_feature (29)..(36): n is a, c, g, or t
gcaaggtttgatgcctatttccaccagannnnnnnncttcagcttcccgatatccgacggtagtgtaaagaagaaatgttgc

SEQ ID NO: 90 - DNA, 82 bp, Artificial Sequence
  smMIP
  misc_feature (22)..(29): n is a, c, g, or t
ccacctgcgggtcatctccatnnnnnnnncttcagcttcccgatatccgacggtagtgtaccacttcctcttcgacaagccc

SEQ ID NO: 91 - DNA, 83 bp, Artificial Sequence
  smMIP
  misc_feature (30)..(37): n is a, c, g, or t
gcgacgacctggaccccaactacgtgctgnnnnnnnncttcagcttcccgatatccgacggtagtgtcgacgaggagtcctac

SEQ ID NO: 92 - DNA, 83 bp, Artificial Sequence
  smMIP
  misc_feature (28)..(35): n is a, c, g, or t
ggactatgagttcatgtggaaccctcannnnnnnncttcagcttcccgatatccgacggtagtgttggtgtgggtcaacgagg

SEQ ID NO: 93 - DNA, 81 bp, Artificial Sequence
  smMIP
  misc_feature (24)..(31): n is a, c, g, or t
gcacaggcggtgtggacacggctnnnnnnnncttcagcttcccgatatccgacggtagtgtctacatcctcacctgcccat

SEQ ID NO: 94 - DNA, 82 bp, Artificial Sequence
  smMIP
  misc_feature (24)..(31): n is a, c, g, or t
gtggtggacggagtgaagctgctnnnnnnnncttcagcttcccgatatccgacggtagtgtaggtgcttaagcggctgcgac

SEQ ID NO: 95 - DNA, 83 bp, Artificial Sequence
  smMIP
  misc_feature (28)..(35): n is a, c, g, or t
ggacttcattcctggaaaaagaagttcnnnnnnnncttcagcttcccgatatccgacggtagtgtgctgactcggtactctct

SEQ ID NO: 96 - DNA, 83 bp, Artificial Sequence
  smMIP
  misc_feature (30)..(37): n is a, c, g, or t
gatctggatgggtatggtcaagatcttttnnnnnnnncttcagcttcccgatatccgacggtagtgtgtgctgtttggcaccg

SEQ ID NO: 97 - DNA, 83 bp, Artificial Sequence
  smMIP
  misc_feature (27)..(34): n is a, c, g, or t
gcacatgagagacagcaagcacatcgnnnnnnnncttcagcttcccgatatccgacggtagtgtaaactggaccgggaggaaa

SEQ ID NO: 98 - DNA, 83 bp, Artificial Sequence
  smMIP
  misc_feature (30)..(37): n is a, c, g, or t
gctggcgcactacaaggacatgggcaagtnnnnnnnncttcagcttcccgatatccgacggtagtgtaacaccgcaaatcttc

SEQ ID NO: 99 - DNA, 83 bp, Artificial Sequence
  smMIP
  misc_feature (30)..(37): n is a, c, g, or t
gtttgacttggagaataacccagagtacgnnnnnnnncttcagcttcccgatatccgacggtagtgtcacctcttctgccttt

SEQ ID NO: 100 - DNA, 82 bp, Artificial Sequence
  smMIP
  misc_feature (28)..(35): n is a, c, g, or t
ggtctctttgggcggaagacaggtcagnnnnnnnncttcagcttcccgatatccgacggtagtgtgcgactaaaaagagaat

SEQ ID NO: 101 - DNA, 83 bp, Artificial Sequence
  smMIP
  misc_feature (30)..(37): n is a, c, g, or t
gggagaggatacactgatggagtatttggnnnnnnnncttcagcttcccgatatccgacggtagtgtaccgttgaaaagggag

SEQ ID NO: 102 - DNA, 83 bp, Artificial Sequence
  smMIP
  misc_feature (30)..(37): n is a, c, g, or t
ggaagaaagggcagacttaatagcttatcnnnnnnnncttcagcttcccgatatccgacggtagtgttacacagccgccaata

SEQ ID NO: 103 - DNA, 83 bp, Artificial Sequence
  smMIP
  misc_feature (29)..(36): n is a, c, g, or t
cttttttatgtgtaccatcctttaatagnnnnnnnncttcagcttcccgatatccgacggtagtgttgatctttgtcggcatt

SEQ ID NO: 104 - DNA, 81 bp, Artificial Sequence
  smMIP
  misc_feature (27)..(34): n is a, c, g, or t
cgggcagctggtcagccagaagagtgnnnnnnnncttcagcttcccgatatccgacggtagtgtgagtacatcgtgccgtg

SEQ ID NO: 105 - DNA, 81 bp, Artificial Sequence
  smMIP
  misc_feature (26)..(33): n is a, c, g, or t
ggaacgggttatgtacgtcatgttgnnnnnnnncttcagcttcccgatatccgacggtagtgtggctgcgaaaccattggg

SEQ ID NO: 106 - DNA, 81 bp, Artificial Sequence
  smMIP
  misc_feature (26)..(33): n is a, c, g, or t
gatagactgctgtttttctggtctgnnnnnnnncttcagcttcccgatatccgacggtagtgtggagatggaagatgtgtg

SEQ ID NO: 107 - DNA, 83 bp, Artificial Sequence
  smMIP
  misc_feature (27)..(34): n is a, c, g, or t
ggtcggtaaagacgtcttctagagccnnnnnnnncttcagcttcccgatatccgacggtagtgtgcatatgctacaaggtacg

SEQ ID NO: 108 - DNA, 83 bp, Artificial Sequence
  smMIP
  misc_feature (27)..(34): n is a, c, g, or t
gtgaatacgaataaatgggataaagannnnnnnncttcagcttcccgatatccgacggtagtgtggtgaaaaaggtgtgaggg

SEQ ID NO: 109 - DNA, 83 bp, Artificial Sequence
  smMIP
  misc_feature (29)..(36): n is a, c, g, or t
gctgtgcccagtggtgcttcaactggtannnnnnnncttcagcttcccgatatccgacggtagtgtagacccagtggctagaa

SEQ ID NO: 110 - DNA, 82 bp, Artificial Sequence
  smMIP
  misc_feature (20)..(27): n is a, c, g, or t
gcggttctcatgctggcaannnnnnnncttcagcttcccgatatccgacggtagtgtttctgggggtgtcccttgccgtctg

SEQ ID NO: 111 - DNA, 83 bp, Artificial Sequence
  smMIP
  misc_feature (28)..(35): n is a, c, g, or t
gcctgaccagctggctgacctgtacaannnnnnnncttcagcttcccgatatccgacggtagtgtttgggaaagctggctaca

SEQ ID NO: 112 - DNA, 83 bp, Artificial Sequence
  smMIP
  misc_feature (29)..(36): n is a, c, g, or t
gccaatggttggggcgtcatggtgtctcnnnnnnnncttcagcttcccgatatccgacggtagtgtacagtgaccaacccaaa

SEQ ID NO: 113 - DNA, 83 bp, Artificial Sequence
  smMIP
  misc_feature (26)..(33): n is a, c, g, or t
cggcaggaacttcagaaaccccttgnnnnnnnncttcagcttcccgatatccgacggtagtgttgacctggttgtggggctgt

SEQ ID NO: 114 - DNA, 82 bp, Artificial Sequence
  smMIP
  misc_feature (19)..(26): n is a, c, g, or t
cccagcccccacccacaannnnnnnncttcagcttcccgatatccgacggtagtgttccacagcctggctgcctactacatc

SEQ ID NO: 115 - DNA, 81 bp, Artificial Sequence
  smMIP
  misc_feature (28)..(35): n is a, c, g, or t
cgtggagcagctgaggaaggagggtgtnnnnnnnncttcagcttcccgatatccgacggtagtgttggcagccgtgggctt

SEQ ID NO: 116 - DNA, 82 bp, Artificial Sequence
  smMIP
  misc_feature (25)..(32): n is a, c, g, or t
gccatccagataggcctcatagacnnnnnnnncttcagcttcccgatatccgacggtagtgtcttccgagattccatcctac

SEQ ID NO: 117 - DNA, 81 bp, Artificial Sequence
  smMIP
  misc_feature (27)..(34): n is a, c, g, or t
gcgttcttcaacgagagcagtgctgannnnnnnncttcagcttcccgatatccgacggtagtgtgcgtgaggtgcttggct

SEQ ID NO: 118 - DNA, 80 bp, Artificial Sequence
  smMIP
  misc_feature (20)..(27): n is a, c, g, or t
gtgctggctgagaaggctgnnnnnnnncttcagcttcccgatatccgacggtagtgtgtgggcattttggtggagacgat

SEQ ID NO: 119 - DNA, 79 bp, Artificial Sequence
  smMIP
  misc_feature (25)..(32): n is a, c, g, or t
gggcctagaggagcgtgtggcagcnnnnnnnncttcagcttcccgatatccgacggtagtgtagagctaccgggcaaag

SEQ ID NO: 120 - DNA, 83 bp, Artificial Sequence
  smMIP
  misc_feature (30)..(37): n is a, c, g, or t
gctgtgggcattaaactcctttgggtagcnnnnnnnncttcagcttcccgatatccgacggtagtgtcaacacattacctcca

SEQ ID NO: 121 - DNA, 82 bp, Artificial Sequence
  smMIP
  misc_feature (26)..(33): n is a, c, g, or t
gccgacctacagatttcggtgcttgnnnnnnnncttcagcttcccgatatccgacggtagtgtgataaagcagttccctgta

SEQ ID NO: 122 - DNA, 83 bp, Artificial Sequence
  smMIP
  misc_feature (19)..(26): n is a, c, g, or t
gcatctataatgccagccnnnnnnnncttcagcttcccgatatccgacggtagtgttggttgggattctgggctgtgcagctg

SEQ ID NO: 123 - DNA, 83 bp, Artificial Sequence
  smMIP
  misc_feature (20)..(27): n is a, c, g, or t
caccctttgccagcctcctnnnnnnnncttcagcttcccgatatccgacggtagtgtttaccttcttcctgttcagcttcgcc

SEQ ID NO: 124 - DNA, 82 bp, Artificial Sequence
  smMIP
  misc_feature (28)..(35): n is a, c, g, or t
cgtcttgtccttctgcaagagtgcggtnnnnnnnncttcagcttcccgatatccgacggtagtgtagagctgcaaggggaaa

SEQ ID NO: 125 - DNA, 83 bp, Artificial Sequence
  smMIP
  misc_feature (30)..(37): n is a, c, g, or t
cagagtgagcccttcttcaaggccaccccnnnnnnnncttcagcttcccgatatccgacggtagtgtacctggccaagaagaa

SEQ ID NO: 126 - DNA, 82 bp, Artificial Sequence
  smMIP
  misc_feature (26)..(33): n is a, c, g, or t
acctgcagagctctgaccggctgtcnnnnnnnncttcagcttcccgatatccgacggtagtgtccaaccgcctcttctacct

SEQ ID NO: 127 - DNA, 83 bp, Artificial Sequence
  smMIP
  misc_feature (25)..(32): n is a, c, g, or t
gtacgtggggaaccccgatggagannnnnnnncttcagcttcccgatatccgacggtagtgttgcagatgctgtgtctggtgg

SEQ ID NO: 128 - DNA, 83 bp, Artificial Sequence
  smMIP
  misc_feature (27)..(34): n is a, c, g, or t
gacgtcttctgcgggagccagatgcannnnnnnncttcagcttcccgatatccgacggtagtgtacaccaagatgatgaccaa

SEQ ID NO: 129 - DNA, 82 bp, Artificial Sequence
  smMIP
  misc_feature (25)..(32): n is a, c, g, or t
ccagtatgagggcacctacaagtgnnnnnnnncttcagcttcccgatatccgacggtagtgtgtgaggcctggcgtattttc

SEQ ID NO: 130 - DNA, 83 bp, Artificial Sequence
  smMIP
  misc_feature (28)..(35): n is a, c, g, or t
gaagagtcgccttgtgagtgccttcaannnnnnnncttcagcttcccgatatccgacggtagtgtaccccaataccactaacc

SEQ ID NO: 131 - DNA, 83 bp, Artificial Sequence
  smMIP
  misc_feature (29)..(36): n is a, c, g, or t
gcacaggtcatcctcgatttttcaaccannnnnnnncttcagcttcccgatatccgacggtagtgtactttcatcacccacac

SEQ ID NO: 132 - DNA, 83 bp, Artificial Sequence
  smMIP
  misc_feature (29)..(36): n is a, c, g, or t
gataaagtgcaatgaaagggggaaaatannnnnnnncttcagcttcccgatatccgacggtagtgtggaagttaagacaaagg

SEQ ID NO: 133 - DNA, 82 bp, Artificial Sequence
  smMIP
  misc_feature (29)..(36): n is a, c, g, or t
gatgtctcctacgacaccggggacaaggnnnnnnnncttcagcttcccgatatccgacggtagtgtaaccctcacaagatga

SEQ ID NO: 134 - DNA, 83 bp, Artificial Sequence
  smMIP
  misc_feature (28)..(35): n is a, c, g, or t
ttccggatggtcatctccaacccagccnnnnnnnncttcagcttcccgatatccgacggtagtgttgtgccagacagccctca

SEQ ID NO: 135 - DNA, 83 bp, Artificial Sequence
  smMIP
  misc_feature (30)..(37): n is a, c, g, or t
ccccttcattgacctcaactacatggtttnnnnnnnncttcagcttcccgatatccgacggtagtgtccacatcgctcagaca

SEQ ID NO: 136 - DNA, 83 bp, Artificial Sequence
  smMIP
  misc_feature (26)..(33): n is a, c, g, or t
gctggcgctgagtacgtcgtggagtnnnnnnnncttcagcttcccgatatccgacggtagtgtcaatatgattccacccatgg

SEQ ID NO: 137 - DNA, 83 bp, Artificial Sequence
  smMIP
  misc_feature (23)..(30): n is a, c, g, or t
ccatcactgccacccagaagacnnnnnnnncttcagcttcccgatatccgacggtagtgtatgagaagtatgacaacagcctc

SEQ ID NO: 138 - DNA, 82 bp, Artificial Sequence
  smMIP
  misc_feature (24)..(31): n is a, c, g, or t
gccaacgtgtcagtggtggacctnnnnnnnncttcagcttcccgatatccgacggtagtgttgtggcgtgatggccgcgggg

SEQ ID NO: 139 - DNA, 83 bp, Artificial Sequence
  smMIP
  misc_feature (29)..(36): n is a, c, g, or t
gcattgccctcaacgaccactttgtcaannnnnnnncttcagcttcccgatatccgacggtagtgtagaaggtggtgaagcag

SEQ ID NO: 140 - DNA, 83 bp, Artificial Sequence
  smMIP
  misc_feature (30)..(37): n is a, c, g, or t
gaaaataaaaaagtccggttggtcctgtcnnnnnnnncttcagcttcccgatatccgacggtagtgtaccacgtgcggcggca

SEQ ID NO: 141 - DNA, 83 bp, Artificial Sequence
  smMIP
  misc_feature (21)..(28): n is a, c, g, or t
cctcgcttcagtaccttaacnnnnnnnncttcagcttcccgatatccgacggtagtgtctctttgcacaataacttcatttcc

SEQ ID NO: 142 - DNA, 83 bp, Artificial Sequence
  smMIP
  misc_feature (30)..(37): n is a, c, g, or t
gatcagtaaatcccgatatgactcaatagnnnnnnnncttcagcttcccgatatccgacggtagtgttctcccttttaccgag

SEQ ID NO: 143 - DNA, 83 bp, Artificial Sequence
  smMIP
  misc_feature (30)..(37): n is a, c, g, or t
cctacaaattggattttctcattccactgnnnnnnnncttcagcttcccgatatccgacggtagtgtccctcctccaaactca

SEQ ID NO: 144 - DNA, 83 bp, Artificial Sequence
  smMIP
  misc_feature (28)..(35): n is a, c, g, or t
gaactaatgacagttgccagatggatgnnnnnnnncttcagcttcccgatatccgacggtagtgtggaaggtgtgtttcctgg

SEQ ID NO: 145 - DNA, 83 bp, Artificial Sequence
  smMIP
  misc_feature (30)..(37): n is a, c, g, or t
gaaatgaaagtttctgcaaaactgttcatnnnnnnnncttcagcttcccgatatccgacggtagtgttgctgaactggggccg

SEQ ID NO: 146 - DNA, 83 bp, Artificial Sequence
  smMIP
  misc_feature (27)..(34): n is a, c, g, or t
gcaaaaagattgttgccataggtaccnnnnnnnncttcagcttcccgatatccgacggtagtgtttcagtccttggagttgca

SEQ ID NO: 147 - DNA, 83 bp, Artificial Sequence
  smMIP
  misc_feature (28)..(35): n is a, c, g, or t
gctttctgaagcaagtttccaagaagcnnnnnnnncttcagcttcccgatatccgacggtagtgtcacaggtaaaaccaaata

SEQ ID NO: 148 - DNA, 83 bp, Artificial Sequence
  smMIP
  misc_feature (29)..(36): n is a, c, g, or t
gctactgcggtattcggtcattgtgaaannnnnnnncttcagcttcccgatatccgacggtagtgtacaatttgacatacagc

SEQ ID NO: 149 - DNA, 83 bp, Artificial Sequence
  smMIP
  misc_feature (30)..(37): n is a, c, g, or t
cttacctgtaatttccttcaatatgagagnnnnnnnncttcagcttcccgatatccgacggtagtgtgagtgggtgccgctgt

SEQ ID NO: 150 - DNA, 83 bp, Artificial Sequence
  smMIP
  misc_feature (26)..(33): n is a, c, g, or t
gcactggatgaagaagttcacaagcnnnnnnnncttcagcttcccgatatccgacggtagtgtggccttcagagactggagta

SEQ ID NO: 151 - DNA, 81 bp, Artificial Sequence
  smMIP
  misc_feature (28)..(35): n is a, c, g, or t
gccctctgttgattggtgtacggagtgnnnnnnnncttcagcttcccgatatccgacggtagtgtgaaagtcaagatacca

SEQ ID NO: 152 - DNA, 83 bp, Artificial Sequence
  smMIP
  misc_feature (28)..(35): n is a, c, g, or t
cactccagatggaactccagcagatcannnnnnnncttcagcttcccgatatccgacggtagtgtatagaacacaccaatcgc

SEQ ID NO: 153 - DNA, 83 bp, Artificial Sequence
  smMIP
  misc_feature (29)..(36): n is a, c, g, or t
gtttgcccttatgatgtgtgatgatcggnnnnnnnncttcagcttcccgatatccgacggtagtgtcacaaacacagttggca

SEQ ID NO: 154 - DNA, 83 bp, Artificial Sequence
  smMIP
  misc_feature (25)..(32): n is a, c, g, or t
gctcttcagcaagtggttgctcggnnnnnnnncttcagcttcccgatatccgacggtagtgttacttatatgcactctgaagg

SEQ ID NO: 155 - DNA, 83 bp, Artificial Sequence
  smMIP
  misc_feature (30)..(37): n is a, c, g, or t
gacggtccctgccaacatccgtttgaaaannnnnnnncttcagcttcccgatatccgacggtagtgtctggagcgccttctgc

SEQ ID NO: 156 - DNA, 82 bp, Artificial Sequence
  smMIP
  misc_feature (29)..(36): n is a, c, g, or t
cagacacggaggggaaggtggaagacttnnnnnnnncttcagcttcccgatatccgacggtagtgtacccacagacaatagc

SEQ ID NO: 157 - DNA, 83 bp, Artificial Sequence
  smMIP
  misc_feature (28)..(35): n is a, c, g, or t
gcatgattccactgggatcctgcaccannnnnnnncttcagcttcccgatatccgacggtagtgtgggtctgtgttcaagagg

SEQ ID NO: 158 - DNA, 82 bp, Artificial Sequence
  smMIP
  misc_feature (28)..(35): n is a, c, g, or t
gccctggagacttcgggtctgatgtctnnnnnnnncttcagcttcccgatatccgacggtagtgtacatacccatccaccaa

SEQ ID NO: 159 - DNA, 83 bp, Artificial Sequence
  smMIP
  misc_feature (29)..(36): n is a, c, g, or t
actgagtcggaggacaaggcagagctggnnnnnnnncttcagcttcccgatatccgacggtagtgtacgagacccttcaaaaa

SEQ ID NO: 160 - DNA, 82 bp, Artificial Sequence
  smMIP
  misc_feature (26)..(33): n is a, c, g, or t
gctgaaggacaagagaaaatacctgnnnnnnnncttcagcttcccgatatccgacggtagtgtaaggacggccccggggaga

SEQ ID NO: 161 - DNA, 83 bp, Artificial Sequence
  smMIP
  misc_feature (30)..(37): n is a, c, g, or t
ggttgcagattatattcctcaactggccannnnnnnncttcagcttcccgatatccgacggtagtgtcagagcaacattgttt

SEQ ID NO: 162 - DNA, 83 bp, Artificial Sequence
  smMIP
  misc_feature (26)..(33): n is a, c, g, or t
gctggagcaattgttgtgacttcacnnnnnnnncttcagcttcccgatatccgacggtagtgtgccattgctgttaatgatct

SEQ ID NO: 163 - DNA, 83 bp, Artificial Sequence
  smMIP
  misc_feature (30)..(37): n is a, c, g, or t
gcagttcgaaatacattgagtttgatgcannnnnnnncttcagcttcccgatatccgacggtagtgttagacttctacttcca

SEQ ID NO: 164 - DNA, 83 bp, Artificial Sequence
  smMIP
  misc_feature (30)..(37): n is a, c, g, or t
gaaggtggtgatcaaaggcattcctttggnnnnnnnncttcagcttcccgatatccgacggtagtgtctctggataagatggg

SEQ ID NO: 165 - DNA, 82 bp, Artificial Sequence
  smMIP
  misc_feature (29)..(36): n is a, c, g, or t
gcggcatcctgcggatcatcaagccctgnnnnnnnncttcagcttcccgatatccgacggtagtgtacgaccccaacttctt

SEQ ID NO: 166 - DNA, 83 bp, Artificial Sequence
  smMIP
  misc_feature (30)..(37): n is a, c, g, or t
gtgagcgggagatgtcctggatcgctgatnnnnnnnncttcagcttcccgatatccgacggtagtgtcaatcccaagaactat

SEQ ID NO: 167 - DNA, 83 bp, Artificial Sequence
  smMIP
  misc_feature (28)..(35): n is a, c, g, or t
gctaaatgtattgctgttggtgagtctnnnnnnnncttcagcttcccgatatccgacggtagtgtcatcaatgaagcttctta

SEQ ID NO: 168 - DNA, 82 bp, Artificial Sequence
  smMIP
  misc_feature (29)..(36): n is a, c, g, or t
gctgacaagatcttcctggagagaaacannnnnnnncttcagcttcccgatatccgacggtagtgtttggaggccgactgtg

SEQ ID NO: 169 - DNA, 83 bp, Artificial Sequence
  smMIP
  misc_feature (30)..(37): n is a, c, g, or t
gcactctggcttggcatacacaatggagcnnnnnnnncttcagcttcccgatatccgacggtagtgtttgctcatgtctgttc

SEQ ID NO: 170 - DNA, 81 bp, Artificial Sequence
  smMIP
  misc_feature (26)..(33): n is a, c, g, or t
gcgaggagcagaagcggaaccgggtnnnnnnnncttcagcttcccgatatccgacggtagtgtactacagcgagttggtgg

SEQ ID NO: 171 - DNA, 83 bp, Artificial Sequence
  smMIP
  misc_feature (30)..(37): n is a, c, g, or t
accgaaaatgaattggaaaagatcacaagnnnnnnnncttcagcttcccgatatccgacggtagtgtgcactgatgtgagtgt

SEQ ID NO: 172 - DNA, 83 bp, Artificial Sequence
  smMIP
  misc_feature (29)..(36): n is a, c, g, or t
gcattttaggaatgacaccagggtttagnnnnnnnncttcagcttcccgatatccgacggtagtgtatgcacacgcctgtgtt

SEQ ID NO: 173 - DNA, 83 bp, Artificial Sequence
  smMIP
  misc_feature (27)..(34): n is a, c, g, or t
tcgactgtgacatactgatcccagctnnnnnnnncttcagcttcccgatatccgacggtagtgtgtctgatgggagtatatgg

SEQ ID NO: 174 - DNA, 83 bp, Artificial Sequence
  smMIP
  misc_feature (30)..(37): n is a, c, g, or t
gccaggcaaattatgcacacagccatgaannnnnnnncttcagcttcccgatatccgacggtagtgtaatttggaaagcatgg

SEQ ID NO: 175 - DNA, 79 bp, Artificial Sequence
  smMIP
  misc_feature (26)..(33): n is a, c, g, or t
gacccccgcaaggtcaacctgggagnnnnnnnncttcagcttcccgatatccgacggtagtgtgggcgtggggtgaaat

SEQ ID NO: 176 - DNA, 81 bp, Artificial Sequence
  smMIP
  misc_feature (20)..(27): n is a, c, g, or t
caaacaacaagaacacaccnnnnnnnncttcagcttcccgatatccgacggtagtgtgtgcttctcgtcttgcccttgggg

SEQ ID NO: 177 - DNA, 82 bp, Artificial Sequence
  smMIP
  misc_feature (27)..(34): n is a, c, g, or t
gactcagcctatcagggcttcgcatcnnnnnnnncttcagcttcccgatatccgacggtagtgtctcctgagttctccattg

SEQ ID NO: 178 - DNA, 83 bp, Artificial Sequence
  smMIP
  misc_feature (20)..(27): n is a, c, g, or t
cctgcaagtcctttcccagnnnnnnnncttcagcttcccgatatccgacggtagtgttgcctgggccattcgctattttgtgt

SEQ ID NO: 179 - DNA, 83 bp, Artificial Sequence
  smMIP
  misc_feature (28)..(35): n is a, c, g, or t
agccctcaaaacccctgggacctggaannnnnnnncttcagcttcccgatatccgacggtagtgtagggagcacgaattgtgg

SEQ ID NO: 180 - DNA, 83 bp, Artificial Sequence
  smMIP
  misc_feature (30)..(37): n is a, c, g, or t
gctttgaccagtggggagtggagctgggannnnnnnncttcagcttcccgatatccgacggtagtgttgaaggaaatcgccca

SEQ ID NO: 181 - DNA, 83 bp, Artificial Sequence
  smMIP
  misc_feature (26)..(33): n is a, c, g, or t
gctcatcaacttcatcaagcagcagnnnnnnnncttcagcttcccgatatccgacggtagtgtcatcatctgggacatcaaca

SEQ ID NO: 182 - DNA, 83 bp, Artificial Sequence
  smMIP
  misc_feature (25)..(32): n is a, c, g, or t
gtgctcatctgcagcctcctctgtnnnnnnnncttcagcttcccgatatccgacggtagtgtaagcagctggctaagaaaata

SEQ ID NO: 183 - DNA, 78 bp, Artificial Sequence
  smMIP
  misc_feature (25)..(32): n is a, c, g, or t
ggagctgcgccagggtgtgaagaannnnnnnncttcagcttcccgatatccgacggtagtgtggagccaggcggtgag

SEQ ID NO: 184 - DNA, 82 bp, Artificial Sequence
  smMIP
  misc_feature (29)..(36): n is a, c, g, or t
gcatggactgagggcgaaggtgctgacgnnnnnnnncttcagcttcccgatatccgacggtagtgtcactaagccagaccca

SEQ ID NO: 185 - DNA, 83 bp, Artificial Sequence
  smMIP
  misc_feature (21)..(28): n is a, c, g, or t
gggcagaggcccatcaccttnnnnnnnncttcagcttcccgatatccgacggtagtgtggagagtggagtacgcagtgcgtgg

SEQ ID NO: 186 - DNA, 82 bp, Artificial Sequence
  smMIP
  misc_feature (23)..(30): n is a, c, g, or t
cgatgccaagaaaagggcggagnnnnnnnncttcagcttcccgatatccgacggtagtgttttcaccgaggtcatccgtgcc

SEQ ID NO: 187 - DNA, 78 bp, Artificial Sequence
  smMIP
  misc_feature (23)..(30): n is a, c, g, or t
gctgggtcgccctggactgtgtnnnnnnnncttcagcttcccgatatccgacggtagtgtaaggcacctaccacttcc

SEQ ID NO: 188 - DNA, 83 bp, Artificial Sequence
  smMIP
  misc_feature (28)..(35): n is a, c, g, or t
tggagaaggactgcgctgcaagacccgnnnnnnnncttcagcttcccgatatccgacggtagtgtaacgaacaccttccacca

SEQ ID NO: 189 - DNA, 83 bp, Artificial Sequence
  smMIP
  misc_feature (23)..(30): n is a, c, g, or t
acatggtgagcaaccagcacccnnnnnnnncttcagcttcccgatatccgacggtagtgttcgtgcctgctgccatgtttcgg

SEQ ID NO: 190 - DNA, 83 bp, Artificial Sequence
  smMIP
  misc_feature (28)..(35): n is a, c, g, or t
gcttgtatgctggagtcaagattgcggnnnnnnnncttcagcttcccgatatccgacggtagtgtacagatgggcaccccttt

SEQ ID NO: 191 - DNA, 82 bp, Artificial Sequence
  smMIP
  misc_feature (26)..(33): n is a, c, g, or t
cgaggaggccattgagaaactaagcnnnnnnnncttcagcttcccgatatccgacggtagtgtgtgtgaagactttggagtg

SEQ ID NO: 192 - DNA, 83 bp, Artificial Sequence
  smMIP
  misc_feature (23)..(30): n is a, c, g, or t
cctcatccgcacgtgtcttctcnnnnnnnncttcagcttcccgatatccgacggtagtgtcttttctgctggtgtagccaatc

SEQ ID NO: 193 - DNA, 82 bp, Artificial Sequence
  smMIP
  misc_feature (23)..(30): n is a, c, g, or t
gctgtcagccagaacgctgcctnnnnnnnncttcagcttcccgatatccgacggtagtgtacctcacaggagcccacttcct

SEQ ID NO: 194 - DNA, 83 bp, Artificial Sequence
  smMIP
  misc_feature (30)..(37): n is a, c, g, or t
gcagcgcagatggctccccagccctgaaannnnnnnncttcagcttcccgatatccgacggtagtgtcagcaccatcaaacag

SEQ ID NO: 195 - DNA, 83 bp, Artificial Sequence
  smMIP
  misc_feature (27)..(34): n is a, c, g, or t
gctgtttgtggatggccaggaaattgnnnnnnnncttcagcttcccgatatccgacggtagtgtgagaaggaaagaaacatat

SEQ ID NO: 196 - DNA, 83 bp, Artificial Sequence
  smMIP
  misc_feature (30)..(37): n is a, c, g, or t
gatgtgggtgaagaaggggaccaggccatnnnnnnnncttcagcttcccgatatccgacggtagtgttggctgggactaagaa

SEQ ID NO: 197 - DNA, 81 bp, Artificial Sequence
  smMIP
  misc_feature (28)..(35): n is a, c, g, or t
gcaggaaaagacactcgtgatgaacaannnnnnnncttcagcttcccgatatccgacggtagtgtcctcctacatcctcat

SEQ ID NO: 198 - DNA, 83 bp, Artificial Sequence
  smMIP
  misc_feature (30)..(37): n is a, c, g, or t
gttttttatgagcttgctcatcagttgccnnnnnnnncttcagcttcccgatatccgacggtagtgtgcggggaccgattcac

SEQ ID NO: 199 - DNA, 83 bp, Artificial Sequence
  smMIP
  misc_feature (27)..(34): n is a, c, g, or t
gcttggtgctgatttgtgaacccattnnnnnnnncttcagcttcccgatatccgacggtagtgtccgaggaagaactatgaac

SEQ ID NO: 200 - DNA, 83 bp, Artificial Sequence
  smMIP
  misc_feature (27)..(34): n is a, c, g, or t
gatgctttaactttgctggccccagcnnnnnnnncttcagcttcccgatatccgacggtagtgtcccttcaacaaacagaatg

SEQ ID NO: 201 - DNA, 83 bp, Artificial Sequence
  smMIP
  misc_feature (30)..(37): n is a, c, g, or t
caccattagaaagcagttccgcaagccctnnnnnnnncttcagcttcccgatatccgacggtagtgttgctgaagacacagaa

SEQ ID NO: 202 - DNA, 83 bp, Artificial Sequence
  smMIP
  misc_feature (30)..(37): n is a, c, g, or t
gcagctactacatcactttcttggaaacgnnnnnnnncttcagcttcccgatatccgacggtagtgtaagaactaaatccaaa

SEQ ID NO: 203 - DNA, 83 bp, Artificial Sequence
  smMIP
  misc_feature (30)..(37): n is a, c, g, or t
gcctccatcatgcgactggcaatcagcttnnnnnnnncttcagcttcccgatatccgacggtagtgtagaggaggaaggagaa

SEQ ID NO: 204 - DNA, 83 bp, Artificial Sequence
  smMIP
  misc_feature (21)..(28): n is a, c, g, or t
cacggtcaccaacagaggccnnnnnnnncttcagcttcccgatatccgacggtagtgtatctttgacttcactcatccctgcg

SEQ ID NO: 205 - DNA, 80 bp, Artificial Sequence
  smMIP
  misc_feature (23)..(30): n is a, c, g, or t
ggaccagactgaatccctgttcnnnnnnnncttcagcttcccgatatccgacggtagtgttgggggctacgtgtggctgg

SEQ ID NO: 206 - DNA, 81 bp, Artificial Sequence
  smMIP
  misc_feature (28)..(35): n is a, c, g, or t
gtctgcaaagggttttggggctcgaggnnnnnnnncttcagcttcccgatatccgacggtagtgtcagatccaccattaca

SEQ ID NO: 207 - DNA, 82 bp, Artificial Sequence
  smMIP
  misc_feature (23)..(30): n is a, c, g, or t
ttccccccacagtgctacgccannnnnnnncttcagcttcccgatatccgacggtagtgtctgagcgcaaatgtacccaatg

SEQ ID NO: 208 - DNA, 83 bp, Artificial Sequence
  smMIP
  misc_feature (28)..(35): n is a, c, g, or t
tggcctctcccgggattttaatccaacnnnnnnnncttcagcttcccgatatccgacggtagtgtcctattacttcacggagc

SEQ ID NO: 209 - DNA, 83 bp, Artificial Sequence
  smMIP
  misc_feature (27)..(34): n is a, c, g, or t
gcacattgatctggtggaaggagacgnnnnnnnncttcagcttcccgatatccgacggtagtgtaacatcgtagctgtggtga

SEQ ID NO: 210 - DNA, 83 bp, Artificial Sequence
  smMIP
  misc_feature (28)..(35): n is a, c, g, or t
gcgcttcctcctctcggagagtggcagnnnnnnnncttcagcttcccgatatccgacggtagtgtataacaagggcacaccca

SEQ ID NO: 211 - DNA, 83 bp, Artificial Sequence
  smMIP
  misc_feature (26)..(33): n is a, c, g, or t
gcgggaatcttgatcacgtggacaannnnnnnncttcagcttcccgatatccgacggtagtgtggggaagagctgtttgatca

SEQ ID NO: 212 - DNA, 83 bp, Artificial Sequence
  smMIP
  misc_feature (27)..(34): n is a, c, g, or t
gctatcctccagcagctaggtctgaannnnnnnncttcagcttcccgatatccgacggtagtgtacttcaccaagaagggatt

SEQ ID NO: 213 - DNA, 83 bp, Artificial Sequence
  smMIP
  misc_feature (23)..(30): n is a, c, g, or t
ctaccacatgcgcctctctgatnnnnnnnncttcagcttcccgatatccgacggtagtgttctcgcgtctccgcctcggtttc

SEQ ID NO: 214 - DNA, 82 bp, Artificial Sequence
  smMIP
  misc_feature (29)..(36): n is a, c, g, or t
gttgggaccatgatgacctgtggttatgnnnnnnnncttcagcttcccgatatccgacggtagtgttcatggaccaagggat

SEQ ID NO: 215 - DNA, 83 bp, Artificial Sequence
  smMIP
  misc_feature (30)..(37): n is a, c, g, or t
gctggtccgtgttcggaatgggaagtgggnnnnnnnncttcagcttcccgatatccgacggtagtgtaaggagactcatgcca

SEQ ID NO: 216 - DNA, 83 bp, Artificial Sequence
  smMIP
  misc_feature (28)..(35): n is a, c, g, or t
gtctcagattgagagtgactgcctggcnnnnnnnncttcagcttcccgatatccgacggtagtgtgtggaatgtacctgggtg

SEQ ID NO: 217 - DNA, 83 bp, Artificial Sequence
  smMIP
  misc_feature (23)..(30): n is a, c, g, or t
gcggcgctcatcactgctgtggnnnnnnnncttcagcttcccgatatccgacggtagtgtgtgggtgtggatgggaccctcta

SEQ ID NO: 218 - DNA, 79 bp, Artificial Sequence
  smMIP
  misc_feature (19)..(26): n is a, c, g, or t
cgtctgtgcggccgtgtgnnnnnnnncttcagcttcccgatatccgacggtagtgttgagccaaggcagcatcctcctg

SEQ ID NO: 219 - DNA, 82 bp, Artificial Sequence
  smMIP
  misc_feature (24)..(31): n is a, c, g, or t
ctctttcccttgtcaccagacggnnnnnnnncttcagcttcccgatatccgacggtagtgttttgtgatcccccaagaggtg

SEQ ID NO: 220 - DNA, 80 bp, Artificial Sequence
  smMIP
  misc_feature (25)..(32): n is a, c, g, or t
cggaggcctgtacctgggtgagctnnnnnnnncttcagcttcccgatatccgacggtagtgttctgcgtcagcgtcgagt

SEQ ID NO: 221 - DNA, 82 bp, Artificial Sequence
  smMIP
  misc_feature (23)..(30): n is a, c, g, or t
ggcctcattgtcggaaccggcannnnnnnncttcagcttcccgatatccgacggtagtgtgatgtcgtgagtctgttgcggg

SEQ ID NO: 222 - DNA, 82 bp, Artificial Sequence
  smMIP
  misc_feature (29)..(36): n is a, c, g, or t
gagatcgaaagtgacagcctggccctgcnnnnnnnncttcagcttcccgatatccgacggtagtgtatgtacctgggggaga

SEQ ID NO: 223 - DNA, 80 bp, Artificial Sequence
  smMIP
  misc_feature (24)..(31): n is a, c, g, or t
gcccagagcaagagaggcatcctnnnnnnnncttcagcttcccgatatccgacggtagtgtgcatgtgcaaggccggctt

SEQ ID NO: 224 - DNA, 83 bp, Artificial Sequence
  smMIP
  misc_feature (30)..(37): n is a, c, g, or t
gaagatgacccagatcatgtttgagacctnnnnnnnncttcagcttcccgatatccgacggtagtgtaccaactgggacgaca

SEQ ID NO: 225 - DNA, 83 bp, Artificial Sequence
  smMIP
  misc_feature (29)..(36): n is a, c, g, or t
gctacgtcgccctggacttcgagcaagannnnnnnncttcagcttcccgatatccgacggtagtgttgcgtctggacctggct

SEQ ID NO: 226 - DNA, 83 bp, Artificial Sequence
  smMIP
  misc_feature (29)..(36): n is a, c, g, or t
accaccatgtaccctggcattgccgacannnnnnnncttcagcttcccgatatccgacggtagtgtttccagccttccttcct

SEQ ID NO: 227 - DNA, 82 bp, Artificial Sequence
  smMIP
  misc_feature (28)..(35): n is a, c, g, or t
gtggatcagcaagcaggagtatgacgannnnnnnncttcagcttcccgatatccgacggtagtgtagaaggagatcactgcc

SEQ ID NO: 228 - DNA, 83 bp, Artificial Sequence
  smMIP
  misc_feature (26)..(33): n is a, c, g, or t
gctcaggtccttttggccagatcttnnnnnnnncttcagcttcccgatatccgacggtagtgtgaggcgagcaaaaaaattaa

SEQ ID NO: 229 - DNA, 83 bp, Artificial Sequence
  smMIP
  misc_feature (25)..(32): n is a, c, g, or t
gccttcacccaaagtgtctgacacnnnnnnnncttcagcttcccgatatccgacggtagtgtactgcctgcagggcttccagc

SEQ ID NO: 230 - DNA, 83 bp, Artificial Sequence
  smMIP
  misc_feature (30)..(37): n is a, c, g, or t
gtccccttcccacgtctccatttctttatnnnnnnnncttcagcttcccgatatccgacggtagtgttgaccacaccaaccta

SEQ ID NO: 231 - DNA, 83 bp, Artificial Sequence
  smMIP
  misc_feature (30)..(37): n is a, c, g, or t
gtggtcggatgtccatgaaggaggtcgatnnnnnnnncttcagcttcccgatatccgacggtagtgtaagccagcagtatcga

SEQ ID NO: 232 - DNA, 83 bp, Artificial Sequence
  smMIP
  misc_feature (26)..(33): n is a, c, g, or t
gccgaagaggaggcctaaggcagagnnnnnnnncttcagcttcccgatatccgacggtagtgttacacaggcgagggcatgga

SEQ ID NO: 233 - DNA, 83 bp, Artificial Sequence
  smMIP
  misc_feature (29)..(36): n is a, c, g, or t
gccattcaaggacctggaggaaagtggannnnnnnncttcagcttcccgatatccgacggtagtgtggtggtgttcagacagt

SEQ ID NO: 234 - DNA, 80 bp, Artificial Sequence
  smMIP
  misc_feature (26)..(33): n is a, c, g, or t
tagcagccggtcacccatctatgaannnnnnnncttcagcttcccgatatccgacggtagtgtttcagtgggaggagcgg

SEQ ID NO: 235 - DNA, 83 bp, Artificial Sequence
  smMIP
  misc_feature (27)..(34): n is a, c, g, or t
ccccttacaccgatgtaaatattgtgnnnnnnnncttcagcttcccgatatccgacggtagtgtgggcttgaaaggccctttg

SEQ ID NO: 236 - DNA, 47 bp, Artificial Sequence
  smMIP
cttcagcttcccgatatccgacggtagtgttgtccgaccatgtgtct

SEQ ID NO: 237 - DNA, 80 bp, Artificial Sequence
  smMIP
  misc_feature (26)..(33): n is a, c, g, or t
cggagcaacgtcacggcggtgcacannnnnnnncttcagcttcccgatatccgacggtagtgtgaaggagaatacagtgg

SEQ ID NO: 238 - DNA, 78 bp, Artificial Sequence
  smMIP
  misc_feature (24)..(31): n is a, c, g, or t
gagatgtaccttgatacagtatgnnnnnnnncttcagcttcccgatatccgacggtagtgtatgcccggaacaaccac

SEQ ID NO: 239 - DNA, 83 bp, Artificial Sequence
  smMIP
  misc_feature (28)..(35): n is a, c, g, or t
gtgacttgtgtgcaggattgatcggagnnnnnnnncttcagcttcccgatatccgacggtagtgtgcagaaagctgtaaagat

SEQ ID NO: 240 - DNA, 83 bp, Artificial Sequence
  smMIP
  misc_feature (22)..(29): n is a, c, g, or t
gtcggttcatgggacggctccnnnnnnnncttcagcttcccgatatccgacggtagtgtcccaatttgatgttcttgttatgc

SEQ ID NO: 241 - DNA, 83 bp, Artificial Sequence
  smMIP
  misc_feature (30)..(37): n is a, c, g, or t
gatgctgcgccacatgggactttttgaccnnnnnnnncttcagcttcccgatatccgacggtagtgtacaccaagtggcaaca

SEQ ID NO: 242 - DNA, 83 bp, Artificial Sequence
  smMIP
  misc_feature (27)..(34): n is a, c, g, or t
gcaaaatgctcagacttcacagaggannnnnnnncttcagcttcccgatatccgacggtagtgttcctgctcagtgccgtgat

SEQ ID NO: 243 - DNA, 81 bp, Artificial Sequence
  smMIP
  misc_feature (28)..(35): n is a, c, g, or t
tctacaactggcatttacatcagtcacnnnnnnnncttcagcttcccgatatccgacggtagtgtgaggctgcgtgttttg

SEQ ID NO: 244 - DNA, 82 bp, Artificial Sequence
  smMIP
  misc_feature (25)..(32): n is a, c, g, or t
gctgagttccatgaaggagaacaannnnnnnncttcagcttcccgatatccgacggtagtgtgtgtggggcctgagctgatg

SEQ ID NO: 245 - DNA, 83 bp, Artificial Sequence
  smMIP
  misc_feature (29)..(36): n is a, c, g, or t
gcggctgaggcgtaagttggacttatttnnnnnnnncttcagcttcccgatatccgacggtagtgtgaatatggcatctgagg

SEQ ID NO: 246 - DNA, 81 bp, Artificial Sequence
  smMIP
  misc_feature (28)..(35): n is a, c, g, or t
gtgatcattcgagagcagacagaagggnnnnnnnncttcagcttcccgatatccgacggtagtgtggagtataagggggag

SEQ ID NO: 247 - DNA, 83 bp, Artificial Sequence
  smMIP
  misc_feature (30)..(37): n is a, c, g, or t
gcggattgcaaagttcgcctttgactatgnnnnnnnncttcagcttcccgatatccgacggtagtgtcacaacaatctagacc

SEQ ID NO: 248 - DNA, 82 bp, Artificial Sequence
  smMIP
  misc_feature (25)..(32): n is a, c, g, or t
gaaacttggggatgggttgttcctnnnnnnnncttcagcttcccgatatccgacggtagtgtggggtgtgattgagtgtttg

SEQ ID NO: 249 - DNA, 83 bp, Artificial Sequence
  smMIP
  misc_feature (30)..(37): n is a, c, g, or t
gtgcagaatccttaccagtttgatgtgctnnnnnnnncttcagcttcccgatatccgacggtagtgtacaaggccaacatcat

SEQ ID NO: 250 - DNA, 83 bp, Artificial Sequence
  smMIP
  misc_feature (26)..(33): n is a, c, g, or t
gctggtgtggtccctggtgagagctnnnnnnnncttcagcttcccgatatccgacggtagtgtgagacaatgatcatagacaa

SEQ ID NO: 251 - DNA, 80 bp, Artificial Sequence
  smMIP
  misc_feature (21)..(28): n is a, c, g, or t
gccatgctgctgtcggcttcnnnnnnnncttcagcttcccgatatccgacggtagtgttggctgctggcctggttggggg

SEQ ID NO: 252 - DNA, 81 bp, Artificial Sequence
  smMIP
  misc_feature (25)..(32): n is a, c, g, or t
gcaaggtgcggactcgagacatggnnnnnnnncttcagcttcccgatatccgacggtagtgtgcagtgggcaggaatatag

SEQ ID NO: 253 - DNA, 83 bp, Artificial Sequence
  smMIP
  misc_feature (30)..(37): n is a, c, g, or t
gccctttatttcttccaaccttgcaaggannnnnnnncttcagcttcccgatatccgacggtagtgtagatgcggtgaagaag

SEQ ID NO: 254 - DNA, 82 bp, Artificial Sequence
  smMIP
  misc_feature (29)..(36): n is a, c, g, or t
gcacacggtgaccatgatcccaggggatnnnnnnnncttcagcttcccgatatccgacggtagtgtgaaggcggtgctcggg

SEQ ID NO: 255 - DNA, 83 bp, Artificial Sequence
  smMIP
  misc_feature (30)..(37): n is a, c, g, or t
gtaccagtggactttgaagaggtgcacgtnnnnnnnncttcagcttcccgatatccgacggtagtgtcagaacaaacaattcc

SEQ ID NO: 256 - DNA, 83 bp, Artificial Sequence
  smMIP
  misc_feature (24)..(31): n is a, c, g, or t
gccctgaagggcaacatcgaaacnnnnnnnncttcagcttcccgatatccgacggtagtgttgctgcatgtcaagtccgtctt

SEQ ID NO: 257 - DNA, 83 bp, Artificial Sequence
  smMIP
  misc_feature (25)..(32): n is a, c, g, or t
cgtcatccactgtaagagccttccnnnnnnnncttcagcttcccgatatccgacggtagtgtatgccatcatggccatccgcc

SEQ ID NO: 258 - DNA, 83 bp, Artificial Sequence
  smMIP
  misc_feature (26)..(33): n is a, c, g, or t
gtacagcagcctggagcatgagagtnnnnnnnncttcagcttcccgatatccgacggtagtgtaaacaacatccttcgcacca

SEQ ID NO: 259 - DNA, 83 bp, Artificial Sequence
  smMIP
  misc_feature (30)..(37): n is a, c, g, or t
gcattgccgagtatgccttcaagctggcgnnnnnnnncttcagcttcccgatatccgacggtagtgtcaaggacatagacatc

SEQ ID NO: 260 - DNA, 83 bp, Artificial Sequence
  smMIP
  misc_feature (30)..(37): n is a, c, g, or t
gcttttcctccagtgctgcagggaggtggnnnnnnnncttcagcttcccgatatccgacggtagtgtaagatcatcaccaagg

SEQ ID NO: 261 - DNA, 83 bp, Artificial Sequence
  smMIP
  misc_feature (30)..(37): n is a, c, g, or t
cggccccagcagtttgatgtcatggtgatnnnnnnnncttcagcttcccgatatccgacggtagtgtccaacatcatgaaact

SEQ ID NO: 262 - DNA, 83 bp, Artificial Sequence
  smMIP
  misc_feature (30)..(37): n is a, c, g, or t
gtggctggggccaactatggccatgtgtannnnnnnncttcagcttcccgatatccgacggtagtgttggataacaccaccat

SEQ ID NO: 263 - DNA, 83 bp, Artificial Sequence
  smMIP
  misc_feature (20)..(27): n is a, c, g, or t
ccaaccccacggccaccctnnnnnnnncttcagcttcccgatatccgacggtagtgttcgtcaacaatgtctgcgcgggactg

SEQ ID NO: 264 - DNA, 83 bp, Artificial Sequence
  smMIP
  misc_feature (28)..(35): n is a, c, g, or t
gctgtcctggcatccatggacaatgagnnnnnnnncttcagcttcccgatatccgacggtagtgttacgaggaacaccggcaa

SEQ ID NO: 265 - DNA, 82 bp, Artificial Sequence
  smMIP
  misc_feature (19)..(26): n is a, c, g, or t
cactgaccacagcccccannnnnnnncttcagcttcccgatatccgacggtagtgttgcactcctatgccacctccatccgt

SEQ ID NO: 266 - DNA, 79 bp, Artificial Sequence
  smMIP
  misc_feature (19)..(26): n is a, c, g, or t
gtcatcgttggtggcggannnnnnnncttcagcttcccgatatccgacggtagtgttttggttggtgcctgcggacggg

SEQ ID NO: 267 - DNA, 83 bp, Artificial Sequence
  smMIP
  misc_feature (30)..(37): n is a, c, g, or t
gttctggaaaaggagaaagatttagctgtnnnnnnnncttcagcttcccgatatccgacggtagtgttgtgtggaggtagccg

SEQ ID NO: 268 - DNA, 83 bp, Artificial Sequence
  smMIP
  misc_feature (26)..(33): n is a, c, g, or t
tgtgtacaaggtgcagccctcctctnnnnnnnncttcagcttcccgatatccgacggtagtgtcatccatcactttctattgg

SEQ ID NO: 269 - DNA, 83 bp, Artificial Sequence
  smMIP
  misc_feature (19)..(26): n is a, c, g, or t
ttcccagacttcaggcccnnnnnnnncttcagcttcccgatatccgacggtagtgttattataaacctgagtctctgaaagcc

SEQ ID NO: 270 - DNA, 83 bp, Artificial Sequence
  smMIP
  misc_feature (30)..(37): n is a, c, g, or t
gccatattgtaggggtctaatggctattgnnnnnnnncttcagcttcccgatatccgacggtagtgtaagcttatagtagctg

SEQ ID NO: 271 - DNA, 78 bp, Artificial Sequence
  smMIP
  misc_feature (19)..(26): n is a, c, g, or t
gcaggtggctctgtcttgnnnnnnnncttcagcttcccgatatccgacggtagtgtgaggctgatccagcaggaggat

SEQ ID NO: 272 - DNA, 83 bp, Artificial Sequence
  smMIP
  misc_feature (30)..(37): n is a, c, g, or t
gaatacaaagggagaggaaattcgatgtcnnnnnnnncttcagcttcccgatatccgacggtagtgtgcccaggatttccaag

SEQ ID NO: 273 - DNA, 83 bp, Artificial Sequence
  smMIP
  misc_feature (26)..(33): n is a, c, g, or t
ggctgcactcctgatcctcgaattgnnnnnnnncttcagcttcccgatatccgacggtagtgtccttcaagaagtatagatgg

SEQ ID NO: 274 - DNA, 83 bp, Artificial Sequence
  smMIP
  misc_feature (25)..(32): n is a, c, g, or t
gccggtttcctttcctaggagttcnnnnnnnncttcagcttcccgatatccgacggtagtgtgaccgtatttcagagttgagt

SEQ ID NO: 275 - DNA, 83 bp, Artificial Sequence
  smMIP
  misc_feature (26)..(33): n is a, c, g, or t
ccctttgacttcagtgccacagatgnnnnnnnncttcagcttcccgatatccgacggtagtgtggaaatatttatccggtccc

SEQ ID NO: 276 - DNA, 83 bp, Artificial Sequence
  smMIP
  misc_feature (29)..(36): n is a, c, g, or t
gcatgttttcttggtgcaacagtgaagtnnnnnnnncttcagcttcccgatatccgacggtagtgtaaacgagagggttacag

SEQ ID NO: 277 - DNA, 83 bp, Artificial Sequence
  smMIP
  misc_feature (24)..(31): n is a, c, g, or t
gcccagctggagtaagagcccagnnnnnnnncttcagcttcccgatatccgacggtagtgtgattaaactggcatcccagaat

SEQ ID NO: 278 - DNA, 83 bp, Artificial Sequence
  smMIP
  misc_feature (29)..(36): n is a, c, g, or t
ggggatattggaaatcgcattcttcatgnnnnnnnncttcagcttcccgatatccgacggtagtgtcttcaaaaattcatccc

SEQ ID NO: 279 - DNA, 82 bp, Artificial Sequence
  smMIP
  misc_feature (26)..(33): n is a, c, g, or t
gcagatgaagtacaacaaagatttgnnnnnnnncttcagcttcccgatatccgacggtagtgtgatggaaatctggtagaag

SEQ ID NO: 280 - DNA, 83 bp, Artificial Sequence
  smMIP
  misc_feature (30)..(37): n is a, c, g, or t
gcaacaagaatgtactaattgcattctttnnnnnnnncttcagcttcccgatatccgacggtagtgtgcaccttctcctgctg

SEQ ID NO: 281 - DNA, 83 bp, Artificial Sequence
  smMIP
  misc_feature (25)..(32): n is a, c, g, or t
gcatggcctgtgccatcagtatctnnnnnnnncttcagcttcccgatatccgacggtagtgtgcattcccgattccttttggt

SEQ ID NO: 282 - DNA, 83 bp, Artificial Sequence
  smMIP
  misc_feature (27)..(34): n is a, c, g, or t
gttattggaagcggttgcaatctggannnnnnnncttcagcttcccgatatccgacggtagtgttgaagggagagatgatgga

SEQ ID NO: 283 - DNA, 83 bp, Artificial Sequence
  smMIP
  misc_feature (28)..(35): n is a, c, g, or t
gcacccagatttagggactgataaagannnnnnnncttcagcttcccgatatccgacggtagtgttaatgggggaaaggctgg

SEQ ID NO: 284 - DNA, 83 bp, Artificial Sequence
  smMIP
  misc_feature (28)..(35): n is a, c, g, or t
ggatgatgtcttccttagtgttccttgnnnnnnnncttcagcttcccgatatccgacggtagtgtaactcaaaggctacacat

SEQ ID NO: 285 - DNA, 83 bp, Artificial Sequence
  smMIP
  misc_feature (29)..(36): n is a, c, g, or t
gcatgttgtcctttttatctgatctgtgnnnnnnnncttcagcttcccgatatccgacggtagtgtggcccgtttgaagaaga

SEQ ID NO: 286 - DNA, 82 bp, Artificial Sequence
  smMIP
  misc_feature (29)..(36): n is a, c, g, or t
gttggtatggcgtgtgctatcagcattcnnnnnnnncttcagcttcccgatatccgacggtagtgtaaaaccaggccctact

SEQ ID NO: 287 - DNA, 83 bp, Artificial Sequence
  smMIP
  misc_feature (27)..(34): n is a, c, g, or t
gcaagaaggggagagtcggctcaatcnnnnnnnncttcagcttcccgatatccgacggtagtgtaaaggagaaatgatggatc

SEQ ID NO: 288 - DNA, 81 bp, Artificial Sequence
  smMIP
  misc_feature (24)..(31): n is a, c, g, or t
gtgtggctgtgtggagtggtgtgnnnnnnnncttcagcttcccgatatccgacggtagtgtcaaacaccgcgtgattggaa

SEQ ID NO: 289 - DNA, 83 bp, Artificial Sequence
  smMIP
  misc_feature (30)..(37): n is a, c, g, or t
gtgtggctgatcttattgaatccatgttgnnnnnnnncttcagcttcccgatatccgacggtagtgtgaatccagaaatggga

SEQ ID NO: 290 - DNA, 83 bp, Artificial Sequence
  smMIP
  misc_feature (30)..(37): n is a, c, g, or t
gctcaagaaaagtgcagataccctgtgggnnnnnnnncttcagcttcccgatatccgacggtagtgtacaatggtaaagggga

SEQ ID NO: 291 - DNA, 83 bp, Artificial Sequence
  smMIP
  misc_feature (30)..(37): n is a, c, g, or t
cctataggctcaggagctcaaggaatagtnnnnnnnncttcagcttcccgatatccgacggtagtgtggatgaagccattaaa

SEQ ID NO: 292 - DNA, 83 bp, Artificial Sequence
  smMIP
  misc_feature (28)..(35): n is a, c, g, or t
gcaaatctttgccaagtgattcagatgnnnnnnnncttcagcttcccgatatccgacggtagtgtcagagagctagttcttat

SEQ ID NO: 293 - DNA, 83 bp, Artificial Sequence
  smMIP
  misc_feature (25)..(32): n is a, c, g, or t
cgttgacatttggtcagttgggtgnnnnnnnncttcagcttcccgatatccgacggtagtgtgcactttgaagattcttgact

SEQ ID NO: 294 - DNA, 83 bp, Artificial Sequence
  smMIP
  misc_feature (30)..(37): n is a, c, g, or t
gctggtaatagatgcatctaaaaggatctnnnnnnnncttcagcttcccgatatccgacggtagtgtgaaaacagacctaaat

SEQ ID NO: 295 - DNA, 83 bp, Artificial Sequence
  smMIP
  misc_feature (30)..(37): n is a, c, g, or t
gctctcagcatccatcatcatcgtcgtctnnnnnnnncttcagcttcccgatatccgacggtagtgtggaacacacaatagaa

SEQ ID NO: 296 - DNA, 83 bp, Artificial Sequence
  smMIP
  misc_feature (24)..(31): n is a, c, g, or t
cgactcggtgcagccgtatttctnnnnnnnncttcagcttcccgatatccgacggtagtgtcttctctgaaaggctctccttg

SEQ ID NO: 297 - DNA, 79 bp, Artificial Sequence
  smMIP
  misc_feature (21)..(28): n is a, c, g, or t
ttcgagctgctgcccaccccnnnnnnnncttcagcttcccgatatccgacggtagtgtacaggaactatgacctcgact

SEQ ID NO: 298 - DNA, 82 bp, Artificial Sequence
  smMIP
  misc_feature (24)..(31): n is a, c, g, or t
tctgtggaaaagaggcaggctccnnnnnnnncttcagcttcccgatatccgacggtagtgttgctctcctcgacggagtcct

SEQ ID NO: 299 - DNA, 80 bp, Artificial Sequence
  smMIP
  misc_feature (26)..(33): n is a, c, g, or t
ctggtcctcaagaggtgccacgtctnnnnnnnncttcagcttcccgatatccgacggtagtgtgaggaggaacaagaaga

SEQ ID NO: 300 - DNA, 83 bp, Artificial Sequence
  smMIP
  misc_feature (28)..(35): n is a, c, g, or t
cagtgtcagagtcctgagacagatcagnnnnnnnncttcagcttcccgatatccgacggtagtgtacagcaaacctcctcaca

SEQ ID NO: 301 - DNA, 83 bp, Artificial Sequence
  smMIP
  misc_feature (30)..(37): n is a, c, g, or t
gcgccagaggaggaacgagctaaaacggannnnnnnncttcagcttcccgatatccgacggtagtgtaagagggtcaagttgg

SEQ ID NO: 302 - DNA, 83 bp, Artificial Sequence
  smMIP
  misc_feature (27)..(34): n is a, c, g, or t
agccacagcatacatcctgtccgtccnnnnnnnncttcagcttcccgatatccgacggtagtgtcgaacacacaacgtcttgg

SEQ ID NO: 303 - DNA, 83 bp, Artificial Sequence
  smMIP
  misc_feature (28)..(35): n is a, c, g, or t
cttgaacagctacggaactcttgtgcgnnnnnnnncttcagcttcccgatatccgacggtagtgtggcccccaaggtagttat

SEQ ID NO: 304 - DNA, 83 bp, Artificial Sequence
  smMIP
  misc_feature (30)..(37): n is a, c, g, or t
ccttctaacagaaatgtcctgagcaatcannnnnnnncttcagcttcccgatatccgacggtagtgtaagcagaggagcaaaa

SEQ ID NO: 305 - DNA, 83 bp, Artificial Sequence
  smMIP
  misc_feature (29)..(36): n is a, c, g, or t
ctttgaatgccgtgaaaagaagacagaannnnnnnncttcagcttcccgatatccgacggtagtgttgtcctccggcccgaga

SEQ ID NO: 306 - DNA, 83 bp, Artificial Sequence
  smMIP
  misc_feature (30)..(37): n is a, c, g, or t
ggaaatgttctcttcacggtggaaaacacnnnnnnnncttcagcttcccgatatccgacggtagtgtacaaagaacatttcca

SEQ ID NO: 307 - DNA, 83 bp, Artificial Sequence
  smMIP
  misc_feature (23)..(30): n is a, c, g, or t
gtagcaggacttgctctaattannnnnnnncttcagcttcccgatatccgacggtagtgtgaaacttctggtaacttagatgg

SEQ ID NO: 308 - DNA, 83 bp, Artificial Sequence
  smMIP
  misc_feature (30)..(37): n is a, c, g, or t
gccaccttatcttagagttattcaaggggnnnnnnnncttcagcttcccgatatccgacggtagtgtacacaggcaccactaa

SEQ ID NO: 309 - DNA, 83 bp, Artificial Sequence
  smMIP
  misc_feature (28)..(35): n is a, c, g, or t
cgccagcagggaattttgttacactggnnnnnnnncttcagcttcccgatatccgacggtagtgtctcttgaattgttccttc

SEQ ID NO: 310 - DNA, 82 bp, Artificial Sequence
  smMIP
  misc_feature (19)..(26): n is a, c, g, or t
gcccaggtggagccactannnnnnnncttcagcttcccgatatccgacggtagtgttttccggctcctgggctctgacgggt

SEQ ID NO: 311 - DNA, 81 bp, Artificial Sequence
  smMIP
  misc_feature (24)..(31): n is a, c, g, or t
gttccaggtgccctggctggagtnnnnnnnncttcagcttcccgatatccgacggtagtgtaacttcctagcagtcgccct

SEQ ID NO: 312 - DNA, 82 bp, Artificial Sequence
  smMIP
  misc_feature (29)..(36): n is a, c, g, or t
gtcattggcattggcaccagtgtggtcannnnnnnncttcagcttcccgatatccgacggtagtgtgtcttccgagctgctg

SEQ ID NO: 313 - DNA, 83 bp, Artificial Sequence
  smMIP
  misc_feature (30)..(37): n is a, c, g, or t
cgaggaccccgagaagcagacgttgcctgnnnnnnnncttcagcttcccgatatccgacggtagtgtgagggcagtgaggtga

SEQ ID NO: 314 - DNA, 83 bp, Artificial Sequence
  smMIP
  misc_feature (26)..(33): n is a, c, g, or t
gccttcctgaaatatgagaaggccgnnnnnnnncttcagcttcccgatatccgacggtagtgttcttccctgttgcctagaag

SEQ ID NO: 315 - DNA, 83 bp, Artificial Sequence
  smMIP
  misc_feature (21)..(28): n is a, c, g, or t
cccatccagtcccgaaacacnnnnnnnncttcagcttcccgatatccgacggtagtgtctattcacatcattgcacacctgtt

SEQ ID NO: 316 - DNA, 83 bp, Artificial Sequence
  smMIP
  misc_feature (26)..(33): n is a, c, g, or t
gcaccggtcattctttatatctgtgnnnnnnnncttcagcttcccgatatccgacggtagtgtgagagcatgaatgagagtca

SEQ ID NO: 317 - DNA, 83 bp, Artificial Sequence
  smMIP
  misc_feature (25)..(32): n is a, c, g, or t
gctggttggagcaggaattggggtnnnnnnnncttcagcttcccgatatccgacggtagtgtcagcaggggactggacagaaa

SEQ ID NO: 318 - DNA, 83 bp, Artificial Sequence
  smMIP
  misc_feature (30)..(37): n is a, c, g, or t
gtctgtagtgggagttttcttatgtggccnnnnnnnncttcagcttcccgatatccgacggtagtgtggtcatgcagcattaa

SEQ ID NO: 319 - DNA, 83 bp, Artificial Sequence
  smMIP
  misc_feature (27)..(34): n is a, c, g, or t
cgagttattttgggttcaacactggcnnnnnnnncttcagcttcccgatatccgacggtagtgttggggtgctggattttgaa

SEQ ID NO: 320 - DNA, 83 bp, Artificial Sequence
  smMIP
  misc_feature (19)..(26): n is a, c, g, or t
ccccacaaacacaaccacnnnnnnnncttcagcttcccgatatccgacggtagtgtacatcgtggcgcatttcttcaacctgg

SEQ ID NO: 321 - DNA, 83 bp, Artificial Sequence
  smMIP
  misc_feature (30)..(37): n is a, c, g, or t
gcgatttcaacaagaagttgtcattaccannnnnnnncttcagcttcccgatatccgacggtagtgtgcagaatggcagacag

SEQ ID NO: 322 - DNA, 82 bp, Artificial Sequence
  smMIP
  misc_feature (28)..(35): n is a, c, g, or t
gcgttgccgcggggatcggagtcactcnnnnnnnncttcagcttcccgatatccgacggtagtgtctactggaggcctttgg

SEQ ID NO: 323 - DNA, 83 bp, Artificial Sequence
  smMIP
  misc_feature (27)..(34): n is a, c, g, or t
caagcagattgcctacaatcaccccannnnnnnncttcagcttcccgatatccgacggtagtgttttcttaccggctgggatg

SEQ ID NO: 324 - DNA, 83 bp, Artificial Sequence
  smMIP
  misc_feature (25)..(32): n is a, c, g, or t
gtcctgcttttctggaaaaccttcnnnnnnnncttcagcttcccgatatccgacggtagtgttccttctcggtccggcgggca

SEQ ID NO: 325 - DNA, 83 bp, Artificial Sequence
  smMIP
  misc_feature (25)..(32): n is a, c, g, or t
acttctcttcacaactgttcctggnnnnnnnncttcagcttcccgatatccgacggtagtgtctatctgtattttctcaggcg

SEQ ID NO: 326 - DNA, 83 bp, Artificial Sequence
  smMIP
  misc_feature (27)..(34): n is a, c, g, or t
gcccagattccaagctaattttccacnnnnnnnncttcagcttcccgatatccgacggtagtgtaccagctctcagaatattt

SEQ ID NO: 327 - DNA, 83 bp, Artificial Sequence
  smMIP
  misc_feature (24)..(31): n is a, c, g, or t
gaaattctgcccttcattcaatcnnnnnnnncttcagcttcccgatatccgacggtagtgtatcatccatttaccctcacaat

SEQ ID NO: 328 - DNA, 83 bp, Artificial Sequence
  smMIP
  misc_feature (30)..(37): n is a, c, g, or t
cggtggaaacttttgtttgatgaaatagcnnnnnnnncttcagcttcccgatatccgacggtagtgtggcaagagaacagacc

SEQ ID NO: 329 - DNA, 83 bp, Artificial Sequence
  smMIP
  misc_feature (27)..(34): n is a, c, g, or t
gcggctttgaagaagaaaggatgggannnnnnnncttcagcttcccgatatccgacggtagtgtaaccacgagcccagccaat

SEQ ID NO: 330 - DNA, 83 bp, Artificial Sequence
  smMIP
  misc_feature (29)..(36): n is a, c, g, or t
gctggaagccgcagaccttgtgatattcnnnnnnnncttcagcttcccgatatccgacggtagtgtcatttccagaaaggaca

SEQ ID NO: 331 - DNA, 83 bp, Artificial Sequence
  smMIP
  misc_feature (29)..(36): n is a, c, g, or t
catcaccactggtggcagtggctccatgnnnnnnnncttcagcttcccgatatccgacggtagtgtgtggtttggagtccctg

SEQ ID NO: 332 - DNA, 83 bp, Artificial Sequence
  smMIP
  misc_feature (30)..(37): n is a, c, g, or t
gcccgaattcaaatcctggaaggatggaannnnnnnncttcagcttcccgatatccgacggtagtgtaagggatccacgggga

SEQ ID NO: 333 - DNA, 83 bp, Artificial Sequence
  smMIP
  misc_feature (24)..(31): n is a, c, g, or t
caagtccatcccaactgacaaccnnnnnnnncttcagcttcccgatatccgacggtagtgtacaccactgtattttgctccaa

SEQ ID NO: 334 - DNA, 83 bp, Artificial Sequence
  smMIP
  misc_feature (27)..(34): n is a, c, g, or t
gattcggtgctattctgcacctgttgnnnnnnnncttcagcttcccgatatccgacggtagtgtaaaaacttcaggacaaaaa

SEQ ID NO: 335 - DNA, 83 bp, Artificial Sequence
  smMIP
  misc_feature (29)..(36): n is a, c, g, or t
gctggaaaaccccaaaagtgtacataagnnnnnnnncttcagcttcccgatatccgacggtagtgtacaaaacagaccagcag

SEQ ID NO: 336 - DNA, 83 bp, Artificial Sequence
  smMIP
  misc_feature (22)..(29): n is a, c, g, or t
ctgcctaccagagtccccttcnnnnnnnncttcagcttcccgatatccgacggtagtgtccctttctcagtgggactagttcg

SEQ ID NO: 337 - DNA, 83 bp, Artificial Sequence
  smMIP
  misc_feature (30)..(37): n is a, c, g, or t
gctgatctggactcctccgtgcccgctgannnnnnnncttcagcttcccgatatccgacggtagtgttagaagcacagcccaa

SEQ ID NO: 338 - DNA, 83 bp, Artificial Sequence
  smMIP
  misc_feature (21)..(28): n is a, c, g, or t
ttccacttgcccaccaccacnnnnnnnncttcagcttcccgatatccgacggtagtgtaccatgtagcacagctggaccccct

SEQ ID NO: 339 - DNA, 83 bp, Artificial Sequence
  smMIP
  misc_feature (28)..(35): n is a, c, g, or t
gcatattggggtggagttcatgttcatnnnnnnnncttcagcttcccgatatccgacggtagtgtatggcctggatgagtctg

SEQ ID NO: 340 - DNA, 81 bp, Artificial Sequence
  smMIP
  misc_feature (28)..(35): n is a, c, g, or t
gcagttcacaaatgaggagaaacggacnnnnnnnncttcagcttcccgatatccgacggtagtgttgcgggagatcatccg

SEQ ID NO: 341 - DNA, 83 bp, Artificial Sequence
  smMIP
  misc_feature (29)..(36): n is a, c, g, or t
gctttggtctagaaggctgcgaggtactnnnnnnnncttcagcttcccgatatccgacggtagtgtagaagtttgagacccct

SEQ ID NO: 342 - DNA, 83 bp, Artificial Sequence
  smMIP
  misc_feature (27)..(34): n is a, c, g, or t
gagggcggctgaacgtgcttgcaaatnnnnnnnncttcagcttcccgatatccgacggtagtgtgaagtggtcctctgagaag

SEQ ID NO: 343 - DNA, 82 bp, Artificial Sequence
  smMIP
  misc_feature (29)..(36): n is a, c, g, or t
gctgatgagggctccggagatgtgaagtnnnnnnnncttcagcttcccgatatccgacggtagtgtgagaatggcgtggact

SEQ ID NO: 344 - DNA, 83 bp, Artificial Sequence
  smMIP
  misc_feature (25)..(32): n is a, c, g, or t
cttgtccttggtggccaacccttcnnnnnnnncttcagcttcccgatatccgacggtagtgtagctggaacagatcttctgtc

SEQ ID NO: 345 - DNA, 83 bp, Artificial Sequence
  smMIP
  misc_feature (29)..(36): n is a, c, g, or t
gcgacactgaagggaaaaaggtaaggccnnnnnnnncttcagcttcccgatatccgacggtagtgtaccgcaggatcaatcgt

SEQ ID NO: 346 - DNA, 83 bp, Artificial Sequence
  smMIP
  misc_feature (29)..(36): n is a, c, g, or t
ggagttccgctcaccaacataacccagannnnnnnncttcagcttcccgatatccgacggtagtgttgaccccgtggtgatgg

SEQ ID NO: 347 - DNA, 83 bp, Artificial Sequence
  smMIP
  misc_feature (20)..(27): n is a, c, g, or t
agcatccccaaggactcgcnnnnnnnncttcagcttcccgatatccgacggtagtgttgtttctaggtcgtggcgtcgggctt

SEQ ID NO: 348 - DNA, 83 bp, Artificial Sequence
  smMIP
  misc_feature (30)..(37): n is a, c, g, or t
gagtggatgaagtggcgaagaagaaatctnnnnnnnncttcagcttcccgatatccgacggtagtgtcgagtacagtgcgagt

SEQ ID NO: 349 - DNA, 83 bp, Artificial Sequence
  smMIP
  misc_feature (30)..(37): n is a, c, g, or t
gcaagggccaggtcaaggaggaaggtatcnnnnnnnncttcagcttcccgatatccgacggtagtgtcaagagccttcaggag

SEQ ID NO: 350 - DNA, 83 bp, Artificial Sequence
  smMIP
  misc_feature (26)..(33): n is a, c, g, or t
gctggacatcgaggtggcctacagtnnnnnnnncttcagcttcccgatatccgacggtagtgtctctcagatcctggatctct

SEQ ID NO: 351 - DNA, 83 bp, Artificial Sequence
  smMIP
  misc_feature (29)..(36): n is a, c, g, or t
ccttcagctaacattagtctggatggtgnnnnnnnncttcagcttcccgatatccgacggtagtgtggcttaatcctgttggg

SEQ ID NO: 352 - DNA, 82 bp, Artificial Sequence
  smMIP
  misc_feature (29)..(36): n is a, c, g, or t
gtggatgtggcagctgattccatgtctgnnnnnnnncttcagcttcccgatatccgacggtagtgtaccatgctggtcagct

SEQ ID NO: 353 - DNA, 83 bp, Artificial Sequence
  smMIP
  misc_feature (23)..(30): n is a, c, g, or t
gcatgagggtggtgcacagctannnnnnnncttcagcttcccgatatccgacggtagtgtttgctgcgggtgttcccgttgtc

SEQ ID NO: 354 - DNA, 83 bp, Artificial Sequence
  smMIP
  misc_feature (27)..(34): n is a, c, g, or t
ccaaaagctgttgcactacctcggccnnnnnnnncttcagcttcccgatatccgacggtagtgtaagaccaacatcgccttcc

SEQ ID NO: 355 - DNA, 82 bp, Artificial Sequence
  smMIP
  misc_feature (28)..(35): n is a, c, g, or t
gcgcgtgtttgactacagtgagtactgnnnnnnnncttcagcttcccgatatccgacggtagtgtaggctggagctgatgtg

SEQ ID NO: 356 - DNA, 82 bp, Artificial Sequence
  smMIP
  misc_feature (29)..(36): n is a, c, g, or t
caaggacacccaggccatgaaggagatgnnnnnnnncttcagcttcccgatatccgacggtagtgttgaggtggagctggag

SEQ ID NO: 357 - DNA, 83 bp, Artificial Sequence
  smMIP
  misc_feature (29)..(36): n is a, c, g, or t
gaaattaagaaatgtgaccttcaccggcnnnnnnnncttcagcttcccgatatccgacggtagtgtcactgcctgtgcttcat

SEQ ID NO: 358 - DNA, 83 bp, Artificial Sequence
  smMIP
  misc_feature (27)..(34): n is a, c, g, or t
gctcacggctttactttcacccggggnnnnnnnncttcagcttcccgatatccgacggtagtgtcagatcagctgtataaaca

SEQ ID NO: 359 - DNA, 83 bp, Artificial Sequence
  smMIP
  misc_feature (30)..(37): n is a, c, g, or t
gtggaaattaccttgtattttcatctgtgnnnnnnnncttcagcttcccgatatccgacggtagtgtctgggcgctgggattg

SEQ ID NO: 360 - DNA, 83 bp, Artificial Sequence
  smMIP
  misc_feature (30)..(37): n is a, c, g, or t
gtagatctgggaaggggcccatcctgatgnnnnnnnncttcagcttcccgatatccgacggtagtgttgttgagagagcggca

SEQ ID NO: 361 - DNA, 83 bp, Artificial Sequence
  smMIP
  misc_feature (30)..(37): n is a, c, g, or t
ggaagagctgggctaccacatctactccannnnnnnncttcagcttcccgatatccgacggtagtgtaaggacaggatggtga

SEQ ID NO: 362 - DNA, 83 bp, Artificial Sequence
  smMIP
  misc_feature (26)..(33): n is a, c, g, or t
gataatcttctcaggacaccatccgnnnnnnnncttcagcttcccgatatccgacggtagtgtctctccatgaagcagttcct

SEQ ID NO: 363 - DNA, 83 bp, Artificial Sequence
  smMIP
  misc_feature (27)..(34): n is a, c, g, or t
gcaagatgatctttacagatactgtgnnnnnnnncttcagcttcccgatatccgacggtagtgtctggtatatccagagtctt

SEQ ID NO: 364 - DNA, 83 bp, Artificial Sequence
  smMIP
  misc_feature (26)..(33): n is a, c, g, or t
gctatgaaaatgctaggcgtctgtgnnnnnnnncttcagcttcccgatatccgacggtagtgtcaattagaatgttactcaat

SEQ ID NO: 365 - DNA, 83 bp, Artificial Sequence
  smMIP
  misc_feature (30)..(37): n is a, c, g, or t
gcgttcctttgaggaaaattgacagacttnnnnnnnncttcagcttcccgatatccgacggtagtgtcaagaatgcaatgaga

SEQ ID NO: 366 - DNA, 83 bp, Artificial Sequence
  smMIP
  misc_feature (26)..(33): n is a, c, g, or t
gcctggaagcattacaacaccaaccnnnnnnnncttcagcttcccgatatccgacggtagtgttacgcacaatacttccaagg

SEQ ID NO: 367 - DNA, 83 bp, Artificial Sequence
  smMIP
  misc_feature (30)..(37): n is a, c, g, or t
tgcgccctggcagccctgaaggatgttcannnnnnnncttcagcttcccgatatccgacggtagtgtaagaaaaaacctctag

SEQ ID NO: 368 - DNA, 83 bp, Artificial Sequence
  smMIP
  misc_feature (30)..(37): n is a, c, g, or t
gaggaactggacagccacctgtcctacatnnnnnnnncttcagcttcccgatatccgacggtagtgtcagaaaacatcaggca

SEQ ID NO: 369 - DNA, 83 bp, Artificial Sequence
  smMIP
  misc_feature (29)..(36): n is a, c, g, or t
gtcacatgaagaggaccatccagacagcnnnnnnnncttcagcttcccgatatccgacggtagtgtaactcaacatcagaggc

SEQ ID NO: 370 - DNA, 83 bp, Artificial Sequence
  smMIP
  misc_feature (30)..(37): n is a, c, g, or t
gctgtcatgcggtgcctcctggcctatttnnnnnnnncttcagcttcccgatatccgacggtagtgtaccaagataaatatcg

SEQ ID NO: 371 - DNA, 83 bp, Artificial Sequence
  smMIP
  misc_feature (20)..(27): n is a, c, g, or t
acatcacccgggaacctgannnnnnnncttcagcttcccgatatccgacggtagtgtagcttccatatctcaagtgccctctg

SEQ ID NO: 372 - DNA, 83 bp, Artificial Sequence
  smMIP
  misc_feature (30)..(37): n is a, c, g, or t
gctggggaagcttctacttccagcatgctnnnnnnnncttcagcttcccgatatccgacggtagtgtaaccgccttcacagca

SEQ ID NO: 373 - DNA, 83 bp, Artificial Sequence
  smMIP
  misc_feature (27)..(34): n is a, c, g, or t
gtggagtgacttgttgagtgacctccnnnnnnnncttcagcttcccgatatccgacggtagtgtaaggactttcgggaacgag

SEQ ID NO: 374 - DNA, 83 bp, Artificial Sequence
  smMIP
  misc_feature (26)..(33): n is a, c, g, or t
gcaggatgggtgtggaagcagtgatnnnnnnnncttcagcttcccgatatccgacggtagtgtaaccaatcacctcagaagac

SEQ ID NO: 375 - DNA, 83 bp, Artificial Sequence
  smMIP
  misc_feature (30)..(37): n is a, c, g, or t
cattgggggctttgaggcttacacaggggnnnnnnnncttcagcttcccgatatccgacggtagtgttatgttgggggctgga

SEQ ID NO: 376 - DNA, 82 bp, Artificial Sequence
  smMIP
  misc_feature (28)..(35): n is a, c, g, or t
gctgaaggaccagacagattttgagcannnnnnnncttcagcttcccgatatccgacggtagtgttgaactggatgtctggg

SEQ ID NO: 377 - DNA, 81 bp, Artificial Sequence
  smMIP
  misc_feature (28)..(35): n is a, c, g, or t
gatgtggctgccagtggtgaggacttgnnnnnnnncttcagcttcccgatatccgacggtagtgtacgaggaggcgaagcg

SEQ ID NO: 378 - DNA, 83 bp, Artificial Sequence
  smMIP
  misc_feature (28)..(35): n is a, c, g, or t
cgcaggtatgcagacctcacagaagatnnnnnnnncttcagcttcccgatatccgacggtagtgtcaataaagcagaaactgc

SEQ ID NO: 379 - DNA, 83 bp, Artificial Sequence
  smMIP
  misc_feature (30)..(37): n is a, c, g, or t
cagatcaaggaggggaaacgtgtactgatnnnnnnnncttcagcttcccgatatccgacggtagtgtacagcaacatcagtaa

SEQ ID NO: 380 - DNA, 82 bp, Artificial Sequence
  smMIP
  misc_feature (26)..(33): n is a, c, g, or t
gcgcaaagccatggaagctgtggctnnnnnnnncttcagcttcccgatatccgacggtagtgtggagggtctctctgaagag

SEQ ID NO: 381 - DNA, 83 bp, Artificial Sequence
  smMIP
  misc_feature (21)..(28): n is a, c, g, or t
gccggcggggaggatactgtnnnnnnnncttcagcttcccgatatccgacggtagtgtggtattcccattgtctatgaattgg

SEQ ID NO: 382 - DNA, 83 bp, Artificial Sequence
  smMIP
  misc_feature (30)..(37): n is a, c, g, or t
gccaatgaggcaaagggaaccaaagtggtnnnnnnnncttcagcttcccgatatccgacggtagtgtaagctgacatcgcgct

SEQ ID NO: 383 - DNA, 83 bp, Artificial Sequence
  smMIP
  misc_feature (23)..(30): n is a, c, g, or t
gctgcaaaagtgggaactggagnnnnnnnncttcagcttcccgatatccgacggtagtgtgccaagggaattttatttgtggg

SEQ ID NO: 384 - DNA, 83 bp, Artificial Sequence
  smMIP
  misc_feature (30)..(37): n is a, c, g, or t
cccgtcaccctcattggagaagctgtcttnnnnnnnncttcagcttcccgatatccgacggtagtgtcagccaatattctcaa

SEQ ID NO: 385 - DNA, 83 bp, Artificial Sequence
  smMIP
  misc_feature (26)..(33): n is a, c, g, or t
gtcagctgttgaaaactgccaggacnnnnnnnncttcagcttcccgatatccgacggtagtgtttatggtggcatcgccctga

SEQ ID NO: 386 - DNA, 83 bp, Artificial Sequence
  smMIP
  misc_feature (24)..(31): n is a, c, g, or t
ccagggcagtttatccacaccaannnnnnnncttcagcttcccgatatccgacggtagtgtttcccatgccctgttttaccac

SEQ ID NO: 387 - DNA, 83 bp, Artificial Sequence
  smMIP
  misc_feature (21)..(28): n is a, c, g, or t
accgcttcaaccacttcagcnnnnnnnncttcagcttcccgatatccgacggtagtgtctcactcagtgtaccttctagtccc

SEQ ID NO: 388 - DNA, 83 bp, Artificial Sequence
  smMIP
  misc_feature (26)..(33): n is a, c, g, or t
gtggtttctccaggcggccaaggatnnnnnnnncttcagcttcccgatatccgacggtagtgtcaacattgatggaactcaca

SEQ ID NO: 389 - DNA, 83 bp, Artificial Sequence
  smMIP
  misc_feature (29)..(36): n is a, c, g, or t
gctgggtatctggtacatcaactgctttnnnnnnnncttcagcttcccgatatccgacggtagtgtactctccattgccctgc

SEQ ID NO: 390 - DNA, 82 bp, Artificial Sequence
  smMIP
  misc_feature (25)..(32): n is a, c, g, or t
gcatcacaagatcctcctggccaannnnnnnncttcagcttcccgatatccgacggtagtgtttgtgtggggggagccaggg

SEQ ID NO: 391 - DNA, 83 bp, Artificial Sequence
  smMIP
  misc_feature (30)..(37): n is a, c, g, or t
gctggctaagaaaatagagcctgagcttgnnnnnnnncttcagcttcccgatatccgacggtagtgtaccaagctcacaccat

SEQ ID NO: 392 - DNA, 83 bp, Artificial Sequence
  smMIP
  misc_feature (27)..(34): n is a, c, g, or t
caaccagaggattaaggctgctgtccnnnnnnnncttcagcttcccgatatccgacggtagtgtcccagctgtatttccaaaa

SEQ ID NO: 393 - DNA, 83 bp, Artificial Sequence
  smMIP
  misc_feature (27)..(34): n is a, c, g, or t
gctggagaacctccgctttcatgtggnnnnnnnncttcagcttcccgatatccgacggtagtgttccttagagccagttgctg

SEQ ID NO: 394 - DNA, 83 bp, Artificial Sequence
  smMIP
  misc_feature (23)..(30): n is a, c, g, or t
gcagacaagatccagctcatcannnnnnnncttcagcttcccgatatccgacggtagtgttccatggtaggagtcaatctgcc

SEQ ID NO: 395 - DNA, 83 bp, Artificial Sequence
  smMIP
  misc_feature (26)..(33): n is a, c, g, or t
gctggctggatgggcttggactgtgnnnnnnnncttcagcttcccgatatccgacggtagtgtaaagacctaatgtccaaagc

SEQ ID NO: 396 - DNA, 83 bp, Artificial Sequence
  smMIP
  misc_feature (30)..(37): n is a, c, g, or t
gtggtgccagtttggagctcctggaaggtnnnnnnnncttcagcttcccgatatccgacggtagtgtcatggatgaggtggtg

SEQ ID NO: 397 - DNA, 83 bp, Artificial Sequence
  smMIP
  misc_feature (27)..(34): n is a, c, g, or t
ccagattacaaacaaccagaggatcannnnnnnncttcagcttcccgatatccgacggtagtgtggtgtcagcctatgtcttt

SEQ ID NO: 398 - DNA, 83 bp, Artificial Sequence
  smMIP
  misc_feature (27)..(34): n is a, c, g, or t
gttcctgaaggactgtgtaggcgcagnnnnnnnncttcagcttcccgatatccgacggtagtgtaatggagccaaggcagtag

SEQ ID NO: 399 - DNA, 83 bp, Artificial Sequence
  smMIP
  misc_feature (28)..(35): n is a, c, g, or t
gctaaagccttggaaaacccagtgagannnnnnnncttcagcttcccgatatccgacggtagtgtgctaggggacgtctatgt

SEQ ID NO: 400 - DNA, 83 bp, Artificial Sequence
  smMIP
  misc_feature (30)..(37): n is a, c, g, or t
gtttgacgagaacgctcaggttggaaaagnnnnnnnncttcagcttcccgatatccgacggtagtgtatggagattggtgctt

SEQ ID NO: 401 - DNA, 83 bp, Artificial Sequence
  smMIP
  misc_feature (27)..(34): n is a, c, g, or t
gataaagtcagccatgtcagcactggnnnnnnnncttcagcttcccgatatccgacggtagtgtgggatgcctttgctaaggg

SEQ ID NO: 402 - DNA, 83 bp, Artificial Sequence
  smMIP
  misc_feature (20)..(27): n is a, c, g, or t
cccaaccccagagaaccaannnnnnnncttcagcttcccgatatccgacggtagtgtagaagtccccagcgccgttccttcca

SEQ ID NO: 403 - DNA, 83 bp, Artificial Sequence
  smMIP
  misc_feature (25)..(32): n is a, c, g, or t
cactaaaggacctgagatccgaacnnnnnnnncttcagcttcccgatatccgacggtagtgttggctcgtctgaacttctctc

SEQ ID NO: 404 - DNA, 83 bp, Artificial Sequence
  smMIP
  misc_feature (27)..(34): n is a, c, g, or t
gcaggatgttgatatggtgtttgcgtnnnnnnnncttcagcttcccgatatccgacggtagtgtctggtgacggaggtggaaa

SEQ ID NO: 405 - DNA, 83 bp, Artificial Sequence
  smMIP
  misc_feature (24)..(31): n is a, c, g, or t
gccaaaggggactatcctctggannnnnnnncttcagcttcccgatatccgacggtagtgtctgtcatctgtgctactcagat

SEQ ID NO: 406 - DNA, 83 bp, Artificial Sequence
  smMIP
  misc_feature (30)..(37): n is a, c, g, or t
acctccgggtgaactttgccatgaatgttnnnnnnnncttcagcttcccgatatccgacggtagtgtacgtgcccccatcatt

SEQ ID NO: 407 - DNA, 83 bp, Artificial Sequence
  smMIP
  misc_feature (26)..(33): n is a, c, g, or t
gttgtgttcttcttttaccctcttgnnnnnnnncttcagcttcccgatatccgacggtagtgtgctgataggaagatgtcttc

SEQ ID NO: 408 - DNA, 83 bp, Artificial Sequence
  smMIP
  misc_feature (28)..(35): n is a, c, g, or t
cgaagcgcaccattgctcaggattatgnnnnnnnncttcagcttcccgatatccgacggtagtgtaagaaactcaactgccaa

SEQ ID NO: 409 - DNA, 83 bp, Artificial Sequence
  smMIP
  misc_feature (27)..(34): n is a, c, g, or t
gcagatcactgtaaatgacctccctgnnnnnnnncttcagcttcccgatatccgacggtagtgtcccatgaacattcctttgg

SEQ ID NO: 410 - DNA, 83 bp, Artificial Sequence
  smMIP
  misc_feature (26)..(33): n is a, c, g, or t
acaaacatggggaagtgtgcccagcnnnnnnnncttcagcttcccgatatccgacggtagtgttctcgttcaggggccttttt

SEQ ID NO: 411 - DNA, 83 bp, Artificial Sequence
  smMIP
  misc_feature (25)..(32): n is a, c, g, or t
gctgggctgttttagtgccaggctnnnnnnnncttcagcttcccgatatccgacggtagtgttagttcaggccttccagttca

SEQ ID NO: 412 - DNA, 83 bp, Artificial Sequence
  smMIP
  misc_feature (27)..(34): n is a, c, g, or t
cagaacctcaagcttttcaggcatccnnnnnnnncttcagcttcccgatatccgacggtagtgttcggcaccttcggcaaagt

SEQ ID NO: 413 - DNA, 83 bp, Artificial Sequence
  smMIP
  misc_feature (27)..(34): n is a, c, g, or t
cacccaactatgctgcaccagaagtannnnnnnncttcagcttcccgatatccgacggtagtgtggtggtccatagagatttg

SEQ ID NO: 414 - DNA, 83 bp, Artificial Sequence
  smMIP
  misc_feature (30)..(37): n is a, c, g, or t
gagtgctcagaagaggaagttctcagctgnnnnnnnncttcagcttcccgatatccgacggtagtgtaagatatcagggaaca

SEQ ID NO: 415 - DNA, 83 bp, Artificial Sequence
  smMIP
  misc_feature (30)..(37): n is a, c, g, or t
ggattatgaatggaaggttgtaaacccatnnnnnnnncttcagcttcccgatatccgacggtagtgtgaattaaatccacaga

SEQ ID NO: 416 - DNA, 83 bp, Artificial Sequence
  smMIP
  misc_feature (30)..(37): n is a, c, g, or t
gtgcaaatctaattaaaattcttgcacaannnnnnnncttcagcttcccgatatccgacggtagtgttgctgaggctcaagga

SEQ ID NO: 417 - DNA, 83 bp, Artificial Sequence
  smMIP
  misc_feature (30)..(37): n is a, c, g, or t
cgagaaattcaaaatctaaaactctttcgnnnnnnnncttcagcttcccgatatccgacggtagtgttgggcgtcggcacctt

SEQ ID NO: 418 - DNA, 83 bp, Artificial Sequence
  smMIP
  misc_feature (30)..(37): n is a, c, g, or t
gccaagatagccgatttcggattatctaannnnnnnncttcagcttcccgatatccgacggtagtgtaagagatggaagccag

SEQ ID NO: 419 - DNA, 83 bp, Artificial Sequence
  smMIP
  misc_feature (19)..(26): n is a, c, g, or t
gctgcaggttgacccactnnnnnnnncttcagcttcccgatatccgacggtagtgttgtatgctcttctttgtggcaccctcc

SEQ ID NO: 420 - DNA, 83 bp, Artificial Sequence
  smMIP
  misc_feature (24)..(31): n is a, c, g, or t
gcagacagccccaaagcaagatgnnnnnnnncttcagcttcccgatatccgacggtagtgtgaataatgaaccaagccagtga

SEQ ID NO: 421 - DNA, 83 bp, Artificial Sequence
  smMIP
  misc_feature (19)..(26): n is a, c, g, or t
ccacaactgcagagagccnnnnnnnncttcagcttcccgatatccgacggtagtgtgttgataacaggagctatcttttggac

SEQ ID NO: 422 - DNA, 82 bp, Artificial Sequence
  smMIP
  misc_feature (26)..(33): n is a, c, g, or t
ctacaattgtccatgctgtgcagcgnnnnnnnncttcagcttcccgatatccgacggtagtgtactccaacagcatctgccc

SEQ ID NO: 423 - DNA, 83 bp, Artificial Sequence
  smMIP
  misc_feature (22)..(29): n is a, c, g, or t
ctcaatctcatcaagggtggcnnnnnnnncttcagcttcccgatatccgacggtagtgtcctcgtctgtattcccacttcctt

SEQ ID NO: 424 - DNA, 83 bp, Artificial Sequence
  smMIP
  misc_feature (23)..(30): n is a, c, g, or t
gctgtgagccagaagtttggggnnnnnnnncttcagcttcccgatatccgacggtagtgtgtggctggctatgctagtcgctt

SEQ ID NO: 425 - DNA, 83 bp, Artificial Sequence
  smMIP
  misc_feature (29)..(36): n is a, c, g, or t
gtttgaccgggtacacaaatggagtgaannnnnnnncttcagcttcccgatatccgacggtagtgtggcctatgtcccagtga

SEQ ID NO: 426 - DNA, 83 bp, Artificial Sequence
  smMIP
  misc_feature (30)..(37): n is a, c, g, or t
ggagcagagtgtgttcaccttgagtctccnnnnnnnncttcagcttcccgatatccgacggtagtgtatcaaaatgatcccag

SEQ ID NO: 427 - DNA, 82 bp, Artificial Sequence
  smMIP
  misc_feature (29)..(36): n is a, c, g, or t
gggcatctgctaaagtttcagattccatnnnnnnnncttcagcttcccgatatccgacggtagtgttgtcgggggtccgggg

SEQ ID NO: 428 - DNA, 83 bp, Artificial Sequence
  smMIP
  misc_feature (28)..(35): n is a, c, g, or t
gcatttggcctttctgaggcagggtttnnnnnnnncttcagcttcccgatatccgacggtagtgtcactgttgatgggaacaa

SEQ ID NO: 429 - DNA, 83 bp, Artificial Sequence
  smMIP
  misc_feature (29)..(36): n is a, c, g, or t
gcacagctagaaaattatggcatgccgtnnnnnnnncttcagcttcccgatatccgacggtagtgttttgatgcagtggtggt

SEQ ID NO: 430 - DNA, 83 bp, Artificial Sequence
  smMIP
  misc_feature (27)..(34): n is a, c, g, or t
gcctcaagtttggaaagggcgggcagnnnnnnnncttcagcttcccgatatccgacggtagtgtagcatgtgttaccaagctg

SEQ ID NO: 431 - DNA, 83 bp, Artificial Sequence
  smMIP
  misc_feature (29)..(36): n is a, c, g, or t
gcgatatgataccagctattttgtggagnnnnnnnncttcagcttcccgatatccgacggtagtgtttatcagcgtgcatttg

SEQ ID NO: 432 - DNA, 82 bp, Artificial Sequence
  smMIP
  misc_feature (24)..(31): n is a, c, g, or t
gcatagaggacgggtccatccatnnnnnnnncttcagcttcccgatatccgacggtagtgtgactggccactcgctattgca

SEQ ID NO: 433 - DNA, 83 bp, Artificial Sequence
  smMIP
  misc_feature (20)..(27): n is a, c, g, or t
cttcagctgcacgtctgccnnnnnnnncttcagcttcccgatatccgacggtagtgtttttgccttggatctcctgatggaga

SEQ ID NO: 434 - DNA, 83 bp, Artificial Sequence
  smMIP
  misc_feature (30)..(37): n is a, c, g, or t
gttcagttccaccctacaggcatatatggnnnnnnnncttcagcttcccgatatccgacggtagtgtctgttgttgccacagg

SEQ ID NO: 435 - DNA, 82 bp, Artificial Sequence
  smMIP
  misc_feature (28)..(35): n is a, c, g, or t
gcgaaaggtttatggagcgatacgcccnnnnnnnncttcagcttcccgatatccgacggtagtgtagggcaggccttccttg

SEQ ID NO: 436 - DNA, 83 bp, Artificial Sequence
  smMIP
  misc_feature (29)..(36): n is a, c, g, or t
cgagaaggaagaggctgtggccctgagannnnnnnncttcagcttcccgatatccgacggtagtgttgtcgtggagagggagg

SEQ ID NO: 437 - DNA, 83 bp, Artificial Sequence
  smMIP
  misc_feature (22)..(29): n is a, c, g, or t
gcctggcatttcagagacagcnnnnnnnncttcagcttcccgatatccgacggtagtgttggcgtctagagatgtggtgtctc

SEQ ID NO: 438 - DNA, 82 bp, Artificial Sequence
  smMIP
  misc_feature (23)..(30): n is a, c, g, or t
gcggcattcccaccaactacaannnnnnnncttcagcttcccgatatccgacggtagtgttgcaccacctacctccagagca

SEQ ID NO: 439 - DNA, 83 bp, Artificial Sequence
  smMIP
  misc_feature (21)..(28): n is a, c, g, or t
gcctcggtacatggtgccaannnnnnnncttcagcttcccgatatccgacggtagtgttccctgtcctccccaccgtgcatta

SEQ ID NO: 440 - DNA, 83 bp, Artificial Sequence
  smMIP
  misc_feature (28)..(35): n is a, c, g, or t
gcctggagataaagtccctccaattaannnnnnnncttcagcttcccgatatccgacggtagtgttgtacgcctgtggggagg

SEQ ID NO: 441 - DNA, 83 bp, Artificial Sequence
  smMIP
  misc_feature (26)..(33): n is a, c, g, or t
gctgatggaagcataagaacatcggnnnnnnnncttcagcttcccgatatccgacggtagtgtctggttgtctttggtcgggc

SEQ ID NO: 442 - DNA, 83 bp, Artificial Sequence
  smMIP
  misc_feature (25)..(32): n is a, c, g, or t
gcgtgttgcaagaaggttgtgggannnnnnnncttcagcttcccgatatccgacggtagtgtgctggggaagaatctgtcatg

SEQ ID NO: 443 - DNA, 83 bp, Artificial Sequence
  smMIP
  misc_feature (26)..(33): n is a, c, g, or t
gtctggaacacggacctggtggagannnnnnnncttcagcttcccgatatccgacggtagtgtatgcagaagtcaatgcaaaa

SEQ ID NO: 444 - DNA, 83 bp, Artificial Sequence
  smMIP
  misc_feature (22)..(29): n is a, c, g, or t
cagaggcacggaaggagtcacnnnnnnnncttcagcttcccgatatccgacggtagtgtatcagcaagctctatggagaccta

SEQ ID NO: 445 - DNA, 83 bp, Artificial Sequence
  smMIP
  misc_feature (25)..(32): n is a, c, g, or t
gcccatccaggggcaacagaagaannnnnnnncttcagcttcccgatatccgacggtagtgttggagctgcagaacctgatgc

SEQ ID NO: 446 - DNA, 83 bp, Artificial Sequence
  smMIP
  misc_feature (30)..(37): n is a, c, g, or t
ggaaggtcactctggaatatagacccgtgnnnnnnnncttcagcttcccgatatccgacggtagtgtggaagactacaaggtg

SEQ ID NO: 447 - DNA, 83 bp, Artificial Sequence
  smMIP
  misc_feature (26)..(33): n is a, c, g, or t
gtggtgatgacagaatcagcttttgnnnnnnnncttcagcttcccgatatccgacggtagtgtgtcctatgtggacgttggca

SEQ ID NO: 448 - DNA, 83 bp, Artificial Sequence
  smMIP
  misc_feature (28)..(35): n is a, c, g, or t
cccagacaaggctggagacaaacctcannnnnnnncttcagcttcccgatatccgacggtagtgttctccttgaggcgccggt

SEQ ID NO: 449 - DNA, 83 bp, Artificial Sequence
  smMIP
  misc_feature (28)..(35): n is a, c, g, or t
tgactctactttgaccttccgaagatcnnnnnnnncttcagcttcccgatatccgacggtagtgtttgccatctatcgatggg

SEQ ID NO: 450 - DNA, 83 bp, Artificial Sequence
  smMIP
  misc_feature (27)..(34): n is a, c, g, or t
cactctagcttgcacccgaaggattgnnnnnnnncttcagcttcccgatatccgacggtagtgttgtggccccatggtattgg

SEQ ID NO: 451 - DNA, 83 bp, Artificial Sequence
  smMIP
  misc_feature (30)..(37): n is a, c, g, or t
gatcttgttcccgatttgagcaacttctannnnnnnncttcagcttcccgatatccgacggtagtgttctgtggctcttgtgc

SEQ ID NO: 452 - DNA, 83 bp, Artificial Sequence
  smMIP
  misc_feature (25)..(32): n is a, c, g, or t
gcagcagtatctgcagtccatagannnnnnnncttcagcttcccgatatccgacggtagtgtcaaaaatctaccctcttccac

SEQ ID NO: 453 - DNA, 83 bp, Artificial Sequence
  smMIP
  misc_feature (30)..(37): n is a, c, g, or t
gctactggtggaacggagacaaatatctgnnnnnnnncttcagcttcccgatatccgacggtagtgtgaagaagaaggatgaa

SEQ ID NO: 454 - DNA, 83 bp, Artificial Sequence
  smMIP
  misc_feature (20)..(27): n is a, c, g, or t
agcgcctggccaagctgcannnnnnnncttcagcttcccgatatccgacggtagtgtagtgcattctctgtgcctgctgtagc

SEQ ID NO: 455 - DNA, 82 bp, Artificial Sequence
  smMIP
  misc_feature (29)..(36): n is a, c, g, or t
gcagagatcaagaaaatgatggcaacctnnnnnnnncttcagcttcccgatatccgacggtagtgtagagatgacttcacag

SEQ ID NO: 456 - DNA, 83 bp, Artificial Sequence
  smMIP
  misc_feature (19)..(26): n is a, c, g, or t
ccagctcagagctgaacannnnnnnncttcagcttcccgatatccgacggtagtgtatgaactgcacaaggacctgtcctaag

SEQ ID NO: 457 - DNA, 82 bp, Artificial Sequence
  smMIP
  misc_feature (29)..(36): n is a, c, g, or t
gctttgagtgcaggggtctctctttttgnnnnnnnncttcagcttcccgatatccgacggtagtgtgaagagatggagcggt

SEQ ID NO: 458 - DNA, 83 bp, Artificial Sequence
  smMIP
  misc_feature (21)..(28): n is a, c, g, or t
gcactgatccacacagctaannnnnnnncttcagcttcccgatatccgacggtagtgttgtccatctgccaccgtggcactgg

SEQ ID NO: 459 - DNA, 83 bp, Artificial Sequence
  smMIP
  misc_feature (24)..(31): n is a, c, g, or t
gcctgaagattccccagctatacnnnnnnnncttcagcttcccgatatccgacggtagtgttttggaacttgtgaagtccctg

SEQ ID NO: 460 - DNA, 83 bp, Artificial Sequence
  smMIP
  misc_feature (23)..(30): n is a, c, g, or t
cccagcatcatcttcctacacannnnnnnncttcagcttcccgatatccgacggtagtgtatccgacacttgatgtgggacct

SEQ ID NO: 461 - DNA, 83 bp, Artificial Sequence
  smMIP
  misc_feature (24)..(31): n is a, c, g, or t
cacttgtcaccgagccaccattcnnnnnnnncttcagcttcccgatatccgacggtagtgttgagtgccgtttgcggtgccct

SEQ ID NO: 462 - DNA, 83 bp, Artificial Sequence
  smMIP
  misc_feature (29)..(36): n is a, c, g, or t
gtctgcttccggctgcttatttgaatccnnnnnnnncttcagcttcccgatatccgacggtagtgtaccgacctatcccagaa

SEQ ID NO: 463 - DNA, 83 bp, Artificial Sequence
  smMIP
  misc_feature (29)..(36): n is a, c, g, or t
gttgttactgactatgttcatggggatgnnnnnnnncttcagcttcccgatatccgacggtagtgtgagagggttgtcagtgt

SEQ ID NO: 464 - DNA, 83 bp, Artificial Sequence
  smMIP
  misc_feature (27)..(34): n is a, c, g, or t
gctatttcaactatcacgatgtgggcnnnnnnnncttcagcttcccgatatccgacggtagtgtcctcactcttcatggtcac

SEQ ID NO: 465 - DNA, 83 bp, Artificial Sequence
  smMIP
  misc_feature (26)..(33): n is a, c, g, or t
gtatgcctctttgcctctgctttgtnnnnnnnncttcagcttcccgatatccgacggtagtgttggcactttcagctttaacc

SEQ ID NO: 466 - DNA, 82 bp, Artificial Sequence
  smMIP
  misc_feature (23)..(30): n is a, c, g, or t
cggcttctcttatgcatttcccnnnnnnnncttcagcttcccgatatccgacggtagtgtttggatttgacctgcattttgg

SEQ ID NO: 467 - DNA, 83 bp, Artificial Sequence
  smMIP
  misc_feature (26)..(33): n is a, c, g, or t
cgtctgtattggagtcattggaggtnnnnnnnncttcagcttcccgatatccgacggtagtgttggaggtcctatcagcagta

SEQ ID NO: 468 - DNA, 82 bp, Artificial Sequence
  smMIP
  misc_feature (28)..(35): n is a, c, g, or t
cagatcttattggaagacaccctaaacnnnnnnnncttcagcttcccgatatccgacggtagtgtgttgctggagccctcat

SEQ ID NO: 469 - DNA, 83 bp, Artificial Sequence
  smMIP
  misc_feature (30)..(37): n is a, c, g, or t
gttggattctgtgtctatgcgggattcttnnnnnnnncttcagcttcccgatatccgacggtagtgtgaccatctatgggact

SEQ ID NO: 470 - DNA, 78 bp, Artificial Sequence
  smMIP
  misc_feature (24)..(31): n is a, c, g, or t
ggagggcccaaggaggaggaaagnnnnnnnncttcagcttcccgatatccgacggtagtgttggcaaaagaacagaaa

SEQ ID NO: 471 - DNA, 83 bp, Artificial Sequence
  smMIP
  misc_feature (19)..(26): n is a, c, g, or t
gcggctttgtgctttacgnnnnnnnncttcagcttcccgatatccgacggtagtgtatcctgggcggcctgctgctcaactgc

SEQ ID NO: 472 - DNA, 81 bp, Artificial Sequence
  smMIP
  misc_feature (23)..(30): n is a, c, g, or t
gctctgcagtgtgtgcgtgaacnnnnnnnncttcagcttcccgatatccgacggtagtgtcttctcctacgccttccccaa

SEQ ID NO: 473 - DNA, 83 bp, Artificial Sequence
  smMIP
  misc_feature (22)..(29): n is a, c, g, or t
cgacaccaaggccgccttcctnnnnnnnncttcagcttcccgatatccgacggtagtgtgcctgctagacctgagcgtcttcc

SEQ ID NO: 474 - DNA, 83 bp, Artificial Sequence
  smMIP
  misc_feature (26)..(33): n is a, c, g, or t
cgacccacgtctacatgtacgtgttnnnnnnnncttcagcttcccgatatccgacggtagtgttgcagttcgaggtgctcatg

SEQ ID NO: 475 - DNA, 83 bp, Artificial Sequence
  smMIP
  misc_feature (30)..(37): n is a, c, g, or t
gcatttcctgaaggctgagcctgagaaaannnnnnnncttcagcttcccgatatccgacggtagtgtctgggcaacttcttct

SEQ ID NO: 476 - DNA, 82 bp, Artificial Sequence
  smMIP
  misc_feature (20)..(27): n is a, c, g, or t
cctatgcattccccaaagcnnnnnnnncttcagcttcccgatatccgacggtagtgtctcttggtgccaacagagttactct

SEQ ID NO: 477 - DNA, 83 bp, Artificial Sequence
  smMIP
  misc_feature (29)..(36): n is a, c, g, or t
gcaacccgccttaaccataattggcaaannnnnnnncttcagcttcccgatatccgacggtagtgtggtggtgatagcaggag

SEQ ID NO: 478 - DNA, 83 bp, Artificial Sequence
  smMIP
  misc_feature (30)..(37): n is a, c, g, or t
ccctttttaagcatagaggatttctgatannnnnnnncttcagcttcccgatatccgacggtagtgtacccaatcaaaccact

SEQ ID NO: 479 - DNA, 83 bp, Artificial Sequence
  smMIP
  misc_feature (22)..(29): n is a, c, g, or t
gtgttagcagtgttctctttgnnnnnnnncttcagcttcccgatatccgacggtagtgtcgacctcgaattcagtacttcttc

SEQ ID NO: 480 - DNA, 83 bp, Artificial Sequence
  smMIP
  misc_feature (26)..(33): n is a, c, g, or t
ccttgagcaaatctaaacattcggannnnnnnncttcagcttcccgatatccgacggtagtgttgtcctgtggggctattgtg

SEQ ID NO: 481 - DNA, 83 bp, Artificial Sequence
  smMIP
  misc_feature (30)..(37): n is a, c, g, or t
gctctggtccctctcagtggccatcttttnnnnnnnncttcagcttcccgatatccgacggtagtgttccctgcagtttggct

SEQ ID NO: 482 - DNA, 83 bp, Artificial Sequence
  smMIP
  misc_feature (19)..(26): n is a, c, g, or t
tcacccacagcccttcgtnnnnnnnncttcagcttcccgatatccgacggtagtgtcttcgtgtccgccgtgctcatgggctt

SEQ ID NO: 483 - DNA, 78 bp, Artificial Sequence
  smMIP
  misc_feature (24)..(31): n is a, c, g, or t
gcatcttcgagaaggcgggggtgnnnnnnnncttcagcttcccgatatccgacggtagtgtggagaagaaggtcacca

SEQ ID NO: 484 - DNA, 81 bp, Artificial Sequence
  smMIP
  misc_feature (25)..(32): n is a, c, g, or t
gccccatcccatggttcatcgtggnnnnnnnncttcagcttcccgatatccgacggtagtgtgctggcatggcgggttgtg

SEQ ID NO: 485 - DNA, 83 bp, Artificial Sequence
  smMIP
  misc_feature (19)..(26): n is a, c, g, or t
ttccatcccctgggggctnnnnnnnncttcagcttcccgatatccgacggtagtgttgtgctcctggttctgttcttcatctt

SEQ ID NO: 486 - DNA, 83 bp, Artificial Sequence
  smMIP
  misc_feature (28)..(35): n is a, c, g, or t
cactggggtcatcaatgctcctgagaannnnnnnncttcagcttcccgatatccgacggtagtgtatcacccctagatctttc

SEQ ID NO: 487 - DNA, 78 bp, Artificial Sequence
  smMIP
  misc_feature (20)..(27): n is a, c, g, or t
gccctgcggggtgcctttgnnnnnnnncttcagcttcccgatatccgacggtagtgtggctgctttatgggactgtgt

SEQ ID NO: 488 - DNA, 83 bp, Artificial Sequence
  smMIP
  misc_feature (27)..(34): n is a, c, g, or t
ccattgtgctccagctctctcagcagnnnnnnnncttcagcttcccgatatccgacggtagtgtcacccaggatgtatcccaa

SEQ ID NO: 489 - DNA, 83 bp, Artificial Sequence
  smMIP
  misc_feature (19)..(26): n is a, c, g, or t
actcttcagccagggcccnnnnnnnncttcagcttcccgatatccgacggtagtgtcgctcatgactgtttctttgttattaa

SEQ ID NO: 490 - DNA, 83 bp, Artificial Sequence
  smMIP
  misc_feature (25)..(32): n is a, c, g, or t
gcctgctaaggagaccaccaccaannnnnnnncttcagcttcccgatatccgacggtagtgtttaccttcttcaaagtccctg

SEQ ID NO: 491 - DNA, 83 bp, Artificial Sequence
  smMIP
  misc_feature (22)..(29): n is a, c, g, or t
cattttcaccaagatctcggcnnnnnnnncttcagcttcccgatatccgacggtagtgtttgtgctgggctggctgtttgtcc

SEQ ID NO: 492 - DNA, 83 bp, Artificial Sequence
  smMIP
  misc_feature (22)..(29): n is a, c, g, or t
catcttccgagatcccctcacnnnnnnnncttcagcttcccgatatccgacggtagtgtttgcttttcacgaagtgggaggct

SEQ ID NO: 493 - DNA, 83 bp, Artificial Sequence
  smMIP
  misc_feature (26)..(33): n is a, c, g, or t
gtcatgctggcctccctcatgagctnnnnnnnncttcagcttcccgatatccgacggtagtgtattgcctgtgtcgtcccttc

SEQ ID NO: 494 - DNA, 83 bp, Artificial Sequence
  smMIP
  misc_feature (20)..(27): n is a, c, g, or t
agcccagcaactgtcccacnnnnnnnncttcagcttcccgatatccgacggtagtgtctgtcttcctgcttgctattttctgg

SEQ ID NO: 495 - DNA, 83 bp, Artificial Sequence
  smMIP
  misc_feature (27)..(34): n is a, c, g, or t
catcctggtgaccgtggctgtcttttnnnnnnnncttcagcttcccgatatccgacggtagtgtttttgtgggctagagcagc

SEQ ID NO: 496 - DNA, 83 bp, Artificial Sequence
  smMIP
  misc_feature (23)..(30): n is a, c, g, or t
gccagcaagcagatcactgcagnnnnnnnncttcagcttcccgatatccgacggtagtgttctttcaacatgacagatgccgc

SEQ ID NO: 497 - DNA, 83 bp, Artificial Sequence
  smMIP
  misc_feature (27)..(34): n is a, c, g, or t
cccagttttggctctactttgcagggnnnnnnnncttcagcttcccgatatccgacggtagtgtgggagttctgtccttctgg

SEQ ID NO: 498 - DNA, 83 bp, Artificial Sequence
  smMIP
  misc_feature (24)..(31): n is a, c, g, or t
gctttaacgtgtctgtgcagggtnnnnnnnncttcagcttcccgatatccgacggtagtgttcttgattttgcccgtacccgt

SEQ ID NO: 499 - DNA, 81 bp, Artificial Sequence
  smMIP
  misc_feature (28)..(35): n is a, c, g, or t
gcatgatgatgcagtcagggcgcaaagnnnnnnnncttcagcttcccgatatccgacggtagtgtgacactgcaaagggaa

SEQ ID NO: 500 - DNA, 83 bp, Artificial Sequence
  smMIP
  misc_feature (30)..(37): n is a, c, g, or t
cataagttatttcctaggatttttcccccnnnnnnnncttcagcttcccgatatccgacggtagtgtctggcggaagattgct

SEQ ID NO: 501 - DNA, 82 bp, Artificial Sequence
  smMIP
  misc_feature (23)..(30): n is a, c, g, or t
cacttttgatctggtggccctcnnnnnnnncttcagcttcccgatatccgacggtagtgtttcccgtcatattccagctctg

SEQ ID NO: 502 - DNA, 83 bp, Artificial Sequence
  smMIP
  misc_feature (23)..(30): n is a, c, g, or t
ccccggcgtgctggctgaaaacnnnnnnnncttcagcttcccgatatccgacggtagtgtcggctggaacttaatcctctcct

SEQ ID NO: 503 - DNA, 83 bp, Artificial Sequence
  smMIP
  misc_feature (22)..(29): n is a, c, g, or t
gctgggaaggtgccaagtacgnnnnnnnncttcagcttcccgatatccgacggtagtgtgtggggatcgtggcgtccctcttg

SEQ ID NO: 504 - DNA, 82 bp, Artificial Sequence
  smMIP
  misc_feature (25)..(32): n is a, c, g, or t
ggcaagcaccaatgattcccagctnnnnnnnncttcagcttcccgatatccgacggtagtgtctctcctggcttactcgttg

SEQ ID NO: 505 - DNA, 83 bp, Artificial Sequence
  smMIP
  misc_feature (24)..(31): n is a, c, g, or t
cgtgaacgtctatctcatgatgcnnnnnnnncttcagcttcccgatatccgacggtagtgttttctgctcgcagggtctgccc

SEQ ID NO: 506 - DNA, 82 bp, Artificial Sequence
  smMIP
  misc_feature (24)..(31): n is a, c, g, or t
tcccctcacagactcttccaccannnnnnnncttcagcttcccgatatccgacggtagtgtcagtgacagccccagctccca

SEQ ID NO: 507 - DNA, 83 bp, Artificial Sequence
  smMIP
  misc_feature (27)..(34): n is a, c, g, or t
gcctcatgaagataggtttccatgtgnnnnnnnncttcagcttcccgatatccgacggtagtgtccgccctgttaatcattcc

SEQ ID NO: 508 - DNA, 79 bp, Artificial Sequence
  smMIP
  misc_feature (23)..(30): n is a, c, g, or t
gcggggtgcttgtgggcgtggtnnnnnnnncttcagcttcccgatatccgacggtagtgtgggagtccttgctcaatga

SEQ ID NO: 509 - DNA, 82 bp, Artificial Sequence
  smMIP
  misc_feature (25)..(32): n is a, c, g, or t
gcccctggtagacctgttggctgtnnnnnnnncttcagcttcccgatatccgacggtagtgtgaggggccatcgccttctct

SEQ ID NO: 510 - DNA, 83 bp, Artificial Sequence
  smMIP
  misc_feature (30)..(37): n is a, c, g, or t
gcagctggagcagaagatcaacaactaccnnnnnnnncttcagcttcccgatatccgacggtagtgtatcctgaggaacaact

SEQ ID NO: 511 - DNA, 82 bp, Artificial Sequence
  smMIP
  misc_feature (27)..(34): n is a, c, g, or t
gcctgtgtgacaagctggggaagaatnnnnnnnncttcagcttcccgatatccgacggtagtgtgggaggagaggttagatg

SEQ ID NO: 512 - DNA, 83 bp, Artificial Sequence
  smMIP
  misc_feature (26)..(33): n is a, c, g, or t
gctggggcctgggaagaagaatgatnnnnnnnncttcagcttcccgatatccgacggtagtgttaaggctagtggccgcttgg

SEQ ID NO: 513 - DNA, 83 bp, Artificial Sequence
  smMIP
  misc_feature (29)..(36): n is a, c, g, or t
gctgatggtggatttcttcaacattttgnnnnnnnncttcagcttcccgatatccgacggtagtgtggaggagactaagatgg

SEQ ID NO: 514 - DNA, 83 bp, Artificial Sequence
  smMIP
  misc_feature (19)..(26): n is a, c, g, or t
cgtccttcctgttggagcnnnnnnnncttcagcttcccgatatccgacggtagtgtccttctccttttttgctggcattttcc

SEQ ID NO: 515 - DNA, 83 bp, Artificial Sequence
  smMIP
  misc_feature (30)..(37): n is a, c, g, or t
cgagtgcatgaagatattgaaatgaccaannnnnnnncttcagcttcccgatatccgacggtagtgttggctgtggactggct

SEQ ID NO: 516 - DNA, 81 bp, Artificial Sequence
  smMIP
  misc_feature (26)..(33): n is a, c, g, or t
ggactgactgaaggcctgcatggatnnnnnnnncttcagcttcccgatatccgacggtagtgtgtggcctagcgagttatg

SEQ ID NO: 517 - DNA, 81 bp, Artificial Sequence
  smMIP
  misc_feature (27)..(34): n is a, c, g, or t
gtgggccaaaggatgaagagaggcatnnnnnnnncttcagcttcccgatatccgacggtagtgtggtgtggggaagcatta

SEQ ID NO: 518 - DNA, 83 bp, Artificial Sequence
  smMIP
  misc_feature (24)..(31): n is a, c, g, or t
tctcactctcaggagaccattgcnnnnnnnncttcagcttcccgatatccgacggtagtgttcctcactttaatcctctatcc

SEQ ID NO: 519 - DNA, 83 bp, Artificial Sequence
  smMIP
  misc_feature (28)..(35): n is a, c, g, or t
gtacaaagacaggaaacgctggaagtcnnnnnnnncttcagcttcccgatatccgacggtagtgtggtgtggccgatgtgtct

SEQ ID NO: 520 - DNA, 83 bp, Artificial Sequence
  smMIP
  misc_feature (28)..(35): n is a, c, g, or t
cccttggatgtagtctgaggccccttannnnnnnncttcagcttcccgatatccgacggtagtgtgcacactggtggtccatg

SEQ ID NO: 521 - DNA, 82 bp, Artificial Sequence
  smMIP
  misc_feature (25)..(32): n is a, c, g, or t
gccctggaacctcacatcaacgcgnnnnnnnncttcagcttcccgatatccgacggtagtgttagcaccagcactagcagca

SEQ ID NO: 522 - DNA, 81 bp, Artificial Sequence
  smMIP
  misc_feature (27)..(34): n is a, c, g, or t
gcgttggccaagggagatgttacagcnnnnnnnncttcagcttcccgatatccgacggtagtgtacctgccctacgactac

SEQ ID NO: 523 - DNA, 82 bp, Artificial Sequence
  smMIP
  misc_feature (26)..(33): n is a, c, g, or t
gctcaggttggggttggcttggtttnnnnnnnncttcagcttcccgatatccgacggtagtgtggacaaacctcagccctaa

SEQ ID NO: 524 - DNA, 83 bp, Artificial Sequence
  smMIP
  misc_feature (30)..(37): n is a, c, g, or t
gggagaatgtaactgaaagatacatggctnnnnnnnncttcagcttcccgatatccgacggtagtgtactgcaaggaacaaca

SEQ ID NO: 525 - DNA, 83 bp, Artificial Sequence
  smMIP
  misc_feature (29)..(36): n is a, c, g, or t
gctgagtatgttaagctctttatgactgnnnnnnnncttcagcttcccgatatccgacggtagtgtcgcttactaccttcagt

SEQ ID NO: 526 - DNA, 78 bp, Artificial Sequence
  smMIP
  misc_feature (23)..(30): n is a, c, g, or t
gaagattccgggccgagtatccnnnnnnnncttcagcttcccgatatccgacggtagtgtgatgcccgcttaccagga

SEQ ID NO: 527 - DNA, 83 bp, Artificial Sequence
  smMIP
  misc_feature (24)..(31): n is a, c, g, or t
tcgaggagcagcacggcatccacnnnnnnnncttcagcttcccgatatccgacggtagtgttgataaagatgcgatggtggcc

SEQ ID NO: 528 - DNA, 82 bp, Artificial Sequence
  smMIP
  misc_feature (29)..(36): n is a, c, g, or t
gtttagctacaaaaccattgtcatgggcnnnnnnnncttcagcttcccgatatccgacggtagtgtcatctccccatttgtt

SEQ ID NO: 529 - DNA, 82 bp, Artificial Sequence
  smMIP
  misc_feature (25)..(32): n is a, c, g, or t
ccacctggatgagaagtctttccgnnnnnnnncttcagcttcccgatatccgacggtagtgtaagcactggccggctgtgac

SEQ ID NO: 530 - DNA, 82 bp, Artificial Sequence
  smMIP
  misc_feature (26)..(33): n is a, c, g, or t
gaggctggactccagatctgcaccgnnnnnnnncttcagcttcccgatatccgacggtagtgtgaggaccagatggctgtgg

SEQ ID NO: 531 - DNA, 83 bp, Artificial Sequence
  smMIP
  misc_feature (28)..(35): n is a, c, g, or t
catgaggacaaagcagaccatgcatggnnnnnnnncttcagcttcccgatatccgacggtagtgtaggagaaaataatccaag

SEQ ID NO: 532 - DNA, 82 bp, Artificial Sequence
  smMIP
  misc_feature (29)..(36): n is a, c, g, or t
cggaggagagacgctggaccaggtgaaannnnnnnncttcagcttcccgatatccgacggtagtgtgacggtaaagtatgac

SEQ ID NO: 533 - DNA, 83 bp, Artificial Sequence
  smMIP
  misc_feature (28)..(35): n is a, c, g, or t
ggattagcagccagtgtcttagttgtgnnnnnnnncttcagcttcccgatatccgacggtagtgtagcggatcaaaaagaaca

SEQ ID NO: 534 - DNA, 83 bp, Artificial Sequence
  smMIP
  misc_feature (30)..(37): n is a, c, g, or t
gaggaaggaagagaagttaaaccaacggtnnnnnnnncttcagcttcccgatatccgacggtagtgttgagaagtctgtttga

SEQ ID NO: 535 - DNA, 82 bp, Artificial Sequence
  smMIP
  misc_feature (23)..(30): n is a, c, g, or t
gtgaagtcctcctgaaggtggcnnnnnnnncttcagcttcccgatatccgacggtagtgtctgccctgtcctgtcctgccct

SEQ ID NO: 536 - DNA, 82 bp, Artificial Sequence
  smMIP
  misc_feature (29)..(36): n is a, c, g, or t
gcaacattttgggacttgaggcatctggnnnnnnnncttcagcttcccgatatccgacggtagtgttgaaggaggtggccaa

SEQ ID NO: 537 - DNA, 83 bp, Artificial Sequence
  smMIP
  misc_feature (24)..(31): n is a, c, g, or t
gccatggctctgctccccggtggnnnnnnnncttcagcttcccgatatccgacggtagtgtacttaatgcagagacaaggcca

SEQ ID NO: 538 - DNA, 83 bp, Artificial Sequence
  smMIP
  misc_feature (28)..(35): n is a, c, g, or t
gctaatccatgcaggactgagtggtgtnnnnnnnncttcagcttcccgatatccgacggtagtgtaagggctcctcatgccta

SEQ ID NO: 539 - DNA, 83 bp, Artificial Sequence
  smMIP
  misc_feature (27)..(34): n is a, c, g, or t
gcttcaaatggcagaaaagcttggagnnnnnnnncttcagcttcccgatatccgacggtagtgtggaaatgttcaggctggag

SEQ ID NO: 540 - DNA, 83 bp, Artificial Sequence
  smMIP
  misc_feature (28)..(35): n is a, c, g, or t
gctggagttaatcttattctagactgcnnnnnnnncttcagcttcccgatatccgacggtagtgttattcctctggtcacagc

SEQ ID NO: 541 - DNA, 83 bp, Artificial Sequence
  smMIP
  misc_feature (29)..(36): n is a, c, g, or t
gtcgatgggttctctatggtctgatgggnnnnnnnncttcagcttcccgatatccgacggtagtgtacaaaaaagaggatttc

SEQ ID NO: 542 - DNA, 83 bp, Artificial Sequence
  smMIP
  misc_feature (27)..(34): n is a, c, g, or t
gctgaggtctagggacaataagtacannnnnnnncttcagcttcccgatatccgacggtagtgtaagaacgtcaactgcctgg

SEQ ID NO: 543 - DNA, 83 bp, Artificial Sequence
  smMIP
  misc_feature (20)..(27): n is a, c, g, or t
agggcccccaacgtctgctnnnnnnnncttcagcttcccgatatccgacggtagtgtggcccctgttttcaaagctacttttt

SEQ ID NO: 544 - DNA, 83 bp, Artificial Sequence
  smMIP
  misc_feature (25)..(32): n is a, c, g, or t
tcgtcctggaactgccccagtgaannnnnnnncttcagcttcccgatatccgacggtagtgtcaaattctgcctcacttctcc

SEQ ID NO: 545 - DNA, 83 bp, Artificial Sequence
  smMIP
  misc_feature (20)..(27): n is a, c, g, or t
cttctcagccacgtggtgtnnnnnnnncttcagcttcccgatatccgacggtagtgttttggatccatttccatcggtcctta

SEQ ID NO: 546 - DNA, 83 bp, Artificial Sequence
  smMIP
  misc_feature (30)..(37): n is a, c, g, or t
gttttttaagaagggacaaaaggtgggtgnnnnnnnncttcagcttcccgatatccgacggtagtgtgcaggtgataaacttg

SEQ ID NO: 547 - DNA, 83 bp, Artificial Sequence
  smMIP
  misc_feature (30)..(37): n is a, c, g, or t
gttttctgaaaatataaccagccattggcnnnnnnnncttcagcttcccgatatccgacggtagtgtagagtgtgaagtcaaa

SEQ ID NO: 548 - DNA, 83 bp, Artificial Sequence
  smMIP
  misc_feature (26)..(33): n is a, c, g, or t
gccgtgatcagttggactaaggatgnnnnnnnncttcagcttcccgatatccgacggtagtgtgtttagttgaggataccaca

SEQ ID NO: 549 - DNA, 83 bp, Artificial Sequence
  smMIP
  misc_feature (30)..(37): n is a, c, g, or t
cgatggtgcggaagattttgtcagtgagannnnnnnncttcagcttcccgatatccgacggtagtgtccacgcctagagactc

SEQ ID NO: 550 - DNA, 83 bp, Artificial Sequence
  smMIP
  misc_feature (25)..(32): n is a, c, g, or t
gccggtgttaacaccacggacaaannnnnnnncttcagcttcccgatatccgacggtagtgtgacgtagagtttgtctgcaag

SEQ ID NO: 551 - DNA, 83 bp, Artificial Sequence
  smMIP
  misc_feature (29)..(36): n is a, c, g, or t
actacctggagatagccatttactgcatnnnnnnnncttcagcttcccgatatccgacggtagtgtttttgaggacgctgggg

SEQ ID NO: 552 - DNA, 83 bp, Artificial Sequence
  smMIP
  misc_feature (29)..(36): n is a, c, g, or t
gcagtgttaaaacatgaatgactgtgtcnnnnnnnncttcagcttcccgatatccgacggtagtgtcgaacagtattcaccta

SEQ ID NO: 553 - DNA, 83 bp, Artificial Sequence
  smMIP
  misc_feature (24)..(31): n is a, c, g, or t
cgtgctgcccgtatggctcaactnnnnnnnncttcagcttcccgatatccgacggtagtgtaagagtccggcccggaggaact

SEQ ID NO: 554 - DNA, 83 bp, Artificial Sequence
  smMIP
  misc_feature (24)..(31): n is a, c, g, or t
gctcttcagagatgcagggacacnnnnnnnncttcagcttcccgatatccgacggtagtgttcttctgcaatcgcagtccgcg

SEQ ID NO: 555 - DNA, 80 bp, Artificial Sequence
  smMIP
  misc_feature (22)..(29): n is a, c, g, or t
gcgccgaggaggagatggaggnnnnnnnncttcagcttcccgatatccgacggtagtgtagaactgggacgaggccgagg

SEQ ID NO: 556 - DNA, 82 bp, Artificial Sequence
  smMIP
  misc_feature (22)..(29): n is a, c, g, or t
agtcgggcgccgaggagtccgnnnnnnnncttcagcttcccgatatccgacggtagtgtggcgtccggcccgggtggtctgg

SEQ ID NO: 557 - DNA, 82 bp, Artificial Sequence
  smMIP
  misc_feature (25)..(32): n is a, c, g, or t
ctcaatgttgacggacagcctattnnnnnnnncttcagcttcccgatatccgacggtagtgttacccaacgctgccgcctgg

SEQ ID NO: 558 - DNA, 83 bp, Artificial Sequence
  smMIP
  misc_feature (30)..(37): n is a, c, g, or t
gtccggagcctagtcaagcctgagaattannnnnnnncttcagcttcccgatatccgacggtagtgtcgatgggcttctggtt

SEQ ID NO: 559 - DNA, 83 bp, Artificial Sequence
  smMIP
  misc_feature (29)..(36): n is a, c, g, or t
gcggctgacacaggagcgcattgcacatnnnnnnnncttcagcttcccgatatccgacggtagtgtaaagagcgatgcctcca

SEQ ID NO: 560 - DNA, 83 bp, Artificial Sequence
  smMIP
  misc_feature (27)..(34): n is a, c, g, or t
gcttttgatggtactgatgagtcttgnnnnnnnncttcagcttcccgatatccgacggtagtgttacgaagatctggaagacc

SEQ ID NO: 561 - DNA, 83 bp, Artificial Sequence
  smMIP
  misc_feature (22)..(29): n is a, c, g, or t
gatgtgcacgcccggctgcaannnnnnnncttcagcttcccgatatccgacggtagtgtaacacggaggcaatcgactgcatc

SEQ ID NO: 562 - DNA, 82 bp, Artificial Sequence
  smMIP
  misc_feature (29)..(36): n is a, c, g, or t
gatggtgtacctggcgggtctgcattttnnnnnnnncttcagcttcccgatatccgacggtagtgtctcaaccgcttcctcc

SEQ ID NO: 563 - DNA, 83 bp, Artificial Sequence
  smMIP
  misc_feature (30)..(37): n is a, c, g, or t
catcgtgaagagtggtctccgtttcgtggnnnnnnnncttcagcttcccgatatccgacggtagtgtatagcctccaccacct

SEQ ID NO: 564 - DNA, 83 bp, Artificial Sequence
  smMIP
  misc_feature (26)..(33): n is a, c, g, or t
gcaaaggctctgggctccaaggccannnnnnnncttcagcttcccgatatccgacggtagtgtcaacaaatgtggacggagaa

SEQ ID NO: 565 - DNA, 83 bp, Artificial Sequence
  smMIP
  misc_feature (25)..(32): n is a, c, g, or t
gcagggatatctacagcaccgactnnnnnnnncttcagcttcccgatatccgacggtagtgttggctagccaggtcgctgcgg

SEQ ID NO: 566 - DNA, 82 bp, Artificial Sequence
  smMIP
  misc_feature (20)..(27): n is a, c, g, or t
agtcaacacctcctcaaccnnnnnnnncttcagcttcccgatatccgacggtagtgtctctatactgccgtgcagcccaatg

SEQ ID NO: 567 - DNA, 80 bp, Artificial Sequence
  smMIP
  misc_feature (22)..(29): n is a, c, g, or t
cggtggtgtgggaacggatgtnnnnnnnncttcagcttcccgatatccgacggtagtgttgctgttgctgtctctcctgt

SEQ ID NO: 568 - DNA, 83 bp, Artificial Sequence
  smMIP
  misc_feature (30)..(37): n is a, c, g, or t
cgtggcttttctggtatctttgaggacagnnnnnnnncttcagcttcccgatatccgacggtagtgtcatctttctcacggaa

SEQ ID NO: 569 - DNA, 83 bp, Artificial Sequence
  smMIP
  misc_feature (28)..(35): n is a, c, g, or t
gtccgagtgctggagctaagtgagagcnnnnnnnncttcagcttcccgatatccgacggtagtgtcggtatgtgtcagagctg

SEQ ID NO: 570 - DNA, 83 bp, Artificial Sequence
  smMIP
  misc_feature (26)..(33): n is a, c, g, or t
gccaatggcatggagtttctggcctnnnnnnnncttcagcttcccgatatccgacggtagtgtccaactacatggccccttac

SEQ ID NO: 571 - DNA, 83 bp, Artificial Sequence
  smMIP
  misc_feature (19)..(26): n is a, c, g, or t
ttcggccccagcccccttnnnnnnnncttcagcttcccgatatccgacggtagtgttccccacacatgaccccagccctctac

SEQ ID NO: 572 - DNA, 83 bp, Artificial Sequence
  smMIP
  misc_feature (21)..(28): n is a, c, g, or t
gccctgggaccagctctttcnnnnnnnncttcagcttcccgatatccgacggtagtgtacaatggcgcctactcgctgaccct

SEQ ID NO: 573 - DNA, 83 bp, Artificial Sequence
  smMIP
  misc_feature (26)..(33): n is a, c, g, or t
cacgattttgtggaaggacatcttcnnnnnnnncttcagcttcccgatatccgacggtagtgtaacaataccacccctgtcac

SEQ ID NO: 574 - DNA, 83 bp, Artificial Sequence
  smMIP
  misc_feature (28)..(35): n is a, c, g, or t
gcaagaagatctttgggagcctggcatnnnnnnnncttcagcttcccgatatccgacggtagtgtatggaacacagcggtgtg

SEQ ID NO: 575 - DNA, 79 bp, Artificial Sequence
  smMIP
  misc_feature (26)..(33): n is a, c, g, or t
gctggctccgatgtatttgatggtgnnnnnnnncttcagcttcccgatatccgacggtagtgtctgggggcatggtcca

SEQ ID NO: 576 - DNA, 83 bp, Artificial Sequence
  smMIP
  misc_feature (29)..(36): n is a, c, g, or t
gcgagagccccacatgaggaagaacatcnnnnnnnncttcagcttcccgatatccgacggtagtgtaaacagccctggtacca

SEQ ID NO: 577 - DNA, 83 bp, Artificial Sequence
  smMIP
  misc_feature (28)..(35): n is a, c, g, or t
gcaacctgcagcacatcaattttacccnnnnnnnncttcagcttcccgatatccgacggtagtgtcaaaccagaaaaggttag

SEQ ID NO: 578 - DNA, 83 bp, Artificial Sequence
  smMIP
  misc_feature (28)..(35): n is a, c, g, or t
gcagatctcttgtgtggcggaaaatctnnnnnnnncttcagcttcccgatatccgacggtagtgtaggtgatccggttcctaa

SEQ ID NO: 579 - DNA, 83 bp, Artificial Sequence
  smMIP
  misc_feature (26)..(33): n is a, c, g, or t
cggggacaccacgaacagaagtaatnnnnnnnncttcagcttcccgatatccgacggtagtgtgagtatgggaaggatgagaa

SEQ ID NO: 580 - DNA, 83 bp, Artificial Sequence
  smMIP
  misc_feature (26)..(33): n is a, c, g, or t
gctgaatgctataacctctgtcctgnnnnnnnncttcagcttcccgatatccgacggtagtgtatccccagtactttggcatc

SEQ ID NO: 581 - DNA, 83 bp, Artificial Sequence
  smMIP
  misc_feature (26)..(33): n is a, c, g, or t
gcacaactgatcccgagactcctgtnnnnnnnncttcagcttcccgatatccgacggtagtgtgaatgagcttgaaggcaggc

SEQ ID NO: 582 - DNA, 82 bp, Artificial Sequence
  smMIP
  misc_feature (28)..(35): n is a, c, g, or t
gtaataatgaaacttcctggactatttnnnnnnnncttcagcttcccgatatccgacggtagtgtcatccattctggacttg

SEQ ID NO: 583 - DNA, 83 bp, Artificial Sequence
  smMIP
  misc_feature (30)..(37): n is a, c, g, or t
cgacatccagagatcactctatgatcgtcnnnnnnnncttcagcttcccgatatccgacggtagtgtaaagcacacggagcta

SEQ ID NO: 584 - DNA, 79 bp, Artificial Sequence
  smMIP
  misc_feature (25)..(32): n is a, c, g, or t
gtgggtaccggatggccaagcctgnnnnnnnncttcagcttcccgatatccgacggtagtgtacaacctctacaccaca

SEQ ID NO: 585 - DNA, 83 bp, Artificial Sequence
  smMIP
  misc_feature (25)..(32): n is a, c, g, or t
catcaagagagaggacgagaccatnnnnnnnncttcagcttcccgatatccgacggtagtgtctacatcattcctctgcctga

SEQ ID NO: 586 - DNA, 82 bp, Artificial Sequence
  smMIP
  misc_feature (29)..(36): n is a, c, g, or t
cgtcaatgtttcagatgctctcccctccnnnnnnnncttcagcttcccgatatccgacggtagtgtgaaagcaaccgcaccc

SEQ ID NO: 587 - DNA, 83 bp, Artificial Sequence
  smMIP
  misc_feature (23)..(30): n is a, c, g, or t
gcatcacaggggaggaggtggannnnnnnncttcagcttcccgatatccgacggtagtgttgtggaagtggagtccttcctgg

SEQ ID NO: 588 - DNA, 83 bp, Artificial Sequence
  smMIP
  misc_feature (19)..(26): n is a, c, g, or t
tggcaaagaattcaaaccnnnnnnnncttcagcttcccgatatccgacggtagtgtgcccgtagctccatattggacatcccc

SEQ ID NO: 589 - DNA, 83 bp, Artificial Sequence
  smMIP
  misc_feature (22)..(29): n is a, c, g, or t
cagataacaccaaaccaaaccnnnnnnnncttcagcttcccgatatccgacggtagtgttctatgcttgcgtaaccagcagcc

SEQ ID NO: 590 - DNA, 83 bp, Artificial Sequence
  smMIP
  misc_feature (24)..(31): n is a, c, g, or t
catccctctgcgcagacaggtaannnnnnnncttcagcttcccgatatccgacggtagtgttcatctattgcacaggggcctt

SEQ ID NO: 591 - DNA, 83 bp, Artificial Sequence
  smMIP
  misc_feature (25)..(32): n is a, c, g, or t
tgtgacaagccgaggcggtgagccnnnnnnnncttcagcttcccgatatccgacggtagtgttccaacatcaccatgcagatt

SEQ ID NO: 592 - DNA, 83 bp, Artificial Sequence
  smMIP
  misc_feature (25)..(32): n is a, c, g, or t
tctctcaccaggaaagactgatacnnnnnnnncttcagcttcccgatatccgacggtagtgttccaacatcaccatgcagatt

SEQ ID NO: 593 - DNA, 78 bp, Artificial Sequence
  smMIP
  misc_feature (25)..(32): n is a, c, g, or t
gaggcggtgagccgggcaggaggannnnnnnncttcagcttcccgatatccgacggtagtgtagagcggagaaagcat

SEQ ID NO: 594 - DNA, 82 bp, Artificial Sequence
  smMIP
  misc_feature (22)..(29): n is a, c, g, or t
cctgtgggccttgctcagagcnnnnnnnncttcagcttcccgatatccgacggtagtgtagtccaacatcaccatgcagatt

SEQ ID NO: 595 - DNA, 83 bp, Artificial Sequence
  smMIP
  misc_feature (23)..(30): n is a, c, g, or t
ccacgctgccgccaccacaccannnnnnnncttcagcttcccgatatccgacggtagtgttccgcagacgtgtaaatgttcct

SEQ ID NO: 596 - DNA, 82 bp, Artificial Sequence
  smMIP
  misc_feature (29)..(36): n is a, c, g, or t
gttcgaggaaagggaaaggggcaaaaacnnnnnnnncttcagcttcccgatatccgacggtagtgtccatgcagattatgcg

SEQ ID NO: 597 - DNA, 79 bp, Artificial Sequence
  smMIP
  misc_feature (24)..(31): n is a, c, g, or t
ggcgtgagccctcccccttgggannnnnnnncttcagcttcccgatatccgacggtagtgtagcaagagctccagagag

SEQ ID NO: 598 - DNA, 81 bp, Artificial Sequence
  smMIP
  misc_feature (27)..(34): n is a, c, g, or t
cgaagtggtgaagttcatggatgtctnnnnnnnncttcagcttcccgatatccgacggtagtgtcctccgaaaccatgaac

SEQ ID NO: 599 - DNA, 83 bp, Artificial Sequence
  smMIP
  misc_feature (25)..(32): n is a, c, g, or t
cgttttaatttatttttgcttgccnnnnnnnncttcagcttcccgatatccgacggtagtgtgttaggtggaccggtcagcgg

SEQ ID NO: 600 - DNA, 83 bp, Artificial Sequence
  smMIP
  misc_feature (24)..(31): n is a, c, g, or t
cactgtggattttggaaaccagcnnnnnnnncttcagcttcccgatatccgacggtagtgtccctcttcttttttcttaaaca

SEQ ID NO: 601 - DNA, 80 bp, Artificial Sequence
  smMIP
  misc_feature (26)..(33): n is a, c, g, or t
ggcgctcggaagccgggctcatggannnnnnnncttcagcttcccgatatccgacggtagtgtgcgcgggggaagccgag

SEQ ID NO: 602 - DNA, 80 bp, Artificial Sequence
  smMIP
  misc_feature (27)..(34): n is a, c, g, or t
gcctggagtgtgtgcccactgaggagnnnnnnnncttcagcttcccgatatccgacggtagtgtagctactgccatccaa

SEQ ID NO: 603 - DNA, 83 bp, Artificial Sequence
  smMIP
  misc_feature (30)..(37): n is a, c, g, or t
cctacagcacaacaaatgtgaatgcagacnnnnnnnncttcagcttcccgatatccgacggtagtgtgatgcgggggctgctg

SEQ ID NO: 604 - DNA, 82 bp, Artificial Sequence
  smMIP
  misc_feature (24)..(31): n is a, c, g, or t
ctgtgggccttgctcagagcggannnnnnnncttcagcttcccgatatccgacggtagtgtccaacatcaccatgcagatta

SEQ ID NO: 605 - DNA, 78 bp, Artificial Sequence
  smMIP
  misc_feature (21)..(28): n is a, c, g, or t
gccagagctgccaggctcggnnnnnnnncttcagcttcccgatatccgacggtagtgtcggaagaggcgcgggctagg

SEQ ID NO: 606 - DNA, 82 bp, Artificial Sequence
  smMIP
  misc_feature (23)..(30): n is a, c, g, or t
caccagccgagtgtctctgaggnnnnnnnncttcagcttcccgatatccgacggtagtgtatccatttccacacgctggtct

SEQ ID NO: 607 - DNA, 83 bp, Artificial Sequence
  smMIP
  misc_feature (22)..(29): n is a, c, g, or t
gtggggccagttaaaagctccnnnnnnnncttcagcttcccgatatccgacggtagtgtggttcttctttgcggtccagttgg

SEQ ID NO: 608 - DNA, 83 bp, Artificial Sequence
  smMIP
  misc_feature (20)..(27): n is a, c, g, or t
ttctcacccagtcagcctcnnnnnnnncttcagcttcccgatatccgacggtagtgttgacatccccactggtattccagttc

SEQ ID NO: 609 - DNA, 79 bp, Artificial Sequence
  smMIP
  misc_feature (25)..(32): n is a, c, g, or t
gcaactgtggatggacactgggccnnnnnnnncttcagcttcccgatatccgacggtagtgtggtgttcctgatgtggg

SEQ ID NO: 610 - DNA, 83 bp, Artificial Sequence
  smMIP
  misc_feature (28)..(35): n is a, c, g, or t
agtcggccctggagtgccaccccgagannnnnnnncttcagcttcccgatatccgacggtagtgtagcaagagactagcccca

SEQ ID NO: 611 - DNA, 83 bp, Artificial Sequence
  smMIP
  misc_feature (20)..(27): n is a, c, g, or t
tggcggcggcggcggcggcnnnnnnnncttcagcttcccgatatccgacggtagtgtagcctgcatggcgcgggtgcagcggg

SEQ ID NO: 612 - DNA, 83 bp, Artificial Sequence
  smMIP
  misc_feature (24)..(31): n is a, c, g, or t
gcttgtacacgtggtcaagtgggnnnnnnnncttcagcttcccgatatccgacggtagtgtgtcagcccatctttctgaatgt

SEQ ID NO: 613 - DNA, 83 bp, Artificial Sequence
  smMIP
  misc_feature (26)..(33): n is a, c, g, or t
gctcatggtgtttgccatgggctggnnnnnnnncttcagcttcccgatatccgacggtagtgttctagcctcaatgaactggg

SEQ ID NO: 614 - DNA, 83 bp, Artificial Sequence
  smMIP
  misc_feature (29)..(36): n is a, c, g, or t
catggtgagcgtggactttccggaaatgnnnnnnnncttcagcttcccgatatccgacggtagtgtaaaagaaaaaatcccac

SEQ ID NO: 615 - DNA, 83 bp, Artificial Sequence
  smMIP
  misc_feature (19)..(26): n is a, c, g, or t
ccacacccagtgaagcatnnnnnnnncttcagcttcccgatatccgacggtagtgtgctgcatcagttcacttttgacctgct

SEQ ID NO: 616 - DNA, 81 bp, Artificial Sequence
  smMIP
  misc_feature (25)..(32): n is a, c, g, or t
gcatctcaaaatgaccagaccctgnnnnnnnncttcagcttcccgatatccgacggtagtgtgtgcgccagcagaaatgat

SEQ ID NO: 617 - DNA, 83 bp, Artificial Sequence
  smMIP
  misc_feature (25)..(32): n is a, c, g, or t
gcagagatcatctctgtgcaagtgnnnnnnnncttcagcttcccgatatccgacggtagtgtagagacagcttgtacacgtgg

SEQ ID NO: 618 - DNA, 83 bp, Artificial Sequence
  smMIP
  misc_feature (21)..(28): n is a, c, g, or t
ccccaaattctcaccagtccnnnnnnnncttcagcttcccgatatccgacggtagtgtccaacttgatttgctgtttgtctcc

SEQ ID NO: 619 - DNA, 83 bp, Artificial Sequence
  smMIP
  misc_feature (30)..(37): n is a, c, g, or t
gacatgtgaatatcctactcttcatgggcnnnnnnnncttcagcttcccgatatccgacggtagtgtggaacagtctacaagg

SEQ ID NO: 620 - DNA, 83 bp, Artificial Sequence
  smMIP
  misc_feature (30)..(37): n is a, c, g, or t
gctacagtgaaatctcgatggagtgggtcnnnnnnnncttcagcttcccgatatccgacggtagtgtacgacagactgcacag

SEQ ID NO: 621 - DNA, 83 bp, Artificial Sequence
  smMIP
  misc_feature (30)..(37): n is a, c, g, or t
catacagctttcagtcagatgtatatgcannnnnnnncttcagcttcccgatatccgacggtagtgtataggtgattttggtc

SEQ ID NO: 622 - DNA, 83 bp, Artificial Sequence
  smMIP
  misc_feature (24)..(31): n is a, c, g, or t
caaacatcaacaacagggaccagnnnnnnnncttcagcttcccgatatccgacggtagtgtggatccattttgtggatggcac

SEQ ID NO: 623 - DNA, 83 bp, Artificial Sequence
  smMIP
  misc_feature (30)..(37): n is a, c, g, or t
cggacatggtctgggacttctggagcctannnnnnnncttcagcttcccgatatccgacggtagtgtgaagatggtaactggg

SEQ ID NO: 624 - DNA, 83 bp, Artificial Sequence
  smMIP
  misc_feature (19)..(26): n is a, c, g, or t
ccagggcatcaaaaacctnnnnnnnncttcagcttcccgatatccgacggtagtgtggtttctttcttgttcagtgatcgggg

SEQ ID NO: 625 - DNA, 82 bp, Artificial Sequence
  smMIP
  misc_feature (28)..(35): n is a, c, g, or t
caccaaggtttggcctcacaaggactannnnnnnncttcagcttcccgatatccgacggtagtgttgactatggcatccggg

SEQ ID NO: 626 - DNA, 83 bp, Artificial Sequence
  smMIP
  misc_feature (29)..(36): n is a, c, g, or t
cctgaaggatgcacaaattttcatccagnnnnnnnncttcagcttcccgatatccgacggtagtgttctggagaagtgcggag

SEQ ID NO: 627 - DNA, 83 bp, Artificial Sequence
  smMIP
  misc_feature (30)..(37): n is a, c, g, or t
gcggcaagggagaaggcaaatctgtgaggnnnnnnnncttcagcttcccgatatccgacggtagtgtcaagaacttcactgag

SEQ ID NO: 628 - DNA, 81 bp, Artificial Sequence
  smMIP
  misc_feature (28)..(35): n is a, c, g, or t
gagggcccggctgttgaaggaccagctnnnnnnnncttcagcttcccgatatccgacggtagtgtcccagtagaaaaacaa

SEQ ID NO: 629 - DNA, 83 bp, Artificial Sequence
  smMIP
  misc_feature (25)..(32): n is a, c, g, or t
tggttgtggatccagtcacctctgnnnnnnnncttcagcttcccgatatccgacggtagtgtagatcacagatgtgaaattgc

SEQ ID NO: 630 - DNA, 83 bp, Artificial Sequence
  smMIP
  misc_feature (29)..(36): n is a, c, g, or t
gcacttttaggagattagatcctgaggannnnnnnncttcagcttcccgatatccgacggtagtgtaagcagtgaccatcaag

SEQ ID NO: 631 - DNA, 83 bp, Artificial Sequence
  smMIP
  misc_feature (29)..(36): n is a, c, g, or t
ggcatccaagatacaaactcaaagaagcnnnnnnnncttcagcttcccgatatccgacggtagtgtcacatcctccaaatgaa

SEQ ID NO: 632 - DNA, 83 bp, Artificial Sequence
  smMIP
  misc_feature (27)..(34): n is a, c, g, or t
cttctgatcttcaagcagggattctcnnnnnnnncttcagcttcccgatatccgacggtagtgtctgacattcatcttccgtt

SEQ ID NO: 633 - DNA, 82 bp, Artificial Sequence
  smMIP
  misc_feature (28)..(35): n is a, c, g, or t
gcaaagcaatgcacgtggcccagcctgnnnnnnnncttcagcttcccgatatccgacggtagtgtaacaccgctcccataaa

SEQ ID NO: 634 - DNA, 83 bp, Artificial Sequence
  smMIP
  misc_feature (27)..(34): n is a, c, g, or t
cttcctagatgattccatctgcacggnnnnnnnncttcagcttcccgatatccgacggtagtgtgctttgtgtgtgagtatgc

SEQ ID NO: 635 - DNA, 83 bp, Artificial Sequence
  smMIP
  misc_feature (26)..(33): n is a, c, g, or t
ttgatccagaaccgtgcccagattcnnnnnnnncttcagcttcccgatatccgacggtagtgttcaagtgaacctcactatcc

SEQ ID NO: 636 - DNA, 82 bp, Artificial Sequence
  smMIP
  misc_feature (19)..(26): n is a, c, g, or t
cccccaacagagccagaannnnnnnncttcagcttcccgatatccgacggtagtgtgacttcctcctctggatccttgcagc

SEQ ID NO: 637 - DNA, 81 bp, Artificial Sequence
  smMIP
  misc_feature (21)..(28): n is a, c, g, or t
cccagctgctcaactcgctcnnnnnnnncttcagcttcccgatatccgacggtagtgtttgcacctgcagccccgcgctct

SEQ ID NO: 638 - DNA, 83 bp, Artificial Sequence
  smMIP
  misc_feature (22)..(29): n is a, c, g, or t
gatccccttgatggatcttccnnnnnnnncttcagcttcccgatatccgacggtagtgtgtcctctccaacgacctggttatg

SEQ ID NO: 639 - DNA, 83 bp, Artificial Sequence
  smMIP
  misc_feature (24)..(31): n is a, c, g, or t
gtccttgccatggactgtggggtnnnnnnnncttcagcttcccgatatccgacggtagtgtcgttggaaccatttttggcatc

SEQ ID NO: 640 - DNA, 83 bp, Artificial Sequence
  smMIP
  misc_feature (27)..(34): n is a, c, g, or t
gctccttatggggcccggtatgtgggnnnnnnnncttcagcttcccgatatccgacggtagtgtggacaaggatgtgaagata

SEQ ID NO: 641 - DNA, 82 bp, Artificial Sequence
  smMIP
  misc_feature (29)..(36): n is a, c, g, or t
ccactgggaaggaggccgtgttagacgtnnnnnnnncttcagcttcccgatatccgacggtagtgttctggtctacggaggg

SEQ ID NO: 642 - DNA, 82 bp, Artificial Sequence
  smMIP
  misc_feature (22)..(29): n is a, c, g, or t
ccacctcctccaggatatgaannnnnnnncttcagcttcccgatatccgacggtagtgttttggcctggattctgttgagct

SEQ ID NO: 643 - DNA, 83 bp, Artificial Sequence
  smMIP
  misc_feature (26)..(33): n is a, c, g, or t
cctctcacaccaggttacccagcaannnnnnnncttcagcttcccgatatccgacggtagtgttcattctctactccgaccct

SEQ ID NO: 644 - DNA, 83 bp, Artificial Sequence
  smMIP
  misc_feature (30)..(37): n is a, c, g, or t
gtggagcagctgttgttcatgaaattgtgnnnnnnnncttcagcttcccgatatccgacggtagtgtaatgaagtgacaagaa

SEQ ID NO: 645 - DNA, 83 bp, Artificial Sequence
  smMIP
  misc_feature (28)..(35): n is a, c, g, or t
gggatctggaaatgattttgaggtgttnnnnnnnncttcagcttcccgatatccgacggtagtgtacacaacctaacaaaaga

SEQ ID NO: 646 - DNA, 83 bp, Artificial Sequence
  smMIP
  misc_feature (29)..(36): n is a, c, g, or t
gttcagtgagagactccaggactttgacnnnnnnnncttcagcttcccgatatccgacggtagtgtagaaagtatgctgacaa

SEQ ID NO: 647 - DNA, 83 bp, Artificial Sequence
  smMIP
  misc_feature (30)..(37): n is a, c, g, or t
ggttctaaaacgaaagtatttgggtagtcnnnnnnnncttcagcttcccgatatccgacggtagtgtcaggtgaagatttggt

SEQ ID NO: 648 - DNA, 83 bp, Artificial Sequence
  smMIP
  misc_feature (30)..(37): n is a, c, g, or t
tattaaccaccaggtggtgccaacactggnnnnnnnncttcagcttcccgatatccgacggtagtgtacaaaatactaacaca

SEQ ID NO: 649 - DNA, 83 bp, Artificial Sequence
  smMIP
  misc_feature (30)..(37): n is a, c, g, or t
ggatatacagtggaagtgcagtttgatggnnnnnnnncttcagcttcccgatatccgacggtagtgttgaactgcaactaacg

SEQ ID NO: 650 - DNA, 83 bp, Artificial Sequence
  smMIP
  misc_feature (30)..(37): n is a, c, g, or t
gtaaaaataaagtatgggaagttcatgcgnnnnnnnncttcagcttcccgatatccgacggtagtgtatttgtgaagaagcat

SEQ ID NO: 651 - DNA, 83 bp, Artificial Sequence
  smMIP
  misc_feature (21)..(28): n is a, c, g, or t
agccagacaccggaaaccccnnnnnnnncttcagcttcccgatatccgacggtagtgtagcagcaacgaagtatcctctcctg

SEQ ID NO: 652 - DNA, 83 bp, Artificial Sequence
  smMIP
  misc_feature (30)..(37): n is a, c, g, or t
gcattgtacattgtatactgcagtgtcgtnnnnnnnncttcagcttcccgatatccgacggtagtgttcctcactgcatttaa

SEQ ID NO: 653 - DNA, 82 bp, Artificial Sequence
  smMIP
  misc_feature (27)..(34): n is a, c, g, or t
gttactgcgacgtgaggtatatgactnnnnnnnncttcagcttcccgatatccgacggtagtgtaccaaaagagaactgcaa

SEQ ID NO: 654 - DNA, 83 bp, Artificial Sequence
  smMIP
  misc_feature (30)..(37): n is a, c, g, or t
gttttattctaaaattagtgagtatagacnnnnnnnncttcagcttcccgatatccgacggtagtgttagaatgtgtgtactg

SEQ ID NO: 655 - DNA, 83 bp, Artificial Sequence
  smMIP
  misc_feature (30)..(37): n is a, c, g, or t
cgttgtgtgatttgttaattaggtgtattnnnnnnnncttcagcttcccgatatccgacggtagtgtagagatgggaatccat

SEQ ID NO: 656 - DNA, 79 bp, Artificial Sequence
  smMIP
  misc_feature (23)..(30): n is a, c, g, or t
gtggaccggtcgatgtatgtctnnnnnnnncttcagcttcccgatatccgacggtagtgtgaacagcaatacaacaaac

SEQ ID NO: 657 - DNA, 82 bp, Artificial Sequence
  smMIP
  misc_feature (29)..(36): n is a, c, g, or t
cgtagacattcgtactttggaagacctgnnnnnnnncttcagcttcccgatatccgacggtagtgtgaggaggaggatgaaa

SEQ ID NO: 658 - DNA, 82 bp, Artificial Sequence
  smMIP
  misc_feature (28)..(35): n is a, c, g, or t
gcggggtggggggggagagagagttttnnnnnnnncttcagcttcccgatatccgacggtagtgtacagccttacgcccaca

SEQ ID NO: 659 - DNA, 80 bp, Artificial Sequence
  smMIP
  misc_feature (27)..(34): n is a, c, g, or t
cattactcggggggccatcaggattgnnnnnnnncttcagcttcccgatatccgacggtagtgtagagcctcggagacct

SEQ ID NO: 660 - DNA, 82 bp, Artificial Sequence
  smMIP
  misc_feature (26)..(33): n is a, c, g, or t
gctcagatgctccaaggatgcaccannnnnnnncttcagcttcccgatatccgacggtagtgtaggagtgcccctcgggctt

SEQ ID NO: 661 - DNA, 81 bp, Artificial Sequence
  smMIP
  misc_feature (27)..(34): n is a, c, g, or t
tctctctctgggaatgggtcgtggacnnnnnnnncttcagcttcccgatatccgacggtagtgtaaaatacggatcacaag

SEQ ID NO: 662 - DNA, 82 bp, Artificial Sequence
  smMIP
  misc_feature (29)..(36): n is a, c, g, or t
ggtatgacgcgagatatctatgagacagnnnnnnnncttcagcttcccgatatccgacggtagtgtagatggccggagagat

SEQ ID NO: 663 - DNA, 83 bp, Artificial Sequence
  smMIP
  misc_feature (24)..(31): n is a, c, g, or t
gcccaataatcagagtggcagtgnnnnnnnncttcagcttcccgatatccgacggtagtgtctgtgggtttgcctagtgtttc

SEQ ID NO: 664 - DNA, 83 bp, Artificial Sequence
  smMIP
  misc_feature (28)..(35): n is a, c, g, or t
cctgtgcagcatccagtgggctgatgannnnnnnncttcagcttcccgatatccgacggtagtgtcgaagcatcagcataaga

SEQ ID NO: 665 - DNA, 81 bp, Artificial Sequence
  smMIP
  misc_feature (25)..(32): n is a, c, g, or t
gcaggagagcgtgtctttgtggtgnnnnnnnncttcagcttcccgatatccgacggtagtgtggcaaatgtgtcagctttg

SEQ ID NO: 666 - DNA, 83 bp, Artificial Sequence
  smMIP
  misc_feature (24)..(31): n is a, c, g, or t
gtgactttggcttggcccgggatnnnnnnnncttcagcttcccgatatccgacggtagtgtgagcatctcatctgttacagct

SEQ ID NO: 667 - DNA, 83 bp, Artificial Sequence
  smMIP
  misc_feature (29)..(36): n is a, c, g, or t
gcagggagtctgtggcatctgaaggctcnnnnnnnncttcagcttcccgatatccgacggtagtgtaaaagtaatcccagatg

SEQ ID NO: 668 - DNA, 83 bp, Artificial Sequence
  smMIP
  misc_feature (28)..(35): n is a, c, g, or t
cagtgtgtggacctccatgttatttccnnnnnnnncttcagcttcccgatatccgacggtagtgtgagctcacggatgctgtg

SEQ ID NO: 669 - DNA, 83 bp, Artificial Sequence
  smMIP
  misc_feature (30)..(37): n is a, c, g, or t
cctgaagaatcgattcctcaggccaggtgnnnnnnnncttcagcttcccgatatccgacggtagtgtactgcatcaggaacaa

SEQ ID NO: 670 - DNA, 83 bp, Artificial Sequence
  smMIP
  misc_feature (28)..(35): n is a, c, g, or t
gcttcaaggtatcacgtcatggggcagnnnnnnnncttcagcttcccgatatccgacggtagtgttgacgtgtgtgcgcaagt

SEQ ID NO: 671 - DNA, 81 bp, Artificial Sequence
  smMIP
  misc_feature (28)..(35): n is a, c, g, or t
gtggatcaaggacaccatcgtggccaannnnnnnncttcagcttcccgatatccgacggtagtgtgggtgattctgggggc

SEQ ID NO: 672 - DNA, 83 bp, Artificial Sequence
  smMIP
  misc_feature (22)..(29): n is a, c, g, or t
ctcaagcctccccagttctacnnnnnnnncttcagcttcccgatatccgacggtagtgtccttccctgtacaccaaggtggtg

SEQ ID NO: 673 - DNA, 82 bp, Artificial Sequence
  smMIP
  misc_feature (29)..(36): n is a, c, g, or t
gatccaacaatagaggattcctacaggannnnnnnncttcagcttcccgatatccgacggtagtgtaggtgcgggagagagg

SEQ ID NO: 674 - DNA, 80 bp, Artificial Sequence
  smMIP
  misc_feature (24)..(31): n is a, c, g, or t
gcaggtcaagaggagtacagtgcnnnnnnnncttcagcttcccgatatccgacggtagtgtgagtgccttgacgatacag

SEQ ID NO: 675 - DNA, 83 bp, Artificial Sequence
  smMIP
  misc_feature (28)..(35): n is a, c, g, or t
catttgaagatattcaccattatagagnnnnnnnncttcagcttcccgatatccgacggtagtgtgagaaacctgtctcttgg

SEQ ID NO: 676 - DNA, 83 bp, Artificial Sequence
  smMIP
  misc_feature (22)..(29): n is a, c, g, or t
gtgatttgccttctagaacagnnnnnnnncttcagcttcccgatatccgacggtagtgtgggagggctttctttgtgtatttg

SEQ ID NO: 677 - DNA, 83 bp, Artificial Sequence
  smMIP
  misc_feature (30)..(37): n is a, c, g, or t
gtggaggatgctttttatacattggtgagnnnnnnnncttcagcttcccgatatccgacggtagtgtggtcctagtaggaaat

SEQ ID NO: 678 - DNA, 83 bp, Artificial Sequence
  smMIP
  misc_feature (29)..(36): n is a, c, g, or t
gcattataatgtaatctgggtgttgatgnnnnnnnncttcagcttcccgatatccgacggtagtgtagcaaagacaagacaga

SEQ ID NO: 679 - DNA, 83 bp, Artificial Sequence
  smMIP
  misc_feature (30)..(37): n is a, c, g, or t
gcaaagatggtaaaaagaagaaaaagaagnnnnnnnncttcagcttcccgatatccgacggtagtgtgcaaagaagaaaagac

SEQ ID NO: 680 - DNA, 78 bp, Artificial Sequence
  smMIP
  misc_feature (22)..(29): n is a, c, g, or t
caaagagagcctgcgggcagannnnnnnncttcagcttcccgatatccgacggtagtgtggactgccgcttccgtgtc

SEQ ID NO: 681 - DNA, 82 bp, Artificial Sequence
  smMIP
  misc_feature (23)..(30): n is a, c, g, or t
ggacactgctcttggcccctctnnnnnnnncttcagcttcccgatatccgacggtagtgttgccctgtgtccctgagcagac

SEQ ID NO: 682 - DNA, 81 bp, Artificial Sequence
  smMIP
  misc_feature (19)..(26): n is a, c, g, or t
taccgcatgagccccagcnnnnnnnncttcagcttcccgatatccgacggtagtgtttcttagactccccagacaggccct

SEQ ID NO: 683 - DNA, 82 bp, Artificial Sequence
  smMIP
  misc_feature (27)..(34): n is a, c, g, or t
ggaggacccctcagccgtgcctgtgtnnnnnnnncttcagcttcccgatatccgacggtagtgttggtggttggtgtcgtgg

SEQ ID NO: 684 - DNA, 79 bp, Artificial Sequence
  smMIP
  misc_feature (22)..(29): n is a, c, g, or t
ccagccggccagttccaaaccnnnnnnnncttcagcttcccgatatccgacggtagtgtggcacctacctctgtggggc

SEQ ID NO: 685 - DNA, 83 bp, Artificial Sequence
  smMIP
  misc_feature (23)..(30): n is a, c, g, or t
tctggcccctcctcagcatcttnnnnnnnncttcagcttcccgatatccgacggtagtgtctgtgggttgattccacaccccc

SEQ ID NO: 686 - DNA, 83 bp, Artificial Sequence
  smMIP
  misc_feature (19)..(26): n is a, c, g, or t
cccctgcaccagccccctnnnnnnnncttcagcttcccgatatccgacggtagtgttggatgatttgatgctgtccccggacg

SEQ ID NO: 687 - DNA, 83 bp, Artificial Sequence
  smMIP
  misc_feature (23)..(30): n is a, c, g, or t
gcctgaggttggctctgactgtnnnnnnnncttcagcttcccgatatccgacggtagtgtgagcgctgctcagatagcgatgg

SEQ ID NO: 688 - DNA, 83 bp, Artificial Sequence
  smMIP
  misc_feature (25)..(32): n is a, c, g, or t
cggcgcacagaggaagagaatctcnnnnnnnncttcagcttcccgatatccgacggtagtgtagttcctgcatgggcggcatg

SEQ ID NO: 689 - DNA, 80 bp, Artificial Sequence
  smMIP
  misc_feature (20)..(27): n is a, c, g, or t
tcccacccccatctctcccnnnnnnnncttcagcttcccgatatccgacggtagtgtagcagggctcactccagccacct

SEQ ID NO: 690 - DNA, 83 bp, Artificial Sequence
  smMIP
  misc_feature (25)..(32): n is a, c, g, or t
gcttcctccacgaatttgaaagacnnnnnnnncttcagcttcccgatatccgacggtagtgttctctccctttcttacctccc

SEQ ID NO: 691 - DNA, 82 bp, Artificial Sequence
  smMIP
  misc_feature (29)..(36): n is a, c, g, or t
gcaacatggcaagacggtggtgggccaannnnnnnncttcagcttcccgatatccgacggtagtgtaaccaagaatgcatct

SEQ ID NO: 692 - DNA, 82 bp, Artificial Sequence
  smMIP
  misc_feature (29)..(36): n is a, c, g, or t
gttcttgatcctgatgagggcatccgttnnnnnnnncttcagcttcccgatatccgacggtagtgtaaggagcaggccagaa

SEQ ID NO: 693 - DNA, 81 bp, Artificial Sequence
  smMIP
  misc_feature (27)..(34): n is a, c, g, or t
gcctgagggcttattttggctgctggnnnnnnnncttcagcttcccgatatccgacggtagtgtggcatgaagggattggt

SEQ ID NO: 694 - DNA, 81 bp, Artificial Sequence
  smMIP
  misc_feature (27)..(34): n is a, c, g, or t
cccatgtggtcaccatgctggacaacnnnnnnnncttcagcttcccgatatccgacggtagtgtggctaagggtggggaag

SEQ ID NO: 695 - DNA, 79 bp, Artificial Sequence
  smMIP
  misc_feature (24)..(31): n is a, c, g, or t
gcccgagcatatgcacagggtatnnnnnnnncttcagcttcccgatatccgacggtagtgtaagagtgggcaaagaggg

SEQ ID NO: 696 - DNA, 82 bp, Artificial Sequence
  smMIP
  misc_feature (23)..(30): n is a, c, g, or t
gctaccttgtgttgcagcaaagnnnnnnnncttcagcttcccgatatccgacggtagtgtgtctcagctcagtgcagctgtt

SEQ ID NO: 697 - DNA, 83 bp, Artificial Sequence
  smMIP
  misc_feature (29)..(36): n is a, c, g, or t
ggactggtctcacaatttcaccaacatgnnnnnnnncttcagcttcccgatatccgacggtagtgtgtactgggagttgattt

SEQ ID NO: 698 - DNA, 81 bp, Artificial Sequence
  smMIP
  misc_feature (28)..(35): n is a, c, g, or t
gtgaccatgagggtggcaatgtaagtgnnnnnnnncttcagcttcccgatatccgacggtagtgtagagaaggcagcggta

SEQ ID NO: 699 - DNA, 83 bp, Artificial Sequence
  smMIP
  misc_feature (30)..(37): n is a, c, g, or t
gcctctccatggactggcaaatcaggaagnnnnnnnncttcagcttcccgatatccgacggtagtgttacctcaccatccaca

SEQ ID NO: 700 - DNA, 83 bp, Artificial Sequence
  smMIP
  misc_feature (30)..(37): n is a, c, g, or t
gttacgagactacatctggaacacactcannnnnnnncttcagcttcccgatatccgacggtagtgtctgtcctttgcagcag

SEQ ID NO: 701 - DNA, 83 bp, Artificial Sequence
  smMIP
  misc_feature (28)..(35): n is a, c, g, or t
gcgatatacctgtcagcgagagtttgcnnnnnnnncttcagcttcccgatatccgacggtagtgtagctgcagaaggaagttg

SEQ ID NO: 702 - DNA, 83 bp, Artificial Sequence
  smMIP
  misc_feature (23)..(30): n is a, c, g, or t
gtgcccaatgtcctcttagagcnnnnnnnncttcagcttcccgatatccgacggtagtgtgttgttccaggctatggccatgc

SEQ ID NO: 703 - DNA, 83 bp, Artificial Sequence
  smMIP
  misc_feature (29)..(36): n is a, c, g, or t
gatgaattactacacggtcctgtttgggnnnnnnnncttcagcttcccgatatccgacggtagtgtgttggttgctcagctgt

SEQ ID NO: 704 - DNA, 82 bp, Artificial Sequence
  smMIP
  misc_feature (27)..(34): n is a, c, g, or t
gaaaggcccaagtccatgagcacagannnnnnnncttcagcttcccgatatccgacggtagtgtgtggggtgctgctccagt

SEQ ID NO: 705 - DNA, 83 bp, Artificial Sequence
  smMIP
  misc_feature (30)..(37): n is a, c, g, or t
gagactgggtgaaagtgactaccagaaagnnnnnnnncttcagcttcccgatatccgacggtagtgtgcattgggtgtactgg

SEQ ID NO: 706 - DNA, 83 bp, Artificial Sequence
  smMIP
  misc_feature (27)..(34): n is a, c, g, or t
gtccccgtctttgacgagatcatcctnnnnnnnncttcagcttcccgatatccgacggtagtgtgctgtagcaaggtgctgct

SEQ ID NO: 707 - DNA, 78 bp, Artificial Sequence
  smMIP
  misc_feature (23)..(30): n is a, c, g, or t
gctgagccggtatgtggaggaannnnnnnncttcagcttcccgatatccgacggtagtgtaggcatggtgggtggcag

SEQ ID NO: 708 - DNA, 83 bp, Artificial Sequence
  smMIP
  misc_feature (22)..(29): n is a, c, g, or t
gctggaggcctgcggtttcttnnnnnnnncttcagcttcccgatatccgacggtagtgtaattctggtttgccaggcgggctg

SEQ ID NO: 709 - DNA, 83 bp, Artificial Sequence
  smMIP
  misc_feature (30)..(37): n is a, c, g, or t
ggaaggacaacacgggctatgacctgaagnnnnnnnncttcagcttcccgatatccgacggtagtgtaaacgtggcaaccaac

SEQ ID NO: 710 - DNA, 83 bp, Artificial Sequence
  smMIP
  misc_feature (22)..(29): n is a, c, g, or t
gctgtgaacgtggctttcctcnnnnnnnncttcagcttcccgatatccgacggtagtgttgtcctggactgcctgacctccct

SEQ ID NO: 711 - DNA, 83 bp, Artificial Sequence
  smMIP
  misc_feature (27)..(34): n is a, c, g, or t
gcattcgagttcatggatgctgtgtgnnnnnnnncttcagcttcccgatatccgacggtagtgtcggtgtccatcttgtgtcc

SEQ ID NO: 712 - DNA, 83 bp, Artificial Sequence
  smMIP
  misc_feature (21)..(28): n is a, c, g, or t
gctccaacgcaggccatgacnnnnnnnncttcagcttcccgatatccgacggtagtgtggggatgctgggtgagatcctgtct

SEQ ID NO: 713 - DNA, 82 bp, Artificial Sequence
  smMIP
  misc_feature (24)..(31): n is a, c, g, or t
gccaccgaccagaggaaagtcaannnnnnnncttcagcttcccgatatccgacggtagtgtcggtgcaagagagtccgtttt

SEQ ID NO: 714 - DNA, 83 bp, Artificial Sequence
  smMIP
  misc_feature (23)..(30): n is a, c, g, or t
tacaagtacgacctctccctccnnnnnnnncttcagcttcccgatatccgacggtagtgtttcctggagcacgcgctgggctc

SEQ ID NO: 715 - DNA, 82 bp, Artificial Sequence
  smMIP
  misc_feature (23)..(30): n is a, c, g, or t
gccaagcacgtggtgggctatgnnnnnnnncttcagcttcccgatatccgacggtagtgttgggccctgagggaaaggatca

SEQ ID NO: 716 - DNA, 83 bp, Artificial Sequence
  smMIP
  misc_feature (28)..(35): n is a, c, g, or t
gcacggagtgggcttcaggaagagggannnnnnnncttcagcttcccgatatccgacggtagtgtaccttggagatggtaacc

SEQ ID NO: 717 - DNA, 80 bp, Artificial Sequence
  smMIP
  misc_feature (23)..(30): n is a, c, g, or t
gtattatggcgcccagaccgtgnnnnnnnncttcagcttcccgatatccgacggtagtgtttcggctcccggcttgggtg

SEQ ID NO: 718 - DNA, 81 bp, Artificial Sequence
  smMIP
  misc_feature (27)..(34): n is a, c, g, or t
gaagcgagcggccgctgaagtaaaccnnnnnnnncttcagcttcccgatatccgacggtagtgtggtgaactaaaggtgcc

SEQ ID NO: 719 - DNA, 83 bp, Artificial Sequence
  smMIP
  misc_feature (30)..(37): n is a, c, g, or t
cattttcctctcgtggtatggcagactggnnnnnnnncttcagcttcccgatatccgacggtagtgtaagcttttggcatctt

SEQ ID NO: 720 - DNA, 83 bp, Artificial Sequence
  smMIP
  misc_feature (28)..(35): n is a, c, g, or t
gtgaacttggcagcaagatacctgtgcnnnnnnnncttcagcttcccgatatccgacggtagtgtgaggtagctgaaggtaaa

SEQ ID NO: 721 - DNA, 83 bp, Artificial Sequence
  smMIP
  misc_feature (29)..(36): n is a, c, g, or t
gctgcaatagaagttcatgaagtactgtnnnnnnnncttcagcttcccgatatccgacggtagtgtagcaatagagcaattga

SEQ ID NO: 722 - DNA, 83 bp, Artificial Sequence
  smMIP
  misc_feature (27)..(34): n is a, c, g, or t
cgtactcatactcaggatgctgttccnnnnnnnncttcagcttcccgatatccgacggtagtgtttcccacagcaatgcacat

SEQ ID NO: 723 - DNA, 83 bp, Artificial Sequence
  smMIP
  misc_feature (30)..(37): n is a, c, g, or t
gccaagaatctatgagctcgcagctggagnnnnnnnncttcagcttcccgatatccgacggtagtgtgcacagatcatcaaga

SEQ ID NO: 724 - DNA, 83 bp, Artificial Sequence
  smMIP
  misc_feature (29)..(36): n is a, c, g, or t
gtggctgcacttacaggcttgccttttgnnnnnnnncttcagcttcccgatatccgacggtagtgtaaatatgcaatgacaag

SEQ ID NO: 725 - DNA, 83 bp, Artificial Sequence
  smMIP
  misc_feature (29)..(36): n is a, c, g, or t
gcctgcagtctgatgaagatagcaaatgnnnnnnnncttcagcttcccgatatccgacggtagtgtgaaaaggttgctgcaaa

SEQ ID NO: 726 - DNA, 83 bp, Artificial Sequence
  smMIP
  misc_feature (29)..(36): n is a, c, g, or t
accaggaagcagtatcatgccaggcaagnnnnnnnncttcagcttcccgatatccgacggtagtgtggttgagctcagtggag

SEQ ID NO: 727 - DNA, 80 bp, Artificial Sequence
  smMIP
  misc_feature (27)..(34): n is a, c, g, or t
gttgctgtcactgtcggaggcagcaannnnnnnncttcagcttcccgatatccgacggtagtgtggtcaggtctgggaga

SEQ ID NO: 728 - DNA, 80 bp, Artificial Sequence
  smMIP
  misc_feature (24)..(31): n is a, c, g, or t
gctgctgggggatgcttcagtttnnnnnnnncttcagcttcccgatatccgacggtagtgtgcaatgaccatggttgcag

SEQ ID NO: 729 - DNA, 83 bp, Artificial Sequence
  smMIP
  misc_feature (30)..(37): n is a, c, g, or t
gctgatgaatgagtctctaatgttggtgannnnnnnncttcagcttcccgatatccgacggtagtgtgttttcaagccaatga

SEQ ID NO: 730 - DNA, 82 bp, Artificial Sequence
  smMIP
  misc_feature (29)..(36): n is a, c, g, or t
gcacacaaaaatggatcaaccttaaaggnnnnnnnncttcagcttcccgatatccgacggtagtgttgcgtggtgggaatcc

SEQ ID NO: 731 - DNA, 83 bp, Artificial Sequence
  smMIP
  misc_feature (28)..(35): n is a, c, g, or t
ggacatgctgggtccaaagtgatttacnnnnnnnncttcagcttcccgatatccgacggtagtgttagggtatgacaaggcag

SEQ ID NO: 732 - DNA, 83 bp, Artificial Sequence
  smMIP
  misc_feature (30)..(37): n is a, c, g, or t
ctacgtggaattggatctacatagctatgnnnnnnnncttcagcttcccgatatccgacggtagtgtctgtcaaggtttattg

SEQ ID NO: 733 - DNA, 83 bp, Artificial Sequence
  smMIP
  misc_feature (29)..(36): n is a, c, g, or t
gcataatgttggcgtcaaatgtgccactnnnnnnnncttcagcttcccgatatccgacggtagtgtgattaaagagaaactca

SEQ ID NO: 734 - DNA, 83 bp, Artificial Sequence
  smMIP
  misc_feature (29)..(36): n is a, c, g, or t
attctgggtggcacggtcttcagagaagnnnnnnnncttcagcttcccgatatccgacggtagtgtgcagaagctataaagaa

SEQ ID NO: 735 - DNA, 83 bp, Artificial Sequence
  smMIP
  misc_feature (29)..(36): n is a, c, g, or t
gcttatggggatcaatacagagcaactgnnnnnnnncttcagcttcccgatatccgacggtagtgtaaatcaccaaatggcac

SEQ ID NO: 736 - DNA, 83 bp, Artificial Sequence
  smMIP
  misc_feature (26)..(33): n is a, c, g, or t
gaacccaaaaggtgacatacctggtnnnnnnnncttcagcttcccgatatccgacggtagtgtggcttgtgagtggatgggta

SEQ ID NO: 737 - DNA, 83 bp, Artificial Sequence
  smMIP
  misc_feature (28)..(35): n is a, c, g, or t
gttccttccaaatggctctgtctaaggnnnnnnnncttcagcttcccgatatccgacggtagtgtataacctacacaccaagt

SEQ ID NO: 738 - DNA, 83 bp, Artificial Sequence
  smMIP
  misc_feature (26)..(33): n is a, c, g, or t
gcgttttaaagacatctttcaggagnnnnnnnncttcagcttcccgatatccgacggtagtgtcatggggatgtataatcaag

SEQ ID NO: 739 - DNA, 83 bp, Artificial Sequence
  smMIP
  misc_feature (29)..(36): n is a, c, g, or t
cgacgacatggtggcccaagctatgaaannnnnnnncttcagcttcccgatatccgacggtagtgtcaccaaaaacactattc

SEQ ID NO: 740 - DNA, 83 bp, Artificial Sequence
  smMIP
  misc_feature (26)..(33): n is a, c, g, or t
gtcggactctgtggcccaagggtatnnnnnnnncttcagcttcccgatatccgacggtagtgtcaagtcccagtttgaagctc

SEQ ID NO: 741 - DNA, 83 bp, Artificial Sequence
  smMIP
  misc_feature (26)..(33): n is a, c, g, or t
gcagaggctgcccacgggactgtaannnnnnnncttcagcttcccgatatccgacggtagtgtggaggcttcatctgggcctg

SEQ ID NO: 742 - DNA, 83 bp, Artificial Sequence
  smMIP
  misc_feature (29)..(36): n is a, c, g, or t
gcttccatttttgcctggaccagagggtnnnnnnnncttcagcttcccgatatccgacggtagtgtatgatgaccagcgtgct

SEQ ID NO: 743 - DNA, 83 bp, Artificial Sequence
  smMIP
  misc_feature (30)..(37): n is a, c, g, or t
gaagtctctattgagacaattgaggctggnnnnnnnncttcagcttcccgatatccgacggtagtgtagaaaggacaggagac

SEQ ID NO: 744 - DNA, 83 bp, Artificial Sequence
  smMIP
  misc_feature (20)..(27): n is a, c, g, or t
gcaacgttctgactacttgnnnnnnnncttcagcttcccgatatccgacggtagtgtcagagcaaagcttgataacaataaag

SEQ ID NO: 745 - DNA, 83 bp, Artificial Sequence
  smMIP
  misc_feature (28)..(35): n is a, c, g, or t
gttcatacctgagctaagaaggataatnnnnnnnncttcagcttcccgatatccgacggtagtgtggacttggctgcttgcat

SEQ ID NO: 746 - DNA, 83 bp, Artificial Sequence
  smMIP
  misc_feature (30)..(37): n is a, c, g, or t
acccctgatgaggcccgtgtggaagagttnnnnnnnncttcagcttcccgatatccgacggtagtgtacatccagctaaagta

SEQ ID NO: 747 - DNA, 83 bp, Artificial Sequence
  smMIP
  misc_feature (27)..(34): n is a, c, g, or t
gagcccatcatctgcaaaaacatcccnnnnnnnncttcagcttcccgatatccgacggtagtgtgtgtggctgtcaagtgtgc

SEQ ID NO: 748 - DNA, 83 bp, Artificial Sequence
  smMIP
  misc_feature (23)..(30): n is a, c, g, or t
gcgaccagtacaaggccacagannnnnnnncttcagcttcccgatatccgacggtagtgtaaagtcccaatggaactatccgg

SEQ ID NO: 749 - DNA, 83 bp, Artificial Sequence
  smMIP
  misc_feature (29)..(36): n is a, c, g, or t
gagtgggaagtgtacaacttccccgcagnnnnnnnncttcagcttcccgatatccgacggtagtgtaccaagcccatcaccat

SEQ ID NO: 750 - DNA, 83 bp, Artificial Sequence
  smMIP
  misc_feature (30)..(37): n is a, c, g, or t
gtatgccatccagaagaaatggccgctgtnnnnnnnncttcagcttcccgatatccgacggtagtgtaccccaaaagatggca

SEQ ID NO: 751 - DNA, 83 bp, Artificial Sequence
  smMIP
  misc_feature (26)..(33): n is a, c, g, or t
gcactataagaccgacttcgacaagnnnnnnnncttcagcttcccgatatccgacggtagtgtttttgcgcacagctgcttcc

SEQ ID NO: 752 - DNA, 83 bp, Artificial Sequence
  smMIP
  misc_feature (28)..(35): n is a, c, g, or t
gctttgtgtgggcctgcaagaactatgnnnnnnnncttcagcttcccgatatccgacggtagtgtttcaaggacatcttccag

SEQ ID NO: 753 - DNA, 83 bp, Artificial Sequence
  smMIP
  misc_feature (23)..(30): n is a, c, g, or t
gccctgatgggaagacgattgannnnnnnncttcagcttcccgatatccgacggtagtgttggtggctcaggtcctcaagtct

SEQ ID NO: 754 - DNA, 83 bp, Artificial Sequence
  smMIP
  misc_feature (19)..(26): n is a, c, g, or t
ccaccagcaccaaccccannnnnnnncttcagcttcccgatatccgacggtagtgtatcctggcccagggctttggctccctt

SEQ ID NO: 755 - DNA, 81 bp, Artificial Sequence
  smMIP
  misc_feature (28)..(35): n is a, c, g, or t
gctggagaaggtgtgcgtggagacggtnnnnnnnncttcagcttcccgatatccgacggtagtgtgggagcaccagaaggg

SEQ ID NO: 756 - DNA, 83 bp, Artificial Sequence
  smMIP
  misc_feature (30)..(37): n is a, c, g, or t
gcaatgtgaagctgaacgagcacttcctgnnnnnnnncttcagcttcccgatatccgacggtagtgtggggaagctggatggg

SEQ ID NO: 757 - DNA, 83 bp, Artificial Sequence
  smMIP
  misc_feature (29)..(36): n is a, c, g, or t
gtgactggagcagctggtcaaattgcatnnnnnnnncttcagcttcccgatatccgacggtagtgttgggagaggagcgatct

SEQ ID NO: 758 - DNA, 83 bp, Artificial Sequence
  smMIP
  misc_feature (28)..(35): n is a, c, g, or t
gctgttggatatcacccccatgatgggnnnnnnnncttcagcttcccgatatccgacggtagtgtaggacgataagtctgaac

SEQ ID NO: 759 - DNA, 83 bp, Artificial Sequence
  smMIP
  misc_feature (27)..(34): n is a, c, g, or t
gcaacagataaagaagacgttgccttnnnnnnnncttcagcttcccgatatccgacggtagtgtctgtctttggtaaagatca

SEQ ID NO: 760 - DNA, 83 bp, Artificial Sequence
  smMIP
  misc_feature (30)..(37): n is a, c, g, or t
gcaaatgtgaaaatcttcaaatcccagggnnnnnnnncttcagcttcccgatatccgacggtagtgtccctcctgaaagatgt

SEQ ID NO: 761 - DNA, 83 bp, Artificial Sequence
  smMIP
  misc_feature (30)..(37): n is a, c, g, or t
gccaataccaactgcctgactgcttccaannnnnnnncttcagcttcccgatatccgacggtagtgtgaagggaaggcatgga

SEQ ID NO: 762 - DNA, 83 bp, Artificial Sequence
  smMIP
  misc_feature (29)..(36): n is a, c, g, or t
gatcacaaccgagctaaagctcaaattgnnnnnnnncttcagcttcccgatatccgacggtagtgtaaatacgccaagaagtc

SEQ ID NO: 763 - DNA, 82 bp, Artificial Sequence
  smMIP
  misc_feature (29)..(36): n is a, c, g, or t
gaaaccattcctcgactcagtatccagannnnnnnncttcagcttcccgatatccgacggtagtgtctccatccatccccaa

SEQ ID NO: 764 - DNA, 83 bp, Artificial Sequence
  smMIP
  misc_feature (29)..(36): n is a, c, g, or t
gctctgaaagatgacagctggctcaaggnnnnnnnncttcagcttcccgatatccgacggtagtgtcttggtgtgactgctaa

SEQ ID NO: 765 - DNA, 83 bp, Artificial Sequence
  smMIP
  misc_feature (30)..(37): n is a, c, g, or t
gccatgtctgctgcaaaagccatctgtgannnnnnnncttcagcttcccgatatccgacggtagtgttgcaaggaaaggaagt

SEQ ID NO: 766 - DNA, 83 bp, Artificial Sequence
  smMIP
  misc_feature (30)..(37): n is a, c, g, or t
gcaactcctatggtgttcctgatgatctgnnnnnnnncttcagcttcccgatatccgacggtagtgtcatcaaggctcgaaaa

SEQ ID NO: 767 - DNA, 83 bp, Artificial Sequence
  smMIP
  misc_feature (28)..(35): n is a, c, g, or t
gatttctcacgtgagaagatggatcttnnnnnnnncttcagcttcccgatatccgacggtagtgtgtttgtgtccatgggtgt

SEQ ID NO: 768 - DNA, 83 bp, Artificial Sequence
  smMIP
  misc_feature (30)..(37): n is a, c, g, or t
gcctgactagacaatgatgttactaaatgnnnnnnnncttcagcttcccgatatccgacggtagtgtagaataagacctggaa

SEQ ID NO: 769 - DNA, 83 bp, Artificial Sequence
  smMIP
  misc_feature (30)..(37): n is a, c, g, or t
gctatacttaaattacttgtgaaaaacaannnnnnnncttcagcttcccgatatccgacggtagtgtgaagaaaaagaaagtg

SEQ ID NO: 770 - DNA, 83 bp, Artificial Sequence
  smMIP
  misc_feature (26)..(33): n is a, c, g, or t
gccactttcacttctcctgaagaacnnnnnnnncttcagcttcccgatatccgacggtagtgttcccgctccagccatgctct

SEQ ID NO: 771 - DNA, 83 bp, Artificial Sequence
  smMIP
  misc_feature (20)..(27): n is a, c, g, or t
ccacatcgagaccaaagccnnnnnnnncttcagcttcccgatatccgacggtagtgttaaagtagctgtgctaggggcctctg

SEQ ID NO: 772 - DNA, 83 bp, Artificial Sequence
  smMIP
  misc_feature (26)..(33): n is a, c, g, or t
gtggtagttattccggctggagtccnnnnnnnncttcagcttcccgatatccgacggtagtgtctgaccctctatgatatcgc

SEQ ID NO: 773 - DNA, 83 bp, Artificial Sequence
  smMIP
  misc_feature (22)..(29): n is a, c, g, or t
gctgcctgtgcccagcactgcnnnnnnnncttcagcttcccgatatccgacggtagtgtacctgaacagctgcctgactgcct

SEQ ID NO: 774 - DNA, 83 bp, Artificial Sequence
  smMIP
  misc_feature (30)..(37): n is a, c, g, or t
cattggtggccatgctgggaagaccatcannnnnnnncttcagcttcccgatatccgacggtagtgtttcaacaccaatgcca

SEQ ID NO: 775 - DNA, 83 bp, Artificial Sequence
  smMIP
  misc_feature (21)..(28): n is a, c, g, or t
cagctgacagcactcactggnnnnnnnncttcagcttcccgatatccgacggtagtgttgatctgcgtcattgccaatccggg

SEQ ID NO: 776 - DNA, 83 bp, Artificial Sequence
  smMIP
  misc_feature (25)..(32): n is a, c, g, or t
gctttgtcttctcccttgtggatgnnnnnnnncttcagcttcccgatatccgacggtagtgtaaggtggactttccccaggac

SEQ ID NO: 777 - DNA, 83 bp, Artificial Sequence
  smMIP
  misc_feature (30)..(37): n is a, c, g, or t
gctgctgcttgggaaaaagggcatcgagannnnnnnncttcagcttcccgatatccgacggtagtgtatggcgtatgccggcg

SEQ ID NO: 778 - DNA, 82 bp, Artificial Sequence
  smMIP
  misc_feature (28)..(35): n is a, c, g, or t
gctgaaggcctccatcaagaagggggannnnnnnncttcagcttcccgatatccgacggtagtgtaggaaacggaatgtacc

SEQ ID NO: 779 - DNA, 83 bp, Artificial Sequence
  smMIP
  misc_feature (30)..(37): n is a, c, g, or t
gcatcatgtcactgcaaagccgttgcagannnnnnnncttcagcttcccgatatccgacggtagtgtaggagaagatgatctc

SEQ ID NO: 780 - DNA, 83 bp, Artificial Sequence
  smMIP
  misc_feature (25)..(32): n is a, c, g, or t
aaagacctggagcggctgacacagnnnnnnnncttcagcttcccgatatccgacggtagtgtccgagtgtatactctgaaaga

SEQ ID NO: 781 - DNA, 83 bp, Artificial Sequence
  smMIP
  misc_feature (29)..(36): n is a, c, g, or t
gcttttgatggtactgatgagtcttgatnnnnnnnncttcagcttcccgatatccgacggtagtgtacgaagatctggaagac

SEQ ID NO: 782 - DNA, 83 bp, Artificial Sequence
  smMIP
  misc_feature (25)..(32): n is a, c, g, or t
gtgacttctgccacattacctgacnnnnnnnncttcagcttcccgatatccgacggtagtgtcctgtagcaagtattttcgcc

SEQ ID NO: 783 - DNA, 83 bp, Artificial Sequence
  smMIP
  misc_feature (26)..(33): n is a, c, g, or t
cacacacacacacacacacaccagcnnnnnnnncttcagcttcccgatatccgacggtagtgtgatagcgctctcatggcttg

SEQ ID NO: 784 - DNA, 83 bp, Artificial Sequence
  smMIP
  misc_feature (29)..(36): n is a, c, g, or t
gcatttgaaggatcaaacaatcaacatcnnnnnnnncttcagcttcccgatatccgacggtagtgtggaaagatacctgataa

SEQ ID NO: 785 - DNA, 83 bp, Artificial Sequence
  smMIP
  misc_feature (24)..(31): n is a, c, g, or t
gccctggggatcggcctcttcatnnnnnnnncttcagcttcccgatatccgacggtagtgtacctgtgccatccaaactgcac

SEQ ID NO: 786 - DNA, 83 bp, Artificial Sequence
  smMIP
  misc_feature (26)..(33): n is a, c, g, or t
gcacggtgtataagggactctggatnnnnnnnncttcagcttcccgatatccgacggtagtgttgctgcaggagagggagctt

SEQ ID NO: 787 - DNA, 83 bp, Artificial Sequence
  smMIP
  misc_feature (27)..(34): n is a, c, g, or t
gccaacaaggaaatcctcgatgaagcnnnnnnnncttcagcttcccgatatccgacggtagtgtaaaagatcaaagtgctggg

SEQ ID NO: 788 - DNA, 83 bp, Artificial Sequence
  smMIP
  misc_feature (23)..(30): n is a, c, g, or t
tctgcctcacctccaccgtgcannnnnnnncttcagcttcccgatatccgacggtagtgtaagttaaaattcccgtcgctatc

SEQ ID NO: 789 - DNA, 82 bp, Artificial Sequence
  smMIP
  misc_feature (29)..(36): n is a, c, g, or t
gctcccagtacctgctcaactggtgtgtnnnnnnnncttcagcttcccgatatccgacggtagtgttggacaacccccacgt

SEQ ID NO: 790 - DNA, 83 bp, Artificial Sequence
  smMIP
  misc_feature (27)..(34): n is a, c, g, or t
gcagaaggaggcaaagtgcctatcaannnnnnnncttcagcttcccgatatccgacggtagtgtaggaccgtcgcttggtgca

SEQ ID NO: 791 - DNA, 83 bp, Artificial Sequence
  smMIP
  misc_feature (27)..(34): n is a, c, g, or t
gagttgatgacctttggatccaagccnnnnnnnncttcagcttcccgatatccgacggtagtgtcggaagagaaagaatacca

SEQ ID NO: 792 - DNA, 83 bp, Artificial Sequence
  smMIP
  misc_feature (23)..(30): n is a, c, g, or t
gccaagtcctacagactccaacnnnnnnnncttcagcttcccgatatccgacggtagtgtcatggtcaagtgctggatgatag

SEQ ID NO: 793 - DNA, 83 bp, Artificial Sequence
  smMIP
  misc_feature (29)..(36): n is a, c, g, or t
gacagcatagacgacaccttcctcccagnnnnnnnncttcagcttcccgatatccgacggtagtgtagtgcaaccagcaacaa

SEQ ID NO: 794 - DNA, 82 bp, Artificial Sequence
  smMIP
  misc_feature (28)..(35): n is a, c, g, or t
gccaccaaattagcctggacaaccctgnnnnnnnncttcagcttcccgatatccgacggtagtgtagagacccacactacca

SEQ ID NO: 795 - DNA, 83 bp, Artificial Sequence
  smMIP
  misc_feature (30)..(37): n is a, c, g, or t
ggaaattacctatgtgcagaggaattatgnnnnnnnncttcagcttcccgatatccgacggtagtgtctggaggaaaagaaag

SEQ ID NO: 796 - DNA, 83 bp, Artificial Sequence
  smMIP
  misc_feature (26)..(33): n is a, c, g, or t
gcaaataaaaccggactgaaggagcnnnnnnnncttcagcttcccgatatccgacggtagtgtgtggctggttatgtcctcat

SEQ ID NO: 797 - DNA, 83 bp, Artificial Sequence
  smMIP
  misc_feature (25)..(32): n is a, c, g, or t
gccctgtgcaacgtggagagcatcnnnnnnnncttcagcttcccgatatccgacggtagtgtctacgaaaattcctatgcctt

SEQ ID NO: 798 - DNA, 83 bp, Artificial Sequence
  smMIP
  misc_feature (30)..(37): n is a, c, g, or t
gcctggtctgccgcaaattccgagacgaannnnnnnncttcagcttcccgatatccgacggtagtgtccagaaactgaccaaa

SEQ ID NO: 799 - DNA, 82 bp, Artificial Sequence
  smMIP
  misc_feature (26)..(33): n is a, c, g, or t
gtcactggtattcatggggacccttnnnnnnnncttcagcttcccgatatccgacggtagtgtcactgacatttgcccaaaa

SEQ ID NO: 800 - DNA, 83 bp, Artificial Sequence
  smMIP
  misc_feature (29)..(36): n is a, c, g, or t
acaacactcttcagcacaatcaaccagannnnnnnncttcagcttcccgatatccgacggtagtgttgcttatcctcaagcaa

SEQ ID NO: 801 - DNA, 82 bp, Artificial Sequence
  smMIP
  misc_feature (29)..(36): n is a, c, g, or t
gtgggctcttcattctggtcattgtgggnnnnnnnncttcagcttcccgatatccgacggtagtgtacccatgccatccaaa

SEQ ID NO: 802 - DNA, 83 bp, Artificial Sequence
  smMIP
  misc_feature (28)..(35): n is a, c, g, or t
gccaattaaatggatggctctggagtgnnnnnnnncttcagcttcccgatatccgacggtagtgtgcagcccgtaatgtctta

SEQ ID NO: 803 - DNA, 83 bp, Artificial Sequence
  smMIP
  misc_feature (25)..(32): n is a, c, g, or t
tgcctcagcctcccatctgcactannnnnnnncttcagcttcccgatatccgacggtagtgtgagtgacgtttggagctatgg

SEQ ID NO: 804 - DNA, 80 bp, Artificial Sequence
  smMIP
  misc_feature (26)..(33): n is a, c, g, or t
gctgagttttcaaggatggctcgagnnnnnnnncttcagcttcccgatatccgacggtagtgtagagaaaggagaacgtt

SEQ ID NO: 805 - DNA, 83 bp, Artificial Sequence
  smMIP
  misc_feature (26)..(33): n is a, c, g, or t
gcttcccagtccaaatgacagcaagnnnnnnnncttcagcttcccgatatccgacggtagtgtgttggatgattgatgctgac

SEQ ID NO: 806 - DNA, 83 bp, Artificial Sequence
  smMIP
  misc_feature (30)..(37): n is a, c, g, or t
gggtgctactgctgagatttttgatgactnnnnnnnncttcagcttcccgatatccgacggtagtgtccatgtcaggaaacca

SEQ ID NO: 807 - DNA, 83 bp, Artificial Sequence
  smMIP
  misc_feature (30)..(37): n is a, c, g, or t
cagtagcacccagaggtacagtgctgaccnnnnnnnncttcagcttcccgatatccgacggtagtgtccaactagcacaattc

SEQ ID NO: 808 - DNA, 83 bp, Artificial Sequence
  smMIP
  misc_feature (30)..(37): n is a, c, g, or t
gcaagatattgttcggaacccatggccttnnnnnnnncttcagcttcccgatatccgacggtagtgtacagaaaagatggaaa

SEQ ID NO: 809 - DNA, 83 bp, Artificial Sequence
  smMIP
  misc_feature (29)..(36): n is a, c, g, or t
gctgagtaatggctcagtcatgatttttnnnnnnnncttcagcttcccgatatccgacggtagtgtcccaccaactgaagtca

SEQ ID NO: 810 - DNA, 82 bp, Artificial Sequence
  smMIP
  misc_feature (29)..(36): n is a, c, g, or t
gtccacaatgctacgtgcacagtgaggannnnnnnncttcagcttcccgatatccgacggtagtgtaagcagcaggatggag

SEQ ID NO: 811 - DNA, 83 bp, Artificial Sequence
  smMIP
  misc_feature (23)..(30): n is a, c, g, or t
aatccttctgtcggcgagccatnnnnnnnncttcagcttcccgatatccgacggtagtgtgtggatttattttgattgggttg

SEQ ID NO: 812 - DNA, 80 bp, Artificial Sequence
  smMIP
  misc_feature (27)..(34): n is a, c, g, or t
gcgagatgacatgactgtctgtgttgnnnnnnnncttcagcttcccgatatccgacggtagtgtacaggaccaaagcata

SEQ ID NO: 813 - DNA, 83 bp, Artificial Sequence
  smMIP
  misc_feature (24)..(31): n is a, c, g, or t
gcctgttaaatggatcgccatagnnnnnnnncttcagcttcccgatatccgacggtagtgtcttcatcgagatttagctgctc

SEQ ID NO: 814 - DNA, 83 bp, Artificial Sequence
  smMIP
  misc_feature (27)..(34): n is a, c, g, or t
gaagactgcctggatgaactgtatgannnnnnnncttcagcttcccgatatccgacggtagtgtgtgatgtgtgggcatttgg

SEQ ID NO: 815 - DNA, 83 bp, Artificial Sequence
  smMIP
  misc_feature (28)..(35): n is a, c, g, or t
ctcttagaaagtttgcctgacgttcggnnnnnnnncttcagcttcccgatatccgacggtagtgttggccacaggttgaagca

SEQ ID NO: 816 - DNA, 82 bp, Artificial Sequence
  smMIP
  misc_feature (26)..(33): n is a, c, g, or t
gctgacgactcctcagaaggctcagnnnnnnnncttcagcttcccgatatccgacggtagtgttctgctgcagtcacagctg

SEQ ID NO: 817 - DNA, 83 bp, Artificial Sequence
  smMIP
  misc_feature (27)..(34): n is a, c, g, or t
tcgcttccttcagcataaccagtgtgnnnnnnnncttcagcttcccgatatccgacggtagtgtctcaatcagtgtacctaat

SEQ ID NO: 818 - DNA, 83 bp, Artificial Sequence
  smMIP
  misc_feature (23)..(30): n is a, c, g, or t
cgaacagcctgaaaaatcccccnnnnnnnncttcagcttcccgatatccgacggtagtgttcctcactttactaagcagcctg

SEQ ID NO: 819 - DNA, 82 bp, Artificial Sequence
  smMIP
  misc_feature (21)..(28): n is a, c, g, or t
ttccgcccttcccccccaacnnnnnnnncttcagcttcccgatatccgacggtagtgtcagcctgcccagcctcagtggcat

SEQ ID NO: 820 - DNA, 83 bp, Artificial Sequence
  smMIP
  misc_feature (22)..(29): n is a, c, g, or t
tcaccatctacgactgcagccnnnnnnnncttcagcttcccgatatccgacggtagtgttttggtcaccagattgcctactgc

SEQ ID NO: 821 - DNA, 83 bp, Artificial Sequence
  smMIP
  misc_feature (22)..(29): n is a, c, g, or t
cagacccctgcacggagctgannnnnnnncttcagcttcccgatatccgacggtagtgtttctcctacgtgctgcccctggtc

SEQ ID NO: 822 - DNA, 81 bp, Artificial Sequence
  smMIP
  misc_feature (25)..(32): n is a, c, g, or t
ccggggagcctctcaccctcgttannnnnnnncttcagcttcccgatatccgacggtagtgtgaggtggctgtggctgagg

SEQ ID NO: 823 - DNA, 83 bp, Artificial Sequence
  smMIP
  misc_feature (30)..(37): n is a, c, g, or t
gctgcgacatccagattgtctctgacagannnnnnnncttcagcttcccgatatccgacggtagtgtggccaagagggagaag

SEQ ID NO: 824 - DNA, 83 bp, Artificial Sequence
  smMIP
  misc_feature (23)..(30): n is a, c, g, or t
ttcaaccagaccatcgccacacnnnnnnnncttcagcttcccgatatccgacggtagtgttaccgggtcaagataggccaagt

SEQ ID NO: 825 - DNA, 83 bp, Artificial Sequence
  smMIP
  misc_feature (25)..(32): n is a, c, g, or t
gcaaaggcttcgctgagctgcagannnnnnnncttcagcttcccgatatccgacggtagtgtgtcctgctgctgctctccgtg

SEQ ID NO: 826 - DNA, 82 bp, Artificial Sequence
  smMIP
  misc_feature (28)..(35): n is a, c, g, or t
ggagtataagcacttcgtgacccgcacnnnnnnnncttcagcttcccgatatccgacggtagtgttgctgcagatggaggag

SEQ ID NO: 827 - DNA, 83 bp, Artificial Sequence
  smMIP
  misc_feature (24)..(31): n is a, c, g, or t
tcccagaccctcaactcccagggnnnnnnnncttcagcttcccgatatccgacggtagtgtacatgacagatctcaccaagga

SEQ ID NO: 828 - DNA, 83 bp, Artificial Sequence
  smMIP
  misc_feature (30)..(37): n is a, c, g, or t
gtccatctgcatgtacagctgtctgcgggnnnnnnnncttcagcttcccgatatccgacggtagtgttactacaccagcatca

SEQ ID NO: 829 - DNA, 83 bp, Artificial Sequence
  smMIP
  misc_feature (20)..(27): n is a, c, g, or t
ttcgcctccagcacacagannnnnnnncttcagcttcccgatatccgacggtagtgtatggactcgctgagcgtgcgggccat

SEQ ID NO: 830 - DNA, 83 bp, Artificial Sequence
  smMIP
  misc_feature (28)..(35): n is a, c, g, or t
ctttttcgacttcctggaggagcaggcnnnnnnnncttcagcttcccgatatccgacggtagtgttgctcccggaaatctacc

SEQ ID NO: 831 - DNA, 83 bp, Artificial Sequence
  smMIP
  misc_feature (26)..(33): n is a, c, g, or t
tcatcgcgcaggccttcatcgacgcnnnnnnnncttcagcttcccgatatccgacggtagtgtaccccgacaccctacacatc

SEQ ID NO: 832 - DNA, 83 bp, Artificial Sequence
  smMIP
  misc_feature (30)..(37): n is a, c, g, or t
ctggccgaggagtcgaggaaataccagaannnnnnnncttcagcttcccgatatccgacggtagtgtattcgccaaccaacaa

SEQ ID NO: 833 - DNA, 83 bp, Artificial Sequence
  smMIP
  misc_feature (30)..(37): n is a, c, g, or t
ggtggtggctttgatggaggacaacatctnnnnnnnncttcagcttcccgatatccgacggtagtgtaacaccaatgtggcca

SEQ ID NO: 834 - DNA, 81 bp, Artificial Sequence
  smMIP
  misc_feature (19)..(26): n is a, c, g, or t
tctcccagcaccaagaccnnnnnnnncttcagcttcccgatatccgacggtagtgtctgccccctgtcctgtgcagtcagc

SEQ ID NO: 835 - DNA, 82 bp, Artificial Sequence
  smMIP
  misc_feature (29)..(36): n is a, c, g, or t
gcttccctgaggaggagaagtgcttctgnnnnnnnncttcagcttcccgatatccgacggtagtgtgcgatgttgtggagac

SEQ ID NO: 836 - DNA, 78 bp, Artificial Sequence
  smMIP
  misc_feature (24)..(31): n is a, c, g, or t
attgtatggggcctgcagccaggnnnnnnnncttcagcttcccgatatccgacggtagtgtaaaggcagagcagggta

SEQ ID NO: 837 - DNA, 82 bp, Artificial Sequence
  smMIP
  misc_feature (24)..(31): n is a, c, g, or t
cggcttgtcccgagatgtttatgnnnnnnnncttcagcttcccgatatccgacggtagtgtctcatctcatttgcctggcag

SEQ ID NO: 838 - DNA, 82 bp, Artificial Sequence
  smMIP
  misc_feature (29)..(36): n is a, c, g, or t
gatcatatctacaccacgcaaagtgatgnnnnnnnncttcagcttcccgatatccgacggtagtgtgaggggcggaagatga

SEQ ID NO: 839 - DNA, 83 bp, Artificial Sequence
  smMIP
  misc_feature (21)..(28): n is a, c, g, or t
ggagatgtaccgcctgatgcnnnnnnnncttcagcttcccgatatccgacggtagtgttggtcttttggtgtcctgctgtggg

SEQ ID NO: 840 - DNA, 82 bp, Artificial Sequence
  smMIP
  misc_feature (27)..(34): n is a, c, g, or t
caccgctggtggactgtaataatgccnnnnnnnncttcagcttcccgatatccgacggtagtgtggtgtttgcggacatcag

SEQ ID NO: 841 - DNA, 83 bp, Artificial Sequence
  smMIP
  misc_feature (28)..(35): n is a, c, g, or t
ggatgctttcaccctcagcggcaaaatnnnnnnnncttcagcttcccgatatccgacggtagtgtgaaaacaaactctatggc

SEQ ID NO: 842 - DNA, 82 bp, Artificial Sequence
  smMIP
  misc_feature (29)..(36): n is a, c, g, or t
gtgaaaggtaatggactcacaaggggaannnnnnnncttcagcttcccgatatccgacggtagtgtacgagagctgatggca

SEQ ID NO: 843 - DNA, 79 bp, Artificial Sequence
  smMIP
  misc_feature (21)..(28): n is a, c, g, or t
gctcctgggagaagctcagtnnnnnnnncttcagcttcccgatatccgacggtagtgttgaggaggtgcccagcttccg

SEQ ID NO: 844 - DNA, 83 bp, Artificial Sequence
  smMIP
  misc_feature (23)..(30): n is a, c, g, or t
tcgtcggaccactgaagctaccnnnnnnnncttcagcttcccgatatccgacggtagtgtctgtgtcctcatcttggctctct

SEQ ID NO: 845 - DNA, 83 bp, Artificial Sequence
  smMIP
  misc_feature (20)..(27): n is a, c, g, or t
tcctcctctattcccggctnnnnnnnncttcagcttcccgatatccgacggtagtgttctgcatgaaggaatttgaccatccc

SEQ ID NO: 846 - DNA, 82 bp, Artificial Sequence
  smMIP
  misc_feature (23)..(30): n is a, c, g, or t
gtccgtgtgtgtggcggacttcnnnnnnnncttcagcttcccgatatccgacggtagtgtagtgtacctgcccactcagatg

SEQ ID NO: 847 - DNA, 83 bp, Artificial Sequence
  smMIP
  misc_feature (29)..(36): n is a, c, g, or t
gccattgagagtctagctgaccgtgtctnnnnnnnncttcagcttcccgatatccgacggtagtgtaggaactgcatgctgaa

SEQ ID NO: 848 - DNA, 83 bp, Artificial Sequence
  smMIP
  misc_feature (28)..(35): n is a, c, g, or t
cgggcgtggagaacagcgagatttatgnnnnnnnncttcagcttcccgatatccgacggtagtgtaagatgccagtcaagtgg

SEQ ID NO: 849 - DNA, 82 bp, Artificial Sequence
  smMIP
  misc_feature (29)..(36): n is a, c, g, or t
ggactgtatgccttgatgtcgcggtgctnnnnnnnncttcagcttcccgatatccgacggtagtgtggggtgacaatgtggg

SEQ ID NO: 850 - DNA, 83 bp, Artificial Sequence
  smMIP
  misc_feature (19)..(26): n is a, c, g, or t
agctgaccccccaacccannnnnnnncttcagcttcccgatatccgacggtagtgtgttttacagagctgcgggaagatttgg

SEQ ID NO: 851 - DNA, 82 bp, Artificial Sequence
  smMIP
  misc_feature (21)..(28): n is a, c, g, or t
ttcccaccccacgccttatcnnnnnnnncttcagcttcccgatatccgacggtagtgttcagcctgctgataggggctcccc

SEQ ID NO: 852 - DNA, 81 bp, Artificial Sequence
  smMIP
  misc_feature (22)..(29): n is a, c, g, or t
gcccgaagacaggactgtggcnnnnnnnncttcagcttcccgatatccgacggtagtgtagctcagaatcacctccctgca

SEQ ID NO: 853 - DNA, 82 bp, Artificial Sequence
  smMIP
  misc_feature (21)..(28): n is a, c, g, or t
tccccctggccacggctccannnnnnnncttcagcttcccgatatccgacggtagtgtgctggagggcttgccttacttcct

SEQ ID NO: 854 - DNA, 80 bp, Artificial Sequence
  smMIP
  misc_feature (24)..(31): n is a, c, g, or t
gccaggtcttgaaggctgtccaannnnnnnncttcagcttcccgatatccgacggtagtgtgtctgccatgccttgtgct

SEQ ID NO: 855 - DNA, 83 bp, Artificial Sequence
  smMIP
  misc_feature (29)..(36): n is a, c, g, or t
gccctggggatcggcctcttcatgcgaannnnnnnncttcagcttcccgatatccgacggtagtgtgacaagtgcaaccttct

SEQ ID NO: 856 - DNA, 81 bp, Artificial Sequence
  smMIP
  misc_feature (24)..(31): n is a, c, g, or t
gtggtgacagatcacggctcgtgnnnnnnnncttcagcttcccgatatccgacggtagtgtgagagccggagcgagctctt

SEQ ID NO: 857 - DNA, 81 bp, Artificial Sequence
  smMIP
  misc_feature (28)..(35): n is a, c, g, or t
gccgcaaagtgtgtaacggaataggtannnnnnnncttcagcttcccgatatccgacggtagtgtctggaggaaaagaaag

SEQ ID NO: 858 - DNA, 83 bp, Artificial Sequence
  smMIP
  misc_feature (30)..(37): n is a, c, g, or t
gccaagggagtttgtggagaactctgagtnnnnnnnncttcagcttcccgatatccgacggtagtgtattctgaaaaccgtaa

SEQ ID NO: 859 - DNA, 83 bp, Artificial Sequence
  smMIP
  misc_feature (29)..(36): n is a, c, g, or t
cggggaccagacaactgtatccagtgtgnnnnnnnncttcagcttcccgatatccgacggtagtgtaacctagaaatcatacg

SEQ ID NO: 860 - DNA, 83 bp, Artificial Sequence
  smMIP
  misc_feature (21)..(28): n is a, c, g, or t
cacaatcagcctctgaacccnnnnnnnncttcagcttcccgatatccgacggtagtgtccaaagttccgtgagttgatcatcg

SEQ ID NO: 861 - DNA, 83 bp, Artificial Sequence
  smMIP
  misc_feature (29)..(36): n is a, c, g, or t
gtttcctaattcatctcagaacggttcannnnnnnncttcagcttcccgatatccgacggtagtgtaatagttcaaccagatc

SEQ ID NO: 862 - DNA, 83 bp, Artificial Sequence
  smMIP
  misc_feature (30)..(37): n is a, c, g, or t
cagtccattactgcaaaatactgtccacannnnnnnncttcagcttcccgatatccgacggtagtgtaaaaagagaaagcaaa

SEQ ID NO: 863 - DNA, 83 bp, Artificial Sequence
  smMIP
  misc_feature (30)..(37): n is a, c, g, or t
gcaggttttcccaaatagtgcaccccttgnnnnnnnncttcagcttcccgatatccgacggtagtgtgctttgcagcgcgttg

SEQ ID NO: 864 - DNA, 83 bp, Artificial Sequence
  smMIP
  misc_feature (28)..(35): n is a, c, g, or t
actagagttctccttggaaatgagagcnnnnnnnncttcagcttcccgatatccgacggtagtgtgacatcagagggtcgctt

SEQ ID NO: 865 - DNA, 83 bp, Artificial Sequence
  smMIP
  misc_feature (26)..(33): n is a, c, g, or t
gcacgatgaatactgtgtcaaacagnnnnnnnncttcagcttcccgatatccgacggtagtgtttgaaggagggacaaggctg

SEQ ID NO: 866 - DNA, 83 bp, Artificial Sequence
  smMIP
  misc_feature (26)..(33): n is a, c, g, or t
gccaaccgagagacaagcatcttcannnnnnnncttcagcttcccgatatccgacggtagtgtaatgagagctgcaccttgac

SEQ ID NO: 867 - DNA, 83 bp, Artificial Sequence
  smMIP
  misc_feature (30)..(37): n is a, c, g, or t
attagtacttggtggaaagaacctctcaannnnnnnncttcagcttcccgatatccgacggtagtgtcccaaaccatttcaac

SEQ ID NO: 868 - DNA, 83 bp, Artificial Sequence
  smMIP
  misc_feature (29)..(36): n is a, c, g, or t
cagttagtgtcccgagaatggtcataaannnnnnnncttcagcttcccgatatccgacggtagtgtaaattcatccaaccaaa

SEQ ID NO: 869 - DNA, 83 bp, Artificial Sequence
  smMIP
  misc_feature (28)..(35): n is a, c, g, or t
gtggtgggagcacaataacaggtgttgnnnnnnnncttcagcttcccgatatccgacggtagtgtcaaaccatttcaactgag

SEQ ID NO: 870 - DNA, 83 bp, Artificial Sequence
  smMIP
  misc_feature (26)..(33): n is a, c, g, or t
gcatgtcaacatcgctctaattcagnnnnnnnncttcagcttcccgatatccgacggtagtgtattcatccaaccaaatcttt

SEQ ID NO: 871 - DNA, 83 bp, Artificial Sequence
  smMIP
  misc_feature (26)..(33): n is a, c, g, or t
gcatgtcaacatcgctctaattcagnnnnnnnncttcagcttcccgatatccgacggtagtgtccaaaccatttcaactgagt

SEQ ID NO: 872 - DNA, 83 bp, Artificial Sequence
  smMIP
  misc_feature (29)..(36): n is a, c, g, or t
gcctttttcatgttagatgggatcctttnnnnnnnncttcagcttcccgatatccgacggtagtgtctatgaaattcatccaa

SEQ ID NO: 873 - DNA, 83 bp, Artificial Sequence
  smMIP
  misc_feature (27)..(34): n is a, c, g, or t
gtcacaacaaccttggaagtagtagannnnnnnncttcagcttcccgatatccgacggtagtgtataatagctggcatcacgg

SEQ ID NO: 874 - DNA, 83 bp, Artificial Sequence
  smMIP
  misc_feature (28)..(35): n is a, c, g, or t
ccgaaggaggcacttacacattcctagnnnnnnnncttcagcttcccgatatccgacggtagtgtgaacaccagcagtggatc

SEQ ID NO: 875 - DNA, 83 bp, Artificial Sequence
  smMIP
  misc_feature (27)..(34): n is a, c, g, or t
gcacaatggcacggttgaatgtaaggnnnnnnnncttcagcttcccgatatccgacggtagtgttgtccaggaactgagcaga

SEQ ID NO: 876 - DNA, 83 bp, Artificial Sequence
  smMIP
  misc_feature (30)..(37): n is a, c, g, or t
caaatgggagtttcccagaaacaggctgannnnnnnncttcagcttcccgatatccgacggtagtgtgtgatgattctgacct

SEQ ID NO: 877 - DNA, 83 bp, Artificial Sequence
  smMIP
  misc_feature (29)..(36): n is a, c, g, or t
gctatggtgatcttttgaattttttgagnnnnnnnncttcagcttcccgatatccgacggtagtgtaacgggaagccctcatg

SEQ ID NO: 878 - DNA, 83 bp, Artificial Sequence
  smMIP
  misc_feature (30)..(37): n is a, c, g, or t
gccgacaaaaggagatctgtgagaataggnnnnnnnncttcagcttcccgatatccgacggtagtgtaagatcatgcagaagc

SEQ ID NO: 879 - DNA, 83 bp, Artificial Sequence
  smMIP
  misc_feature (26)..(33): n is a, c, g, or t
gtgaagtggatggcacctgaaagcannnnnnnncttcagcttcccgatatccgacggtagtgtagagacttggcagccagaaa

SEQ ID NO: 880 - DNA, 83 bp, Artificial Sequence
  smMIP
  misc_feature (25)..(32): n is a, c, g, or t
gctgttatgcactgatccgggcttnnnnnnnncttcagcttcccgatatccgacggtagtgtttctgctcctactgcttcgcg

SEQ ID NO: 881 - DNA, 83 bp, Artificial Sequence
  smMIP
  misc_feature (29)..(36): n is a, c, g, or t
tgccaagcttttccttgttgaccgctccnnnnnnnncttcagcttcccgatatccgacggtagtgtaacgaatgagaataagc

SEQ ID NO: 882 - DNA, 83 bp, Artificial Sequence
  smMIP
  misc_feature (23)..(30): n is a, c, g, or t
gctgtgcctgttgtgtctgtgtnnnnnnnncttcagcttcccgatatccgacggtagtgtaaggcgggcatcatgatcaaaag

SEQ ID NO: 883 - DNA, 83 bp, Artificial Sequence
  smMIP
  misc_feature (30)..(37): n is a, c, g, or t
ggattcaaagcataaaaaccattacaagannnnnnnncttcagcttcccgatatccgacggtagtgtcaagaggatggattcg

SEQ ID NO: 884 - DNA, 83 bp, Artificial Sequence
  smMIP
  misc_feature (30)..(37): n is a, c, g, or t
catgttgcagcaattcactgtaaagctggnnnnnnnncttcagcttcccgatatccgacggtagtgtacaccgccaaatttaa

SEQ ID NO: 885 - DNA, 83 bp, Artificial Sequence
  smMIP
  misc_feature (28)..(35): n is a, c, g, or t
gccctagatttctatggggaagtaaggnnnnnnnncttcagcttcccgatatccgacggtagtgtccaatggctaagtgaaga

SEQ ID NO: 886 - DNA, 82 bp, Artificial Sequence
  smMIP
  misc_feature (29)..(36): n is a, c, g, or t
ggattatagaccagtggcactgttgtttnnnnnnnncttcagcttcccgatatccgacggtagtgttttaaaggcacaagag

SEQ ID NO: 887 - DNA, 82 bp, Artificial Sequence
  smMIP
  misc_feature (24)..(31): n is a, c, g, or t
cctcagtttgtggtctgccagctnnnnnnnncttcagcttcccgatatccgacggtagtgtgtcagaggcgctatgtgtatt

SEQ ID NO: 888 - DNA, 81 bp, Artificial Sequence
  smMIP
  misc_feature (25)..(32): n is a, c, g, or t
gccgttacctgtgtgtggtgatatnnnnnnnncttcagcttcccgatatccgacggtagtgtttccaatgttcagtggcgg

SEQ ID NO: 889 - DNA, 83 bp, Artificial Sequence
  smMIP
  misc_feature (23)..(30): n is a, c, g, or t
accaggaccagaggaaacctcannnnnnnncttcagcttcccgatatccgacggtagtgtgttcatgtactttgagttccctc

SEQ ID NO: 890 - DNA, 83 bp, Artificial Sequence
  smMIP
  misc_feature (24)..(31): n is a, c, g, or t
agccaaccgatacttttctccaannnnnnnncttcagcttcccgatatccgacggtagtgtgtagaaaatggaagtctatgtg

SEQ ID NO: 891 - DNA, 83 bp, Artificial Sequence
  smMIP
  misc_feature (30)..(37): n is a, c, g, or t
gatcagcatacacaaattacaaaagtctgnnnnnnnncttcagcttcccgatatccgacggtagtgtcgtcaaatccagaggc

SEQ ID NO: 892 - DNA, 83 bp, Artificial Sequence
  smMIP
  misc_feature (29)..(36): n is a, c, g, or t
ggacctttttttttttaatggcaataggnnnnnnnncttcagcttcccgatatccgacggtagtgttgactctgatccagaga

SEQ ID NO: 893 - DNA, 83 bp, Artificial Sequence
  smMIP
  misc_feature (26)..(33): n is a, c, g, or t
ttgctcctcctgagcgcaagtactcnnnnnnnncttcagcttcccgatatccgacggtagtgtacatccgcaaagacctgtac

SEQ ID NO: 894 - DNA, 81 bp, Artificial Sequence
  smMIP
  misc_feature (25)..(32): n is a, c, g, or t
tctggcaccacaccttctacaatgnnnnnnnncttcagcttcccgatatccgacggtagtgtgtgatggtgggcatgggtc

SEQ ID NO: 895 - DNA, 83 bp, Artificial Sequence
  smMIP
  misc_feature (21)..(28): n is a, c, g, or t
aaccccaaggccaaccgcgannnnnnnncttcagcttcccgatatccgacggtagtgttgaagtaccccatcgagcacggcat

SEQ ID NO: 896 - DNA, 83 bp, Artificial Sequence
  smMIP
  misc_feature (27)..(34): n is a, c, g, or t
ggtcatcaccattggcaatgagcggtnnnnnnnncttcagcttcccgatatccgacggtagtgtgagcgggaaatcgtgcgtg

SEQ ID NO: 897 - DNA, 82 bp, Artificial Sequence
  smMIP
  misc_feature (25)..(32): n is a, c, g, or t
ggcatccacgaaactaccttcaacnnnnnnnncttcagcttcccgatatccgacggtagtgttgcttccagctcctccctgg

SEQ ID NO: 898 - DNA, 82 bp, Artificial Sequence
  smMIP
  misc_feature (24)..(31): n is a, c, g, or t
ccaccatgtaccctggcattgccnnnnnnnncttcagcttcccgatatccgacggtagtgtactcttccagccttccttcct

SEQ ID NO: 899 - DNA, 83 bp, Artificial Sequence
  smMIP
  misc_feature (30)..(37): n is a, c, g, or t
gttccataaactctggattgcattcctacnnnnnnnncttcagcttcccgatatccgacggtagtgtcagagattcttacccc

SEQ ID NO: 900 - DNA, 83 bp, Artificial Sequence
  smMIP
  misc_feature (27)..(34): n is a, c, g, or t
gtgagatgtctccagcatttttacggnnnnnnnncttcagcttcccgatatccgacggtagtgtttcttttcggggtgttcgc

SEQ ID NO: 901 - DNA, 83 bp, Artificial Sequence
  smMIP
  misc_feature (19)..(26): n is a, c, g, or t
actcccatccagtgtctcnnnnnnnncttcagcttcccgatatccgacggtagtgtctcttaacatctatatccaccttcatt

SEQ ID NO: 902 - DNA, 83 bp, Artificial Sequence
  smMIP
  misc_feature (30)..(37): n is a, c, g, or t
gctgaccatatgtggctgggactttggatnnnnnnnncttcagcttcccgatatccgacggtagtgttggtgccacgacaaat

SEQ ID NO: 903 - DNA, 83 bp, Artificial Sequence
  smMIP
  misc_feature (24)..(31): n is a, c, g, or t
gctggtggcactttacttactttnnnnnnnncttcagcttcccgatatccgacggtagtgtgtcctgccatgaataagcattt

SEQ ID NO: 904 - DNA, 81 bp, Artificial Sequence
  smMIP
  misc_feature (27)..(34): n is a, c, g, or t
gctttgccagtggtgggagcacaatannnnnnnncttcagcttcccgatatccgacggtagtgtagccaaccgagagacaa

SEQ ID NO: 905 - DNA, 83 bp, Artificial Sequence
  smMIP
  misc_feature (26)..(33): n is a, c, g, or t
gccttttgaaaagccagtgatgatcnnnnnnnncttcagcttcccgatatccgacggtagtgtttgtaccactccttccctgc

SEQ ID NO: 906 - DNA, 83 bp, Artificial Sequence
  smMIP
  misc_feature (30)..(37): n is a, c, g, or t
gcaaattaaagatctgggcagtgaattagnnnnnnnncttcagcttcccgatatccgacggtagtgtgtccttggaaaagtaa

SEQ ID NO: 907 - DNA, 83 bp, Artificial Sequence
  smMIP
  misc_feature (27)..(34): n is a, c, g, or t
cccaactacagaaatggtttcaaatgnnnnnnnncttcagcttcccgatatccgacggtagtgtcttgggtttttcctgtggc

SEQ ID NO: 908 - DNA, 83 bp, Artificial Sequence
  smMIP
  misc_feature (26)..(33): n is a, c, g, or t
gcagtatcctctgacagacatgtccnnnnnnnncttcagcttcccgatatccgacggtagtgtgcttgtaagtgcccgaagtg

SEQ ID NO: 909 - DNA, 83 bp, Artificial Sequence
  smMIP
  misc_feature (29)..(36): n is a, c, g, or t
caatttctgaccgagggaatcatcatgannnnnnnncttcagcttcccgatatccgacggtagtgtgaagtcataggaagagg

SEQ ID NO: 910 - DNA, 83 bp, Artificial Sequence
  smMIP
  misc_feature (28)..(35): n is a, c, g, or t
gcaaactcaaaagtttaccaccaagtcnnnnnnnncttcagcttcccgatatccgacggtagtgtaaaaattcacagtcaagg

SEQ ID NO: 911 - DNA, 83 bp, Artificial Sequence
  smMIP
  misc_feature (28)..(35): n is a, c, g, or t
actttcattggggagcactatgtccatnnnnnnnncttcagcttcccgatatccgacggtagtgtactcctacaacccgaata

SEQ ID NO: 912 - DNA, 83 bp, Artificial Sequence
  smMIP
  misc_feature (29)..(36): n is a, c, g, or t
gtattgttatttaaattactggattctannnnnnnncttcagcttcccgatatccgacggtagtgtcatagtgctagtactat

SEQ ID NO: 913 - DNA, 83 bp, Artificial Sequence
  smMIP
  misc_feature (24)..(31): n is a, c, g, or t
cggttcatcaacttctttgtaggnnnnnnnncttcagcttcccgatatccgacggtagtgtacaatcatactgctgacataca

SEQ ID NO: 914 - DNA, 83 bp, Artificial Sequence
  smMIP
  misc_feature (25)..(32): n is a, c, g, or t
tccgccggaaggccttcctccactnnnnnnnncttcagcttcccgatatccgacggtagtgtaacaatgtcaagacagccgtc

SEQ ID NO: 915 - DNA, 83 bp, Artificial Sequence
  smMIP
  misc_feature (20)..(27): n is a, c, g, or t
cagctgacccactcactggnnnnnnnncttcagcttcccgatatccgacggtagtgttttgtatttggtcagtctggggcagg

SEQ ID NO: 916 - DNA, 83 bp, Artificial Sequence
  smMIP
  misc_feature (21)..(28): n is a, c, g, or t
ccaccttgtctcagccaccannnnnnnncttcagcttcccgatatccgacggtagtgttacaatgccaccctctccgtccatc

SEQ ID NO: 917 - DNA, 83 bp, Artificial Sequence
  smMIP
  misc_feature (19)..(26): n is a, c, g, or t
tacctcaccgtggctgctnnnnnnnncttcagcttcccgatatccgacggtagtgtctttatgcctggctttgcccctctcac

SEQ ID NO: 918 - DNA, 83 bp, Artificial Sequence
  smMIP
  misc_feature (26)..(33): n is a, c, g, or t
ccaccactctttgaactggaccaagnnnnnnnncttcagcttcccgatatccgacggtagtgtcggggcttctcattgttgat

SEQ ID NO: 919 - DNA, 81 bp, Artificial Sequence
  smMIP
  misc_feature (22)..(29): n is a, c, g, or t
gccgaggaggtgtctgtgtgannnnnnnncttcagcttcccgatatccgacggtagtgtagacatcaagcataatcggccg

SEQ ID NO: 920 - DNA, 83 bp, Artificial Sequence
  smMIP
  misc_feature (27)..(34): n is a, c, g, or t
cccatctgacaatggctttgacagtgnnnnnnnncttcagcttcccgatatccgacggtagtgtcccaatctacaagtaccca

SEQ ID NO: 921 - DNA, 81 bp, Artificial Sequence
  smMIP
  misc_feature (27)..(34): n is a, c, g, or t
gccaagggaatgtactaccttgaggannnnnnnncttcagcttcccgatatccgacggtagtgtgtcatctctgcagcttg

SEQ ID NO: 922 - DNA, 83 bp, Artificial Sequence
  smMIP
  misc_feature (28)..(35): n is a, c, g, or t
gtggatggcccttgagagtatccacttnnnnnnnncttcagcttcccgatatccgacggtagtgtaaacgtgctactcaagtc

SEQ ID NO: 923 - DNA, 81 bp, Artificial Sequence
  smMIP
  misc_feature (28)..(35): n is a, c, g, or t
gcagtttctgggagcagtgaacggtgcnnnnnnnncttcagcttcccgatatccgacggtagtgtagcctaccagttggaa

SEQ ID NO: 924 - DNA, 79 bp, Artificial Sequence
  smMIP
  misc_feature (24)..(31): n is a, c, g, or t
tactccctcctcccgggaaggcannnnnnnncttcagcttcccgatatccgacggtagtgtcggagatagcgcctacca

SEQ ID NO: 925 - DNA, 80 bp, Artificial Sequence
  smMIP
  misc_feature (22)..(29): n is a, c, g, or t
ctcctgctccctgtggcactcnnnnnnnncttcagcttcccgatatccgacggtagtgtccccccatgtccattatgccc

SEQ ID NO: 926 - DNA, 82 bp, Artificial Sequence
  smMIP
  misc_feature (28)..(35): n is a, c, g, or t
gctttgtcacatggacacaattgactgnnnnnnnncttcagcttcccgatatccgacggtagtgtggaagtttgccatcttc

SEQ ID NO: 927 - DNA, 82 bp, Artificial Sequence
  smMIP
  misc_feature (29)..(36): n is a, c, g, or t
gcctgccggcacttcaatgacagtggagnnnnnnnncttcagcttcccgatatccgacggtagtgtacattgaccaagacca

SEQ ID NO: 928 - DNA, 83 bp, Artificial Sequence
  smMIP
  misc_feature (19)..(26): n is a, c, g, or t
ttttgccccaacccgcctnnnnnnnncttcagcttcccgatatccgacggtagtgtccccaactctgtcgtctgtgccttccc

SEQ ID NO: 929 - DNA, 82 bp, Artificial Sequence
  smMIP
  misc_feature (24)..(31): n is a, c, g, or t
aactggagcccttgggcacccagnnnnnnnncttcagcttcccgatatccgacggtagtgtttctggtctggtgcctgaggg

SEQ ID NO: 930 - DNA, 83 bp, Artificial Sequence
  smMIP
  misc_feature (25)..(32): n is a, c, g, or t
ggcacctgtctcactcttgaaggcnnnnnnnncttcagcttcccgatatccgacggtagtgtcctcaccgtgactaacatgcc

SEQ ID NO: 931 - DNA, 83 bp, Artificial Sequence
  smMIP
  misc_feature (27)..(34): n is a, c, g, or t
ggagctgctggctttacactgcctggnnnnnnnncttcagcttcccgatatccgacggtagtgtggcttagggcagtggaaag

SEQ ID NO: 932 - DNA, 79 bp, Artificial Sequence
  smMIP
  misc_feature (25)..(32): n is a, c, g, or t
gcactggtcttcagctactggtggnnnnnnnncttcagcttcccgatatccgacggtagtgtggtctgcgtagatggtg

SEQ ID NO: 933 - DNA, 83 bp, Artificial Sequence
  smMIP
  misc_feature (27)..(34): n is a, c, g, or t
gtggaggccttcctgcgagaggggctnnnnnnnncttcagcttcccgatatccgacggtagtgtacagtgaccgagtcattgg

SEQ ID NO: 934 - DNA, 82 bp, Artificial Sequence
  smMIP
  misc_feature (26)..(33): n is a, c, g, or t
cctcatcagctttggcctgcaggtannnnnnnncttcagcttcccgatatccgacggtagtgttgtgctggctctcattggt

SEQ ID NO: 935 - DNA, 79 bp, Artificial Sequence
  smMIP
  misc_feature (25)..(32): n is a, c, g, or t
cagtcaaggtggctgactttggttnnnnnnnncttcagcttcccgatatccgacggtagtgtaaccccaccgtgaagga

SEQ ID NO: 936 - DNA, 79 bp, Artificial Sequence
  smMIP
  misc_feature (25)..(32): n is a, c, g, or t
ctcacccatgccagggaatgtacgnnnnnnnncttcagcttcccgatatccgacggtagtgttgggggaggtggagcag

SEQ ID NO: 937 - DNA, 82 bp, Artificial Sequence
  smMIP
  misc_feature (24)..(31): n is a, c, g, or t
ccacacgggagccttcgtatactnnnnnnnncttcagcttcccgatatccgacggtagtgtttcagcccacgctcagtgtct

SEQ ID NO: 938 - DNA, 82 bp, Artificial Sequence
  smMIP
  misc_feature (22)..(29): n is a, c, g, or t
tgcccagaggctcctttctccnnnnnnnncttcagcttcccgatatccgacggtagtgttgtgagctggagtattcccctcc

SEQ ID NO: 939 - DNA, 83 bp, Artificial Sequence
  smMIP
  misc_feature (29)..(36): n is a, c, g, or t
gtggaaaccgcagcttgtctgcagtggannnnnnnncttcagcttcccgatatccgacggtagtgtacaacgaggctgcaaga

SEQ ID NO: 940 - DNA, 81 bp, Artificial Sequence
  smMIP
  misc_feature (21)..(28): n is a, c, g, or t
cgtgtccttggtgctagtggnnnnnnnncttcagcttcccgatatccgacggtagtgtttgctcagtaccactgatgtccc

SEQ ID NO: 941 - DNA, 80 bp, Artificial Sequence
  smMIP
  misc_feature (24)..(31): n is a, c, g, or t
gcctgtggcagtggatggtgttgnnnnnnnncttcagcttcccgatatccgacggtagtgttgagctccgaatgtcctgg

SEQ ID NO: 942 - DNA, 82 bp, Artificial Sequence
  smMIP
  misc_feature (28)..(35): n is a, c, g, or t
gcgggaaaggcgggaagaacaccatgannnnnnnncttcagcttcccgatatccgacggtagtgtaacaacgcctaccagaa

SEQ ID NO: 943 - DNA, 83 bp, Artificial Sequence
  smMIP
  misc_feature (29)..(36): n is a, c, g, or t
gctgtacatcctggttgggcagcagggannnnnnnncttcagcttcccgatatccgacggtagtgtagccaccgacacctaca

SEQ ID NO: 944 - DNA, 82 bp, Artificial Sequence
  smMIP
  misc_feature (24)..(31): n is a, c, g, or t
ctgccccggttcatcctgctggannnnnnnncttcagcttcccgatatccgacggtagtgttggctgtgaagacgctgcctg

SEQ ID NO: 945 - DNA, 80 bp, Artificial Sequence
  smMIP
  misc_feature (27)..(34): n is a, c, g, or t
cagacacatggtcctttggagtgctgnnnnnnnncttcagcttcccgatatccgacggtagtgttggagacttcgggatg

SEQ ID NO: 946 - DNA, 83 bp, Artificial Sequence
  smMIP
  misc_feature (22)..(29): n is a, c, g, or t
cccaacgtacggctcctggttnnnnnnnncttcagcttcccgatatccgacggtagtgttctctgttcgagtccctagagggc

SEQ ID NO: 947 - DNA, 83 bp, Artificial Sequence
  smMIP
  misc_feature (24)..(31): n is a, c, g, or t
gcccctggagctggtcattacgannnnnnnncttcagcttcccgatatccgacggtagtgttgctcctagagccctcttcgct

SEQ ID NO: 948 - DNA, 83 bp, Artificial Sequence
  smMIP
  misc_feature (30)..(37): n is a, c, g, or t
gcctgtggggccgacagctatgagatggannnnnnnncttcagcttcccgatatccgacggtagtgttctacaaccccaccac

SEQ ID NO: 949 - DNA, 83 bp, Artificial Sequence
  smMIP
  misc_feature (30)..(37): n is a, c, g, or t
cgtaaaggaaatcacagggtttttgctgannnnnnnncttcagcttcccgatatccgacggtagtgtaaacacttcaaaaact

SEQ ID NO: 950 - DNA, 20 bp, Artificial Sequence
  forward primer HMBSFw
ctggtaacggcaatgcggct

SEQ ID NO: 951 - DNA, 20 bp, Artificial Sequence
  reverse primer HMBSRv
ttcttctccagggcatgttc